SEQUENCE LISTING (623 sequences)

SEQ ID NO: 1 - DNA, 57 bp, artificial
  synthetic
ccatctcatccctgcgtgtctcgactcagbbbbbbbbagagtttgatcmtggctcag

SEQ ID NO: 2 - DNA, 53 bp, artificial
  synthetic
cctatcccctgtgtgccttggcagtctcagbbbbbbbbctgctgcctyccgta

SEQ ID NO: 3 - DNA, 17 bp, artificial
  synthetic
cctacgggaggcagcag

SEQ ID NO: 4 - DNA, 17 bp, artificial
  synthetic
attaccgcggctgctgg

SEQ ID NO: 5 - DNA, 19 bp, artificial
  synthetic
agtggcgcacgggtgagta

SEQ ID NO: 6 - DNA, 19 bp, artificial
  synthetic
gtgtccgttcaccctctca

SEQ ID NO: 7 - DNA, 18 bp, artificial
  synthetic
tgctgacgagtggcgaac

SEQ ID NO: 8 - DNA, 18 bp, artificial
  synthetic
gtggctggtcgtcctctc

SEQ ID NO: 9 - DNA, 18 bp, artificial
  synthetic
tgcggaacacgtgtgcaa

SEQ ID NO: 10 - DNA, 20 bp, artificial
  synthetic
ccgttacctcaccaactagc

SEQ ID NO: 11 - DNA, 216 bp, artificial
  synthetic
gatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcg

SEQ ID NO: 12 - DNA, 368 bp, artificial
  synthetic
gacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacgggtcggcatcgaccagagggaaaaggagcaatccgctttgagatggcctcgcgtccgattagctagttggtgaggtaacggcccaccaaggcgacgatcggtagccggactgagaggttgaacggccacattgggactgagacacggcccagactcctacggaaggcagcagggttggttttttttctccaaggcacacaggggatagg

SEQ ID NO: 13 - DNA, 277 bp, artificial
  synthetic
gatgaacgctggcggcgtgcttaacacatgcaagtcgaacgaagcaccttgatttgatttcttcggaatgaagatcttggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagta

SEQ ID NO: 14 - DNA, 277 bp, artificial
  synthetic
gatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagc

SEQ ID NO: 15 - DNA, 266 bp, artificial
  synthetic
aatgaacgctggcggcatgcctaacacatgcaagtcgaacgaaggcttcggccttagtggcgcacgggtgcgtaacgcgtgggaatctgccctcaggttcggaataacagcgagaaattgctgctaataccggatgatatcgcgagatcaaagatttatcgcctgaggatgagcccgcgtaggattagctagttggtgtggtaaaggcgcaccaaggcgacgatccttagctggtctgagaggatgatcagccacactgggactga

SEQ ID NO: 16 - DNA, 289 bp, artificial
  synthetic
atgaacgctggcggcgtgcttaacacatgcaagtcgaacgaagcactttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagag

SEQ ID NO: 17 - DNA, 276 bp, artificial
  synthetic
gatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatggcatcatgagttcacatgtccgcatgattaaaggtattttccggtagacgatggggatgcgttccattagatagtaggcggggtaacggcccacctagtcaacgatggataggggttct

SEQ ID NO: 18 - DNA, 275 bp, artificial
  synthetic
gatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatgactgctaataccgcataagaccacagtaccgcatggtacagtggtaaaaactccggtggtatgagatggacccgcgtctgattaggtagttggtggggtaacggcctaccaagccgacgatcagta

SEQ ID NO: 19 - DNA, 304 bp, artificial
  synthetic
gatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgaga

SEQ ID NO: 20 - DNA, 256 bp, artificial
  synthetic
gatgaacgctggcggcgtgcctaatacatgcaagtcgaacgcttcacttcggtgaagagtggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagcttgttggtagggtaacggcctaccaaggcgacgatgcgtagcc

SEQ ID NO: 21 - DNA, 295 bp, artificial
  synthetic
gatgaacgctagctacaggcttaacacatgcaagtcgaggggcagcatggtcttagcttgctaaggccgatggcgaccggcgcacgggtgagtaacgcgtatccaacctgccttacactcttggacagccttctgaaagggagattaatacaagatgttatcatgagtaagcattttcgcatgattaaaggtttaccggtgtaagatggggatgcgttccattagatagtaggcggggtaacggcccacctagtcttcgatggataggggttctgagaggaaggtcccccacatt

SEQ ID NO: 22 - DNA, 314 bp, artificial
  synthetic
gatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagac

SEQ ID NO: 23 - DNA, 300 bp, artificial
  synthetic
gatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgagaagcaaaaggaggaatccgcccgaggaggggctcgcgtctgattagctagttggtgaggtaatagcttaccaaggcgatgatcagtagctggtccgagaggatgatcagccacactgggactgagacacggcccagactcctacggaaggcagcag

SEQ ID NO: 24 - DNA, 259 bp, artificial
  synthetic
gatgaacgctggcggcatgcctaatacatgcaagtcgaacgagaggaaggaaagcttgcttttctgaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgac

SEQ ID NO: 25 - DNA, 332 bp, artificial
  synthetic
gatgaacgctggcggcgtgcttaacacatgcaagtcgaacgaagcacttttatcgatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctac

SEQ ID NO: 26 - DNA, 315 bp, artificial
  synthetic
gatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgag

SEQ ID NO: 27 - DNA, 330 bp, artificial
  synthetic
attgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacggaaggcagcag

SEQ ID NO: 28 - DNA, 331 bp, artificial
  synthetic
gacgaacgctggcggcgcgcctaacacatgcaagtcgaacgagagagaaggagcttgcttcttcaatcgagtggcgaacgggtgagtaacgcgtgaggaacctgcctcaaagagggggacaacagttggaaacgactgctaataccgcataagcccacggctcggcatcgagcagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcag

SEQ ID NO: 29 - DNA, 343 bp, artificial
  synthetic
gatgaacgctggcggcgtgcttaacacatgcaagtcgagcgaagcagttaagttgatttcttcggattgattcttaactgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacggaaggcagca

SEQ ID NO: 30 - DNA, 321 bp, artificial
  synthetic
gatgaacgctggcggcgtgcttaacacatgcaagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggccc

SEQ ID NO: 31 - DNA, 344 bp, artificial
  synthetic
gacgaacgctggcggtatgcttaacacatgcaagtcgaacgagaaagttcagttttaaagacttcggtcttaaaaactgaactggaaagtggcgaacgggtgagtaacgcgtgggcaacctgccctttagacggagatagcctttggaaacgaagagtaatacccgatgccttcagaaaaggacatccttttctgaagaaagctaaagcgctaaaggatgggcccgcgtatcattaggtagttggtgagataacagcccaccaagccaacgatgattagccggtctgagagggcgaacggccacattggaactgagagacggtccaaactcctacggaaggcag

SEQ ID NO: 32 - DNA, 322 bp, artificial
  synthetic
attgaacgctggcggcatgccttacacatgcaagtcgaacggtaacaggtcttcggatgctgacgagtggcgaacgggtgagtaatacatcggaacgtgcccagacgtgggggataacgaggcgaaagctttgctaataccgcatacgatctaaggatgaaagcaggggaccgcaaggccttgcgcgtttggagcggccgatggcagattaggtagttggtgggataaaagcttaccaagccgacgatctgtagctggtctgagaggacgaccagccacactgggactgagacacggcccagactcctacggaaggcagcag

SEQ ID NO: 33 - DNA, 313 bp, artificial
  synthetic
gatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgag

SEQ ID NO: 34 - DNA, 313 bp, artificial
  synthetic
gatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgag

SEQ ID NO: 35 - DNA, 329 bp, artificial
  synthetic
gatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacggaaggcagca

SEQ ID NO: 36 - DNA, 314 bp, artificial
  synthetic
gatgaacgctggcggcgtgcttaacacatgcaagtcgagcgaagcacttaggaaagattcttcggatgatttcctatttgactgagcggcggacgggtgagtaacgcgtgggtaacctgcctcatacagggggataacagttagaaatgactgctaataccgcataagaccacagcaccgcatggtgcagtggtaaaaactccggtggtatgagatggacccgcgtctgattagttagttggtggggtaacggcctaccaaggcgacgatcagtagccgacctgagagggtgaccggccacattgggactgaga

SEQ ID NO: 37 - DNA, 312 bp, artificial
  synthetic
gatgaacgctggcggcgtgcctaacacatgcaagtcgaacgaagcaccttaccggattcttcggatgaaagtttggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgaga

SEQ ID NO: 38 - DNA, 310 bp, artificial
  synthetic
gatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggt

SEQ ID NO: 39 - DNA, 345 bp, artificial
  synthetic
gatgaacgctggcggcgtgcttaacacatgcaagtcgaacgaagctgcttaactgatcttcttcggaattgacgttttgtagactgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacggaaggcagca

SEQ ID NO: 40 - DNA, 313 bp, artificial
  synthetic
gatgaacgctggcggcgtgcttaacacatgcaagtcgaacgggaaatatttcattgagacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctagttggtggggtaacggcccaccaaggcgacgatccatagccggcctgagagggtgaacggccacattgggactgaga

SEQ ID NO: 41 - DNA, 297 bp, artificial
  synthetic
gatgaacgctggcggcgtgcttaacacatgcaagtcgaacggacgatgaagagcttgcttttcagagttagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactga

SEQ ID NO: 42 - DNA, 296 bp, artificial
  synthetic
gatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagaca

SEQ ID NO: 43 - DNA, 343 bp, artificial
  synthetic
gatgaacgctggcggcgtgcttaacacatgcaagtcgaacgaagcactttggacagattcttcggatgaagtccttagtgactgagtggcggacgggtgagtaacgcgtgggtaacctgcctcatacagggggataacagttagaaatggctgctaataccgcataagcgcacggtactgcatggtacagtgtgaaaaactccggtggtatgagatggacccgcgttggattagctagttggcagggtaacggcctaccaaggcgacgatccatagccggcctgagagggtggacggccacattgggactgagacacggcccagactcctacggaaggcagca

SEQ ID NO: 44 - DNA, 313 bp, artificial
  synthetic
gatgaacgctggcggcgtgcttaacacatgcaagtcgaacgaagcacttatctttgattcttcggatgaagaggtttgtgactgagtggcggacgggtgagtaacgcgtgggtaacctgcctcatacagggggataacagttagaaatgactgctaataccgcataagaccacggagccgcatggctcagtgggaaaaactccggtggtatgagatggacccgcgtctgattaggtagttggtggggtaacggcctaccaagccaacgatcagtagccgacctgagagggtgaccggccacattgggactgag

SEQ ID NO: 45 - DNA, 326 bp, artificial
  synthetic
gatgaacgctggcggcgtgcttaacacatgcaagtcgaacgggaaatactttattgaaacttcggtggatttaatttatttctagtggcggacgggtgagtaacgcgtgggtaacctgccttatactgggggataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagact

SEQ ID NO: 46 - DNA, 335 bp, artificial
  synthetic
gatgaacgctagctacaggcttaacacatgcaagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacggaaggcagcagg

SEQ ID NO: 47 - DNA, 325 bp, artificial
  synthetic
gatgaacgctagcgggaggcctaacacatgcaagccgagcggtattgtttcttcggaaatgagagagcggcgtacgggtgcggaacacgtgtgcaacctgcctttatctgggggatagcctttcgaaaggaagattaatactccataatatattgaacggcatcgtttaatattgaaagctccggcggatagagatgggcacgcgcaagattagctagttggtgaggtaacggctcaccaaggcgatgatctttagggggcctgagagggtgatcccccacactggtactgagacacggaccagactcctacggaaggcagcagg

SEQ ID NO: 48 - DNA, 323 bp, artificial
  synthetic
attgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacggaaggcagca

SEQ ID NO: 49 - DNA, 322 bp, artificial
  synthetic
gatgaacgctggcggcgcgcctaacacatgcaagtcgaacggcacccctctccggatggaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacggaaggcagca

SEQ ID NO: 50 - DNA, 323 bp, artificial
  synthetic
gatgaacgctggcggcgtgcttaacacatgcaagtcgaacgggaaaccttttattgaagcttcggcagatttagattgtttctagtggcggacgggtgagtaacgcgtgggtaacctgccttatacagggggataacagtcagaaatgactgctaataccgcataagcgcacagagctgcatggctcggtgtgaaaaactccggtggtataagatggacccgcgttggattagctagttggtgaggtaacggcccaccaaggcgacgatccatagccggcctgagagggtgaacggccacattgggactgagacacggcccag

SEQ ID NO: 51 - DNA, 329 bp, artificial
  synthetic
gacgaacgctggcggcgcgcctaacacatgcaagtcgaacggagcttagagagcttgctttttaagcttagtggcgaacgggtgagtaacgcgtgagtaacctgccctagagtgggggacaacagttggaaacgactgctaataccgcataagcccacggtaccgcatggtactgagggaaaaggatttattcgctttaggatggactcgcgtccaattagctagttggtgaggtaacggcccaccaaggcgacgattggtagccggactgagaggttgaacggccacattgggactgagacacggcccagactcctacggaaggcagc

SEQ ID NO: 52 - DNA, 336 bp, artificial
  synthetic
gatgaacgctagctacaggcttaacacatgcaagtcgaggggaaacgacatcgaaagcttgcttttgatgggcgtcgaccggcgcacgggtgagtaacgcgtatccaacctgcccaccacttggggataaccttgcgaaagtaagactaatacccaatgacgtctctagaagacatctgaaagagattaaagatttatcggtgatggatggggatgcgtctgattagcttgttggcggggtaacggcccaccaaggcaacgatcagtaggggttctgagaggaaggtcccccacattggaactgagacacggtccaaactcctacggaaggcagca

SEQ ID NO: 53 - DNA, 342 bp, artificial
  synthetic
gatgaacgctggcggcgtgcttaacacatgcaagtcgagcgaagcgatttaagtgaagttttcggatggatcttaaattgactgagcggcggacgggtgagtaacgcgtggataacctgcctcacacagggggataacagttagaaatgactgctaataccgcataagcgcacggtaccgcatggtacagtgtgaaaaactccggtggtgtgagatggatccgcgtctgattaggtagttggtgaggtaacggcccaccaagccgacgatcagtagccgacctgagagggtgaccggccacattgggactgagacacggcccaaactcctacggaaggcagc

SEQ ID NO: 54 - DNA, 343 bp, artificial
  synthetic
gatgaacgctggcggcgtgcctaacacatgcaagtcgaacggagctgctttgatgaagttttcggatggatttaaaacagcttagtggcggacgggtgagtaacgcgtgggtaacctgcctcacactgggggataacagttagaaatagctgctaataccgcataagcgcacggttccgcatggaacagtgtgaaaaactccggtggtgtgagatggacccgcgtctgattagccagttggcggggtaacggcccaccaaagcgacgatcagtagccggcctgagagggtgaacggccacattgggactgagacacggcccaaactcctacggaaggcagcag

SEQ ID NO: 55 - DNA, 292 bp, artificial
  synthetic
cctccagcatagctggtattacaaatcctgccaccatgcccagctaatttttctaatttttagtagagacacggtttcactatgttggccaggctggtctcgcactcctgacctcaggtgatccgcccgcctcgacttcccaaagtactgggattacgggcgtgagcgctaccacgcctggccggatctagcattaagggcaacgaaacgtgaagctggttttagttacccgatatacttgaaagttaaatgtcgggtcttctttaagagcacggatacggaaggcagcagg

SEQ ID NO: 56 - DNA, 337 bp, artificial
  synthetic
gatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagca

SEQ ID NO: 57 - DNA, 334 bp, artificial
  synthetic
gacgaacgctggcggcgtgcctaacacatgcaagtcgaacggagaattttatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacggg

SEQ ID NO: 58 - DNA, 335 bp, artificial
  synthetic
gatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataattagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacggaaggcagca

SEQ ID NO: 59 - DNA, 327 bp, artificial
  synthetic
attgaacgctggcggcatgctttacacatgcaagtcgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatgcccgattagctagttggtgaggtaaaggctcaccaaggcgacgatcggtagctggtctgagaggacgaccagccacactgggactgagacacggcccagactcctacggaaggcagca

SEQ ID NO: 60 - DNA, 328 bp, artificial
  synthetic
attgaacgctggcggcatgctttacacatgcaagtcgaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacggaaggcagcag

SEQ ID NO: 61 - DNA, 312 bp, artificial
  synthetic
attgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactc

SEQ ID NO: 62 - DNA, 345 bp, artificial
  synthetic
gatgaacgctggcggcgtgcttaacacatgcaagtcgaacgaagaaggttagaatgagagcttcggcaggatttctttccatcttagtggcggacgggtgagtaacgtgtgggcaacctgccctgtactggggaataatcattggaaacgatgactaataccgcatgtggtcctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacggaaggcagc

SEQ ID NO: 63 - DNA, 311 bp, artificial
  synthetic
attgaacgctggcggcaggcctaatacatgcaagtcgaacggtaacataaaagaagcttgcttcttttgatgacgagtggcggacgggtgagtaatatttgggaaactacctgatagagggggacaacagttggaaacgactgctaataccgcataaagcctgagggtgaaagcagcaatgcgctatcagatgtgcccaaacgggattagctagtaggtgaggtaaaggctcacctaggcgacgatctctagctggtctgagaggatgatcagccacattgggactgagacacggcccagactcctacggg

SEQ ID NO: 64 - DNA, 345 bp, artificial
  synthetic
gatgaacgctggcggcgtgcttaacacatgcaagtcgaacgaagcactttacctgatttccttcggggatgattgttctgtgactgagtggcggacgggtgagtaacgcgtggataacctgcctcacacagggggataacagttggaaacgactgctaataccgcataagcgcacagcaccgcatggtgcagtgtgaaaaactccggtggtgtgagatggatccgcgtctgattagctagttggcggggtaacggcccaccaaggcgacgatcagtagccggcctgagagggcgaccggccacattgggactgagacacggcccagactcctacggaaggcagca

SEQ ID NO: 65 - DNA, 337 bp, artificial
  synthetic
gatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacggaaggcagcagg

SEQ ID NO: 66 - DNA, 323 bp, artificial
  synthetic
attgaacgctggcggcatgccttacacatgcaagtcgaacggtaacaggtcttcggacgctgacgagtggcgaacgggtgagtaatacatcggaacgtgcccagtcgtgggggataactactcgaaagagtagctaataccgcatacgatctgaggatgaaagcgggggaccttcgggcctcgcgcgattggagcggccgatggcagattaggtagttggtgggataaaagcttaccaagccgacgatctgtagctggtctgagaggacgaccagccacactgggactgagacacggcccagactcctacggaaggcagcagg

SEQ ID NO: 67 - DNA, 342 bp, artificial
  synthetic
gatgaacgctggcggcgtgcctaacacatgcaagtcgaacgaagcaccttttaagattcttcggatgattgattggtgactgagtggcggacgggtgagtaacgcgtgggtaacctgccctgtacagggggataacagttggaaacggctgctaataccgcataagcgcacgagaggacatcctcttgtgtgaaaaactccggtggtacaggatgggcccgcgtctgattagctggttggcagggtaacggcctaccaaggcgacgatcagtagccggtctgagaggatgaacggccacattggaactgagacacggtccaaactcctacggaaggcagcag

SEQ ID NO: 68 - DNA, 317 bp, artificial
  synthetic
gatgaacgctggcggcgtgcctaacacatgcaagtcgagcgattctcttcggagaagagcggcggacgggtgagtaacgcgtgggtaacctgccctgtacacacggataacataccgaaaggtatgctaatacgggataacataagaaattcgcatgtttttcttatcaaagctccggcggtacaggatggacccgcgtctgattagctagttggtgaggtaacggctcaccaaggcgacgatcagtagccgacctgagagggtgatcggccacattggaactgagacacggtccaaactcctacggaaggcagcag

SEQ ID NO: 69 - DNA, 321 bp, artificial
  synthetic
attgaacgctggcggcaggcttaacacatgcaagtcgagcggagatgaggtgcttgcaccttatcttagcggcggacgggtgagtaatgcttaggaatctgcctattagtgggggacaacattccgaaaggaatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacggaaggcag

SEQ ID NO: 70 - DNA, 323 bp, artificial
  synthetic
gacgaacgctggcggcgcgcctaacacatgcaagtcgaacggagtcaagaggagcttgcttttcttgacttagtggcgaacgggtgagtaacgcgtgagtaacctgccctggagtgggggacaacagttggaaacgactgctaataccgcataagcccacggatccgcatggatctgcgggaaaaggatttattcgcttcaggatggactcgcgtccaattagctagttggtgaggtaacggcccaccaaggcgacgattggtagccggactgagaggttgaacggccacattgggactgagacacggcccagactcctacgg

SEQ ID NO: 71 - DNA, 333 bp, artificial
  synthetic
gataaacgctggcggcgcacataagacatgcaagtcgaacggacttaatcgattcttttagattcgaagcggttagtggcggactggtgagtaacacgtaagcaacctgcctgttagaggggaataacagtgagaaatcactgctaaaaccgcatatgccgtgaacatcgcatgatgaaaacgggaaaagagcaatctgctaatagatgggcttgcgtctgattagctagttggtgtggtaaaggcataccaaggcaacgatcagtagccggactgagaggttgaacggccacattgggactgagatacggcccagactcctacggaaggcag

SEQ ID NO: 72 - DNA, 313 bp, artificial
  synthetic
gacgaacgctggcggcacgcttaacacatgcaagtcgaacgagagaagagaagcttgcttttctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactc

SEQ ID NO: 73 - DNA, 337 bp, artificial
  synthetic
ggtgaacgctagctacaggcttaacacatgcaagtcgaggggcagcgatgaagaaagcttgctttcttcaggcggcgaccggcgcacgggtgagtaacgcgtatcgaacctgcctcatactcgggaatagccttgcgaaagtaagattaatgcccgatgttattaggatatcacatgatgttttaattaaagatttatcggtatgagatggcgatgcgtcccattagtttgttggcggggtaacggcccaccaagacatcgatgggtaggggttctgagaggaaggtcccccacattggaactgagacacggtccaaactcctacgggaggcagcag

SEQ ID NO: 74 - DNA, 310 bp, artificial
  synthetic
attgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagac

SEQ ID NO: 75 - DNA, 339 bp, artificial
  synthetic
gatgaacgctggcggcgtgcttaacacatgcaagtcgaacgaagcacttaagattgattcttcggatgacgtcttttgtgacttagtggcggacgggtgagtaacgcgtgggtaacctgcctcacacagggggataacagttagaaatgactgctaataccgcataacccgataggatcgcatgatccagacggaaaagatttatcggtgtgagatggacccgcgtctgattagctagttggtgaggtaacggcccaccaaggcgacgatcagtagccggcctgagagggtgaacggccacattgggactgagacacggcccaaactcctacggaaggc

SEQ ID NO: 76 - DNA, 337 bp, artificial
  synthetic
gacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacggaaggcagcagg

SEQ ID NO: 77 - DNA, 347 bp, artificial
  synthetic
gacgaacgctggcggcgtgcctaatacatgcaagtcgagcgagcagaactaacagatctacttcggtagtgacgtttcggaagcgagcggcggatgggtgagtaacacgtgggtaacctgcccttaagtctgggataccatttggaaacaggtgctaataccggataacaacattgatcgcatgatcgatgcttgaaaggcggcgtaagctgtcgctaaaggatggacccgcggtgcattagctagttggtaaggtaacggcttaccaaggcgacgatgcatagccgagttgagagactgatcggccacattgggactgagacacggcccaaactcctacggaaggc

SEQ ID NO: 78 - DNA, 343 bp, artificial
  synthetic
gatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatgactgctaataccgcataagaccacaggatcgcatgatccagtggtaaaaactccggtggtatgagatggacccgcgtctgattagctagttggtggggtaacggctcaccaaggcgacgatcagtagccgacctgagagggtgaccggccacattgggactgagacacggcccaaactcctacggaaggcagca

SEQ ID NO: 79 - DNA, 333 bp, artificial
  synthetic
gatgaacgctggcggcgtgcctaacacatgcaagtcgaacggacttacattgaagcctagcgatatgtaagttagtggcggacgggtgagtaacgcgtgggtaacctgccttgtactgggggacaacagttggaaacgactgctaataccgcataagcgcacagcttcgcatgaagcagtgtgaaaaactccggtggtacaagatggacccgcgtctgattagctggttggtgaggtaacggcccaccaaggcgacgatcagtagccggcctgagagggtgaacggccacattgggactgagacacggcccaaactcctacgggaggcagcag

SEQ ID NO: 80 - DNA, 298 bp, artificial
  synthetic
aacgaacgctggcggcatgcctaatacatgcaagtcgaacgagatcttcggatctagtggcgcacgggtgcgtaacgcgtgggaatctgcccttgggttcggaataacttctggaaacggaagctaataccggatgatgacgtaagtccaaagatttatcgcccaaggatgagcccgcgtaggattagctagttggtggggtaaaggcccaccaaggcgacgatccttagctggtctgagaggatgatcagccacactgggactgagacacggcccagactcctacggaaggcagcag

SEQ ID NO: 81 - DNA, 346 bp, artificial
  synthetic
gatgaacgctggcggcgtgcttaacacatgcaagtcgagcgaagcactttacttggatttcttcggaatgacgagtattgtgactgagcggcggacgggtgagtaacgcgtgggtaacctgcctcatacagggggataacagttagaaatgactgctaataccgcataagaccacagcaccgcatggtgcagaggtaaaaactccggtggtatgagatggacccgcgtctgattagctggttggtggggtaacggcctaccaaggcgacgatcagtagccggcctgagagggcgaccggccacattgggactgagacacggcccaaactcctacggaaggcagcag

SEQ ID NO: 82 - DNA, 325 bp, artificial
  synthetic
attgaacgctggcggcatgccttacacatgcaagtcgaacggtaacgggtccttcgggatgccgacgagtggcgaacgggtgagtaatatatcggaacgtgcccagtagtgggggataactgctcgaaagagcagctaataccgcatacgacctgagggtgaaagggggggatcgcaagacctctcgctattggagcggccgatatcagattagctagttggtggggtaaaggcctaccaaggcaacgatctgtagttggtctgagaggacgaccagccacactgggactgagacacggcccagactcctacggaaggcagcagg

SEQ ID NO: 83 - DNA, 336 bp, artificial
  synthetic
gacgaacgctggcggcgtgcctaatacatgcaagttgagcgatgaagattggtgcttgcaccaatttgaagagcagcgaacgggtgagtaacgcgtggggaatctgcctttgagcgggggacaacatttggaaacgaatgctaataccgcataacaactttaaacataagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcag

SEQ ID NO: 84 - DNA, 345 bp, artificial
  synthetic
gacgaacgctggcggcgtgcctaacacatgcaagtcgaacggaacttctttaaaggatttcttcggaatgaatttgattaagtttagtggcggacgggtgagtaacgcgtgagtaacctgcctctaagaggggaataacattctgaaaagaatgctaataccgcataatatatatttatcgcatggtagatatatcaaagatttatcgcttagagatggactcgcgtccgattagttagttggtgaggtaacggctcaccaagaccgcgatcggtagccggactgagaggttgaacggccacattgggactgagacacggcccagactcctacggaaggcagcag

SEQ ID NO: 85 - DNA, 329 bp, artificial
  synthetic
agtgaacgctggcggtaggcctaacacatgcaagtcgaacggcagcacagaggagcttgctccttgggtggcgagtggcggacgggtgaggaatacatcggaatctactctgtcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacggaaggcagca

SEQ ID NO: 86 - DNA, 327 bp, artificial
  synthetic
gataaacgctggcggcgtgcttaacacatgcaagtcgaacgaagtttttctttcgggaggaacttagtggcggacgggtgagtaacgcgtgggtaacctgccctgtacagggggacaacagctggaaacggctgctaataccgcataagcccttagcactgcatggtgcatagggaaaaggagcaatccggtacaggatggacccgcgtctgattagccagttggcagggtaacggcctaccaaagcgacgatcagtagccgatctgagaggatgtacggccacattgggactgagacacggcccagactcctacggaaggcagcag

SEQ ID NO: 87 - DNA, 329 bp, artificial
  synthetic
gatgaacgctggcggcgcgcctaacacatgcaagtcgaacggacgatgaagagcttgcttttcaaagttagtggcggacgggtgagtaacgcgtgggtaacctgcctcatacaggggaatagcagctggaaacggctgataaaaccgcataagcgcacagtgccgcatggcacagtgtgaaaaactccggtggtatgagatggacccgcgtctgattagctggttggcggggtaacggcccaccaaggcgacgatcagtagccggcctgagagggtgatcggccacattgggactgagacacggcccaaactcctacggaaggcagcag

SEQ ID NO: 88 - DNA, 335 bp, artificial
  synthetic
gacgaacgctggcggcgtgcctaatacatgcaagtagaacgctgactactttagcttgctagagtagaaggagttgcgaacgggtgagtaacgcgtaggtaacctgcctactagcgggggataactattggaaacgatagctaataccgcataacagtgtttaacacatgttagatgcttgaaagatgcaattgcatcactagtagatggacctgcgttgtattagctagttggtggggtaacggcccaccaaggcgacgatacatagccgacctgagagggtgatcggccacactgggactgagacacggcccagactcctacggaaggcagca

SEQ ID NO: 89 - DNA, 346 bp, artificial
  synthetic
gatgaacgctggcggcgtgcttaacacatgcaagtcgagcgaagcactctgctttgattctttcgggatgaagagcattgtgactgagcggcggacgggtgagtaacgcgtgggtaacctgcctcatacagggggataacagttagaaatggctgctaataccgcataagaccacagcaccgcatggtgcaggggtaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagcag

SEQ ID NO: 90 - DNA, 347 bp, artificial
  synthetic
gatgaacgctggcggcgtgcttaacacatgcaagtcgaacgaagatacttaaaatgagagcttcggcaggatttttatttatcttagtggcggacgggtgagtaacgtgtgggcaacctgccctatactggggaataatcactggaaacggtgactaataccgcatgtcattacggaagggcatccttctgtaagaaaaggagtaattcggtataggatgggcccgcatctgattagctagttggtgagataacagcccaccaaggcgacgatcagtagccgacctgagagggtgatcggccacattgggactgagacacggcccaaactcctacggaaggcagcag

SEQ ID NO: 91 - DNA, 299 bp, artificial
  synthetic
gatgaacgctggcggcatgcttaacacatgcaagtcgaacgggaagtggtgtttccagtggcgaacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagca

SEQ ID NO: 92 - DNA, 348 bp, artificial
  synthetic
gatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacggaaggcagcagggtt

SEQ ID NO: 93 - DNA, 315 bp, artificial
  synthetic
gatgaacgctggcggcgtgcttaacacatgcaagtcgagcgaagcgctttggaaagattcttcggatgatttcctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagac

SEQ ID NO: 94 - DNA, 328 bp, artificial
  synthetic
attgaacgctggcggcaggcttaacacatgcaagtcgagcggtaacaggggagcttgctcctgctgacgagcggcggacgggtgagtaacgcgtaggaatctacctagtagagggggacaacatgtggaaacgcatgctaataccgcatacgccctaagggggaaaggaggggacttttcggagccttccgctattagatgagcctgcgtaagattagctagttggtagggtaaaggcctaccaaggcgacgatctttaactggtctgagaggatgaccagtcacactgggactgagacacggcccagactcctacggaaggcagcag

SEQ ID NO: 95 - DNA, 330 bp, artificial
  synthetic
gatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatcttcttgttattaaagaatttcgataaccgatggggatgcgttccattagtttgttggcggggtaacggcccaccaagacatcgatggataggggttctgagaggaaggtcccccacattggaactgagacacggtccaaactcctacggaaggcagcag

SEQ ID NO: 96 - DNA, 317 bp, artificial
  synthetic
gatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctggttatgaaagctatatgcgctgtgagagagctttgcgtcccattagttagttggtgaggtaacggctcaccaagacgatgatgggtagccggcctgagagggtgaacggccacaaggggactgagacacggcccttactcctacggaaggcagcag

SEQ ID NO: 97 - DNA, 327 bp, artificial
  synthetic
gacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacggaaggcagca

SEQ ID NO: 98 - DNA, 316 bp, artificial
  synthetic
gacgaacgctggcggcgtgcctaatacatgcaagtggaacgcatgattgataccggagcttgctccaccattaatcatgagtcgcgaacgggtgagtaacgcgtaggtaacctacctcatagcgggggataactattggaaacgatagctaataccgcataacagtatttatcgcatggtaaatgcttgaaaggagcaactgcttcactatgagatggacctgcgttgtattagctagttggtggggtaacggctcaccaaggcatcgatacatagccgacctgagagggtgatcggccacactgggactgagaca

SEQ ID NO: 99 - DNA, 335 bp, artificial
  synthetic
gatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatatcatcattaaagatttatcggtgtacgatggggatgcgttccattaggtagtaggcggggtaacggcccacctagccaacgatggataggggttctgagaggaaggtcccccacattggaactgagacacggtccaaactcctacggaaggcagcag

SEQ ID NO: 100 - DNA, 341 bp, artificial
  synthetic
gatgaacgctggcggcgtgcttaacacatgcaagtcgaacgaagcgcttaaatggatttcttcggattgaagtttttgtgactgagtggcggacgggtgagtaacgcgtgggtaacctgcctcatacagggggataacagttagaaatgactgctaataccgcataagcgcacagtgctgcatggcacagtgtgaaaaactccggtggtatgagatggacccgcgtctgattagctagttggtggggtaacggcctaccaaggcgacgatcagtagccggcctgagagggtgaacggccacattgggactgagacacggcccaaactcctacggaaggcag

SEQ ID NO: 101 - DNA, 351 bp, artificial
  synthetic
gatgaacgctggcggcgtgcctaatacatgcaagtcgaacgaaacttctttatcaccgagtgcttgcactcaccgataaagagttgagtggcgaacgggtgagtaacacgtgggcaacctgcccaaaagagggggataacacttggaaacaggtgctaataccgcataaccatagttaccgcatggtaactatgtaaaaggtggctatgctaccgcttttggatgggcccgcggcgcattagctagttggtggggtaaaggcttaccaaggcaatgatgcgtagccgaactgagaggttgatcggccacattgggactgagacacggcccaaactcctacggaaggcagca

SEQ ID NO: 102 - DNA, 336 bp, artificial
  synthetic
gacgaacgctggcggcgtgctttaggcatgcaagtcgaacgcgaaagccccttcgggggtgagtagagtggcgaacgggtgagtaacacgtgggtaacctgcccctcgcagggggataaccgggggaaaccccggctaataccccgtacgcttgctggggcgcatgctccggcaaggaaaggtagcttcggccatccggcgagggaggggcccgcggcccattagctagttggtggggtaacggcccaccaaggcgacgatgggtagctggcctgagagggtggtcagccacactgggactgagacacggcccagactcctacggaaggcagcagg

SEQ ID NO: 103 - DNA, 334 bp, artificial
  synthetic
gatgaacgctagcggcaggcttaacacatgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacggaaggcagcagg

SEQ ID NO: 104 - DNA, 332 bp, artificial
  synthetic
gacgaacgctggcggcgtgcctaacacatgcaagtcgaacgagggtttggaaagcttgctttccgaaacctagtggcggacgggtgagtaacgcgtgagtaacctgcctttcagaggggaataacgttctgaaaagaacgctaataccgcataacgtacagttaccgcatggtagcagtaccaaaggagcaatccgctgaaagatggactcgcgtccgattagctagttggtggggtaaaggcctaccaaggcgacgatcggtagccggactgagaggttgaacggccacattgggactgagacacggcccagactcctacggaaggcagca

SEQ ID NO: 105 - DNA, 346 bp, artificial
  synthetic
gacgaacgctggcggcgtgcctaacacatgcaagtcgaacggaactatttcgaaagatttcttcggaatgattttggtttagtttagtggcggacgggtgagtaacgcgtgagtaacctgccttcaagagggggataacgttttgaaaagaacgctaataccgcataacatatcgaagccgcatgactttgatatcaaagattttatcgcttgaagatggactcgcgtccgattagttagttggtgaggtaacggctcaccaagaccgcgatcggtagccggactgagaggttgaacggccacattgggactgagacacggcccagactcctacggaaggcagcag

SEQ ID NO: 106 - DNA, 322 bp, artificial
  synthetic
attgaacgctggcggcatgccttacacatgcaagtcgaacggtaacaggtcttcggatgctgacgagtggcgaacgggtgagtaatacatcggaacgtgcccgatcgtgggggataacgaggcgaaagctttgctaataccgcatacgatctacggatgaaagcgggggatcttcggacctcgcgcggacggagcggccgatggcagattaggtagttggtgggataaaagcttaccaagccgacgatctgtagctggtctgagaggatgatcagccacactgggactgagacacggcccagactcctacggaaggcagcag

SEQ ID NO: 107 - DNA, 322 bp, artificial
  synthetic
attgaacgctggcggcaggcctaacacatgcaagtcgagcggatgacgggagcttgctccttgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagc

SEQ ID NO: 108 - DNA, 344 bp, artificial
  synthetic
gatgaacgctggcggcgtgcttaacacatgcaagtcgaacgggaaaccttttaatgaagcttcggcagatttagctggtttctagtggcggacgggtgagtaacgcgtgggtaacctgccttatacagggggataacagtcagaaatgactgctaataccgcataagcgcacaggaccgcatggtccggtgtgaaaaactccggtggtataagatggacccgcgttggattagcttgttggtggggtaacggctcaccaaggcgacgatccatagccggcctgagagggtgaacggccacattgggactgagacacggcccagactcctacggaaggcagcagg

SEQ ID NO: 109 - DNA, 337 bp, artificial
  synthetic
gatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacggaaggcagca

SEQ ID NO: 110 - DNA, 337 bp, artificial
  synthetic
gatgaacgctagctacaggcctaacacatgcaagtcgaggggcagcgggaaggaagcttgctttcttcgccggcgaccggcgcacgggtgcgtaacgcgtatcgaacctaccctttactcgggaacagccttgcgaaagcaagattaatgcccgatgttccgcgtttgctgcatggcaaattcggcaaagatttattggtaaaggatggcgatgcgtcccattagttagttggcggggtaacggcccaccaagacgacgatgggtaggggttctgagaggaaggtcccccacattggaactgagacacggtccaaactcctacggaaggcagcaggg

SEQ ID NO: 111 - DNA, 331 bp, artificial
  synthetic
attgaacgctggcggcaggcctaacacatgcaagtcgagcggtaacagagagtagcttgctactttgctgacgagcggcggacgggtgagtaatgcttgggaatatgccttttggtgggggacaacagttggaaacgactgctaataccgcatgatgtctacggaccaaagtgggggaccttcgggcctcacgccaaaagattagcccaagtgggattagctagttggtaaggtaatggcttaccaaggcgacgatccctagctggtttgagaggatgatcagccacactgggactgagacacggcccagactcctacggaaggcagcagg

SEQ ID NO: 112 - DNA, 344 bp, artificial
  synthetic
gatgaacgctggcggcgtgcttaacacatgcaagtcgaacgggaattactttattgaaacttcggtcgatttaatttaattctagtggcggacgggtgagtaacgcgtgggtaacctgccttgtacagggggataacagtcagaaatgactgctaataccgcataagcgcacaggaccgcatggtccggtgtgaaaaactccggtggtataagatggacccgcgttggattagctagttggtgaggtaacggcccaccaaggcgacgatccatagccggcctgagagggtgaacggccacattgggactgagacacggcccagactcctacggaaggcagcagg

SEQ ID NO: 113 - DNA, 344 bp, artificial
  synthetic
gatgaacgctggcggcgtgcttaacacatgcaagtcgagcgaagcgcttttgcggatttcttcggattgaagcaactgtgactgagcggcggacgggtgagtaacgcgtgggtaacctgcctcatacagggggataacagttggaaacggctgctaataccgcataagcgcacagtaccgcatggtaccgtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagcag

SEQ ID NO: 114 - DNA, 343 bp, artificial
  synthetic
gatgaacgctggcggcgtgcttaacacatgcaagtcgagcgaagcggctgaattgattcttcggatgattttcagttgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagg

SEQ ID NO: 115 - DNA, 343 bp, artificial
  synthetic
gatgaacgctagctacaggcttaacacatgcaagtcgaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacggaaggcagcagggttggt

SEQ ID NO: 116 - DNA, 342 bp, artificial
  synthetic
gatgaacgctggcggcgtgcttaacacatgcaagtcgaacgggaaacattttattgaagcttcggcagatttggtttgtttctagtggcggacgggtgagtaacgcgtgggtaacctgccttatactgggggataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacggaaggcagca

SEQ ID NO: 117 - DNA, 344 bp, artificial
  synthetic
gacgaacgctggcggcgtgcctaacacatgcaagtcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttttgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagc

SEQ ID NO: 118 - DNA, 342 bp, artificial
  synthetic
gatgaacgctggcggcgtgcctaacacatgcaagtcgagcgaagcgatttagcggaagttttcggatggaagttaaattgactgagcggcggacgggtgagtaacgcgtgggtaacctgcctcatacagggggataacagttagaaatgactgctaataccgcataagcgcacagcatcgcatgatgcagtgtgaaaaactccggtggtatgagatggacccgcgtctgattagctagttggtgaggtaacggctcaccaaggcgacgatcagtagccgacctgagagggtgatcggccacattgggactgagacacggcccaaactcctacggaaggcagc

SEQ ID NO: 119 - DNA, 306 bp, artificial
  synthetic
gatgaacgctggcggcgtgcctaatacatgcaagtcgagcgaaccacttcggtggtgagcggcgaacgggtgagtaacacgtaggtgatctgcccatcagacggggacaacgattggaaacgatcgctaataccggataggacgaaagtttaaagatgcttctggcaccgctgatggatgagcctgcggcgcattagctagttggtagggtaaaggcctaccaaggcgacgatgcgtagccgacctgagagggtgaacggccacactgggactgagacacggcccagactcctacggaaggcagca

SEQ ID NO: 120 - DNA, 321 bp, artificial
  synthetic
gatgaacgctggcggcgtgcctaacacatgcaagtcgaacgaagcaccttttgagattcttcggatgatcgatcggtgactgagtggcggacgggtgagtaacgcgtgggtaacctgccctgtacagggggataacagctggaaacggctgctaataccgcataagcgcacgaggagacatctcctagtgtgaaaaactccggtggtacaggatgggcccgcgtctgattagctggttggcagggtaacggcctaccaaggcaacgatcagtagccggtctgagaggatgaacggccacattggaactgagacacggtcca

SEQ ID NO: 121 - DNA, 316 bp, artificial
  synthetic
gatgaacgctggcggcgtgcctaacacatgcaagtcgagcgatttacttcggtaaagagcggcggacgggtgagtaacgcgtgggtaacctgccctgtacacacggataacataccgaaaggtatgctaatacgagataatatacttttatcgcatggtagaagtatcaaagcttttgcggtacaggatggacccgcgtctgattagctagttggtaaggtaacggcttaccaaggcgacgatcagtagccgacctgagagggtgatcggccacattggaactgagacacggtccaaactcctacggaaggcagca

SEQ ID NO: 122 - DNA, 327 bp, artificial
  synthetic
gacgaacgctggcggcgtgcctaacacatgcaagtcgaacggggttgagtggttcggtattcaacttagtggcgaacgggtgagtaacgcgtgaagaacctgcctttcagtgggggacaacagttggaaacgactgctaataccgcataacactgatgaggggcatcccttatcagtcaaagctttatgtgctgaaagatggcttcgcgtctgattagcttgttggcggggtaacggcccaccaaggcgacgatcagtagccggtctgagaggatgaacggccacattgggactgagatacggcccagactcctacgggaggcagca

SEQ ID NO: 123 - DNA, 327 bp, artificial
  synthetic
gatgaacgctagcggcaggcttaacacatgcaagtcgaggggcagcacagggagcaatcctgggtggcgaccggcggaagggtgcgtaacgcgtgagcaacttgcccgtatctgggagataaccgatggaaacgtcgactaatatcccataacacattttgtggcatcgcagattgttaaaagagaatcggatacggataggctcgcgtgacattagctagttggagtggtaacggcacaccaaggcgacgatgtctaggggttctgagaggaaggtcccccacactggaactgagacacggtccagactcctacgggaggcagcag

SEQ ID NO: 124 - DNA, 341 bp, artificial
  synthetic
gacgaacgctggcggcgtgcttaacacatgcaagtcgaacggggtgtttatttcggtaaacaccaagtggcgaacgggtgagtaacgcgtaagcaatctaccttcaagatggggacaacacttcgaaaggggtgctaataccgaatgaatgtaagagtatcgcatgagacacttactaaaggaggcctctgaaaatgcttccgcttgaagatgagcttgcgtctgattagctagttggtgagggtaaaggcccaccaaggcgacgatcagtagccggtctgagaggatgaacggccacattgggactgagacacggcccagactcctacggaaggcagcag

SEQ ID NO: 125 - DNA, 344 bp, artificial
  synthetic
gatgaacgctggcggcgtgcttaacacatgcaagtcgaacgaagcacttttatttgatttcttcggaatgaagattttagtgactgagtggcggacgggtgagtaacgcgtgggtaacctgcctcatacagggggataacagttagaaatgactgctaataccgcataagaccacagtaccgcatggtacaggggtaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagc

SEQ ID NO: 126 - DNA, 345 bp, artificial
  synthetic
gatgaacgctggcggcgtgcttaacacatgcaagtcgaacgaagcattttggaaggaagttttcggatggaattccttaatgactgagtggcggacgggtgagtaacgcgtggggaacctgccctatacagggggataacagctggaaacggctgctaataccgcataagcgcacagaatcgcatgattcggtgtgaaaagctccggcagtataggatggtcccgcgtctgattagctggttggcggggtaacggcccaccaaggcgacgatcagtagccggcttgagagagtggacggccacattgggactgagacacggcccaaactcctacggaaggcagca

SEQ ID NO: 127 - DNA, 337 bp, artificial
  synthetic
attgaacgctggcggaacgctttacacatgcaagtcgaacggtaacgcggagagaagcttgcttctctccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacggaaggcagcagg

SEQ ID NO: 128 - DNA, 333 bp, artificial
  synthetic
attgaacgctggcggcatgccttacacatgcaagtcgaacggcagcacgggtgcttgcacctggtggcgagtggcgaacgggtgagtaatacatcggaacatgtcctgtagtgggggatagcccggcgaaagccggattaataccgcatacgatccatggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacggaaggcagcagggttggt

SEQ ID NO: 129 - DNA, 347 bp, artificial
  synthetic
gatgaacgctggcggcgtgcttaacacatgcaagtcgaacggaaatgatacgctgatgcgatttcgatcaaatcttgtgtcattttagtggcggacgggtgagtaacgcgtgggtaacctgccttacactgggggataacacttagaaataggtgctaataccgcataagcgcacgagaccgcatggtctagtgtgaaaaactccggtggtgtaagatggacccgcgtctgattagctagttggcggggtaacggcccaccaaggcgacgatcagtagccggcctgagagggtggacggccacattgggactgagacacggcccaaactcctacggaaggcagcagg

SEQ ID NO: 130 - DNA, 328 bp, artificial
  synthetic
gacgaacgctggcggcgtgcttaacacatgcaagtcgaacggtgatgtcagagcttgctctggcggatcagtggcgaacgggtgagtaacacgtgagtaacctgcccccgactctgggataactgctagaaatggtagctaataccggatatgacgactggccgcatggtctggtcgtggaaagaatttcggttggggatggactcgcggcctatcaggttgttggtgaggtaatggctcaccaagcctacgacgggtagccggcctgagagggtgaccggccacactgggactgagacacggcccagactcctacggaaggcagcag

SEQ ID NO: 131 - DNA, 298 bp, artificial
  synthetic
gatgaacgctggcggtatgcttaacacatgcaagtcgaacggatgttttcggacattagtggcggacgggtgagtaacgcgtgagaatctagcttcaggttggggacaacagttggaaacgactgctaataccgaatgtgccgagaggtgaaagattaattgcctgaagaagagctcgcgtctgattagctagttggtggggtaagagcttaccaaggcgacgatcggtagctggtctgagaggacgatcagccacactgggactgagacacggcccagactcctacgggaggcagca

SEQ ID NO: 132 - DNA, 338 bp, artificial
  synthetic
gatgaacgctagctacaggcttaacacatgcaagtcgaggggcagcatggactcagctttgctgagtttgatggcgaccggcgcacgggtgagtaacgcgtatccaacctgccctttactccgggatagtctcctgaaagggagtttaataccggatgtgtttgtttttccgcatgggagcgacaaataaagattaattggtaaaggatggggatgcgtcccattagcttgttggcggggtaacggcccaccaaggcgacgatgggtaggggttctgagaggaaggtcccccacattggaactgagacacggtccaaactcctacggaaggcagcagg

SEQ ID NO: 133 - DNA, 345 bp, artificial
  synthetic
gatgaacgctggcggcgtgcctaacacatgcaagtcgaacggagtcgttttggaaaatccttcgggattggaattctcgacttagtggcggacgggtgagtaacgcgtgagcaatctgcctttaagagggggataacagtcggaaacggctgctaataccgcataaagcatcaaattcgcatgtttttgatgccaaaggagcaatccgcttttagatgagctcgcgtctgattagctggttggcggggtaacggcccaccaaggcgacgatcagtagccggactgagaggttgaacggccacattgggactgagacacggcccagactcctacgggaggcagcag

SEQ ID NO: 134 - DNA, 359 bp, artificial
  synthetic
gacgaacgctggcggcgtgcctaatacatgcaagtcgagcgagcttgcctagatgattttagtgcttgcactaaatgaaactagatacaagcgagcggcggacgggtgagtaacacgtgggtaacctgcccaagagactgggataacacctggaaacagatgctaataccggataacaacactagacgcatgtctagagtttgaaagatggttctgctatcactcttggatggacctgcggtgcattagctagttggtaaggtaacggcttaccaaggcaatgatgcatagccgagttgagagactgatcggccacattgggactgagacacggcccaaactcctacggaaggcagcag

SEQ ID NO: 135 - DNA, 327 bp, artificial
  synthetic
gatgaacgctggcggcatgcctaatacatgcaagtcgaacgaaccgcttttataggcggagagtggcgaacgggtgagtaacacgtagggaacctacccatgcgagggggacaacttctggaaacggaagctaataccgaataaggaaatggaaggcatcttcgatttcttaaaggaggcgtaagccttgcgcaaggatggacctgcggtgcattagctggttggtaaggtaacggcttaccaaggcgacgatgcatagccggcctgagagggcggacggccacactgggactgagacacggcccagactcctacggaaggcagcag

SEQ ID NO: 136 - DNA, 333 bp, artificial
  synthetic
attgaacgctggcggcaggcctaacacatgcaagtcgagcggcagcgggaaagtagcttgctacttttgccggcgagcggcggacgggtgagtaatgcctgggaaattgcccagtcgagggggataacagttggaaacgactgctaataccgcatacgccctgctttggaaagcaggggaccttcgggccttgcgcgattggatatgcccaggtgggattagctagttggtgaggtaatggctcaccaaggcgacgatccctagctggtctgagaggatgatcagccacactggaactgagacacggtccagactcctacgggaggcagcagg

SEQ ID NO: 137 - DNA, 348 bp, artificial
  synthetic
gatgaacgctggcggcgtgcttaacacatgcaagtcgaacgaagcaccttattctgatttcttcggaatgaaaaatttggtgactgagtggcggacgggtgagtaacgcgtgggtaacctgcctcatacagggggataacagttagaaatgactgctaataccgcataagaccacagcaccgcatggtgcaggggaaaaaactccggtggtatgagatggacccgcgtctgattaggtagttggtgaggtaacggcttaccaaggcgacgatcagtagccgacctgagagggtgaccggccacattgggactgagacacggcccaaactcctacgggaggcagcaggg

SEQ ID NO: 138 - DNA, 333 bp, artificial
  synthetic
gatgaacgctagctacaggcttaacacatgcaagtcgcggggcagcatgtcggttgcttgcaaccgatgatggcgaccggcgcacgggtgagtaacgcgtatccaacctgcccttcaccacggaataatccagtgaaaattggtctaataccgtatgaggtcatacgatggcatcagaatatgacgaaaggtttagcggtgaaggatggggatgcgtctgattagcttgttggtgaggtaacggctcaccaaggcgacgatcagtaggggttctgagaggaaggtcccccacattggaactgagacacggtccaaactcctacggaaggcagc

SEQ ID NO: 139 - DNA, 351 bp, artificial
  synthetic
gatgaacgctggcggcgtgcttaacacatgcaagtcgaacgaagcactttatttgatttcttcggaatgaagattttgtgactgagtggcggacgggtgagtaacgcgtgggtaacctgcctcatacagggggataacagttagaaatgactgctaataccgcataagcgcacaggattgcatgatctggtgtgaaaaactccggtggtataagatggacccgcgtctgattagctagttggtgaggtaacggcccaccaaggcgacgatcagtagccgacctgagagggtgaccggccacattgggactgagacacggcccaaactcctacggaaggcagcagggttggt

SEQ ID NO: 140 - DNA, 343 bp, artificial
  synthetic
gatgaacgctggcggcgtgcttaacacatgcaagtcgaacgaagcaatttagcggaagttttcggatggaagctagattgactgagtggcggacgggtgagtaacgcgtgggtaacctgcctcacactgggggacaacagttagaaatgactgctaataccgcataagcgcacaggaccgcatggtccggtgtgaaaaactccggtggtgtgagatggacccgcgtttgattagctagttggtggggtaacggcctaccaaggcgacgatcaatagccgacctgagagggtgaccggccacattgggactgagacacggcccaaactcctacggaaggcagca

SEQ ID NO: 141 - DNA, 345 bp, artificial
  synthetic
gatgaacgctggcggcgtgcctaacacatgcaagtcgagcgaagcggttttaaggaagttttcggatggaattaaaactgactgagcggcggacgggtgagtaacgcgtgggtaacctgcctcatacagggggataacagttagaaatggctgctaataccgcataagcacacagcttcgcatggagcagtgtgaaaaactccggtggtatgagatggacccgcgtctgattagctagttggtaaggtaacggcttaccaaggcgacgatcagtagccgacctgagagggtgaccggccacattgggactgagacacggcccaaactcctacggaaggcagcagg

SEQ ID NO: 142 - DNA, 330 bp, artificial
  synthetic
tgaacgctagcgacaggcttaacacatgcaagtcgaggggcagcgggggtagcgatacccgccggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcaggg

SEQ ID NO: 143 - DNA, 326 bp, artificial
  synthetic
gacgaacgctggcggcgtgcctaacacatgcaagtcgaacggagttactccttcgggggtaacttagtggcgaacgggtgagtaacgcgtgaggaacctgcctttcagtgggggacaacagttggaaacgactgctaataccgcatgatgtgtcttggaggcatctccgggacaccaaagctttatgtgctgaaagatggcctcgcgtctgattagatagttggcggggtaacggcccaccaagtcgacgatcagtagccggtctgagaggatgaacggccacattgggactgagatacggcccagactcctacggaaggcagcag

SEQ ID NO: 144 - DNA, 328 bp, artificial
  synthetic
gacgaacgctggcggcgcgcctaacacatgcaagtcgaacggtgaagaggagcttgctcctcggatcagtggcggacgggtgagtaacacgtgagcaacctggctctaagagggggacaacagttggaaacgactgctaataccgcatgatgtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacggaaggcagcag

SEQ ID NO: 145 - DNA, 329 bp, artificial
  synthetic
gacgaacgctggcggcgtgcctaacacatgcaagtcgagcgagtggatctccttcgggagtgaagctagcggcggacgggtgagtaacacgtgggcaacctgcctcatagaggggaatagcctcccgaaagggagattaataccgcataagattgtagcttcgcatgaagtagcaattaaaggagcaatccgctatgagatgggcccgcggcgcattagctagttggtgaggtaacggctcaccaaggcgacgatgcgtagccgacctgagagggtgatcggccacattgggactgagacacggcccagactcctacggaaggcagcag

SEQ ID NO: 146 - DNA, 326 bp, artificial
  synthetic
gatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagacgtgatcgcatggtcgtgtttggaaagatttttcggtctgggatgggctcgcggcctatcagcttgttggtgaggtaatggctcaccaaggcgtcgacgggtagccggcctgagagggtgaccggccacactgggactgagacacggcccagactcctacggaaggcagcag

SEQ ID NO: 147 - DNA, 333 bp, artificial
  synthetic
gatgaacgctagctacaggcttaacacatgcaagtcgaggggcagcatgacggaagcttgcttccgatgatggcgaccggcgcacgggtgagtaacgcgtatccaacctgcccttgtccatcggataacccgtcgaaaggcggactaacacgatatgcagtccacagcaggcatctaacgtggacgaaatgtgaaggagaaggatggggatgcgtctgattagcttgttggcggggtaacggcccaccaaggcgacgatcagtaggggttctgagaggaaggtcccccacattggaactgagacacggtccaaactcctacggaaggcagcag

SEQ ID NO: 148 - DNA, 294 bp, artificial
  synthetic
gtataggggcaagtggataaatgggctagagtagataaaggaggtggttggggatatggattctagagcgattggttaacatttgaaaggtgagctcctgagagagtttgttactatctctaggaattagctaagcctgggaatggagtagcagtctgcagtaacagcaaggccccagacatcaaagcatcagaaatacagaaactaaagagacatagttaacagagtcggggcttagtggggttggtgtggggagcagagtgggctgtggaggctccctacggaaggcagcag

SEQ ID NO: 149 - DNA, 327 bp, artificial
  synthetic
attgaacgctggcggcatgccttacacatgcaagtcgaacggcagcacgggtgcttgcacctggtggcgagtggcgaacgggtgagtaatgcatcggaacgtaccctggagtgggggataactatccgaaaggatagctaataccgcatattctatgagtaggaaagcgggggatcttcggacctcgcgctccgggagcggccgatgtcagattagctagttggtggggtaaaggcctaccaaggctacgatctgtagcgggtctgagaggatgatccgccacactgggactgagacacggcccagactcctacgggaggcagcagg

SEQ ID NO: 150 - DNA, 332 bp, artificial
  synthetic
gacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagg

SEQ ID NO: 151 - DNA, 333 bp, artificial
  synthetic
gacgaacgctggcggcgcgcctaacacatgcaagtcgaacgaagtcaagaggagcttgctctttttgacttagtggcgaacgggtgagtaacgcgtgaggaacctgcctcaaagagggggacaacagttggaaacgactgctaataccgcataagcccacagctccgcatggagcagagggaaaaggagcaatccgctttgagatggcctcgcgtccgattagctagttggtgaggtaacggcccaccaaggcgacgatcggtagccggactgagaggttgatcggccacattgggactgagacacggcccagactcctacgggaggcagcag

SEQ ID NO: 152 - DNA, 346 bp, artificial
  synthetic
gatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcag

SEQ ID NO: 153 - DNA, 342 bp, artificial
  synthetic
gatgaacgctggcggcgtgcctaacacatgcaagtcgaacgaagcaattaaaggaagttttcggatggaatttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacggaaggcagcag

SEQ ID NO: 154 - DNA, 347 bp, artificial
  synthetic
gatgaacgctggcggcgtgcttaacacatgcaagtcgaacgaagcactttaacctgattcttcggatgaaggtttttgtgactgagtggcggacgggtgagtaacgcgtgggtaacctgcctcatacagggggataacagttagaaatgactgctaataccgcataagaccacggagccgcatggctcagtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagggt

SEQ ID NO: 155 - DNA, 298 bp, artificial
  synthetic
agcgaacgctggcggcaggcttaacacatgcaagtcgaacgggcgtagcaatacgtcagtggcagacgggtgagtaacgcgtgggaacgtaccttttggttcggaacaacacagggaaacttgtgctaataccggataagcccttacggggaaagatttatcgccgaaagatcggcccgcgtctgattagctagttggtgaggtaacggctcaccaaggcgacgatcagtagctggtctgagaggatgatcagccacattgggactgagacacggcccaaactcctacggaaggcagc

SEQ ID NO: 156 - DNA, 337 bp, artificial
  synthetic
attgaacgctggcggcaggcctaacacatgcaagtcgagcggaaacgacactaacaatccttcgggtgcgttaatgggcgtcgagcggcggacgggtgagtaatgcctaggaaattgccttgatgtgggggataaccattggaaacgatggctaataccgcatgatgcctacgggccaaagagggggaccttcgggcctctcgcgtcaagatatgcctaggtgggattagctagttggtgaggtaatggctcaccaaggcgacgatccctagctggtctgagaggatgatcagccacactggaactgagacacggtccagactcctacgggaggcag

SEQ ID NO: 157 - DNA, 321 bp, artificial
  synthetic
gatgaacgctggcggcgtgcctaatacatgcaagtcgaacgcatcacttcggtgatgagtggcgaacgggtgagtaatacataagtaacctggcccatacagggggataactgctggaaacggcagctaagaccgcatatgtgtagagatcgcatgaactctatatgaaaagtgctacggcactggtaagggatggacttatggcgcattagctagttggcagggtaaaggcctaccaaggcgacgatgcgtagccgacctgagagggtgaccggccacactgggactgagacacggcccagactcctacggaaggcagca

SEQ ID NO: 158 - DNA, 345 bp, artificial
  synthetic
gatgaacgctggcggcgtgcttaacacatgcaagtcgaacgaagcatttaagacagattacttcggtttgaagtcttttatgactgagtggcggacgggtgagtaacgcgtgggtaacctgcctcatacagggggatagcagctggaaacggctggtaataccgcataagcgcacggtatcgcatgatacagtgtgaaaaactccggtggtatgagatggacccgcgtctgattagctggttggtgaggtaacggcccaccaaggcgacgatcagtagccgacctgagagggtgaccggccacattgggactgagacacggcccagactcctacggaaggcagca

SEQ ID NO: 159 - DNA, 330 bp, artificial
  synthetic
attgaacgctggcggcaggcttaacacatgcaagtcgaacggtaacataaagaagcttgcttctttgatgacgagtggcggacgggtgagtaatgcttgggaatctagcttatggagggggataactacgggaaactgtagctaataccgcgtagtatcgagagatgaaagtgtgggaccttcgggccacatgccataggatgagcccaagtgggattaggtagttggtgaggtaaaggctcaccaagccgacgatctctagctggtctgagaggatgaccagccacactgggactgagacacggcccagactcctacggaaggcagcag

SEQ ID NO: 160 - DNA, 346 bp, artificial
  synthetic
gatgaacgctggcggcgtgcttaacacatgcaagtcgaacggaggacccttgacggagttttcggacaacggataggaatccttagtggcggacgggtgagtaacgcgtgaggaacctgccttggagaggggaataacacagagaaatttgtgctaataccgcatgatgcagttgggtcgcatggctctgactgccaaagatttatcgctctgagatggcctcgcgtctgattagctagttggtagggtaacggcctaccaaggcgacgatcagtagccggactgagaggttgaccggccacattgggactgagacacggcccagactcctacggaaggcagcagg

SEQ ID NO: 161 - DNA, 334 bp, artificial
  synthetic
gacgaacgctggcggcgcgcctaacacatgcaagtcgaacggagctgagaggagcttgcttttcttagcttagtggcgaacgggtgagtaacgcgtgagtaacctgccctggagtgggggacaacagttggaaacgactgctaataccgcataagcccacgatccggcatcggattgagggaaaaggatttattcgcttcaggatggactcgcgtccaattagctagttggtgaggtaacggcccaccaaggcgacgattggtagccggactgagaggttgaacggccacattgggactgagacacggcccagactcctacggaaggcagcagg

SEQ ID NO: 162 - DNA, 343 bp, artificial
  synthetic
gacgaacgctggcggcgtgcttaacacatgcaagtcgaacgagaatctactgaaagagttttcggacaatggaagtagaggaaagtggcggacgggtgagtaacgcgtgaggaacctgccttgaagagggggacaacagttggaaacgactgctaataccgcatgacgcataggggtcgcatgatctttatgccaaagatttatcgcttcaagatggcctcgcgtctgattagctagttggcggggtaacggcccaccaaggcgacgatcagtagccggactgagaggttgaacggccacattgggactgagatacggcccagactcctacggaaggcagcag

SEQ ID NO: 163 - DNA, 345 bp, artificial
  synthetic
gatgaacgctggcggcgtgcttaacacatgcaagtcgaacgaagcactttattacgatttcttcggaatgacgatttagtgactgagtggcggacgggtgagtaacgcgtgggtaacctgcctcatacagggggataacagttggaaacgactgctaataccgcataagcgcacagtatcgcatgatacagtgtgaaaaactccggtggtatgagatggacccgcgtctgattagctagttggtggggtaacggcctaccaaggcaacgatcagtagccgacctgagagggtgaccggccacattgggactgagacacggcccaaactcctacggaaggcagcag

SEQ ID NO: 164 - DNA, 343 bp, artificial
  synthetic
gacgaacgctggcggcgtgcttaacacatgcaagtcgaacgagaatctgcggatcgaggattcgtccaagtgaagtagaggacagtggcggacgggtgagtaacgcgtgaggaacctgcctttcagagggggacaacagttggaaacgactgctaataccgcatgacacattggggtcgcatggccctgatgtcaaagatttatcgctgaaagatggcctcgcgtctgattagctagttggtgaggtaacggcccaccaaggcgacgatcagtagccggactgagaggttgaccggccacattgggactgagatacggcccagactcctacgggaggcagcag

SEQ ID NO: 165 - DNA, 346 bp, artificial
  synthetic
gatgaacgctggcggcgtgcttaacacatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacaggatcgcatgatccggtgtgaaaaactccggtggtatgagatggacccgcgtctgattagccagttggcagggtaacggcctaccaaagcgacgatcagtagccgacctgagagggtgaccggccacattgggactgagacacggcccaaactcctacgggaggcagcag

SEQ ID NO: 166 - DNA, 346 bp, artificial
  synthetic
gatgaacgctggcggcgtgcttaacacatgcaagtcgaacgaagcactttaccggatttcttcgggatgaaagttttgtgactgagtggcggacgggtgagtaacgcgtgggtaacctgcctcatacagggggataacagttagaaatgactgctaataccgcataagaccacaggattgcatgatccggtggtaaaaaactccggtggtatgagatggacccgcgtctgattaggtagttggtggggtaacggctcaccaagccgacgatcagtagccgacctgagagggtgaccggccacattgggactgagacacggcccaaactcctacggaaggcagcagg

SEQ ID NO: 167 - DNA, 340 bp, artificial
  synthetic
gatgaacgctagcgacaggcctaacacatgcaagtcgaggggcaacggggatgttagcttgctaatatctgccggcgaccggcgcacgggtgagtaacgcgtatgcgacctgcccgtcacagggggataatccggagaaatccggtctaataccgcataatatcgtgaatctgcatggatttgcgattaaaggagcgatccggtgacggatgggcatgcgtgacattagctagtcggcggggtaacggcccaccgaggcgacgatgtctaggggttctgagaggaaggtcccccacactggtactgagacacggaccagactcctacggaaggcagcagg

SEQ ID NO: 168 - DNA, 334 bp, artificial
  synthetic
gatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatagcaccattaaagatttattggtaaacgatggggatgcgttccattaggtagtaggcggggtaacggcccacctagccgacgatggataggggttctgagaggaaggtcccccacattggaactgagacacggtccaaactcctacgggaggcagca

SEQ ID NO: 169 - DNA, 335 bp, artificial
  synthetic
gataaacgctggcggcgcacataagacatgcaagtcgaacggacttaaccattagtttactattggagcggttagtggcggactggtgagtaacacgtaagcaacctgcctatcagaggggaacaacagttagaaatgactgctaataccgcatatgccttaattaccacatggtacaagagggaaaggagcaatccgctgatagatgggcttgcgtctgattagatagttggtaaggtaacggcttaccaagtcgacgatcagtagccggactgagaggttgaacggccacattgggactgagatacggcccagactcctacggaaggcagcag

SEQ ID NO: 170 - DNA, 343 bp, artificial
  synthetic
gatgaacgctggcggcgtgcttaacacatgcaagtcgaacgaagcacttaagtttgattcttcggatgaagacttttgtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattagctggttggtggggtaacggcctaccaaggcgacgatcagtagccgacctgagagggtgaccggccacattgggactgagacacggcccaaactcctacgggaggcagca

SEQ ID NO: 171 - DNA, 334 bp, artificial
  synthetic
gatgaacgctagctacaggcttaacacatgcaagtcgaggggcagcatgtcggttgcttgcaaccgatgatggcgaccggcgcacgggtgagtaacgcgtatccaacctacccttgtccatcggataacccgtcgaaaggcggcctaacacgatatgcggttctcagcaggcatctaacgagaacgaaatgtgaaggagaaggatggggatgcgtctgattagcttgttggcggggtaacggcccaccaaggcgacgatcagtaggggttctgagaggaaggtcccccacattggaactgagacacggtccaaactcctacggaaggcagcagg

SEQ ID NO: 172 - DNA, 351 bp, artificial
  synthetic
gacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcaggg

SEQ ID NO: 173 - DNA, 344 bp, artificial
  synthetic
gatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatggctgctaataccgcataagaccacagtactgcatggtacagtggtaaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagca

SEQ ID NO: 174 - DNA, 344 bp, artificial
  synthetic
gacgaacgctggcggcgtgcttaacacatgcaagtcgaacgagaatcactggaaggagttttcggacaacggaaggtgaggacagtggcggacgggtgagtaacgcgtgaggaacctgcctttcagagggggacaacagttggaaacgactgctaataccgcatgacacatagagatcgcatggtttttatgtcaaagatttatcgctgaaagatggcctcgcgtctgattagctagttggtgaggtaacggcccaccaaggcgacgatcagtagccggactgagaggttgaccggccacattgggactgagatacggcccagactcctacggaaggcagcagg

SEQ ID NO: 175 - DNA, 331 bp, artificial
  synthetic
gatgaacgctagcggcaggcctaacacatgcaagtcgaggggcatcacgaggtagcaatactttggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacggaaggcagcagg

SEQ ID NO: 176 - DNA, 352 bp, artificial
  synthetic
gatgaacgctggcggcgtgcttaacacatgcaagtcgagcgaagcactttgctttgatttcttcgggatgaagagcttagtgactgagcggcggacgggtgagtaacgcgtgggtaacctgcctcatacagggggataacagttagaaatgactgctaataccgcataaggccacagcaccgcatggtgcaggggtaaaaactccggtggtatgagatggacccgcgtctgattaggtagttggcggggtaacggcccaccaagccgacgatcagtagccgacctgagagggtgaccggccacattgggactgagacacggcccaaactcctacggaaggcagcagggtttt

SEQ ID NO: 177 - DNA, 329 bp, artificial
  synthetic
gataaacgctggcggcatgcctaatacatgcaagtcgtacggatatcttttgatatcagtggcgaacgggtgagtaacacgtagggaacctgcccgcagccgggggatacgctctggaaacggagtctaaaaccccataggcagaaagacggcatcgtctttctgtgaaaaggacttttgtcctggcggcggatggacctgcggtgcattagtcagttggtgaggtaacggctcaccaagaccatgatgcatagccggcctgagagggcggacggccacactgggactgagacacggcccagactcctacggaaggcagcagggttggt

SEQ ID NO: 178 - DNA, 343 bp, artificial
  synthetic
gatgaacgctggcggcgtgcttaacacatgcaagtcgaacgaagcacttaaatttgattcttcggatgaagatttttgtgactgagtggcggacgggtgagtaacgcgtgggtaacctgcctcatacagggggataacagttagaaatgactgctaataccgcataagcgcacagggtcgcatgacctggtgtgaaaaactccggtggtatgagatggacccgcgtctgattagctagttggtgaggtaacggcccaccaaggcgacgatcagtagccgacctgagagggtgatcggccacattgggactgagacacggcccaaactcctacggaaggcagca

SEQ ID NO: 179 - DNA, 338 bp, artificial
  synthetic
ggacgaacgctggcggcgtgcctaatacatgcaagtagaacgctgaaggaaggagcttgctctttccggatgagttgcgaacgggtgagtaacgcgtaggtaacctgcctggtagcgggggataactattggaaacgatagctaataccgcataatagtagatgttgcatgacatttgcttaaaaggtgcaattgcatcactaccagatggacctgcgttgtattagctagttggtgaggtaacggctcaccaaggcgacgatacatagccgacctgagagggtgatcggccacactgggactgagacacggcccagactcctacggaaggcagcagg

SEQ ID NO: 180 - DNA, 337 bp, artificial
  synthetic
gatgaacgctagcgacaggcttaacacatgcaagtcgaggggcatcatgaggtagcaataccttgatggcgaccggcgcacgggtgagtaacgcgtatgcaacctgcctgataccggggtatagcccatggaaacgtggattaacaccccatagtacttttatcctgcatgggatgtgagttaaatgttcaaggtatcggatgggcatgcgtcctattagttagttggcggggtaacagcccaccaagacgatgataggtaggggttctgagaggaaggtcccccacattggaactgagacacggtccaaactcctacgggaggcagcagggttggt

SEQ ID NO: 181 - DNA, 343 bp, artificial
  synthetic
gatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacggaaggcagcagggttggt

SEQ ID NO: 182 - DNA, 337 bp, artificial
  synthetic
gatgaacgctagcgacaggcctaacacatgcaagtcgaggggcagcggagaggtagcaatacctttgccggcgaccggcgcacgggtgagtaacacgtatgcaatccacctgtaacagggggataacccggagaaatccggactaataccccataatatgggcgctccgcatggagggctcattaaagagagcaattttggttacagacgagcatgcgctccattagccagttggcggggtaacggcccaccaaagcgacgatggataggggttctgagaggaaggtcccccacattggaactgagacacggtccaaactcctacggaaggcagcag

SEQ ID NO: 183 - DNA, 346 bp, artificial
  synthetic
gacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacggaaggcagca

SEQ ID NO: 184 - DNA, 328 bp, artificial
  synthetic
gatgaacgctggcggcgtgcttaacacatgcaagtcgagcgatgaagtttccttcgggaaacggattagcggcggacgggtgagtaacacgtgggtaacctgcctcatagagtggaatagccttccgaaaggaagattaataccgcataatgttgaaagatggcatcatcattcaaccaaaggagcaatccgctatgagatggacccgcggcgcattagctagttggtggggtaacggcctaccaaggcgacgatgcgtagccgacctgagagggtgatcggccacattgggactgagacacggcccagactcctacggaaggcagca

SEQ ID NO: 185 - DNA, 329 bp, artificial
  synthetic
agcgaacgttggcgatgcgtcttaagcatgcaagtcgagcgggcttattcgggcaactggataagttagcggcgaactggtgagtaacacgtaggtaatctgccgtagagtgggggataacccatggaaacatggactaataccgcatatactcttgacgctaaagcgtagtagaggaaaggagcaatccgctttacgatgagcctgcggcctattagcctgttggtgagataaaagcccaccaaagctacgataggtagccgacctgagagggtgaccggccacattgggactgagatacggcccagactcctacggaaggcagcagg

SEQ ID NO: 186 - DNA, 328 bp, artificial
  synthetic
attgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcag

SEQ ID NO: 187 - DNA, 343 bp, artificial
  synthetic
gatgaacgctggcggcgtgcctaacacatgcaagtcgaacgggtgtacgggatggaaggctccggccggaagccccgtgcatgagtggcggacgggtgagtaacgcgtgggcaacctgccctgtacagggggacaacacttagaaataggtgctaataccgcataacggggggagccgcatggctttcccctgaaaactccggtggtacaggatgggcccgcgtctgattagctagttggcagggtaacggcctaccaaggcgacgatcagtagccggcctgagagggcggacggccacactgggactgagacacggcccagactcctacggaaggcagcagg

SEQ ID NO: 188 - DNA, 345 bp, artificial
  synthetic
gatgaacgctggcggcgtgcctaacacatgcaagtcgaacgaagcgatcttaatgaagttttcggatggatttgagattgacttagtggcggacgggtgagtaacgcgtgggtaacctgccttgtacagggggataacagttagaaatgactgctaataccgcataacccgctaaggtcgcatgacctggacggaaaagatttatcggtacaagatggacccgcgtctgattagctagttggtgaggtaacggcccaccaaggcgacgatcagtagccggcctgagagggtgaacggccacattgggactgagacacggcccaaactcctacgggaggcagcagg

SEQ ID NO: 189 - DNA, 346 bp, artificial
  synthetic
gatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagg

SEQ ID NO: 190 - DNA, 343 bp, artificial
  synthetic
gatgaacgctggcggcgtgcttaacacatgcaagtcgagcgaagcacttatgaaagattcttcggatgaattcatttgtgactgagcggcggacgggtgagtaacgcgtgggtaacctgcctcatacagggggataacagttggaaacgactgctaataccgcataagcgcacagtaccgcatggtacagtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagca

SEQ ID NO: 191 - DNA, 343 bp, artificial
  synthetic
gatgaacgctagctacaggcttaacacatgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagggttggt

SEQ ID NO: 192 - DNA, 343 bp, artificial
  synthetic
gatgaacgctggcggcgtgcctaacacatgcaagtcgagcgaagcattttaaaggaagttttcggatggaatttgaaatgactgagcggcggacgggtgagtaacgcgtgggtaacctgcctcatacagggggataacagttagaaatgactgctaataccgcataagcacacagtaccgcatggtacagtgtgaaaaactccggtggtatgagatggacccgcgtctgattagctggttggcggggtaacggcccaccaaggcgacgatcagtagccgacctgagagggtgaccggccacattgggactgagacacggcccaaactcctacggaaggcagca

SEQ ID NO: 193 - DNA, 337 bp, artificial
  synthetic
gatgaacgctagctacaggcttaacacatgcaagtcgaggggcagcattttagtttgcttgcaaactaaagatggcgaccggcgcacgggtgagtaacacgtatccaacctgccgataactcagggatagcctttcgaaagaaagattaatacctgatggcataggattatcgcatgataatcctattaaagaatttcggttatcgatggggatgcgttccattaggcagttggtgaggtaacggctcaccaaaccttcgatggataggggttctgagaggaaggtcccccacattggaactgagacacggtccaaactcctacggaaggcagcagg

SEQ ID NO: 194 - DNA, 343 bp, artificial
  synthetic
gatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacggaaggcagca

SEQ ID NO: 195 - DNA, 348 bp, artificial
  synthetic
gacgaacgctggcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacggaaggcagcag

SEQ ID NO: 196 - DNA, 332 bp, artificial
  synthetic
gacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacggtgccgcatggcacagagggaaaaggagcaatccgctttgagatggcctcgcgtccgattagctagttggtgaggtaatggcccaccaaggcaacgatcggtagccggactgagaggttgaacggccacattgggactgagacacggcccagactcctacgggaggcagcagg

SEQ ID NO: 197 - DNA, 348 bp, artificial
  synthetic
gatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacggaaggcagcagggttggt

SEQ ID NO: 198 - DNA, 344 bp, artificial
  synthetic
gatgaacgctggcggcgtgcctaacacatgcaagtcgaacgaggtattttgattgaagttttcggatggatttcaagataccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtggggtaacggcccaccaaggcgacgatcagtagccgacctgagagggtgaccggccacattgggactgagacacggcccagactcctacggaaggcagcagg

SEQ ID NO: 199 - DNA, 323 bp, artificial
  synthetic
attgaacgctggcggcatgccttacacatgcaagtcgaacggtaacaggtcttcggatgctgacgagtggcgaacgggtgagtaatacatcggaacgtgcccgatcgtgggggataacgaagcgaaagctttgctaataccgcataagatctacggatgaaagcaggggaccgcaaggccttgcgcgaacggagcggccgatggcagattaggtagttggtgggataaaagcttaccaagccgacgatctgtagctggtctgagaggacgaccagccacactgggactgagacacggcccagactcctacgggaggcagcagg

SEQ ID NO: 200 - DNA, 333 bp, artificial
  synthetic
gacgaacgctggcggcgtgcctaacacatgcaagtcgaacggagttgaaaggagcttgcttttttcaactcagtggcggacgggtgagtaacgcgtgagtaacctgcctttcagagggggataacgttctgaaaagaacgctaataccgcataagattgtagcttcgcatggagcagcaatcaaaggagcaatccgctgaaagatggactcgcgtccgattagatagttggcggggtaacggcccaccaagtcgacgatcggtagccggactgagaggttgatcggccacattgggactgagacacggcccagactcctacggaaggcagcag

SEQ ID NO: 201 - DNA, 328 bp, artificial
  synthetic
gacgaacgctggcggcgtgcctaacacatgcaagtcgaacggagttaagcccttcgggacttaacttagtggcgaacgggtgagtaacgcgtgagaaacctgcctttcagtgggggacaacagttggaaacgactgctaataccgcatgatacttcttgagggcatccttgagaagtcaaagctttatgtgctgaaagatggtctcgcgtctgattagctagttggtggggtaacggcccaccaaggcgacgatcagtagccggtctgagaggatgaacggccacattgggactgagatacggcccagactcctacggaaggcagcag

SEQ ID NO: 202 - DNA, 298 bp, artificial
  synthetic
aacgaacgctggcggcatgcctaacacatgcaagtcgaacgatgccttcgggcatagtggcgcacgggtgcgtaacgcgtgggaatctgcccctgggttcggaataacagcgagaaattgctgctaataccggatgatgacgaaagtccaaagatttatcgcccagggatgagcccgcgtaggattagctagttggtgaggtaagagctcaccaaggcgacgatccttagctggtctgagaggatgatcagccacactgggactgagacacggcccagactcctacggaaggcagcag

SEQ ID NO: 203 - DNA, 336 bp, artificial
  synthetic
gatgaacgctagctacaggcttaacacatgcaagtcgaggggcagcatggtcgaagcttgcttcgactgatggcgaccggcgcacgggtgcgtaacgcgtatcgaacctgccctatacacggggatagccttgcgaaagtaagattaatacccgatgccaagttgagatcgcatgattttgatttgaaagaaattcggtataggatggcgatgcgtctgattaggtagatggcggggtaacggcccaccatgccgacgatcagtaggggttctgagaggaaggtcccccacattggaactgagacacggtccaaactcctacggaaggcagcaggg

SEQ ID NO: 204 - DNA, 337 bp, artificial
  synthetic
gatgaacgctagcggcaggcttaacacatgcaagtcgaggggcagcggggagtagcaatactccgccggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacggaaggcagcagggttggt

SEQ ID NO: 205 - DNA, 329 bp, artificial
  synthetic
attgaacgctggcggcaggcctaacacatgcaagtcgagcggtaacatttctagcttgctagaagatgacgagcggcggacgggtgagtaatgcttgggaatatgccttttggtgggggacaacagttggaaacgactgctaataccgcatgatgtctacggaccaaagtgggggaccttcgggcctcacgccaagagattagcccaagtgggattagctagttggtaaggtaatggcttaccaaggcgacgatccctagctggtttgagaggatgatcagccacactgggactgagacacggcccagactcctacgggaggcagcagg

SEQ ID NO: 206 - DNA, 333 bp, artificial
  synthetic
gatgaacgctagctacaggcttaacacatgcaagtcgaggggcatcagggtgtagcaatacaccgctggcgaccggcgcacgggtgagtaacacgtatccaacctgccctttactcggggatagcctttcgaaagaaagattaatacccgatggcataacatgacctcctggttttgttattaaagaatttcggtagaggatggggatgcgttccattaggcagttggcggggtaacggcccaccaaaccttcgatggataggggttctgagaggaaggtcccccacattggaactgagacacggtccaaactcctacggaaggcagcagggt

SEQ ID NO: 207 - DNA, 361 bp, artificial
  synthetic
gatgaacgccggcggtgtgcctaatacatgcaagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacggaaggcagc

SEQ ID NO: 208 - DNA, 343 bp, artificial
  synthetic
gatgaacgctggcggcgtgcttaacacatgcaagtcgaacgaagcacttgagattgattcttcggaagatttctcttgtgacttagtggcggacgggtgagtaacgcgtgggtaacctgcctcacacagggggataacagttggaaacgaccgctaataccgcataacccgctagggccgcatggcccggacggaaaagatttatcggtgtgagatggacccgcgttggattagctagttggcagggtaacggcctaccaaggcgacgatccatagccggcctgagagggtgaacggccacattgggactgagacacggcccaaactcctacggaaggcagca

SEQ ID NO: 209 - DNA, 335 bp, artificial
  synthetic
gacgaacgctggcggcgcgcctaacacatgcaagtcgaacggagttgtgttgaaagcttgctggatatacaacttagtggcggacgggtgagtaacacgtgagtaacctgcctctcagagtggaataacgtttggaaacgaacgctaataccgcataacgtgagaagagggcatcctctttttaccaaagatttatcgctgagagatgggctcgcggccgattaggtagttggtgagataacagcccaccaagccgacgatcggtagccggactgagaggttgatcggccacattgggactgagacacggcccagactcctacggaaggcagcag

SEQ ID NO: 210 - DNA, 319 bp, artificial
  synthetic
agtgaacgctggcggcgtgcctaatacatgcaagtcgaacgatgaagctctagcttgctagagtggattagtggcgcacgggtgagtaatgcatagataacatgccctttagtctaggatagccattggaaacgatgattaatactggatactccttacgagggaaagtttttcgctaaaggattggtctatgtcctatcagcttgttggtgaggtaatggctcaccaaggctatgacgggtatccggcctgagagggtgaacggacacactggaactgagacacggtccagactcctacggaaggcagcagggttggt

SEQ ID NO: 211 - DNA, 343 bp, artificial
  synthetic
gatgaacgctggcggcgtgcttaacacatgcaagtcgagcgaagcgcttacgacagaaccttcgggggaagatgtaagggactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattagctagttggaggggtaacggcccaccaaggcgacgatcagtagccggcctgagagggtgaacggccacattgggactgagacacggcccagactcctacggaaggcagca

SEQ ID NO: 212 - DNA, 343 bp, artificial
  synthetic
gatgaacgctggcggcgtgcttaacacatgcaagtcgaacgaagcacctggatttgattcttcggatgaagatccttgtgactgagtggcggacgggtgagtaacgcgtgggtaacctgcctcatacagggggataacagttagaaatgactgctaataccgcataagaccacggagtcgcatgactcagtgggaaaaactccggtggtatgagatggacccgcgtctgattaggtagttggtggggtaacggcctaccaagccgacgatcagtagccgacctgagagggtgaccggccacattgggactgagacacggcccaaactcctacggaaggcagca

SEQ ID NO: 213 - DNA, 325 bp, artificial
  synthetic
attgaacgctggcggcaggcctaacacatgcaagtcgagcgaatgaggggagcttgctccctgatttagcggcggacgggtgagtaatgtatagggagctgcccgatagagggggataccagttggaaacgactgttaataccgcataatgtctacggaccaaagtgtgggaccttcgggccacatgctatcggatgcacctatatgggattagctagttggtggggtaacggctcaccaaggcgacgatccctagctggtttgagaggatgatcagccacactggaactgagacacggtccagactcctacggaaggcagcagg

SEQ ID NO: 214 - DNA, 354 bp, artificial
  synthetic
gacgaacgctggcggcgtgcttaacacatgcaagtcgaacgaaaagagggaaagagcttgctctttccggaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcaggg

SEQ ID NO: 215 - DNA, 346 bp, artificial
  synthetic
gatgaacgctggcggcatgcctaatacatgcaagtcgaacgaagtctttaggaagcttgcttccaaagagacttagtggcgaacgggtgagtaacacgtaggtaacctgcccatgtgcccgggataactgctggaaacggtagctaaaaccggataggtatgagggaggcatcttcctcatattaaagcaccttcgggtgtgaacatggatggacctgcggcgcattagctggttggtgaggtaacggcccaccaaggcgatgatgcgtagccgacctgagagggtgaacggccacattgggactgagacacggcccaaactcctacggaaggcagcagggttggt

SEQ ID NO: 216 - DNA, 334 bp, artificial
  synthetic
gatgaacgctagcgacaggcctaacacatgcaagtcgaggggcaacatggtgtcagcttgctgataccgatggcgaccggcgcacgggtgagtaacgcgtatgtaatctacctgtaacagagggataacccggagaaatccggactaatacctcatagtacatattattcgcatgagttttatgttaaagagattcggttacagatgaacatgcgttccattagctagttggcggggttacggcccaccaaggcaacgatggataggggttctgagaggaaggtcccccacactggtactgagacacggaccagactcctacgggaggcagcag

SEQ ID NO: 217 - DNA, 337 bp, artificial
  synthetic
gacgaacgctggcggcatgcctaacacatgcaagtcgaacggagattacttcggtaatcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacggaaggcagcag

SEQ ID NO: 218 - DNA, 323 bp, artificial
  synthetic
gatgaacgctggcggcgtgcctaatacatgcaagtcgaacgcaccgcttcggtggtgagtggcgaacgggtgagtaatacataagtaacctggcctttcgagggggataactattggaaacgatagctaagaccgcataggcataattctcgcatgagagttatgttaaatatcctatgggatagcgagaggatggacttatggcgcattagctagttggtgagggtaacggcccaccaaggcgacgatgcgtagccgacctgagagggtggacggccacactgggactgagacacggcccagactcctacggaaggcagcag

SEQ ID NO: 219 - DNA, 345 bp, artificial
  synthetic
gacgaacgctggcggcgcgcctaatacatgcaagtcgagcggggaggcaagcggaagccttcgggcggaagcttatctcctagcggcggacgggtgagtaacacgtgggcaacctgcctgtcagactgggataacgctgggaaaccggcgctaataccggatacgcttctttggccgcatggctggaggaggaaaggcgctttggcgctgctgacagatgggcctgcggcgcattagctagttggtgaggtaacggctcaccaaggcgacgatgcgtagccgacctgagagggtggccggccacactgggactgagacacggcccagactcctacggaaggcagc

SEQ ID NO: 220 - DNA, 323 bp, artificial
  synthetic
gatgaacgctggcggcatgcctaatacatgcaagtcgaacggcatcttcggatgcagtggcgaacgggtgaggaacacgtagggaacctggccatgcctgggggataatttctggaaacggaaactaagaccgcataggtggaaaggaagcattttcttttcattaaaggagcttcacagcttcgggaatggatggacctgcgctgcattagctggctggtgaggcaacggctcaccagggcgatgatgcatagccggcctgagagggcggacggccacactgggactgagacacggcccagactcctacggaaggcagcagg

SEQ ID NO: 221 - DNA, 343 bp, artificial
  synthetic
gatgaacgctggcggcgtgcctaacacatgcaagtcgaacgaagcacttatgtttgattcttcggatgaagatatatgtgactgagtggcggacgggtgagtaacgcgtgggtaacctgcctcatacagggggataacagttagaaatggctgctaataccgcataagcacacagtgttgcatgacacagtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagca

SEQ ID NO: 222 - DNA, 329 bp, artificial
  synthetic
gatgaacgctggcggcgtgcctaatacatgcaagtcgaacgcttcacatatgtgaagagtggcgaacgggtgagtaatacataagtaacctggcctttacagggggataactattggaaacgatagctaagaccgcataggtgtcataaccgcatggagatgatatgaaatatgctacggcataggtagaggatggacttatggcgcattagctagttggaggggtaacggcccaccaaggcgacgatgcgtagccgacctgagagggtgaccggccacactgggactgagacacggcccagactcctacgggaggcagcagggttggt

SEQ ID NO: 223 - DNA, 336 bp, artificial
  synthetic
gacgaacgctggcggcgtgcctaatacatgcaagttgagcgctgaaggttggtacttgtaccaactggatgagcagcgaacgggtgagtaacgcgtggggaatctgcctttgagcgggggacaacatttggaaacgaatgctaataccgcataaaaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacggaaggcagcag

SEQ ID NO: 224 - DNA, 342 bp, artificial
  synthetic
gatgaacgctggcggcgtgcctaacacatgcaagtcgaacgaggtaaatgagatgaagttttcggatggattcttatttaccgagtggcggacgggtgagtaacgcgtgggtaacctgcctcatacagggggataacgattggaaacgattgctaataccgcataagcgcacagtaccacatggtacagtgtgaaaaactccggtggtatgagatggacccgcgtctgattagctagttggtgaggtaacggcccaccaaggcaacgatcagtagccgacctgagagggtgaccggccacattgggactgagacacggcccagactcctacggaaggcagca

SEQ ID NO: 225 - DNA, 343 bp, artificial
  synthetic
gatgaacgctggcggcgtgcttaacacatgcaagtcgaacgaagcacttcataaagcttgctttaagaagtgacttagtggcggacgggtgagtaacgcgtgggtaacctgccttacacagggggataacagttagaaatgactgctaataccgcataaaacagcagagtcgcatgactcaactgtcaaagatttatcggtgtaagatggacccgcgtctgattagctggttggtggggtaacggcctaccaaggcgacgatcagtagccggcctgagagggtgaacggccacattgggactgagacacggcccaaactcctacgggaggcagcagggttggt

SEQ ID NO: 226 - DNA, 309 bp, artificial
  synthetic
aacgaacgctggcggcatgcctaacacatgcaagtcgaacgatgctttcgggcatagtggcgcacgggtgcgtaacgcgtgggaatctgccctttggtctgggataactgttggaaacgacagctaataccggatgatgacgtaagtccaaagatttatcgccagaggatgagcccgcgtcggattagctagttggtggggtaaaggcctaccaaggcgacgatccgtagctggtctgagaggatgatcagccacactgggactgagacacggcccagactcctacgggaggcagcagggttggttttt

SEQ ID NO: 227 - DNA, 341 bp, artificial
  synthetic
gatgaacgctagctacaggcctaacacatgcaagtcgaggggcatcgggagggaagcttgcttcccttgccggcgaccggcgcacgggtgagtaacgcgtatcgaacctgccccatacccggggatagccttgcgaaagtaagattaatacccgatggcttccttttatctcctgatagagggaataaagaatttcggtatgggatggcgatgcgtccgattagctggttggcggggtaacggcccaccaaggcgacgatcggtaggggttctgagaggaaggtcccccacattggaactgagacacggtccaaactcctacggaaggcagcagggttggt

SEQ ID NO: 228 - DNA, 343 bp, artificial
  synthetic
gatgaacgctggcggcgtgcttaacacatgcaagtcgagcgaagcacttatcattgactcttcggaagatttgatatttgactgagcggcggacgggtgagtaacgcgtgggtaacctgcctcatacaggggaataacagttagaaatggctgctaatgccgcataagcgcacaggaccgcatggtctggtgtgaaaaactgaggtggtatgagatggacccgcgtctgattaggtagttggtggggtaacggcctaccaagccgacgatcagtagccggcctgagagggtgaacggccacattgggactgagacacggcccagactcctacggaaggcagca

SEQ ID NO: 229 - DNA, 334 bp, artificial
  synthetic
gatgaacgctagcggcaggcttaacacatgcaagtcgaggggcagcgcggagtagcaatactctggcggcgaccggcggaagggtgcgtaacacgtgagcgacttgcccgtcacagggagataaccgctggaaacggcgactaatatcccatatgatggcgatctgcatggattgtcattgaaagattcatcggtgacggataggctcgcggggcattagctagacggcggggcaacggcccaccgtggcgacgatgcctaggggttctgagaggaagaacccccacactgggactgagacacggcccagactcctacggaaggcagcagggtt

SEQ ID NO: 230 - DNA, 324 bp, artificial
  synthetic
gacgaacgctggcggcgtgcttcatacatgcaagtcgaacgcgaatcggtagcttgctaccgaggaaagtggcggacgggtgagtaatatgtagagaatctgccctagagagggggacaacagctggaaacggttgctaataccccatatgagcgtacctgaaatggtattcttgaaaactccggtgctctaggatgagtctgcatctgattagctagttgggggtgtaatggaccaccaaggcgacgatcagtagctggtttgagaggatgatcagccacaatgggactgagacacggcccatactcctacggaaggcagcag

SEQ ID NO: 231 - DNA, 301 bp, artificial
  synthetic
gatgaacgctggcggtatgcttaacacatgcaagtcgaacggagtgcttcggcacttagtggcggacgggtgagtaacgcgtgagaatctagcttcgggttcgggacaacagtgggaaactgctgctaataccggatgtgcctaaaggtaaaagattaattgcccgaagatgagctcgcgtccgattagctagttggtgtggtaagagcgcaccaaggcgtcgatcggtagctggtctgagaggacgatcagccacactgggactgagacacggcccagactcctacggaaggcagcaggg

SEQ ID NO: 232 - DNA, 337 bp, artificial
  synthetic
gatgaacgctagctacaggcttaacacatgcaagtcgaggggcagcattatcgaagcttgctttgatagatggcgaccggcgcacgggtgagtaacgcgtatccaaccttccccatagtcaggaatagcccggcgaaagtcgaattaatgcctgatgttttccacggacggcatctgatgtggaacaaagattcatcgctatgggatggggatgcgtctgattagcttgttggcggggtaacggcccaccaaggcaacgatcagtaggggttctgagaggaaggtcccccacattggaactgagacacggtccaaactcctacgggaggcagcaggg

SEQ ID NO: 233 - DNA, 331 bp, artificial
  synthetic
gatgaacgctgacagaatgcttaacacatgcaagtctacttgaattctcttcggagatagtaaggtggcggacgggtgagtaacacgtaaagaacttgccctgcagtctgggacaactattggaaacgatagctaataccggatattatgcgagtgccgcatggcactttcatgaaagctatatgcgctgcaggagagctttgcgtcccattagttagttggtgaggtaacggctcaccaagaccgcgatgggtagccggcctgagagggtgaacggccacaaggggactgagacacggcccttactcctacgggaggcagcagggttggt

SEQ ID NO: 234 - DNA, 339 bp, artificial
  synthetic
gatgaacgctagctacaggcttaacacatgcaagtcgcggggcagcatgttactggcttgccagtaatgatggcgaccggcgcacgggtgagtatcgcgtatccaaccttcccatatccacgggatagcctgccgaaaggcagattaataccgtatgttgtcgcacgctggcatcaaaatgcgacgaaaggcttagcggatatggatggggatgcgtccgattagcttgacggcggggtaacggcccaccgtggcgacgatcggtaggggttctgagaggaaggtcccccacattggaactgagacacggtccaaactcctacgggaggcagcagggtt

SEQ ID NO: 235 - DNA, 342 bp, artificial
  synthetic
gatgaacgctagctacaggcttaacacatgcaagtcgaggggcagcatgacgatagcttgctattgtcgatggcgaccggcgcacgggtgagtaacgcgtatccaaccttcccgttgctaggggataaccttgcgaaagtaagactaataccctatgaagttcttcgcaggcatctaaagagaacgaaagatttatcggcaacggatggggatgcgtctgattaggttgttggcggggtaacggcccaccaagcccacgatcagtaggggttctgagaggaaggtcccccacattggaactgagacacggtccaaactcctacgggaggcagcagggttggt

SEQ ID NO: 236 - DNA, 341 bp, artificial
  synthetic
gatgaacgctagcggcaggcttaacacatgcaagtcgaggggcagcatggagagtagcaatactctctgatggcgaccggcgcaagggtgcgtaacgcgtgagcaacttgccctcatcaggggaataatcgctggaaacggcgtctaatgccccatggtgatggaatcaggcatctgatttcatctaaagatccgtcggatgaggataggctcgcgtgacattagctagacggcggggtaacggcccaccgtggcgacgatgtctaggggttctgagaggaaggtcccccacactggaactgagacacggtccagactcctacggaaggcagcagggttgg

SEQ ID NO: 237 - DNA, 342 bp, artificial
  synthetic
gatgaacgctggcggcgtgcctaacacatgcaagtcgaacgaagcaccttacctgattcttcggatgaaggtctggtgactgagtggcggacgggtgagtaacgcgtgggtaacctgccctgtacagggggataacagttggaaacggctgctaataccgcataagcgcacgagaggacatcctcttgtgtgaaaaactccggtggtacaggatgggcccgcgtctgattagctggttggcagggtaacggcctaccaaggcgacgatcagtagccggtctgagaggatgaacggccacattggaactgagacacggtccaaactcctacgggaggcagcag

SEQ ID NO: 238 - DNA, 244 bp, artificial
  synthetic
gcaacaggttgttgtctttctctgtgaagctacaggtctcatgctgctgttcctgctgctgcagctctgtctcctctacacaggttcaatatagatttaaatcacaatgtatcagtcacttctggggcaggagtgaggggaacaaggtggacatggcttctagattattgactgaacttggccaagtcgcttaacctctccaaacctgagtttcatcatcgtgacgggaggcagcagggttggt

SEQ ID NO: 239 - DNA, 334 bp, artificial
  synthetic
gatgaacgctggcggcgtgcctaacacatgcaagtcgaacgatgaaaccgccctcgggcggacatgaagtggcgaacgggtgagtaacacgtgaccaacctgccccttgctccgggacaaccttgggaaaccgaggctaataccggatactcctcgcccccctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacggaaggcagcagggttggt

SEQ ID NO: 240 - DNA, 343 bp, artificial
  synthetic
gatgaacgctggcggcgtgcctaacacatgcaagtcgaacgggtgtacagaagggaagattacggtcggaagatctgtgcatgagtggcggacgggtgagtaacgcgtgggcaacctggcctgtacagggggataacacttagaaataggtgctaataccgcataacgggagaagccgcatggctttttcctgaaaactccggtggtacaggatgggcccgcgtctgattagccagttggcagggtaacggcctaccaaagcgacgatcagtagccggcctgagagggcggacggccacactgggactgagacacggcccagactcctacggaaggcagcagg

SEQ ID NO: 241 - DNA, 336 bp, artificial
  synthetic
gacgaacgctggcggcgcgcctaacacatgcaagtcgaacggagctatgttgaaagcttgctggatatatagcttagtggcggacgggtgagtaacacgtgagcaacctgcctttcagagtggaataacgtttggaaacgaacgctaataccgcataacgtcagaggacggcatcgtcttttgaccaaagatttatcgctgaaagatgggctcgcggccgattaggtagttggtgagataatagcccaccaagccgacgatcggtagccggactgagaggttgaacggccacattgggactgagacacggcccagactcctacggaaggcagcagg

SEQ ID NO: 242 - DNA, 330 bp, artificial
  synthetic
attgaacgctggcggcatgccttacacatgcaagtcgaacggcagcatggagagcttgctctctgatggcgagtggcgaacgggtgagtaatacatcggaacgtgtccgtttgtgggggacaaccgtccgaaaggatggctaataccgcataagacctgagggtgaaagccggggaccgcaaggcccggcgcagacggagcggccgatgattgattagctagttggggaggtaaaggctcaccaaggcgacgatcaatagctggtctgagaggacgaccagccacactggaactgagacacggtccagactcctacggaaggcagcaggg

SEQ ID NO: 243 - DNA, 344 bp, artificial
  synthetic
gatgaacgctggcggcgtgcttaacacatgcaagtcgagcgaagcagttaagaagattcttcggatgaaacttgattgactgagcggcggacgggtgagtaacgcgtgggtgacctgccccataccgggggataacagctggaaacggctgctaataccgcataagcgcacagagctgcatggctcagtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcaggg

SEQ ID NO: 244 - DNA, 345 bp, artificial
  synthetic
gatgaacgctggcggcgtgcttaacacatgcaagtcgaacgaagcgctttggatagatttcttcggattgatatccttagtgactgagtggcggacgggtgagtaacgcgtgggtaacctgcctcatacagggggataacagttagaaatggctgctaataccgcataagcgcacagtaccgcatggtacggtgtgaaaaactccggtggtatgagatggacccgcgtctgattagctagttggcagggtaacggcctaccaaggcgacgatcagtagccgacctgagagggtgatcggccacattgggactgagacacggcccaaactcctacggaaggcagca

SEQ ID NO: 245 - DNA, 336 bp, artificial
  synthetic
attgaacgctggcggcgtgcttaacacatgcaagtcgtacgcgaaagggacttcggtcccgagtaaagtggcgcacgggtgagtaacacgtggataatctgcctctatgatggggataacagttggaaacgactgctaataccgaatacgctcatgatgaactttgtgaggaaaggtggcctctgcttgcaagctatcgcatagagatgagtccgcgtcccattagctagttggtggggtaacggcctaccaaggcaacgatgggtagccgatctgagaggatgatcggccacactggaactgaaacacggtccagactcctacggaaggcagcag

SEQ ID NO: 246 - DNA, 337 bp, artificial
  synthetic
gatgaacgctagctacaggcttaacacatgcaagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatgagagaactattaaagaatttcggttatcgatggggatgcgttccattaggcagttggcggggtaacggcccaccaaaccgacgatggataggggttctgagaggaaggtcccccacattggaactgagacacggtccaaactcctacggaaggcagcagggt

SEQ ID NO: 247 - DNA, 344 bp, artificial
  synthetic
gatgaacgctagctacaggcttaacacatgcaagtcgaggggaaacgatattggaagcttgcttccgataggcgtcgaccggcgcacgggtgagtaacgcgtatccaacctgcccaccacttgggaataaccttgcgaaagtaagactaatgcccaatgacatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagggttggt

SEQ ID NO: 248 - DNA, 339 bp, artificial
  synthetic
gatgaacgctggcggcgtgcttaacacatgcaagtcgagcgaagcacttatgaagattcttcggatgattcatttgtgactgagcggcggacgggtgagtaacgcgtgagtaacctgcctcatacaggggaataacagttagaaatgactgctaatgccgcataagcgcacaggaccgcatggtctggtgtgaaaaactccggtggtatgagatggactcgcgtctgattagctagttggtgaggtaacggcccaccaaggcgacgatcagtagccggcctgagagggtgaacggccacattgggactgagacacggcccaaactcctacggaaggcag

SEQ ID NO: 249 - DNA, 336 bp, artificial
  synthetic
gacgaacgctggcggcgtgcctaacacatgcaagtcgaacggaagttagagcttcggttttaactttagtggcgaacgggtgagtaacgcgtgaagaacctgcctttcagtgggggacaacagttggaaacgactgctaataccgcatgatactttcgaggggcatcccttgaaagtcaaagctttatgtgctgaaagatggcttcgcgtctgattagctagttggtggggtaacggcccaccaaggcgacgatcagtagccggtctgagaggatgaacggccacattgggactgagatacggcccagactcctacggaaggcagcagggttggtt

SEQ ID NO: 250 - DNA, 365 bp, artificial
  synthetic
gatgaacgctggcggcgtgcctaatacatgcaagtcgaacgctttgtggttcaactgatttgaagagcttgctcagatatgacgatggacattgcaaagagtggcgaacgggtgagtaacacgtgggaaacctacctcttagcaggggataacatttggaaacagatgctaataccgtataacaatgacaaccgcatggttgttatttaaaagatggttctgctatcactaagagatggtcccgcggtgcattagctagttggtaaggtaatggcttaccaaggcgatgatgcatagccgagttgagagactgatcggccacaatgggactgagacacggcccatactcctacggaaggcagcag

SEQ ID NO: 251 - DNA, 325 bp, artificial
  synthetic
gatgaacgctagcgggaggcctaacacatgcaagctgagcggtagagatctttcgggatcttgagagcggcgtacgggtgcggaacacgtgtgcaacctgcctttatcagggggatagcctttcgaaaggaagattaataccccataatattttgagtggcatcacttgaaattgaaaactgaggtggataaagatgggcacgcgcaagattagatagttggtgaggtaacggctcaccaagtcgatgatctttagggggcctgagagggtgatcccccacactggtactgagacacggaccagactcctacggaaggcagcagg

SEQ ID NO: 252 - DNA, 334 bp, artificial
  synthetic
gatgaacgctggcggcgtgcttaacacatgcaagtcgaacgattaacccgccctcgggcggttatagagtggcgaacgggtgagtaacacgtgaccaacctgccccttgctccgggacaacccagggaaacctcggctaataccggatactccgcgcagcccgcatgggaggcgcgggaaagcccagacggcaagggatggggtcgcggcccattaggtagacggcggggtaacggcccaccgtgcccgcgatgggtagccgggttgagagaccgatcggccacattgggactgagatacggcccagactcctacggaaggcagcagggttggt

SEQ ID NO: 253 - DNA, 336 bp, artificial
  synthetic
gataaacgctggcggcgcacataagacatgcaagtcgaacggacttaaccattagtttactattggagcggttagtggcggactggtgagtaacacgtaagcaacctgcctatcagaggggaacaacagttagaaatgactgctaataccgcatatgcctaagtatcacatggtacaatagggaaaggagcaatccgctgatagatgggcttgcgtctgattagatagttggtggggtaacggcctaccaagtcgacgatcagtagccggactgagaggttgaacggccacattgggactgagatacggcccagactcctacgggaggcagcaggg

SEQ ID NO: 254 - DNA, 340 bp, artificial
  synthetic
gatgaacgctagctacaggcttaacacatgcaagtcgaggggcagcatgttggttgcttgcaaccaacgatggcgaccggcgcacgggtgagtaacgcgtatccaacctgcccttgtccatcggataacccgtcgaaaggcggactaacacgatatgcagtccacagcaggcatctaacgtggacgaaatgtgaaggagaaggatggggatgcgtctgattagcttgttggcggggtaacggcccaccaaggcgacgatcagtaggggttctgagaggaaggtcccccacattggaactgagacacggtccaaactcctacgggaggcagcagggttggt

SEQ ID NO: 255 - DNA, 344 bp, artificial
  synthetic
gatgaacgctagctacaggcttaacacatgcaagtcgaggggcagcatttcattttgcttgcaaagtgaagatggcgaccggcgcacgggtgagtaacacgtatccaacctgccgataactcggggatagcctttcgaaagaaagattaatatccgatagcatatcaatcccgcatgagattgatattaaagaatttcggttatcgatggggatgcgttccattagtttgttggcggggtaacggcccaccaagactacgatggataggggttctgagaggaaggtcccccacattggaactgagacacggtccaaactcctacgggaggcagcagggttggtt

SEQ ID NO: 256 - DNA, 338 bp, artificial
  synthetic
gatgaacgctagcggcaggcttaacacatgcaagtcgaagggcagcgtgggaagtgcttgcacttcccgacggcgactggcgcacgggtgagtaacacgtatgcaacctgccctccacagggggacaaccttccgaaagggaggctaatcccgcgtatatctaccggaggcatctccggtagaggaaagattcatcggtggaggatgggcatgcggcgcattagctagttggcggggcaacggcccaccaaggcgacgatgcgtaggggttctgagaggaaggtcccccacactggtactgagacacggaccagactcctacggaaggcagcagggtt

SEQ ID NO: 257 - DNA, 344 bp, artificial
  synthetic
gatgaacgctagctacaggcttaacacatgcaagtcgcggggtaacatctggtagagcttgctcttccaggatgacgaccggcgcacgggtgagtaacgcgtatccaacctggcccataccacgggatagcccgtcgaaaggcggattaataccgtatgctgtcattaagatgcatatattgatgacgaaaggactggcggtatgggatggggatgcgtctgattagcttgttggcggggtaacggcccaccaaggcgacgatcagtaggggttctgagaggaaggtcccccacattggaactgagacacggtccaaactcctacgggaggcagcagggttggt

SEQ ID NO: 258 - DNA, 343 bp, artificial
  synthetic
gatgaacgctggcggcgtgcttaacacatgcaagtcgaacgaagcgctttaccggatttcttcggaatgaaagttttgtgactgagtggcggacgggtgagtaacgcgtgggtaacctgcctcatacagggggataacagttagaaatgactgctaataccgcataagaccacaggaccgcatggtgcggtggtaaaaaatccggtggtatgagatggacccgcgtctgattagctagttggtaaggtaacggcttaccaaggcgacgatcagtagccgacctgagagggtgaccggccacattgggactgagacacggcccaaactcctacgggaggcagca

SEQ ID NO: 259 - DNA, 331 bp, artificial
  synthetic
gacgaacgctggcggcgcgcctaacacatgcaagtcgaacgagagagagggagcttgcttcctcaatcgagtggcgaacgggtgagtaacgcgtgaggaacctgcctcaaagagggggacaacagttggaaacgactgctaataccgcataagcccacaggtcggcatcgaccagagggaaaaggagcaatccgctttgagatggcctcgcgtccgattagctagttggtgaggtaatggcccaccaaggcaacgatcggtagccggactgagaggttgaacggccacattgggactgagacacggcccagactcctacggaaggcagcag

SEQ ID NO: 260 - DNA, 339 bp, artificial
  synthetic
gatgaacgctagcgacaggcttaacacatgcaagtcgaggggcagcgagggcgtagcaatacgcctgtcggcgaccggcgcactggtgagtaacacgtatgcgacctgccccctacaggggtataacccgtagaaatgcggactaatcccccatagtcctgggggctgcatggcattcaggggaaaggccagccggtaggggatgggcatgcggcgcattagctagttggcggggcaacggcccaccaaggcgacgatgcgtaggggttctgagaggaaggtcccccacactggtactgagacacggaccagactcctacgggaggcagcagggttggt

SEQ ID NO: 261 - DNA, 332 bp, artificial
  synthetic
gatgaacgctagctacaggcttaacacatgcaagtcgaggggcatcatgatctagcaataggttgatggcgaccggcgcacgggtgagtaacacgtatccaacctgcccttaactcggggatagcctcttgaaagagagattaatacccgatagtatatggttttcgcatgataaccatattaaagaatttcggttacggatggggatgcgttccattagatagtaggcggggtaacggcccacctagtcttcgatggataggggttctgagaggaaggtcccccacactggtactgagacacggaccagactcctacggaaggcagcaggg

SEQ ID NO: 262 - DNA, 337 bp, artificial
  synthetic
gatgaacgctagctacaggcttaacacatgcaagtcgaggggcagcaggatctagcaatagattgctggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacggaaggcagcagggttggt

SEQ ID NO: 263 - DNA, 331 bp, artificial
  synthetic
attgaacgctggcggcatgctttacacatgcaagtcggacggcagcataaaagagcttgctcttttgatggcgagtggcgaacgggtgagtaatgcatcggaacgtaccgagtagtgggggataactgtccgaaaggatggctaataccgcatattctctgaggaggaaagcaggggaccttagggccttgcgctatttgagcggccgatgtctgattagctagttggtggggtaagagcccaccaaggcgacgatcagtagcgggtctgagaggatgatccgccacactgggactgagacacggcccagactcctacggaaggcagcagg

SEQ ID NO: 264 - DNA, 345 bp, artificial
  synthetic
gatgaacgctggcggcgtgcttaacacatgcaagtcgagcgaagcggtttcaatgaagttttcggatggatttgaaattgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacggaaggcagcagg

SEQ ID NO: 265 - DNA, 345 bp, artificial
  synthetic
gatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacggaaggcagcagg

SEQ ID NO: 266 - DNA, 379 bp, artificial
  synthetic
gataaacgctggcggcgcacataagacatgcaagtcgaacggaagtcgttgtaatgatatagacatggacagagaacttgttcgaagaaagtgaaagttgacttacaacaatggctttagtggcggactggtgagtaacacgtaagcaacctgcctatcagaggggaacaacagttagaaatgactgctaataccgcatatgcctaagtaccacatggtacaatagggaaaggagcaatccgctgatagatgggcttgcgtctgattagatagttggtggggtaacggcctaccaagtcaacgatcagtagccggactgagaggttgaacggccacattgggactgagatacggcccagactcctacgggaggcagcag

SEQ ID NO: 267 - DNA, 342 bp, artificial
  synthetic
gacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacggaaggcagcagggttgg

SEQ ID NO: 268 - DNA, 323 bp, artificial
  synthetic
aacgaacgctggcggcaggcttaacacatgcaagttgaacgtgatttgctgggtgcttgcaccgagcaatgaaagtagcgcactggtgagtaacacgtgggaacgtaccttttggtggggaacaacagttggaaacgactgctaataccgcataagccctgagggggaaagatttatcgccgaaagaacggcccgcggaagattaggtagttggtggggtaacggcctaccaagccgacgatctatagctggtctgagaggacgatcagccacattggaactgagacacggtccaaactcctacggaaggcagcagggttggt

SEQ ID NO: 269 - DNA, 322 bp, artificial
  synthetic
attgaacgctggcggcatgctttacacatgcaagtcgaacggtaacaggccgcaaggtgctgacgagtggcgaacgggtgagtaatgcatcggaacgtgcccagtcgtgggggataactactcgaaagagtagctaataccgcatacgatctatggatgaaagcgggggaccgtaaggcctcgcgcgattggagcggccgatgtcagattaggtagttggtggggtaaaggctcaccaagccaacgatctgtagctggtctgagaggacgaccagccacactgggactgagacacggcccagactcctacggaaggcagcag

SEQ ID NO: 270 - DNA, 324 bp, artificial
  synthetic
gatgaacgctggcggcgtgcctaatacatgcaagtcgaacgcgagcagcaatgctcgagtggcgaacgggtgagtaatacataagtaacctgccctagacagggggataactgctggaaacggcagctaagaccgcataggtatggacactgcatggtgaccatattaaaagtgccaaggcactggtaggaggatggacttatggcgcattagctggttggtgaggtaacggctcaccaaggcgacgatgcgtagccgacctgagagggtgaccggccacactgggactgagacacggcccagactcctacggaaggcagcagg

SEQ ID NO: 271 - DNA, 327 bp, artificial
  synthetic
attgaacgctggcggaatgctttacacatgcaagtcgaacggcagcacgggggcaaccctggtggcgagtggcgaacgggtgagtaatacatcggaacgtgcccaatcgtgggggataacgtagcgaaagctacgctaataccgcatacgatctacggatgaaagcgggggatcgcaagacctcgcgcgagtggagcggccgatggcagattaggtagttggtggggtaaaggctcaccaagcctgcgatctgtagctggtctgagaggacgaccagccacactgggactgagacacggcccagactcctacggaaggcagcagggt

SEQ ID NO: 272 - DNA, 348 bp, artificial
  synthetic
gatgaacgctggcggcgtgcttaacacatgcaagtcgaacgaagcgcataagacagattctttcgggatgatgttttctgtgactgagtggcggacgggtgagtaacgcgtgggcaacctgccttatacagggggataacagttagaaatgactgctaataccgcataagaccacagtaccgcatggtacaggggtaaaagctcttgcagtataagatgggcccgcgtctgattagctggttggtgaggtaacggctctaccaaggcaacgatcagtagccggcttgagagagtggacggccacattgggactgagacacggcccaaactcctacgggaggcagcagg

SEQ ID NO: 273 - DNA, 339 bp, artificial
  synthetic
gatgaacgctagctacaggcttaacacatgcaagtcgaggggcagcatgaaaggaacttcggttctttttgatggcgaccggcgcacgggtgcgtaacgcgtatcgaacctgcctcatactcggggatagccttgcgaaagtaagattaatacccggcggtctcggatggccgcatgactatccgagtgaagatttatcggtatgagatggcgatgcgtcccattagctagttggtgaggtaacggctcaccaaggcatcgatgggtaggggttctgagaggaaggtcccccacattggaactgagacacggtccaaactcctacggaaggcagcaggg

SEQ ID NO: 274 - DNA, 322 bp, artificial
  synthetic
attgaacgctggcggcatgccttacacatgcaagtcgaacggtaacaggtcttcggatgctgacgagtggcgaacgggtgagtaatacatcggaacgtgcctagtagtgggggataactactcgaaagagtggctaataccgcatgagatctacggatgaaagcaggggatcgcaagaccttgtgctactagagcggccgatggcagattaggtagttggtgggataaaagcttaccaagccgacgatctgtagctggtctgagaggacgatcagccacactgggactgagacacggcccagactcctacggaaggcagcag

SEQ ID NO: 275 - DNA, 299 bp, artificial
  synthetic
aacgaacgctggcggcatgcctaacacatgcaagtcgaacgatgctttcgggcatagtggcgcacgggtgcgtaacgcgtgggaatctgcccttaggttcggaataacagttagaaatgactgctaataccggatgatgacgtaagtccaaagatttatcgccttgggatgagcccgcgtaggattagctagttggtgtggtaaaggcgcaccaaggcgacgatccttagctggtctgagaggatgatcagccacactgggactgagacacggcccagactcctacggaaggcagcagg

SEQ ID NO: 276 - DNA, 341 bp, artificial
  synthetic
gatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtatttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacggaaggcagcagggttggt

SEQ ID NO: 277 - DNA, 335 bp, artificial
  synthetic
gatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacggaaggcagcagggt

SEQ ID NO: 278 - DNA, 339 bp, artificial
  synthetic
gatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacggaaggcagcaggg

SEQ ID NO: 279 - DNA, 342 bp, artificial
  synthetic
gatgaacgctggcggcgtgcctaacacatgcaagtcgaacggagttatgcagaggaagttttcggatggaatcggcgtaacttagtggcggacgggtgagtaacgcgtgggaaacctgccctgtaccgggggataacacttagaaataggtgctaataccgcataagcgcacagcttcacatgaggcagtgtgaaaaactccggtggtacaggatggtcccgcgtctgattagccagttggcagggtaacggcctaccaaagcgacgatcagtagccggcctgagagggtgaacggccacattgggactgagacacggcccaaactcctacggaaggcagca

SEQ ID NO: 280 - DNA, 334 bp, artificial
  synthetic
gacgaacgctggcggcgtgcctaacacatgcaagtcgaacggggttatttcttcggagataacttagtggcgaacgggtgagtaacgcgtgagaaacctgcctttcagtgggggacaacagttggaaacgactgctaataccgcatgatactttctgggggcatccctggaaagtcaaagctttatgtgctgaaagatggtctcgcgtctgattagctagttggtggggtaacggcccaccaaggcgacgatcagtagccggtctgagaggatgaacggccacattgggactgagatacggcccagactcctacggaaggcagcagggttggtt

SEQ ID NO: 281 - DNA, 329 bp, artificial
  synthetic
attgaacgctggcggcatgccttacacatgcaagtcgaacggtaacaggtcttcggacgctgacgagtggcgaacgggtgagtaatgcatcggaacgtgcccagtcgtgggggataactgctcgaaagagcagctaataccgcatacgacctgagggtgaaagcgggggaccgtaaggcctcgcgcgattggagcggccgatgtcagattagctagttggtggggtaaaggcctaccaaggcaacgatctgtagttggtctgagaggacgaccagccacactgggactgagacacggcccagactcctacggaaggcagcagggttggt

SEQ ID NO: 282 - DNA, 346 bp, artificial
  synthetic
gatgaacgctggcggcgtgcctaacacatgcaagtcgaacggagttactttggtgaagttttcggatggatctgatttaacttagtggcggacgggtgagtaatgcgtgagcaacctgcccttcagagggggacaacagttggaaacgactgctaataccgcataatgtattcggaaggcatcttctgaataccaaaggagaaatccgctgagggatgggctcacgtctgattagttagttggtgaggtaacggctcaccaagactgcgatcagtagccggactgagaggttgaacggccacattgggactgagatacggcccagactcctacggaaggcagcagg

SEQ ID NO: 283 - DNA, 305 bp, artificial
  synthetic
aacgaacgctggcggcatgcctaacacatgcaagtcgaacgaaggcttcggccttagtggcgcacgggtgcgtaacgcgtgggaatctgcccttgggtctgggataacagcgggaaacggctgctaataccggatgatgacgtaagtccaaagatttatcgcccagggatgagcccgcgtaggattagctagttggtggggtaaaggcccaccaaggcgacgatccttagctggtctgagaggatgatcagccacactgggactgagacacggcccagactcctacgggaggcagcagggttggt

SEQ ID NO: 284 - DNA, 351 bp, artificial
  synthetic
gatgaacgctggcggcgtgcttaacacatgcaagtcgaacgaagcaccttgatttgattcttcggatgaagatcttggtgactgagtggcggacgggtgagtaacgcgtgggtaacctgcctcatacagggggataacagttagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacggaaggcagcagggttggt

SEQ ID NO: 285 - DNA, 335 bp, artificial
  synthetic
gatgaacgctggcggcgtgcttaacacatgcaagtcgaacgaagcacctggatttcttcggattgataggtgactgagtggcggacgggtgagtaacgcgtgggtaacctgcctcatacagggggataacagttagaaatgactgctaataccgcataagcgcacagtgctgcatggcacagtgtgaaaaactccggtggtatgagatggacccgcgtctgattagctagttggtgaggtaacggcccaccaaggcgacgatcagtagccggcctgagagggtgaccggccacattgggactgagacacggcccaaactcctacgggaggcagca

SEQ ID NO: 286 - DNA, 332 bp, artificial
  synthetic
gacgaacgctggcggcgtgcctaacacatgcaagtcgaacggacgaggaggagcttgcttctccaagtcagtggcggacgggtgagtaacgcgtgagtaacctgcccttcagagggggataacgtctggaaacggacgctaataccgcatgacatatttccatcgcatggtggagatatcaaaggagcaatccgctgaaggatggactcgcgtccgattagatagttggcggggtaacggcccaccaagtcgacgatcggtagccggactgagaggttgaacggccacattgggactgagacacggcccagactcctacggaaggcagcagg

SEQ ID NO: 287 - DNA, 343 bp, artificial
  synthetic
gatgaacgctggcggcgtgcctaacacatgcaagtcgagcgaagcgatcgagatgaagttttcggatggattcctgattgactgagcggcggacgggtgagtaacgcgtgggtaacctgcctcatacagggggataacagttagaaatgactgctaataccgcataagcgcacagtaccacatggtacggtgtgaaaaactccggtggtatgagatggacccgcgtctgattagctagttggtggggtaacggcccaccaaggcgacgatcagtagccgacctgagagggtgatcggccacattgggactgagacacggcccaaactcctacggaaggcagca

SEQ ID NO: 288 - DNA, 338 bp, artificial
  synthetic
gatgaacgctagctacaggcttaacacatgcaagtcgaggggaaacgatagggaaagcttgctttccctaggcgtcgaccggcgcacgggtgagtaacgcgtatccaacctgcccatgtctggggaataacccgtcgaaaggcggactaactccccatggtctccgatgaggacatctgaattggagtaaagcttcgcggacatggatggggatgcgtctgattaggtagtaggcggggtaacggcccacctagcctacgatcagtaggggttctgagaggaaggtcccccacattggaactgagacacggtccaaactcctacggaaggcagcaggg

SEQ ID NO: 289 - DNA, 335 bp, artificial
  synthetic
gatgaacgctggcggcgtgcttaacacatgcaagtcgaacgaagatgccggagtgcttgcactttggcagactgagtggcggacgggtgagtaacgcgtgggtaacctgccttatacagggggataacagttggaaacgactgctaataccgcataagcgcacggtatcgcatgatacagtgtgaaaaactccggtggtataagatggacccgcgtctgattagcttgttggtgaggtaacggcccaccaaggcgacgatcagtagccggcctgagagggtgaacggccacattgggactgagacacggcccaaactcctacgggaggcagcagg

SEQ ID NO: 290 - DNA, 332 bp, artificial
  synthetic
attgaacgctggcggcatgctttacacatgcaagtcgaacggtaacagggtgcttgcaccgctgacgagtggcgaacgggtgagtaatgcatcggaacgtacccagtcgtgggggataacgtagcgaaagttacgctaataccgcatacgtcctgagggagaaagcgggggaccgtaaggcctcgcgcgattggagcggccgatgtcggattagctagttggtagggtaaaggcctaccaaggcgacgatccgtagcgggtctgagaggatgatccgccacactgggactgagacacggcccagactcctacggaaggcagcagggttggtt

SEQ ID NO: 291 - DNA, 340 bp, artificial
  synthetic
gacgaacgctggcggcgtgcttaacacatgcaagtcgaacggagaacttatttcggtaagttcttagtggcgaacgggtgagtaacgcgtgggcaacctgccctccagttggggacaacattccgaaagggatgctaataccgaatgtgctccctcctccgcatggaggagggaggaaagatggcctctgcttgcaagctatcgctggaagatgggcccgcgtctgattagctagttggtggggtaacggctcaccaaggcgatgatcagtagccggtctgagaggatgaacggccacattgggactgagacacggcccaaactcctacggaaggcagca

SEQ ID NO: 292 - DNA, 343 bp, artificial
  synthetic
gatgaacgctggcggcgtgcctaatacatgcaagtagaacgctgaagcttggtgcttgcaccgagcggatgagttgcgaacgggtgagtaacgcgtaggtaacctgcctcttagcgggggataactattggaaacgatagctaataccgcataaaagtcgacattgcatgatgttgacttgaaaggtgcaattgcatcactaagagatggacctgcgttgtattagctagttggtgaggtaacggctcaccaaggcgacgatacatagccgacctgagagggtgatcggccacactgggactgagacacggcccagactcctacggaaggcagcagggttggt

SEQ ID NO: 293 - DNA, 341 bp, artificial
  synthetic
gatgaacgctagcgacaggcctaacacatgcaagtcgaggggcagcgggggcgaagcttgctttgcctgccggcgaccggcgcacgggtgagtaacacgtatggaacctgcccgccacagggggataaccggtagaaatgccgactaataccccgtatgcccacaggggcgcatgccctggtggggaaacattcatgggtggcggatggccatgcggcgcattagctggttggcggggtaacggcccaccaaggcgacgatgcgtaggggttctgagaggaaggtcccccacattggtactgagacacggaccaaactcctacgggaggcagcagggttgg

SEQ ID NO: 294 - DNA, 190 bp, artificial
  synthetic
gagacgcttggaccaaaagggctgaggcagtgcatatctgcactgggagggaagagctgggaagccagcacagacatgctgtcgggggttccagggggctagaaagggatagtgtagactctgggctttgagcaggctctggagtaaggggtaagataagaagtggctgtacggaaggcagcagggttgg

SEQ ID NO: 295 - DNA, 342 bp, artificial
  synthetic
gatgaacgctggcggcgtgcctaacacatgcaagtcgaacgggtgtacggggaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacggaaggcagcag

SEQ ID NO: 296 - DNA, 338 bp, artificial
  synthetic
gatgaacgctagctacaggcttaacacatgcaagtcgaggggcatcatggcggtcgcttgcgaccgctgatggcgaccggcgcacgggtgcgtaacgcgtatcgaacctgccgcatacccggggatagccttgcgaaagcaagattaatacccgatgttctcactgttccgcatgttacggtgagcaaagaatttcggtatgcgatggcgatgcgtcccattagctagttggcggggtaacggcccaccaaggctccgatgggtaggggttctgagaggaaggtcccccacactggaactgagacacggtccagactcctacgggaggcagcagggtt

SEQ ID NO: 297 - DNA, 350 bp, artificial
  synthetic
gatgaacgctggcggcgtgcctaacacatgcaagtcgagcgagaagctcatgacggacgcttcggttgaagtcacgagtggaaagcggcggacgggtgagtaacgcgtaggcaacctgccctttgcagagggatagcctcgggaaaccgggattaaaacctcataataccgcagtgagacatctcacggcggtcaaagatttatcggcaaaggatgggcctgcgtctgattagttagttggtggggtaacggcctaccaaggcgacgatcagtagccgacctgagagggtgatcggccacattggaactgagacacggtccaaactcctacgggaggcagcagggttggt

SEQ ID NO: 298 - DNA, 346 bp, artificial
  synthetic
gatgaacgctggcggcgtgcttaacacatgcaagtcgaacggagtgctcatgacagaggattcgtccaatggattgagttacttagtggcggacgggtgagtaacgcgtgaggaacctgcctcggagtggggaataacacaacgaaagctgtgctaataccgcatgatacagttgggtcgcatgactctgactgtcaaagatttatcgctctgagatggcctcgcgtctgattagctagttggcggggtaacggcccaccaaggcgacgatcagtagccggactgagaggttgaccggccacattgggactgagacacggcccagactcctacggaaggcagcagg

SEQ ID NO: 299 - DNA, 345 bp, artificial
  synthetic
gatgaacgctggcggcgtgcttaatacatgcaagtcgagcgaagcgctttaacggaattcttcggaaggaagataaagtgactgagcggcggacgggtgagtaacgcgtgggtaacctgcctcatacagggggataacagttagaaatggctgctaataccgcataagcgcacaggaccgcatggtctggtgtgaaaaactccggtggtatgggatggacccgcgttggattagctagttggtggggtaacggcctaccaaggcgacgatccatagccggcctgagagggtggacggccacattgggactgagacacggcccagactcctacggaaggcagcagg

SEQ ID NO: 300 - DNA, 342 bp, artificial
  synthetic
gatgaacgctagctacaggcttaacacatgcaagtcgaggggaaacgacatcgaaagcttgcttttgatgggcgtcgaccggcgcacgggtgagtaacgcgtatccaacctgcccaccacttggggataaccttgcgaaagtaagactaatacccaatgatatcccacgaaggcatccgaatgggattaaagatttatcggtgatggatggggatgcgtctgattagcttgttggcggggtaacggcccaccaaggcaacgatcagtaggggttctgagaggaaggtcccccacattggaactgagacacggtccaaactcctacgggaggcagcagggttg

SEQ ID NO: 301 - DNA, 345 bp, artificial
  synthetic
gatgaacgctggcggcgtgcttaacacatgcaagtcgaacggggattattttgacagagacttcggttgaagtcgttataatcctagtggcggacgggtgagtaacgcgtgggtaacctgcctcacactgggggataacagtcagaaatgactgctaataccgcataagcgcacgggattgcatgatccagtgtgaaaaactccggtggtgtgagatggacccgcgttggattagccagttggcagggtaacggcctaccaaagcgacgatccatagccggcctgagagggtggacggccacattgggactgagacacggcccagactcctacggaaggcagcag

SEQ ID NO: 302 - DNA, 338 bp, artificial
  synthetic
gatgaacgctagcggcaggcctaacacatgcaagtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacggaaggcagcagggttggtt

SEQ ID NO: 303 - DNA, 350 bp, artificial
  synthetic
gatgaacgctggcggcgtgcttaacacatgcaagtcgagcgaagcacttatgatgattcttcggatgaatcatttgtgactgagcggcggacgggtgagtaacgcgtgagtaacctgcctcatacaggggaataacagttagaaatgactgctaatgccgcataagcgcacagggccgcatggcccggtgtgaaaaactccggtggtatgagatggactcgcgtctgattagctagttggcagggtaacggcctaccaaggcgacgatcagtagccggcctgagagggtgaacggccacattgggactgagacacggcccaaactcctacgggaggcagcagggtttttt

SEQ ID NO: 304 - DNA, 337 bp, artificial
  synthetic
gatgaacgctagctacaggcttaacacatgcaagtcgaggggcagcattacggtagcttgctactgtagatggcgaccggcgcacgggtgagtaacgcgtatccaacctgcccatgacttggggataaccttgcgaaagcaagcctaatacccgatacgcttcatagatggcatctgaaatgaagtaaaggtttacggttatggatggggatgcgtccgattagcttgttggcggggtaacggcccaccaaggcatcgatcggtaggggttctgagaggaaggtcccccacattggaactgagacacggtccaaactcctacggaaggcagcagggt

SEQ ID NO: 305 - DNA, 348 bp, artificial
  synthetic
gacgaacgctggcggcgtgcttaacacatgcaagtcgaacggagaactttcttcggaatgttcttagtggcgaacgggtgagtaacgcgtaggcaacctgccctctggttggggacaacattccgaaagggatgctaataccgaatgagatcctctttccgcatggagagaggatgaaagatggcctctacttgtaagctatcgccagaagatgggcctgcgtctgattagcttgtaggtgaggtaacggctcacctaggcgatgatcagtagccggtctgagaggatgaacggccacattgggactgagacacggcccaaactcctacggaaggcagcagggttggt

SEQ ID NO: 306 - DNA, 354 bp, artificial
  synthetic
gatgaacgctggcggcgtgcctaacacatgcaagtcgaacggaactattttggaaactccttcgggagcggaatctttagtttagtggcggacgggtgagtaacgcgtgagcaatctgcctttaagagggggataacagtcggaaacggctgctaataccgcataaagcatcaaattcgcatgtttttgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagggttggttt

SEQ ID NO: 307 - DNA, 335 bp, artificial
  synthetic
gacgaacgctggcggcgtgcctaatacatgcaagtcgagcgaatcgacgggagcttgctccctgagattagcggcggacgggtgagtaacacgtgggcaacctgcctataagactgggataacttcgggaaaccggagctaataccggatacgttcttttctcgcatgagagaagatggaaagacggtttacgctgtcacttatagatgggcccgcggcgcattagctagttggtgaggtaatggctcaccaaggcgacgatgcgtagccgacctgagagggtgatcggccacactgggactgagacacggcccagactcctacgggaggcagca

SEQ ID NO: 308 - DNA, 337 bp, artificial
  synthetic
gatgaacgctagcggcaggcttaacacatgcaagtcgaggggcagcgtggagcagcaatgctctgacggcgaccggcggaagggtgcgtaacacgtgagcgacttgcccgtcacagggggataaccggcggaaacggcgactaataccccatatgatgtgatgctgcatggcattgcattgaaagattcatcggtggcggataggctcgcgggacattagctagacggcggggcaacggcccaccgtggcgacgatgtctaggggttctgagaggaagaccccccacactgggactgagacacggcccagactcctacgggaggcagcagggttggt

SEQ ID NO: 309 - DNA, 348 bp, artificial
  synthetic
gatgaacgctggcggcgtgcttaacacatgcaagtcgaacgaggaattttaaatgagacttcggtggattttaaaattcttagtggcggacgggtgagtaacgcgtgggcaacctgccctgtacagggggacaacagctggaaacggctgctaataccgcataagcgcacagcatcgcatgatgccgtgtgaaaaactccggtggtacaggatgggcccgcgttggattaggtagttggtgaggtaacggcccaccaagccgacgatccatagccgacctgagagggtgaccggccacattgggactgagacacggcccaaactcctacgggaggcagcagggttggt

SEQ ID NO: 310 - DNA, 341 bp, artificial
  synthetic
gatgaacgctggcggcgtgcttaacacatgcaagtcgagcgaagcggtttaaatgagacttcggtggattttaaattgactgagcggcggacgggtgagtaacgcgtggataacctgcctcacacagggggataacagttagaaatgactgctaataccgcataagcgcacggcatcgcatgatgcagtgtgaaaaactccggtggtgtgagatggatccgcgtctgattaggtagttggtggggtaacggcccaccaagccgacgatcagtagccgacctgagagggtgaccggccacattgggactgagacacggcccaaactcctacgggaggcagca

SEQ ID NO: 311 - DNA, 351 bp, artificial
  synthetic
gatgaacgctggcggcgtgcttaacacatgcaagtcgaacgaagcacttgaaacagattcttcggatgaagtttcctgtgactgagtggcggacgggtgagtaacgcgtgggtaacctgcctcatacagggggataacagttagaaatggctgctaataccgcataagcgcacggcatcgcatgatgcagtgtgaaaaactccggtggtatgagatggacccgcgttggattagctagttggcagggtaacggcctaccaaggcgacgatccatagccggcctgagagggtggacggccacattgggactgagacacggcccagactcctacggaaggcagcagggttggt

SEQ ID NO: 312 - DNA, 346 bp, artificial
  synthetic
gatgaacgctggcggcgtgcttaacacatgcaagtcgagcgaagcacttttacggatttcttcggattgaagtgaaagtgactgagcggcggacgggtgagtaacgcgtgggtaacctgcctcatacagggggataacagttagaaatgactgctaataccgcataagcgcacagtaccgcatggtacggtgtgaaaaactccggtggtatgagatggacccgcgtctgattaggtagttggtggggtaacggcctaccaagccaacgatcagtagccgacctgagagggcgaccggccacattgggactgagacacggcccaaactcctacgggaggcagcaggg

SEQ ID NO: 313 - DNA, 344 bp, artificial
  synthetic
gacgaacgctggcggcgtgcttaacacatgcaagtcgaacgagaatctgctgaaggaggattcgtccaacggaagtagaggaaagtggcggacgggtgagtaacgcgtgaggaacctgccttgaagagggggacaacagttggaaacgactgctaataccgcatgacgcatagaggtcgcatgatctttatgccaaagatttatcgcttcaagatggcctcgcgtctgattagctggttggcggggtaacggcccaccaaggcgacgatcagtagccggactgagaggttgaacggccacattgggactgagatacggcccagactcctacgggaggcagcagg

SEQ ID NO: 314 - DNA, 351 bp, artificial
  synthetic
gacgaacgctggcggcgtgcttaacacatgcaagtcgaacggagcacccttgattgaggtttcggccaaatgataggaatgcttagtggcggactggtgagtaacgcgtgaggaacctgcctttcagagggggacaacagttggaaacgactgctaataccgcatgacgcattttgatcgcatggtcgaaatgccaaagatttatcgctgaaagatggcctcgcgtctgattagctagttggtgaggtaacggcccaccaaggcgacgatcagtagccggactgagaggttgaccggccacattgggactgagatacggcccagactcctacggaaggcagcagggttggt

SEQ ID NO: 315 - DNA, 348 bp, artificial
  synthetic
gatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagggtt

SEQ ID NO: 316 - DNA, 185 bp, artificial
  synthetic
tatcatatgaggacccagggaggcagccagacatggctagctgcctccagctctaggttcatgctgagcagggtggagaagaagggaggcctggagctgaggatgtctgctgagaacttctcggaggatctgaggatcttctgtgcttaccaatacagagtcacacgggaggcagcagggttggt

SEQ ID NO: 317 - DNA, 349 bp, artificial
  synthetic
gacgaacgctggcggcgtgcttaacacatgcaagtcgaacggggcgaagtgatagcttgctattatggagcctagtggcaaacgggtgagtaacgcgtaggcaacctgcccttcagatggggacaacacctcgaaaggggtgctaataccgaatgacgtttcctggtcgcatgacctggaaaccaaaggccgggcaaccggtcactgaaggatgggcctgcgtctgattagctagttggtggggtaacggcccaccaaggcgacgatcagtagccggtctgagaggatgaacggccacattgggactgagacacggcccaaactcctacgggaggcagcagggttggtt

SEQ ID NO: 318 - DNA, 345 bp, artificial
  synthetic
gatgaacgctggcggcgtgcttaacacatgcaagtcgagcgaagcgctttacttagatttcttcggattgaagagttttgcgactgagcggcggacgggtgagtaacgcgtgggtaacctgcctcatacagggggataacagttagaaatgactgctaataccgcataagaccacggtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagca

SEQ ID NO: 319 - DNA, 344 bp, artificial
  synthetic
gatgaacgctagcgacaggcttaacacatgcaagtcgaggggcagcggggagtgaggcttgccttactttgccggcgaccggcgcacgggtgagtaacacgtatgcaacctaccctttacagcgggataaccggaagaaatttcgcctaataccgcatatactccttgggaggcatcttccgagggggaaagaatttcggtgaaggatgggcatgcgtcgcattagttagttggcggtgtaacggaccaccaagacgacgatgcgtaggggttctgagaggaaggtcccccacactggtactgagacacggaccagactcctacgggaggcagcagggttggtt

SEQ ID NO: 320 - DNA, 333 bp, artificial
  synthetic
gatgaacgctggcggcgtgcctaatacatgcaagtcgaacgcgatagtttactatcgagtggcgaacgggtgagtaatacataagtaacctgccctagacagggggataactattggaaacgatagctaataccgcataggtgtagacactgcatggtgactgcattaaagatgctttaaagcatcggtagaggatggacttatggcgcattagctagttggtagggtaaaggcctaccaaggcgacgatgcgtagccgacctgagagggtgaccggccacactgggactgagacacggcccagactcctacgggaggcagcagggttggttt

SEQ ID NO: 321 - DNA, 353 bp, artificial
  synthetic
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagcttgttggcagggtaacggcctaccaaggcgacgatcagtagccggcctgagagggtgaacggccacattgggactgagacacggcccaaactcctacggaaggcagcagggttggt

SEQ ID NO: 322 - DNA, 330 bp, artificial
  synthetic
gatgaacgctggcggcgcgcctaacacatgcaagtcgaacggcacctaccctcgggtagaagcgagtggcgaacggctgagtaacacgtggagaacctgccccctcccccgggatagccgcccgaaaggacgggtaataccggatacccccgggcgccgcatggcgcccgggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagggttggt

SEQ ID NO: 323 - DNA, 333 bp, artificial
  synthetic
attgaacgctggcggcaggcctaacacatgcaagtcgaacggtagcagaaagaagcttgcttctttgctgacgagtggcggacgggtgagtaatgtctgggaaactgcccgatggagggggataactactggaaacggtagctaataccgcataacgtcttcggaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagggt

SEQ ID NO: 324 - DNA, 331 bp, artificial
  synthetic
gatgaacgctggcggcatgcctaatacatgcaagtcgaacgaatcacattagtgattagtggcgaacgggtgagtaacacgtagggaacctggccgcattcgggggatacgctctggaaacggagtctaaaaccccataggaaatgagaaggcatcttctcattttgaaacatgctttcaagcatggaaggcggatggacctgcggtgcattagcttgttggcaaggtaaaagcttaccaaggcgatgatgcatagccggcctgagagggcggacggccacactgggactgagacacggcccagactcctacggaaggcagcagggttggt

SEQ ID NO: 325 - DNA, 334 bp, artificial
  synthetic
gatgaacgctggcggcgtgcttaacacatgcaagtcgaacgattaagacggcttcggccgtgtatagagtggcgaacgggtgagtaacacgtgaccaacctgccccgcgctccgggacaaccgctggaaacggcggctaataccggatgctccggggaggccccatggcctccccgggaaagccttaacggcgcgggatggggtcgcggcccattaggtagacggcggggtaacggcccaccgtgcccgcgatgggtagccggactgagaggtcgaccggccacattgggactgagatacggcccagactcctacggaaggcagcagggttggt

SEQ ID NO: 326 - DNA, 338 bp, artificial
  synthetic
gatgaacgctagcggcaggcctaacacatgcaagtcgaggggcatcgggattgaagcttgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacggaaggcagcagggtt

SEQ ID NO: 327 - DNA, 344 bp, artificial
  synthetic
gatgaacgctagctacaggcttaacacatgcaagtcgaggggaaacgatagagaaagcttgctttttctaggcgtcgaccggcgcacgggtgagtaacgcgtatccaacctgcccaccacttggggataaccttgcgaaagtaagactaatacccaatgacgtctctagaagacatctgaaagggattaaagatttatcggtgatggatggggatgcgtctgattagcttgttggcggggtaacggcccaccaaggcaacgatcagtaggggttctgagaggaaggtcccccacattggaactgagacacggtccaaactcctacgggaggcagcagggttggt

SEQ ID NO: 328 - DNA, 331 bp, artificial
  synthetic
gatgaacgctggcggcgcgcctaacacatgcaagtcgaacggcacccaccctcgggtggaagcgagtggcgaacggctgagtaacacgtggagaacccgccccctcctccgggatagcctcgggaaaccgtgggtaataccggatgaccccgcagggccgcatggccccgcgggcaaagcccagacgggaggggatggctccgcggcccatcaggtagacggcggggtgacggcccaccgtgccgacgacgggtagccgggttgagagaccgaccggccagattgggactgagacacggcccagactcctacggaaggcagcagggttggt

SEQ ID NO: 329 - DNA, 298 bp, artificial
  synthetic
aacgaacgctggcggcaggcttaacacatgcaagtcgaacgccccgcaaggggagtggcagacgggtgagtaacgcgtgggaacataccctttcctgcggaatagctccgggaaactggaattaataccgcatacgcctacggggaagattatcggggaaggattggcccgcgttggattagctagttggtggggtaaaggcctaccaaggcgacgatccatagctggtctgagaggatgatcagccacattgggactgagacacggcccaaactcctacgggaggcagcagggttgg

SEQ ID NO: 330 - DNA, 339 bp, artificial
  synthetic
gatgaacgctagctacaggcttaacacatgcaagtcgaggggcatcatgcttatagcaatataagtgatggcgaccggcgcacgggtgagtaacacgtatccaacctgccgataactcggggatagcctttcgaaagaaagattaatacccgatggcataatagaaccgcatggtttgattattaaagaatttcggttatcgatggggatgcgttccattaggcagttggtggggtaacggcccaccaaaccttcgatggataggggttctgagaggaaggtcccccacattggaactgagacacggtccaaactcctacggaaggcagcagggttggt

SEQ ID NO: 331 - DNA, 321 bp, artificial
  synthetic
agtgaacgctggcggcgtgcctaatacatgcaagtcgaacgatgaagcttttagcttgctagaagtggattagtggcgcacgggtgagtaatgcataggttatgtgcctcatagtctaggatagccattggaaacgatgattaatactggatactccctacgggggaaagtttttcgctatgagatcagcctatgtcctatcagcttgttggtgaggtaatggctcaccaaggctatgacgggtatccggcctgagagggtgaacggacacactggaactgagacacggtccagactcctacggaaggcagcagggttggt

SEQ ID NO: 332 - DNA, 343 bp, artificial
  synthetic
gatgaacgctggcggcgtgcttaacacatgcaagtcgaacgaagcactttatttgatttcttcggaatgaagattttgtgactgagtggcggacgggtgagtaacgcgtgggtaacctgcctcatacagggggataacagttggaaacgactgctaataccgcataagcgcacggcatcgcatgatgcagtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagca

SEQ ID NO: 333 - DNA, 338 bp, artificial
  synthetic
gacgaacgctggcggcgcgcctaacacatgcaagtcgaacggagcttaaagagcttgctttttaagcttagtggcgaacgggtgagtaacgcgtgagtaacctgccctggagtgggggacaacagttggaaacgactgctaataccgcataagcccacggattcgcatggatctgcgggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagggttggt

SEQ ID NO: 334 - DNA, 345 bp, artificial
  synthetic
gacgaacgctggcggcgtgcttaacacatgcaagtcgaacggagcacccttgactgaggtttcggccaaatgataggaatgcttagtggcggactggtgagtaacgcgtgaggaacctaccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacggaaggcagcag

SEQ ID NO: 335 - DNA, 340 bp, artificial
  synthetic
gatgaacgctagcgacaggcttaacacatgcaagtcgaggggcagcgagggagtgcttgcacttttgtcggcgaccggcgcacgggtgagtaacacgtatgcaacctgcccttgtcagggggataagcgagggaaacctcgtctaataccgcatatatgatttggaggcatctcttaatcaggaaagaattatcggacaaggatgggcatgcgggacattaggtagttgggggtgtaacggaccaccaagccgacgatgtctaggggttctgagaggaaggtcccccacactggtactgagacacggaccagactcctacggaaggcagcagggttggtt

SEQ ID NO: 336 - DNA, 353 bp, artificial
  synthetic
gatgaacgctggcggcgtgcttaacacatgcaagtcgaacgaagcatttgcgacagattttttcggaatgaagttgcttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacggaaggcagcagggttggt

SEQ ID NO: 337 - DNA, 325 bp, artificial
  synthetic
attgaacgctggcggcatgccttacacatgcaagtcgaacggtaacaggttaagctgacgagtggcgaacgggtgagtaatgtatcggaacgtgcccagtcgtgggggataactgctcgaaagagcagctaataccgcatacgacctgagggtgaaagcgggggatcgcaagacctcgcgcgattggagcggccgatatcagattaggtagttggtgaggtaaaggctcaccaagccgacgatctgtagctggtctgagaggacgaccagccacactgggactgagacacggcccagactcctacggaaggcagcagggttggtt

SEQ ID NO: 338 - DNA, 361 bp, artificial
  synthetic
gatgaacgctggcggcgtgcctaacacatgcaagtcgaacggagccttaaccaaagaagcagccagcttgctggaagcggaaaagggaaaggcttagtggcggacgggtgagtaacgcgtgggtaacctggcccatacagggggataacacttagaaataggtgctaataccgcataacaacagaaagcgcatgctttttgtttgaaaaccaaggtggtatgggatggacccgcgtctgattagctggttggcggggtaaaggcccaccaaggcgacgatcagtagccggcctgagagggtggacggccacattgggactgagacacggcccaaactcctacggaaggcagcagggttggt

SEQ ID NO: 339 - DNA, 281 bp, artificial
  synthetic
ctgatcctagaggaactgaaagagtccattcagtttctattcacaacgtgaggagctattatggtatgggtagatctcaatagcagaaaactgggtcaggcagttaagaaatatttccattaagaaagtttttggtcaggagcgatggctcatgcctgtaatcccagcatgttgggaggccaaggtgggaggatttcttgagcccaggagtttgagactagcctggccaacatgatgatacctcatttctaccagaaagtacggaaggcagcagggttggt

SEQ ID NO: 340 - DNA, 333 bp, artificial
  synthetic
gatgaacgctggcggcgcgcctaacacatgcaagtcgaacgagtaagacgccttcgggcgtggatagagtggcgaacgggtgagtaacacgtgaccaacctgccccctcctccgggacaacctcgggaaaccgaggctaataccggatactccgggccccccgcatggggagcccgggaaagccctggcgggaggggatggggtcgcggcccatcaggtagacggcggggtaacggcccaccgtgcctgcaacgggtagccgggctgagaggccgatcggccacattgggactgagacacggcccagactcctacgggaggcagcagggttgg

SEQ ID NO: 341 - DNA, 324 bp, artificial
  synthetic
aacgaacgctggcggcaggcttaacacatgcaagttgaacgggatttgtgtggtgcttgcaccatataatgagagtagcgcactggtgagtaacacgtgggaacgtaccttttggtgggggacaacatctggaaacggatgctaataccgcataagacctgagggtgaaagatttatcgccgaaagaacggcccgcggaagattaggcagttggtggggtaaaggcctaccaaacctacgatctatagctggtctgagaggacgatcagccacactggaactgagacacggtccagactcctacgggaggcagcagggttggtt

SEQ ID NO: 342 - DNA, 354 bp, artificial
  synthetic
gacgaacgctggcggcgtgcctaacacatgcaagtcgaacggagttatattgaaagaagttttcggatggaatttgatataacttagtggcggacgggtgagtaacgcgtgagtaacctgcccataagagggggataacgttctgaaaagaacgctaataccgcatgacatatcgaagccgcatggctatgatatcaaaggagcaatccgcttatggatggactcgcgtccgattagctagttggtgaggtaacggctcaccaaggcgacgatcggtagccggactgagaggttgaacggccacattgggactgagacacggcccagactcctacgggaggcagcagggttggt

SEQ ID NO: 343 - DNA, 337 bp, artificial
  synthetic
gatgaacgctagctacaggcttaacacatgcaagtcgaggggcagcgggattgaagcttgctttaattgccggcgaccggcgcacgggtgagtaacgcgtatccaaccttccgcttactcggggatagcctttcgaaagaaagattaatacccgatggtatcttaagcacgcatgagattaagattaaagatttatcggtaagcgatggggatgcgttccattaggcagttggcggggtaacggcccaccaaacctacgatggataggggttctgagaggaaggtcccccacattggaactgagacacggtccaaactcctacggaaggcagcaggg

SEQ ID NO: 344 - DNA, 337 bp, artificial
  synthetic
attgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagggttggt

SEQ ID NO: 345 - DNA, 375 bp, artificial
  synthetic
gatgaatgctggcggcgtgcctaatacatgcaagtcgagcggtagcagggcttcaccgttctcttgtcgcacttggcctttgagcaagtggtctgatacgagggaacagtgaagctgctgacgagcggcggacggctgagtaacgcgtgggaacataccccaaactgagggataactactcgaaagagtggctaataccgcatgtgatcttcggattaaagcatttatgcggtttgggaatggcctgcgtacgattagatagttggtgaggtaaaggctcaccaagtcgacgatcgttagatggtttgagaggatgatcatccagactgggactgagacacggcccagactcctacgggaggcagcagggttggt

SEQ ID NO: 346 - DNA, 352 bp, artificial
  synthetic
gacgaacgctggcggcgtgcctaatacatgcaagtaggacgcacagtttataccgtagcttgctacaccatagactgtgagttgcgaacgggtgagtaacgcgtaggtaacctacctattagagggggataactattggaaacgatagctaataccgcataacagtatgtaacacatgttagatgcttgaaagatgcaattgcatcgctagtagatggacctgcgttgtattagctagtaggtagggtaatggcctacctaggcaacgatacatagccgacctgagagggtgatcggccacactgggactgagacacggcccagactcctacggaaggcagcagggttggtt

SEQ ID NO: 347 - DNA, 322 bp, artificial
  synthetic
aacgaacgctggcggcaggcttaacacatgcaagttgaacgggatttgtgtggtgcttgcaccatataatgagagtagcgcactggtgagtaacacgtgggaacgtgccttttagtgggggataacatctggaaacggatgctaataccgcatacgccctgagggggaaagatttattgctaagaggtcggcccgcggtagattaggcagttggtggggtaaaggcctaccaaacctacgatctatagctggtctgagaggacgatcagccacattggaactgagacacggtccagactcctacggaaggcagcagggttgg

SEQ ID NO: 348 - DNA, 334 bp, artificial
  synthetic
gacgaacgctggcggcgtgcttaacacatgcaagtcgaacgatgaagcccagcttgctgggtggattagtggcgaacgggtgagtaacacgtgagtaacctgcccctgactctgggataagcgttggaaacgacgtctaatactggatatgactactggtcgcatggcctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagggttggt

SEQ ID NO: 349 - DNA, 369 bp, artificial
  synthetic
gatgaacgccggcggtgtgcctaatacatgcaagtcgtacgcactggcccaactgaaagatggtgcttgcaccagcttgacgatggatcaccagtgagtggcggacgggtgagtaacacgtgggtaacctgccccggagcgggggataacatttggaaacagatgctaataccgcataactacaaagaccacatggtctttgtataaaagatggctctgctatcactctgggatggacccgcggtgcattagctagttggtaaggtaacggcttaccaaggcgatgatgcatagccgagttgagagactgatcggccacaatggaactgagacacggtccatactcctacgggaggcagcagggttggt

SEQ ID NO: 350 - DNA, 317 bp, artificial
  synthetic
agagggggtatggttttgggatgattcaagcgcattacatttattgtgcactttatttctattattatattgtaacatataatgaaataattatgcaactcactgtaatgtagaatcagtgggagccctgagcttgtttttctgcaactagatggtcccatctgggggtgatgggagacagtgacagatcatcaggcattagattctcataaggagcatgcaacctagatccctggcttgcgcagttcacaatagggttcacgctcttgtgagactctaacacccctgttcatgcgacgggaggcagcagggttggt

SEQ ID NO: 351 - DNA, 344 bp, artificial
  synthetic
gatgaacgctagctacaggcttaacacatgcaagtcgaggggcagcggggaggatgcttgcattctctgccggcgaccggcgcacgggtgagtaacgcgtaccgaacctgcctcatactcgggaatagccttgcgaaagtaagattaatgcccggtgttccgcgagggccgcatggccttttcggcaaagttatttacggtatgagatggcggtgcgtcccattagcttgttggcggggtaacggcccaccaaggcgacgatgggtaggggttctgagaggaaggtcccccacattggaactgagacacggtccaaactcctacggaaggcagcagggttggtt

SEQ ID NO: 352 - DNA, 325 bp, artificial
  synthetic
gatgaacgctggcggcatgcctaatacatgcaagtcgaacggagcacttcggtgctcagtggcgaacgggtgagtaacacgtggggaacctgtccatgcgcccgggacaacatctggaaacggatgctaaaaccggatagttcatacggcggcatcgtcgtatgattaaatgaacaactgttcagagcatgggtggacctgcggtgcattagctggatggtgaggcaacggctcaccatggcgacgatgcatagccggcctgagagggcggacggccacattgggactgagacacggcccaaactcctacggaaggcagcagggt

SEQ ID NO: 353 - DNA, 367 bp, artificial
  synthetic
gatgaacgctggcggcgtgcttaacacatgcaagtcgaacgaagcacctctcccgaagattgacacagcttgctgtagattgattcatttgaggtgactgagtggcggacgggtgagtaacgcgtgggtaacctgcctcatagagggggacaacagttggaaacgactgctaataccgcatagtaagaagaattcgcatgttttcttcttgaaagatttatcgctatgagatggacccgcgtctgattagctagttggtaaggtaacggcttaccaaggcgacgatcagtagccggcttgagagagtgaacggccacattgggactgagacacggcccaaactcctacggaaggcagcagggttggt

SEQ ID NO: 354 - DNA, 348 bp, artificial
  synthetic
gacgaacgctggcggcgtgcctaacacatgcaagtcgaacgagaatctttgaacagatcttttcggagtgacgttcaaagaggaaagtggcggacgggcgagtaacgcgtgagtaacctgcccataagagggggataatccatggaaacgtggactaataccgcatattgtagttaagttgcatgacttgattatgaaagatttatcgcttatggatggactcgcgtcagattagatagttggtgaggtaacggctcaccaagtcaacgatctgtagccgaactgagaggttgatcggccgcattgggactgagacacggcccagactcctacgggaggcagcagggt

SEQ ID NO: 355 - DNA, 335 bp, artificial
  synthetic
gatgaacgctggcggcgtgcctaacacatgcaagtcgaacggagttacgatgaaacctagtgattcgtaacttagtggcggacgggtgagtaacgcgtggataacctgccttgcactgggggataacacttagaaataggtgctaataccgcataagcgcacagagccgcatggctcagtgtgaaaaactccggtggtgtaagatggatccgcgtctgattagctggttggcggggtagaagcccaccaaggcgacgatcagtagccggcctgagagggtgaacggccacattgggactgagacacggcccaaactcctacggaaggcagcaggg

SEQ ID NO: 356 - DNA, 334 bp, artificial
  synthetic
gatgaacgctggcggcgtgcctaatacatgcaagtcgaacgaatcttcttcggaagatgagtggcgaacgggtgagtaatacataggtaacctgcccctgtgcgggggataacaggaggaaactcctgctaataccgcatagccatgagcaccgcatggagctcatgccaaatatcctttcggggatagcgcagggatggacctatggcgcattagctggttggcggggcaacggcccaccaaggcaacgatgcgtagccggcctgagagggcggacggccacattgggactgagacacggcccagactcctacgggaggcagcagggttggtt

SEQ ID NO: 357 - DNA, 184 bp, artificial
  synthetic
agtggggtggtggcagtggggtgatggaaattggtcagaatctggatctatcagccacaggacttgctgatgagttggggtgtagggcgagaaacaggggtggaagctggtgctcaggctttcctgtgagccactgcaaggacagacttccatccttcaaggtacggaaggcagcagggttggt

SEQ ID NO: 358 - DNA, 330 bp, artificial
  synthetic
attgaacgctggcggcatgctttacacatgcaagtcgaacggcagcacagggagcttgctcctgggtggcgagtggcgcacgggtgagtaatacatcggaacgtgtcctatcgtgggggataactgatcgaaagatcagctaataccgcataagacctgagggtgaaagtgggggatcgcaagacctcacgcgattggagcggccgatgcccgattagctagttggtgaggtaaaggctcaccaaggcgacgatcggtagctggtctgagaggacgaccagccacactgggactgagacacggcccagactcctacggaaggcagcaggg

SEQ ID NO: 359 - DNA, 350 bp, artificial
  synthetic
gatgaacgctggcggcgtgcttaacacatgcaagtcgaacggggaacattttatggaagcttcggtggaaatagcttgttcctagtggcggacgggtgagtaacgcgtgggtaacctgcctcacactgggggataacagtcagaaatgactgctaataccgcataagcgcacgggattgcatgatccagtgtgaaaaactccggtggtgtgagatggacccgcgttggattagccagttggcagggtaacggcctaccaaagcgacgatccatagccggcctgagagggtggacggccacattgggactgagacacggcccagactcctacgggaggcagcagggttttt

SEQ ID NO: 360 - DNA, 337 bp, artificial
  synthetic
gatgaacgctggcggcgtgcttaacacatgcaagtcgaacggactcatattgaaacctagtgatgtatgagttagtggcggacgggtgagtaacgcgtggagaacctgccgtatactgggggataacacttagaaataggtgctaataccgcataagcgcacagcttcgcatgaagcagtgtgaaaaactccggtggtatacgatggatccgcgtctgattagcttgttggcggggtaaaggcccaccaaggcgacgatcagtagccggcctgagagggtgaacggccacattgggactgagacacggcccaaactcctacgggaggcagcagggtt

SEQ ID NO: 361 - DNA, 340 bp, artificial
  synthetic
gatgaacgctagctacaggcttaacacatgcaagtcgaggggcagcatttttctagcaatagaagagatggcgaccggcgcacgggtgagtaacacgtatccaacctaccgataactccggaatagcctttcgaaagaaagattaataccggatagtatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagggttggtt

SEQ ID NO: 362 - DNA, 289 bp, artificial
  synthetic
ccaggggaattgagggagtggatggagcccaaaggacccacataaagggtttgaagtccagccaggaagggagccagggaggagctcacatcctggagctagaggtggccctgtctgtccaatcctgcccagagatgaagtgagaacatacccgctaaatgtcctttgggtttagagataacaaagtcactggtgaccttggaggaacggtttctgtggagtagtggagttggggagtgagtgggaggtgaggacagggatggaggggtgtgaacatctctttaagaag

SEQ ID NO: 363 - DNA, 346 bp, artificial
  synthetic
gatgaacgctggcggcgtgcttaacacatgcaagtcgagcgaagcactttggtcagattcttcggatgaagactttagtgactgagcggcggacgggtgagtaacgcgtgggtaacctgcctcatacagggggataacagttagaaatgactgctaataccgcataagaccacagggccgcatggcccggtggtaaaaactccggtggtatgagatggacccgcgtctgattagctggttggtggggtaacggcctaccaaggcgacgatcagtagccgacctgagagggtgaccggccacattgggactgagacacggcccaaactcctacggaaggcagcaggg

SEQ ID NO: 364 - DNA, 339 bp, artificial
  synthetic
gacgaacgctggcggcgtgcctaacacatgcaagtcgagcggggaacggatggtagcttgctactgtttgtttctagcggcggacgggtgagtaacgcgtgagcaacctgcctttatcagggggataacgcatcgaaagatgtgctaataccgcgtaagaccacagcttcacatggagcgggggtcaaaggagcaatccggataaagatgggctcgcgtccgattagctagttggtgagataacagcccaccaaggcgacgatcggtagccgacctgagagggtgatcggccacattggaactgagagacggtccagactcctacggaaggcagcaggg

SEQ ID NO: 365 - DNA, 331 bp, artificial
  synthetic
gacgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggagccatttttagtgtgatggttggaaagttttttcggtgtaggatgagctcgcggcctatcagcttgttggtggggtaatggcctaccaaggcggcgacgggtagccggcctgagagggtggacggccacattgggactgagatacggcccagactcctacgggaggcagcagggttggt

SEQ ID NO: 366 - DNA, 305 bp, artificial
  synthetic
aacgaacgctggcggcatgcctaacacatgcaagtcgaacgagaccttcgggtctagtggcgcacgggtgcgtaacgcgtgggaatctgcccttgggttcggaataacagtgagaaattactgctaataccggatgatgtcttcggaccaaagatttatcgcccagggatgagcccgcgtaggattagctagttggtggggtaatggcctaccaaggcgacgatccttagctggtctgagaggatgatcagccacactgggactgagacacggcccagactcctacggaaggcagcagggttggt

SEQ ID NO: 367 - DNA, 358 bp, artificial
  synthetic
gatgaacgctggcggcgtgcttaacacatgcaagtcgaacggaactgtcatttttgaagcaattagcttgctaagggtggaaatgttggcagtttagtggcggacgggtgagtaacgcgtgggtaacctgccttacacagggggataacacctggaaacaggtgctaataccgcataacaggaagaggcgcatgccactttcttgaaaactccggtggtgtaagatggacccgcgtctgattagcttgttggcggggtaacggcccaccaaggcgacgatcagtagccggcctgagagggtggacggccacattgggactgagacacggcccaaactcctacggaaggcagcagggtt

SEQ ID NO: 368 - DNA, 343 bp, artificial
  synthetic
gatgaacgctggcggcgtgcttaacacatgcaagtcgaacgaagcactttatttgatttcttcggaatgaagattttgtgactgagtggcggacgggtgagtaacgcgtgggtaacctgcctcatacagggggataacagttggaaacgactgctaataccgcataagcgcacagggtcgcatgacctggtgtgaaaaactccggtggtatgagatggacccgcgtctgattagccagttggtggggtaacggcctaccaaagcgacgatcagtagccgacctgagagggtgaccggccacattgggactgagacacggcccaaactcctacggaaggcagca

SEQ ID NO: 369 - DNA, 338 bp, artificial
  synthetic
gacgaacgctggcggcgcgcctaacacatgcaagtcgaacgagagagaaggagcttgcttcttcgatcgagtggcgaacgggtgagtaacgcgtgaggaacctgcctcaaagagggggacaacagttggaaacgactgctaataccgcataagcccacgggtcggcatcgacctgagggaaaaggagtgatccgctttgagatggcctcgcgtccgattagctagttggtgaggtaacggcccaccaaggcgacgatcggtagccggactgagaggttgaacggccacattgggactgagacacggcccagactcctacggaaggcagcagggttggt

SEQ ID NO: 370 - DNA, 344 bp, artificial
  synthetic
gacgaacgctggcggcgtgcttaacacatgcaagtcgaacgagaatctgtggaacgaggattcgtccaagtgaagcagaggacagtggcggacgggtgagtaacgcgtgaggaacctgcctttcagagggggacaacagttggaaacgactgctaataccgcatgacacatagatgtcgcatggcatttatgtcaaagatttatcgctgaaagatggcctcgcgtctgattagctagttggtgaggtaacggcccaccaaggcgacgatcagtagccggactgagaggttgaacggccacattgggactgagatacggcccagactcctacggaaggcagcagg

SEQ ID NO: 371 - DNA, 325 bp, artificial
  synthetic
attgaacgctggcggcaggcctaatacatgcaagtcgaacggtaacataagaaagcttgctttcttgatgacgagtggcggacgggtgagtaatatttgggaagctacctgatagagggggacaacagttggaaacgactgctaataccgcatatagcctgagggtgaaagcagcaatgcgctatcagatgcgcccaaacgggattagctagtaggtgaggtaaaggctcacctaggcgacgatctctagctggtctgagaggatgatcagccacattgggactgagacacggcccagactcctacggaaggcagcagggttggt

SEQ ID NO: 372 - DNA, 331 bp, artificial
  synthetic
gatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagca

SEQ ID NO: 373 - DNA, 338 bp, artificial
  synthetic
gacgaacgctggcggcgcgcctaacacatgcaagtcgaacgagagagagggagcttgcttcctcaatcgagtggcgaacgggtgagtaacgcgtgaggaacctgcctcaaagagggggacaacagttggaaacgactgctaataccgcataagcccacgacccggcatcgggttgagggaaaaggagcaatccgctttgagatggcctcgcgtccgattagctagttggtgaggtaatggcccaccaaggcgacgatcggtagccggactgagaggttgaacggccacattgggactgagacacggcccagactcctacggaaggcagcagggttggt

SEQ ID NO: 374 - DNA, 355 bp, artificial
  synthetic
gacgaacgctggcggcgtgcctaatacatgcaagtcgagcgcaggaagctcacggaactcttcggagggaagtgaagggaatgagcggcggacgggtgagtaacacgtaaggaacctgccccaaggattgggataactccgagaaatcggagctaataccgaatagttcttcagaccgcatggtctgatgatgaaaggcgctccggcgtcaccttgggatggccttgcggtgcattagctagttggtggggtaatggcccaccaaggcgacgatgcatagccgacctgagagggtgatcggccacactgggactgagacacggcccagactcctacggaaggcagcagggttggt

SEQ ID NO: 375 - DNA, 329 bp, artificial
  synthetic
gacgaacgctggcggcgtgcctaacacatgcaagtcgaacggggttgagtgcttcggtattcaacttagtggcgaacgggtgagtaacgcgtgaagaacctgcctttcagtgggggacaacagttggaaacgactgctaataccgcatgacacttctgaggggcatcccttagaagtcaaagctttatgtgctgaaagatggcttcgcgtctgattagctagatggcggggtaacggcccaccatggcgacgatcagtagccggtctgagaggatgaacggccacattgggactgagatacggcccagactcctacggaaggcagcagg

SEQ ID NO: 376 - DNA, 334 bp, artificial
  synthetic
gatgaacgctggcggcgtgcttaacacatgcaagtcgaacggacaggtaatgaaacctagtgatttacctgttagtggcggacgggtgagtaacgcgtgggtaacctgccgtatacagggggataacacttagaaataggtgctaaaaccgcataagcgcacaggatcgcatggtctggtgtgaaaaactccggtggtatacgatggacccgcgtctgattagctggttggcggggtagaggcccaccaaggcgacgatcagtagccggcctgagagggtgaacggccacattgggactgagacacggcccaaactcctacggaaggcagcagg

SEQ ID NO: 377 - DNA, 331 bp, artificial
  synthetic
attgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacggaaggcagcagggttggt

SEQ ID NO: 378 - DNA, 352 bp, artificial
  synthetic
gatgaacgctggcggcgtgcttaacacatgcaagtcgaacggagtgccttagaaagaggattcgtccaattgataaggttacttagtggcggacgggtgagtaacgcgtgaggaacctgcctcggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacggaaggcagcagggttggt

SEQ ID NO: 379 - DNA, 338 bp, artificial
  synthetic
gatgaacgctagcggcaggcttaacacatgcaagtcgaggggcatcgggagggaagcttgctttccttgccggcgaccggcgcacgggtgagtaacacgtatgcaacctgccctcttcagggggacaaccttccgaaagggaggctaatcccgcgtatatcggtttcgggcatccgagatcgaggaaagattcatcggaagaggatgggcatgcggcgcattagctagacggcggggtaacggcccaccgtggcgacgatgcgtaggggttctgagaggaaggtcccccacactggtactgagacacggaccagactcctacgggaggcagcagggtt

SEQ ID NO: 380 - DNA, 363 bp, artificial
  synthetic
gacgaacgctggcggcgtgcctaatacatgcaagtcgagcgagctgaaccagcagattcacttcggtgatgacgctgggaacgcgagcggcggatgggtgagtaacacgtgggtaacctgccctaaagtctgggataccacttggaaacaggtgctaataccggataacaacaatagctgcatggctattgcttaaaaggcggcgaaagctgtcgctaaaggatggacccgcggtgcattagctagttggtaaggtaatggcttaccaaggcgacgatgcatagccgagttgagagactgatcggccacattgggactgagacacggcccaaactcctacgggaggcagcagggttggttttt

SEQ ID NO: 381 - DNA, 328 bp, artificial
  synthetic
ttgaacgctggcggcatgctttacacatgcaagtcgaacggtaacaggtcttcggatgctgacgagtggcgaacgggtgagtaatacatcggaacgtgcctagtagtgggggataactactcgaaagagtagctaataccgcatgagatctacggatgaaagcaggggaccttcgggccttgtgctactagagcggctgatggcagattaggtagttggtggggtaaaggcttaccaagcctgcgatctgtagctggtctgagaggacgaccagccacactgggactgagacacggcccagactcctacggaaggcagcagggttggt

SEQ ID NO: 382 - DNA, 344 bp, artificial
  synthetic
attgaacgctggcggcaggcctaacacatgcaagtcgagcggcagcgacaacattgaaccttcgggggatttgttgggcggcgagcggcggacgggtgagtaatgcctgggaaattgccctgatgtgggggataaccattggaaacgatggctaataccgcatgatagcttcggctcaaagagggggaccttcgggcctctcgcgtcaggatatgcccaggtgggattagctagttggtgaggtaagggctcaccaaggcgacgatccctagctggtctgagaggatgatcagccacactggaactgagacacggtccagactcctacggaaggcagcagggtt

SEQ ID NO: 383 - DNA, 351 bp, artificial
  synthetic
gacgaacgctggcggcgtgcttaacacatgcaagtcgaacggagcacccttgaaagagatttcggtcaatggataggaatgcttagtggcggacgggtgagtaacgcgtgaggaacctgcctttcagagggggacaacagctggaaacggctgctaataccgcataacacataggtgtcgcatggcatttatgtcaaagatttatcgctgagagatggcctcgcgtctgattagctagttggtagggtaacggcctaccaaggcgacgatcagtagccggactgagaggttggccggccacattgggactgagatacggcccagactcctacgggaggcagcagggttggt

SEQ ID NO: 384 - DNA, 346 bp, artificial
  synthetic
gatgaacgctggcggcgtgcctaacacatgcaagtcgaacgaagcaatcagaatgaagttttcggatggatttctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcaggg

SEQ ID NO: 385 - DNA, 326 bp, artificial
  synthetic
attgaacgctggcggcaggcctaacacatgcaagtcgagcggatgagtggagcttgctccctgattcagcggcggacgggtgagtaatgcctaggaatctgcctggtagtgggggacaacgtttcgaaaggaacgctaataccgcatacgtcctacgggagaaagtgggggatcttcggacctcacgctatcagatgagcctaggtcggattagctagttggtgaggtaaaggctcaccaaggcgacgatccgtaactggtctgagaggatgatcagtcacactggaactgagacacggtccagactcctacggaaggcagcaggg

SEQ ID NO: 386 - DNA, 311 bp, artificial
  synthetic
agcgaacgctggcggcaggcttaacacatgcaagtcgaacgggcatcttcggatgtcagtggcagacgggtgagtaacacgtgggaacgtacccttcggttcggaataactcagggaaacttgagctaataccggatacgcccttttggggaaaggtttactgccgaaggatcggcccgcgtctgattagcttgttggtggggtaacggcctaccaaggcgacgatcagtagctggtctgagaggatgatcagccacactgggactgagacacggcccagactcctacggaaggcagcagggttgtttttt

SEQ ID NO: 387 - DNA, 347 bp, artificial
  synthetic
gacgaacgctggcggcgtgcctaacacatgcaagtcgaacgaagctctgcgatacaagacttcggtcaagttgaacggatgacttagtggcggacgggtgagtaacgcgtgaggaacctgcctttcagtgggggacaacagttggaaacgactgctaataccgcataatgtattctgaccgcatgatcggaataccaaagatttattgctgaaagatggcctcgcgtctgattagatagttggtgaggtaacggcccaccaagtcgacgatcagtagccggactgagaggttgaacggccacattgggactgagatacggcccagactcctacggaaggcagcaggg

SEQ ID NO: 388 - DNA, 342 bp, artificial
  synthetic
gacgaacgctggcggcgtgcctaatacatgcaagtcgagcggaccggattggggcttgccttgattcggtcagcggcggacgggtgagtaacacgtgggcaacctgcccgcaagaccgggataactccgggaaaccggagctaataccggataacaccgaagaccgcatggtcttcggttgaaaggcggcctttgggctgtcacttgcggatgggcccgcggcgcattagctagttggtgaggtaacggctcaccaaggcgacgatgcgtagccggcctgagagggtgaccggccacactgggactgagacacggcccagactcctacggaaggcagcaggg

SEQ ID NO: 389 - DNA, 343 bp, artificial
  synthetic
gatgaacgctggcggcgtgcttaacacatgcaagtcgaacgaagcactctatttgatttcttcggaatgaagattttgtgactgagtggcggacgggtgagtaacgcgtgggtaacctgcctcatacagggggataacagttggaaacgactgctaataccgcataagcgcacagtaccgcatggtacagtgtgaaaaactccggtggtatgagatggacccgcgtctgattagctagttggtgaggtaacggcctaccaaggcgacgatcagtagccgacctgagagggtgaccggccacattgggactgagacacggcccaaactcctacgggaggcagca

SEQ ID NO: 390 - DNA, 334 bp, artificial
  synthetic
gacgaacgctggcggcgtgcctaacacatgcaagtcgaacggagtcggccccgcaagggactgacttagtggcgaacgggtgagtaacgcgtgagcaacctgcctttcagtgggggacaacagttggaaacgactgctaataccgcataatgcattttgaccgcatgatcgagatgccaaagatttattgctgaaagatgggctcgcgtctgattagatagttggtgaggtaacggcccaccaagtcgacgatcagtagccggactgagaggttgaacggccacattgggactgagatacggcccagactcctacggaaggcagcagggttggt

SEQ ID NO: 391 - DNA, 343 bp, artificial
  synthetic
gatgaacgctagcgacaggcttaacacatgcaagtcgaggggcagcggggagagaagcttgcttttctccgccggcgaccggcgcacgggtgagtaacacgtatgcaacctggccgtgacagatggataaccgggagaaatcccgcctaatacagcatgacgcccttgagggacatcccttgagggccaaaggaggcgactccggtcacggatgggcatgcggcgcattaggtagttggtggggcaacggcccaccaagccgacgatgcgtaggggttctgagaggaaggtcccccacattggaactgagacacggtccaaactcctacgggaggcagcaggg

SEQ ID NO: 392 - DNA, 344 bp, artificial
  synthetic
gacgaacgctggcggcatgcctaacacatgcaagtcgaacggagattatttcggtaatcttagtggcgaacgggtgagtaacgcgtaggcaacctgccctttagttggggacaacatcccgaaaggggtgctaataccgaatgtgatcgctttctcgcatgagagagcgatgaaagatggcctctatttataagctatcgctaaaggatgggcctgcgtctgattagctagttggtggggtaacggcctaccaaggcgatgatcagtagccggtctgagaggatgaacggccacattgggactgagacacggcccagactcctacgggaggcagcagggttggt

SEQ ID NO: 393 - DNA, 351 bp, artificial
  synthetic
gatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagcagggttgtt

SEQ ID NO: 394 - DNA, 349 bp, artificial
  synthetic
gatgaacgctagcgacaggcttaacacatgcaagtcgaggggcagcggtgtagagagcttgctctcttcagccggcgaccggcgcacgggtgagtaacacgtatgcaacctgcccttgacactgggataacccggagaaatccggactaataccgggcgacaccttcggaggacatcctccttaggtcgaaggaagcgattccggtcaaggatgggcatgcggcgcattagctagttggcggggtaacggcccaccaaggcgacgatgcgtaggggttctgagaggaaggtcccccacattggtactgagacacggaccaaactcctacgggaggcagcagggttggtt

SEQ ID NO: 395 - DNA, 324 bp, artificial
  synthetic
aacgaacgctggcggcaggcttaacacatgcaagttgaacgggaacatacgatagcttgctatagtatgtgagagtggcgcacgggtgagtaatacatgggaacataccttaaagtgggggataacttctggaaacggatgctaataccgcatataccctgagggggaaagatttatcgctttaagattggcccatggcagattaggtagttggtggggtaaaggcctaccaagccgacgatctgtagctggtctgagaggacgatcagccacattgggactgagacacggcccagactcctacggaaggcagcagggttggtt

SEQ ID NO: 396 - DNA, 351 bp, artificial
  synthetic
gatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagcagggttggt

SEQ ID NO: 397 - DNA, 336 bp, artificial
  synthetic
gatgaacgctggcggcgtgcttaacacatgcaagtcgaacggacttttgatgaaacctagtgattcaaaagttagtggcggacgggtgagtaacgcgtgggtaacctgcctcgtacagggggataacacttagaaataggtgctaataccgcataagcgcacagcttcgcatgaagcagtgtgaaaaactccggtggtacgagatggacccgcgtctgattagctagttggtgaggtaacggcccaccaaggcgacgatcagtagccggcctgagagggtgaacggccacattgggactgagacacggcccaaactcctacggaaggcagcagggt

SEQ ID NO: 398 - DNA, 344 bp, artificial
  synthetic
attgaacgctggcggaacgctttacacatgcaagtcgaacggtaacgtggggaggagcttgctccaccccgacgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagggttggtt

SEQ ID NO: 399 - DNA, 334 bp, artificial
  synthetic
gatgaacgctagcgacaggcttaacacatgcaagtcgaggggcagcacgggcagcaatgcctggtggcgaccggcgcacgggtgagtaacgcgtatgcaacttgcctatcagaggggaacagcccggcgaaagtcggattaatgccccataaaacagggatcccgcatggggttatttgttaaagattcatcgctgatagataggcatgcgttccattaggcagttggcggggtaacggcccaccaaaccgacgatggataggggttctgagaggaaggtcccccacattggaactgagacacggtccaaactcctacggaaggcagcagggtt

SEQ ID NO: 400 - DNA, 346 bp, artificial
  synthetic
gatgaacgctggcggcgtgcttaacacatgcaagtcgaacgaagcactttacctgatcccttcggggtgattgttctgtgactgagtggcggacgggtgagtaacgcgtgggtaacctgcctcatacagggggataacagttggaaacgactgctaataccgcataagcgcacagcatcgcatgatgcagtgtgaaaaactccggtggtatgagatggacccgcgttggattagctagttggaggggtaacggcccaccaaggcgacgatccatagccgacctgagagggtgaccggccacattgggactgagacacggcccaaactcctacggaaggcagcaggg

SEQ ID NO: 401 - DNA, 342 bp, artificial
  synthetic
gatgaacgctagctacaggcttaacacatgcaagtcgaggggcagcgggaagcaagcttgcttgctttgccggcgaccggcgtacgggtgcgtaacgcgtaccgaacctacctcacactccgggatagccctgcgaaagcaggattaataccgggtagcctcactatatcgcatgatattatgagtaaagatttattggtgtgagatggcggtgcgtcccattagttagttggcggggtaacggcccaccaagacgacgatgggtaggggttctgagaggaaggtcccccacattggaactgagacacggtccaaactcctacgggaggcagcagggttggt

SEQ ID NO: 402 - DNA, 351 bp, artificial
  synthetic
gatgaacgctggcggcgtgcttaacacatgcaagtcgaacgaagatatttagaatgagagcttcggcaggatttttatctatcttagtggcggacgggtgagtaacgtgtgggcaacctgccctgtactggggaataatcattggaaacgatgactaataccgcatgtggtcctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacgggaggcagcagggtt

SEQ ID NO: 403 - DNA, 333 bp, artificial
  synthetic
gatgaacgctggcggcgtgcttaacacatgcaagtcgaacgatgatccggtgcttgcatcggggattagtggcgaacgggtgagtaacacgtgagtaacctgcccttgactctgggataagcctgggaaactgggtctaataccggatatgaccttccatcgcatggtggttggtggaaagcttttgtggttttggatggactcgcggcctatcagcttgttggtggggtaatggcctaccaaggcgacgacgggtagccggcctgagagggtgaccggccacactgggactgagacacggcccagactcctacgggaggcagcagggttggt

SEQ ID NO: 404 - DNA, 355 bp, artificial
  synthetic
gcagtgaggagaggtgtatgcaggtctttgatagcagggacaggtgtgtgcaggtctcaggcattggggacaggtgtgtgcaggtctctgatagcagagacgggtgtgtgcacaccagggtcaaaagggagcttgtccaaggaagggactgaggatgctgtggacagctatggaggggacatacatgggtgcagcattcttttagccgcagacacagaagagggccagcctctgctggggccgcagggctggaggtgtggaagagctcctgggagctcccggggcaagagtcaagcctccgaggtgggcagaggcccagtctccctcaccccggggacggaaggcagcagggttg

SEQ ID NO: 405 - DNA, 450 bp, artificial
  synthetic
gacgaacgctggcggcgcgcctaacacatgcaagtcgaacggagttattttcttcactccgagctttttgccagttagccggagcggcaatcggttttgcccagcataaaaagcaaaaccgatttaggctcataaagtaaggctaacacagagggcagagagttgggagtgaagaaaataacttagtggcgaacgggtgagtaacgcgtgagtaacctgccctggagtgggggacaacagttggaaacgactgctaataccgcataagcccacggcccggcatcgggctgcgggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagggtt

SEQ ID NO: 406 - DNA, 355 bp, artificial
  synthetic
gatgaacgctggcggcgtgcttaacacatgcaagtcgaacggagatatcattttcgaagcgattagtttactaagagcggagatgttgctatcttagtggcggacgggtgagtaacgcgtgggtaacctgccttacactgggggataacacttagaaataggtgctaataccgcataacaggagaagacgcatgtcttttccttgaaaactccggtggtgtaagatggacccgcgtctgattagcttgttggcggggtaacggcccaccaaggcgacgatcagtagccggcctgagagggtgaacggccacattgggactgagacacggcccaaactcctacgggaggcagcagg

SEQ ID NO: 407 - DNA, 325 bp, artificial
  synthetic
gatgaacgctggcggcgtgcctaacacatgcaagtcgagcgattctcttcggagaagagcggcggacgggtgagtaacgcgtgggtaacctgccctgtacacacggataacataccgaaaggtatgctaatacgagataatatgcttttatcgcatggtagaagtatcaaagctccggcggtacaggatggacccgcgtctgattagctagttggtgaggtaacggctcaccaaggcgacgatcagtagccgacctgagagggtgatcggccacattggaactgagacacggtccaaactcctacgggaggcagcagggttggtt

SEQ ID NO: 408 - DNA, 342 bp, artificial
  synthetic
gataaacgctggcggcgcacataagacatgcaagtcgaacgaacttaacctttagtttactaatggagcggttagtggcggactggtgagtaacgcgtaagcaatctgcctatcagaggggaataacagtgagaaatcattgctaataccgcatatgctgtgagaatcgcatgattcaaacaggaaaggagaaatccgctgatagatgagcttgcgtctgattagttagttggtgaggtaatggctcaccaagacgatgatcagtagccggactgagaggttgaacggccacattgggactgagatacggcccagactcctacggaaggcagcagggttggt

SEQ ID NO: 409 - DNA, 332 bp, artificial
  synthetic
attgaacgctggcggcatgccttacacatgcaagtcgaacggcagcatgatctagcttgctagattgatggcgagtggcgaacgggtgagtaatacatcggaacgtgccctgtagtgggggataactagtcgaaagattagctaataccgcatacgacctgagggtgaaagtgggggaccgcaaggcctcatgctataggagcggccgatgtctgattagctagttggtggggtaaaggcccaccaaggcgacgatcagtagctggtctgagaggacgatcagccacactgggactgagacacggcccagactcctacggaaggcagcaggg

SEQ ID NO: 410 - DNA, 351 bp, artificial
  synthetic
gatgaacgctggcggcgtgcttaacacatgcaagtcgaacgggaaacctattattgaaacttcggtcgatttaatttgtttctagtggcggacgggtgagtaacgcgtgggtaacctgccctatacagggggataacaaccagaaatggttgctaataccgcataagcgcacgggaccgcatggtccagtgtgaaaaactccggtggtataggatggacccgcgttggattagctagttggtggggtaacggcccaccaaggcgacgatccatagccggcctgagagggtgaacggccacattgggactgagacacggcccagactcctacggaaggcagcagggttggtt

SEQ ID NO: 411 - DNA, 342 bp, artificial
  synthetic
gatgaacgctagcggcaggcctaacacatgcaagtcgaggggcagcacgataaagagtttactctttatggtggcgaccggcggacgggtgcgtaacgcgtatgcaacctgcctgacacagggggataatccgaagaaatttggtctaataccccataataccgatgtaggcatctatgttggttgaaaactccggtggtgtcagatgggcatgcgttgtattagctagttggtgaggtaacggctcaccaaggcgacgatacatagggggactgagaggttaaccccccacactggtactgagacacggaccagactcctacgggaggcagcagggttggt

SEQ ID NO: 412 - DNA, 334 bp, artificial
  synthetic
gatgaacgctggcggcgtgcttaacacatgcaagtcgaacgattaacccaccttcgggtggttatagagtggcgaacgggtgagtaacacgtgaccaacctgccccagacatcggaacaaccattggaaacgatggctaataccggatactatccttctttcacatgatggagggatgaaagctcaggcggtctgggatggggtcgcggcccatcaggtagtaggcggggtaacggcccacctagcttatgacgggtagccggactgagaggtcgaccggccacattgggactgagatacggcccagactcctacgggaggcagcagggttggt

SEQ ID NO: 413 - DNA, 305 bp, artificial
  synthetic
agcgaacgctggcggcaggcttaacacatgcaagtcgagcgggcccttcggggtcagcggcggacgggtgagtaacgcgtgggaacgtgccttctggttcggaataaccctgggaaactagggctaataccggatacgcccttttggggaaaggtttactgccggaagatcggcccgcgtctgattagctagttggtggggtaacggcctaccaaggcgacgatcagtagctggtctgagaggatgatcagccacactgggactgagacacggcccagactcctacggaaggcagcagggttggt

SEQ ID NO: 414 - DNA, 338 bp, artificial
  synthetic
gatgaacgctagctacaggcttaacacatgcaagtcgaggggcagcagggaggatgcttgcattctccgctggcgaccggcgcacgggtgcgtaacgcgtatcgaacctgcctcatactcgggaacagccttgcgaaagtaagattaatgcccgatgttctggtttccccgcatgaggagtccagcaaagaaattcggtatgagatggcgatgcgtcccattagctggttggcggggtaacggcccaccaaggcatcgatgggtaggggttctgagaggaaggtcccccacattggaactgagacacggtccaaactcctacggaaggcagcagggtt

SEQ ID NO: 415 - DNA, 345 bp, artificial
  synthetic
gatgaacgctggcggcgtgcttaacacatgcaagtcgagcgaagcacttaagaatgattcttcggatgaaatcttttgtgactgagcggcggacgggtgagtaacgcgtgggtaacctgcctcatacagggggataacagttagaaatgactgctaataccgcataagcgcacagtgccgcatggcaccgtgtgaaaaactccggtggtatgggatggacccgcgttggattaggcagttggcggggtaacggcccaccaaaccgacgatccatagccggcctgagagggtggacggccacattgggactgagacacggcccaaactcctacggaaggcagcagg

SEQ ID NO: 416 - DNA, 343 bp, artificial
  synthetic
gatgaacgctagctacaggcttaacacatgcaagtcgaggggcagcatgggagtttgcttgcaaacttccgatggcgaccggcgcacgggtgagtaacacgtatccaacctgccgataactcggggatagcctttcgaaagaaagattaatatccgatagcatatcaagatcgcatgatcttgatattaaagaatttcggttatcgatggggatgcgttccattagttagttggcggggtaacggcccaccaagacgacgatggataggggttctgagaggaaggtcccccacattggaactgagacacggtccaaactcctacggaaggcagcagggttggt

SEQ ID NO: 417 - DNA, 315 bp, artificial
  synthetic
gatgaacgctggcggcgtgcctaatacatgcaagtcgagcgaaccacttcggtgggaagcggcgaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagggttggtt

SEQ ID NO: 418 - DNA, 344 bp, artificial
  synthetic
gatgaacgctggcggcgtgcttaacacatgcaagtcgaacgggaaacatttcattgaggcttcggcggatttggtctgtttctagtggcggacgggtgagtaacgcgtgggtaacctgccttatactgggggataacagccagaaatggctgctaataccgcataagcgcacgggaccgcatggtcctgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagg

SEQ ID NO: 419 - DNA, 298 bp, artificial
  synthetic
gacgaacgctggcggtgtgcttcacacatgcaagtcgaacgaggtagcaatacctagtggcggacgggtgagtaacgcgtgggaatctgcctttagatgggggataacgggccgaaaggtccgctaataccgcatatgccgagaggtgaaaggagtaatccgtctaaagatgagcccgcgtccgattagctagttggtagagtaagagcctaccaaggcgacgatcggtagctggtctgagaggatgatcagccacaatgggactgagacacggcccatactcctacggaaggcagca

SEQ ID NO: 420 - DNA, 336 bp, artificial
  synthetic
gacgaacgctggcggcgtgcttaacacatgcaagtcgagcgaacaaagctccttcgggagtggagttagcggcggacgggtgagtaacacgtgggtaacctgccttatagagggggatagccttccgaaaggaagattaataccgcataagaagtagaagtcgcatggctttagctttaaaggagcaatccgctataagatggacccgcggcgcattagctagttggtgaggtaacggctcaccaaggcgacgatgcgtagccgacctgagagggtgatcggccacattggaactgagacacggtccagactcctacgggaggcagcagggttggt

SEQ ID NO: 421 - DNA, 336 bp, artificial
  synthetic
gacgaacgctggcggcgtgcctaacacatgcaagtcgaacggggtgaagcccttcgggacttcacttagtggcgaacgggtgagtaacgcgtgaggaacctgcctttcagtgggggacaacagttggaaacgactgctaataccgcataacactttttggggacatccctgaaaagtcaaagctttatgtgctgaaagatggcctcgcgtctgattagctagttggcggggtaacggcccaccaaggcgacgatcagtagccggtctgagaggatgaacggccacattgggactgagatacggcccagactcctacgggaggcagcagggttggtt

SEQ ID NO: 422 - DNA, 308 bp, artificial
  synthetic
aacgaacgctggcggcatgcctaacacatgcaagtcgaacgagtgccttcgggtgctagtggcgcacgggtgcgtaacgcgtgggaatctgccccttggttcggaataacagttagaaatgactgctaataccggatgatgacgtaagtccaaagatttatcgccatgggatgagcccgcgtaggattagctagttggtgtggtaaaggcgcaccaaggcgacgatccttagctggtctgagaggatgatcagccacactgggactgagacacggcccagactcctacgggaggcagcagggttggtt

SEQ ID NO: 423 - DNA, 341 bp, artificial
  synthetic
gacgaacgctggcggcgcgcctaacacatgcaagtcgaacggaatcaagaggagcttgcttttcttgatttagtggcgaacgggtgagtaacgcgtgagtaacctgccctggagtgggggacaacagttggaaacgactgctaataccgcataagcccacggtgctgcatggcactgcgggaaaaggatttattcgctctaggatggactcgcgtccaattagctagttggtgaggtaacggcccaccaaggcgacgattggtagccggactgagaggttgaacggccacattgggactgagacacggcccagactcctacggaaggcagcagggttggtt

SEQ ID NO: 424 - DNA, 347 bp, artificial
  synthetic
gacgaacgctggcggcgtgcttaacacatgcaagtcgaacgagaatctgatgacggagttttcggacaactgattcagaggacagtggcggacgggtgagtaacgcgtgaggaacctgcctttcagagggggacaacagttggaaacgactgctaataccgcatgaagcgtattgatcgcatggtcgatacgccaaagatttatcgctgaaagatggcctcgcgtctgattagctagttggtgaggtaacggcccaccaaggcgacgatcagtagccggactgagaggttgaacggccacattgggactgagatacggcccagactcctacggaaggcagcagggtt

SEQ ID NO: 425 - DNA, 338 bp, artificial
  synthetic
gatgaacgctagcggcaggcttaacacatgcaagtcgaggggcagcggggagtagcaatactctgccggcgaccggcgaaagggtgcgtaacgcgtgagcgacatacccgtgactggggcataaccgatggaaacgttgactaattccccataacacatcgtgctgcatggcatggtgttgaaaatttcgatggtcacggattggctcgcgtctgattagctagttggtggggtaacggctcaccaaggcgacgatcagtaggggttctgagaggaaggtcccccacaatggaactgagacacggtccatactcctacggaaggcagcagggttggtt

SEQ ID NO: 426 - DNA, 350 bp, artificial
  synthetic
gatgaacgctggcggcgtgcttaacacatgcaagtcgaacggggattatttcattgaagcttcggcagatttggcttaatcctagtggcggacgggtgagtaacgcgtgggtaacctgccttgtacagggggataacagtcagaaatgactgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacggaaggcagcagggttggt

SEQ ID NO: 427 - DNA, 353 bp, artificial
  synthetic
gatgaacgctggcggcgtgcctaacacatgcaagtcgaacgaagcgtgcggatggaatttcttcggagaggaagcctgcatgactgagtggcggacgggtgagtaacgcgtgggcaacctgccctgcacagggggacaacagccggaaacggctgctaataccgcataagcgcacagcttcgcatggagcggtgtgaaaagctgcggcggtgcaggatgggcccgcgtctgattagctggttggcggggcaacggcccaccaaggcgacgatcagtagccggcctgagagggtggacggccacattgggactgagacacggcccagactcctacggaaggcagcagggttggt

SEQ ID NO: 428 - DNA, 338 bp, artificial
  synthetic
agtgaacgctggcggtaggcctaacacatgcaagtcgaacggcagcacagtaagagcttgctcttacgggtggcgagtggcggacgggtgaggaatgcatcggaatctactctgtcgtgggggataacgtagggaaacttacgctaataccgcatacgacctacgggtgaaagcaggggatcttcggaccttgcgcgattgaatgagccgatgcccgattagctagttggcggggtaagagcccaccaaggcgacgatcggtagctggtctgagaggatgatcagccacactggaactgagacacggtccagactcctacggaaggcagcagggttgg

SEQ ID NO: 429 - DNA, 331 bp, artificial
  synthetic
gatgaacgctggcggcgtgcttaacacatgcaagtcgaacgatgaacctgtgcttgcacgggggattagtggcgaacgggtgagtaacacgtgagtaacctgcccttgacttcgggataagcctgggaaactgggtctaatactggatacgacctctcatcgcatggtgtgggggtggaaagtttttgcggttttggatggactcgcggcctatcagcttgttggtgaggtaatggctcaccaaggcgacgacgggtagccggcctgagagggtgaccggccacactgggactgagacacggcccagactcctacggaaggcagcagggtt

SEQ ID NO: 430 - DNA, 348 bp, artificial
  synthetic
gatgaacgctggcggcgtgcttaacacatgcaagtcgagcgaagcgcttaagtttgattcttcggatgaagacttttgtgactgagcggcggacgggtgagtaacgcgtgggtaacctgcctcatacagggggataacagttagaaatgactgctaataccgcataagaccacaaggtcgcatgaccaggtggtaaaaaactccggtggtatgagatggacccgcgtctgattaggtagttggtgaggtaacggctcaccaagccgacgatcagtagccgacctgagagggtgaccggccacattgggactgagacacggcccagactcctacgggaggcagcagggt

SEQ ID NO: 431 - DNA, 342 bp, artificial
  synthetic
gacgaacgctggcggcgtgcctaatacatgcaagtagaacgctgaagagaggagcttgctcttctggatgagttgcgaacgggtgagtaacgcgtaggtaacctgcctggtagcgggggataactattggaaacgatagctaataccgcataaaattgattattgcatgataattaattgaaaggtgcaattgcatcactaccagatggacctgcgttgtattagctagttggtgaggtaacggctcaccaaggcgacgatacatagccgacctgagagggtgatcggccacactgggactgagacacggcccagactcctacggaaggcagcagggttggt

SEQ ID NO: 432 - DNA, 344 bp, artificial
  synthetic
gataaacgctggcggcgcacataagacatgcaagtcgaacggacttaactcattctttacgattgagagcggttagtggcggactggtgagtaacacgtaagcaacctgcctattagaggggaataacagtgagaaatcattgctaataccgcatatgctcacagtatcacatgatacagtgaggaaaggagcaatccgctaatagatgggcttgcgtctgattagctagttggtggggtaacggcctaccaaggcgacgatcagtagccggactgagaggttgaacggccacattgggactgagatacggcccagactcctacggaaggcagcagggttggtt

SEQ ID NO: 433 - DNA, 348 bp, artificial
  synthetic
gatgaacgctggcggcgtgcctaacacatgcaagtcgaacgaagcatgcaggaagaagcccttcggggcaggcgcctggatgactgagtggcggacgggtgagtaacgcgtgggcaacctgccccatacagggggataacagccggaaacggctgctaataccgcataaaccagggaggcgcatgcctctttggggaaagctccggcggtatgggaggggcccgcgtctgattagctggttggcggggcagcggcccaccaaggcgacgatcagtagccggcctgagagggcggacggccacattgggactgagacacggcccaaactcctacgggaggcagcagggt

SEQ ID NO: 434 - DNA, 353 bp, artificial
  synthetic
gacgaacgctggcggcgtgcctaatacatgcaagtggaacgcaacttttctcccgtttcttcggaaacacctgagaagttgagtcgcgaacgggtgagtaacgcgtaggtaacctacctcttagcgggggataactattggaaacgatagctaataccgcataacagtgtttaacacatgttaaacatttgaaagaagcaactgcttcactaggagatggacctgcgttgtattagctagttggtagggtaacggcctaccaaggctccgatacatagccgacctgagagggtgatcggccacactgggactgagacacggcccagactcctacggaaggcagcagggttggt

SEQ ID NO: 435 - DNA, 328 bp, artificial
  synthetic
attgaacgctggcggcaggcttaacacatgcaagtcgaacgatgactctctagcttgctagagatgattagtggcggacgggtgagtaacatttaggaatctacctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacggaaggcagcagggttggtt

SEQ ID NO: 436 - DNA, 319 bp, artificial
  synthetic
aatcaacgctggcggcgtgcctaacacatgcaagtcgaacgagaaagtggagcaatccatgagtaaagtggcgaccgggtgagtaacacgtgactaacctacctccgagtggggaataactccgggaaaccggggctaataccgcataacatcgcaagatcaaagcagcaatgcgcttggagagggggtcgcggctgattagctagttggcggggtaacggcccaccaaggcgaagatcggtatccggcctgagagggcgcacggacacactggaactgaaacacggtccagactcctacggaaggcagcagggttggt

SEQ ID NO: 437 - DNA, 345 bp, artificial
  synthetic
gataaacgctggcggcgcacataagacatgcaagtcgaacggacttaatcgaaatatttatattttgaagcggttagtggcggactggtgagtaacgcgtaaggaacctgcctattagaggggaataacagtgagaaatcattgctaataccgcatatgccatagatatcacatgataacagtgggaaaggagcaatccgctaatagatggccttgcgtctgattagatagttggtggggtaacggcctaccaagtcgacgatcagtagccggactgagaggttgaacggccacattgggactgagatacggcccagactcctacggaaggcagcagggttggtt

SEQ ID NO: 438 - DNA, 342 bp, artificial
  synthetic
gatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagcatagtttccccgcatggggttactattaaaggattccggttatcgatggggatgcgttccattaggcagttggcggggtaacggcccaccaaacccacgatggataggggttctgagaggaaggtcccccacattggaactgagacacggtccaaactcctacgggaggcagcagggttggtt

SEQ ID NO: 439 - DNA, 346 bp, artificial
  synthetic
gatgaacgctggcggcgtgcttaacacatgcaagtcgaacgaagcaccttgatttgattcttcggatgaagatcttggtgactgagtggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccaaactcctacggaaggcagcaggg

SEQ ID NO: 440 - DNA, 343 bp, artificial
  synthetic
gatgaacgctggcggcgtgcttaacacatgcaagtcgaacgaagcactttaccagatttcttcggaatgaaagttttgtgactgagtggcggacgggtgagtaacgcgtgggtaacctgcctcatacagggggataacagttggaaacgactgctaataccgcataagcgcacggtatcgcatgatacagtgtgaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagca

SEQ ID NO: 441 - DNA, 340 bp, artificial
  synthetic
gatgaacgctagcggcaggcttaacacatgcaagtcgaggggcagcgggaggagtgcttgcactccttgccggcgaccggcgcacgggtgagtaacacgtatgcaacctaccctaatcagggggacaacccggcgaaagccgggctaatcccgcgtacatctccttagggcatccttaggagaggaaaggcttcggccggattaggatgggcatgcggcgcattaggtagtcgggggtgtaacggaccaccgagccgacgatgcgtaggggttctgagaggaaggtcccccacactggtactgagacacggaccagactcctacggaaggcagcagggtt

SEQ ID NO: 442 - DNA, 342 bp, artificial
  synthetic
gatgaacgctagcgacaggcttaacacatgcaagtcgaggggcagcgggtagatgagcttgctcatttatgccggcgaccggcgcacgggtgagtaacacgtatgcaacctgcccgtctcagggggataatcgtcggaaacggcgtctaataccccgtatgaagccggacggcatcgtctggttttgaaagaatatcggagacggatgggcatgcggcgcattagctagttggcggggtaacggcccaccaaggcgacgatgcgtaggggttctgagaggaaggtcccccacactggtactgagacacggaccagactcctacgggaggcagcagggttggt

SEQ ID NO: 443 - DNA, 347 bp, artificial
  synthetic
gacgaacgctggcggcgtgcctaatacatgcaagtcgagcgaagtttttctggtgcttgcactagaaaaacttagcggcgaacgggtgagtaacacgtaaagaacctgcctcatagacggggacaactattggaaacgatagctaataccggataacagcataaatcgcatgatatatgtttaaaagttggtttcggctaacactatgagatggctttgcggtgcattagctagttggtggggtaaaggcctaccaaggcgacgatgcatagccgacctgagagggtgatcggccacactgggactgagacacggcccagactcctacgggaggcagcagggttggt

SEQ ID NO: 444 - DNA, 331 bp, artificial
  synthetic
attgaacgctggcggcaggcttaacacatgcaagtcgagcggggtgaggtgcttcggtactgatcctagcggcggacgggtgagtaatgcttaggaatctgccatttagtgggggacaacattccgaaagggatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagggttggt

SEQ ID NO: 445 - DNA, 340 bp, artificial
  synthetic
gatgaacgctagcgacaggcttaacacatgcaagtcgaggggcagcgaggggtagcaatactctgtcggcgaccggcgcacgggtgagtaacgcgtatgcaacctacctatcagaggggaataacccggcgaaagtcggactaataccgcataaaacaggggtcccgcatggggatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagggttggtt

SEQ ID NO: 446 - DNA, 354 bp, artificial
  synthetic
gatgaacgctggcggcgtgcctaacacatgcaagtcgaacggagttcttgaggaatgcggcttcggccaaatgacttttgaacttagtggcggacgggtgagtaacgcgtgagcaacctgcctttcagagggggataacatttggaaacagatgctaataccgcataagattacagtaccgcatgatacagtgatcaaaggagcaatccgctgaaagatgggctcgcgtccgattagatagttggtgaggtaacggctcaccaagtcgacgatcggtagccggactgagaggttgaacggccacattgggactgagacacggcccagactcctacggaaggcagcagggttggt

SEQ ID NO: 447 - DNA, 335 bp, artificial
  synthetic
gatgaacgctagcgacaggcttaacacatgcaagtcgaggggtaacaggatgtagcaatacattgctgacgaccggcgcacgggtgagtaacgcgtatgcaacctgtccgttacagggggatagcccatggaaacgtggattaacactccataatataatatagaggcatctttgtattattaaatattcataggtaacggttgggcatgcgtcctattagatagttgggggggtaacggcccaccaagtcgatgataggtaggggttctgagaggaaggtcccccacactggtactgagacacggaccagactcctacggaaggcagcagggtt

SEQ ID NO: 448 - DNA, 334 bp, artificial
  synthetic
gacgaacgctggcggcgtgcctaacacatgcaagtcgaacggggtaatgacttcggttattacttagtggcgaacgggtgagtaacgcgtgaggaacctgcctttcagtgggggacaacagttggaaacgactgctaataccgcatgacacttcttaatggcatcattgagaagtcaaagctttatgtgctgaaagatggcctcgcgtctgattagctagttggtggggtaacggcccaccaaggcgacgatcagtagccggtctgagaggatgaacggccacattgggactgagatacggcccagactcctacggaaggcagcagggttggtt

SEQ ID NO: 449 - DNA, 330 bp, artificial
  synthetic
tttatcccaggctgggagttctcctttcccaggaactggatggggcctcatgagtggcactggcagtttcagttggcacctgagcaactttatgtgttatctttggtgatggggtgtgggatctgttctttcttgctggtgcatcctactttgaaacagtactggacttgccagcccagattgatgctggccaatcagatgtgcctctcaggattttcaggttaacaagcccacgctcaactccacactattacatcttccagtctttcatgtttctgttgatagagatgaagagcaccactcagggaaccacacggaaggcagcagggt

SEQ ID NO: 450 - DNA, 334 bp, artificial
  synthetic
gacgaacgctggcggcgtgcctaacacatgcaagtcgaacgggactcagcccttcgggactgagtttagtggcgaacgggtgagtaacgcgtgaggaacctgcctttcagtgggggacaacagttggaaacgactgctaataccgcataatgctttttgatggcatcattgaaaagccaaagatttattgctgaaagatggcctcgcgtctgattagctggttggtgaggtaacggcccaccaaggcgacgatcagtagccggtctgagaggatgaacggccacattgggactgagatacggcccagactcctacgggaggcagcagggttggt

SEQ ID NO: 451 - DNA, 340 bp, artificial
  synthetic
gatgaacgctagcgacaggcttaacacatgcaagtcgaggggcagcacagggtagcaatacttgggtggcgaccggcgcacgggtgagtaacgcgtatgcaacttacctatcagagggggatagcccggcgaaagtcggattaataccccatgaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagggttggtt

SEQ ID NO: 452 - DNA, 343 bp, artificial
  synthetic
gatgaacgctggcggcgtgcttaacacatgcaagtcgaacgaagcactttatttgatttcttcggaatgaagattttgtgactgagtggcggacgggtgagtaacgcgtgggtaacctgcctcatacagggggataacagttggaaacgactgctaataccgcataagcgcacagtatcgcatgatacagtgtgaaaaactccggtggtatgagatggacccgcgttggattagctagttggtgaggtaacggcccaccaaggcgacgatccgtagccgacctgagagggtgaccggccacattgggactgagacacggcccaaactcctacgggaggcagca

SEQ ID NO: 453 - DNA, 352 bp, artificial
  synthetic
gatgaacgctggcggcgtgcctaacacatgcaagtcgaacggggttattttggaaaatccttcgggattggaattcttaacctagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacggaaggcagcagggttggt

SEQ ID NO: 454 - DNA, 337 bp, artificial
  synthetic
gacgaacgctggcggcgtgcctaacacatgcaagtcgaacggagatgtttatttcggtagatatcttagtggcgcacgggtgagtaacgcgtgaataacctgacccgaagagggggataacacctggaaacaagtgctaataccgcataagaccacggaccggcatcggtcagaggtcaaaggaggaatccgctttgggaggggttcgcgtcccattaggtagttggtgaggtaacggcccaccaagccgacgatgggtagccgagctgagaggctgatcggccacactggaactgagacacggtccagactcctacggaaggcagcagggttggtt

SEQ ID NO: 455 - DNA, 330 bp, artificial
  synthetic
gacgaacgctggcggcgtgcttaacacatgcaagtcgaacggaaaggcccagcttgctgggtgctcgagtggcgaacgggtgagtaacacgtgggtgatctgcccctaacttcgggataagcttgggaaactgggtctaataccggataggacaatcgtttagtgtcggttgtggaaagttttttcggttagggatgagcccgcggcctatcagcttgttggtggggtaatggcctaccaaggcgtcgacgggtagccggcctgagagggtggacggccacattgggactgagatacggcccagactcctacgggaggcagcagggttgg

SEQ ID NO: 456 - DNA, 340 bp, artificial
  synthetic
gacgaacgctggcggcatgcctaatacatgcaagttgaacgatgatgttctctgcttgcagagaattgaagagtagcgaacgggtgagtaacacgtggggaacctgcccatgagagggggataacattcggaaacggatgctaataccgcataatgctttggaccgcctggtccagagacgaaagacggcttttgctgtcactcatggatggccccgcgccgtattagctagttggtgaggtaatggctcaccaaagctgtgatacgtagccgacctgagagggtgatcggccacactgggactgagacacggcccagactcctacgggaggcagcaggg

SEQ ID NO: 457 - DNA, 347 bp, artificial
  synthetic
atgaacgctggcggcgtgcctaacacatgcaagtcgagcgaagcactttgcttagattcttcggatgaagagttttgtgactgagcggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacggaaggcagcagggtt

SEQ ID NO: 458 - DNA, 374 bp, artificial
  synthetic
aaaaaataaataaataaagtgtcatctgggactgtggtcatctcaaggcttgactgggaaggatgtgcttccaagctcacactcatgtggttgctggcagcattcagttcctcaagggttgctggctagagtctgctctcttgtttttggccacatggggctctatatagggcaactcacaacatggcctctgacttcatcagaaggcacaaagatgagcaagatagaagccagagactttatgtaacctaatctcagaagtgacattgcatcagttttgctatattctactggttagacgcaagactctagccacagcccatactcagagagagggaattacatagggcaatacggaaggcagcagggttggt

SEQ ID NO: 459 - DNA, 331 bp, artificial
  synthetic
gatgaacgctggcggcatgcctaatacatgcaagtcgaacggtatccttcgggatacagtggcgaacgggtgagtaacacgtagggaacctgcccgcgcaccgggaatacgctctggaaacggagaacaaatcccgatggacagaaacagggcatcctgattctgtgaaacatccttctgggatggggcgcggatggacctgcggtgcattagttagttggcgagggtaacggcccaccaagacgatgatgcatagccggcctgagagggcggacggccacattgggactgagacacggcccagactcctacggaaggcagcagggttggt

SEQ ID NO: 460 - DNA, 346 bp, artificial
  synthetic
gatgaacgctggcggcgtgcctaacacatgcaagtcgaacgaagcattttagatgaagttttcggatggattctgagatgactgagtggcggacgggtgagtaacacgtggataacctgcctcacactgggggacaacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacggaaggcagcaggg

SEQ ID NO: 461 - DNA, 333 bp, artificial
  synthetic
gacgaacgctggcggcgtgcttaacacatgcaagtcgaacggaaaggccctgcttgcagggtgctcgagtggcgaacgggtgagtaacacgtgggtgatctgccccttactttgggataagcctgggaaactgggtctaatactggataggaccatgctgtaggtggtgtggtggaaagattagtttcggtaagggatgagctcgcggcctatcagcttgttggtggggtaatggcctaccaaggcgtcgacgggtagccggcctgagagggtggacggccacattgggactgagatacggcccagactcctacgggaggcagcagggttggt

SEQ ID NO: 462 - DNA, 348 bp, artificial
  synthetic
gatgaacgctggcggcgtgcctaacacatgcaagtcgaacgaagttacacagaggaagttttcggatggaatcggtataacttagtggcggacgggtgagtaacgcgtgggaaacctgccctgtaccgggggataacacttagaaataggtgctaataccgcataagcgcacggaaccgcatggttctgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacggaaggcagcagggttg

SEQ ID NO: 463 - DNA, 335 bp, artificial
  synthetic
gacgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacggctcggcatcgagcagagggaaaaggagcaatccgctttgagatggcctcgcgtccgattagctagttggtgaggtaacggcccaccaaggcgacgatcagtagccgacctgagagggtgaccggccacattgggactgagacacggcccaaactcctacgggaggcagcagggtt

SEQ ID NO: 464 - DNA, 349 bp, artificial
  synthetic
gatgaacgctagcgacaggcttaacacatgcaagtcgaggggcagcggggcggcaggcttgcctgccgttgccggcgaccggcgcacgggtgagtaacacgtatgcaacctgcccgtggcagggggataagcgggggaaaccccgtctaataccgcgtaacgcggcctagggacatcccaaggccgccaaagggagcaatcccggccacggatgggcatgcggcgcattagctagtcggcggggtaacggcccaccgaggcgacgatgcgtaggggttctgagaggaaggcccccccacactggtactgagacacggaccagactcctacgggaggcagcagggttggt

SEQ ID NO: 465 - DNA, 350 bp, artificial
  synthetic
gacgaacgctggcggcgtgcttaacacatgcaagtcgaacgggatttagtagacagaagcctcggtggaagattactaatgagagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagggttggt

SEQ ID NO: 466 - DNA, 336 bp, artificial
  synthetic
gatgaacgctggcggcgtgcttaacacatgcaagtcgagcggagataaattgaaagcttgcttttaatttatcttagcggcggacgggtgagtaacgtgtgggcaacctgcctcatacagagggataatcatgtgaaaacgtgactaataccgcatgtcgtttcgggagggcatcctcctgaaagaaaaggagcaatccggtatgagatgggcccgcatctgattagctagttggtgagataacagcccaccaaggcgacgatcagtagccgacctgagagggtgatcggccacattgggactgagacacggcccaaactcctacgggaggcagca

SEQ ID NO: 467 - DNA, 331 bp, artificial
  synthetic
attgaacgctggcggcaggcttaacacatgcaagtcgagcgggggaaggtagcttgctactggacctagcggcggacgggtgagtaatgcttaggaatctgcctattagtgggggacaacatctcgaaagggatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagggttggt

SEQ ID NO: 468 - DNA, 350 bp, artificial
  synthetic
gatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacagtaccgcatggtacagtgtgaaaaactccggtggtatgagatggacccgcgtctgattagctagttggtggggtaacggcctaccaaggcgacgatcagtagccgacctgagagggtgaccggccacattgggactgagacacggcccaaactcctacggaaggcagcagggtt

SEQ ID NO: 469 - DNA, 348 bp, artificial
  synthetic
gatgaacgctggcggcgtgcctaacacatgcaagtcgaacgaagcgctttgattgattccttcgggatgatttcaaagtgacttagtggcggacgggtgagtaacgcgtgggtaacctgccttgtacagggggataacagttagaaatgactgctaataccgcataacccgctaaggtcgcatgacctggacggaaaagatttatcggtacaagatggacccgcgtctgattagctagttggtgaggtaacggcccaccaaggcgacgatcagtagccggcctgagagggtgaacggccacattgggactgagacacggcccaaactcctacgggaggcagcagggtt

SEQ ID NO: 470 - DNA, 346 bp, artificial
  synthetic
gatgaacgctggcggcgtgcttaacacatgcaagtcgaacgaagcaccttgatttgattcttcggatgaagatcttggtgactgagtggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacggaaggcagcaggg

SEQ ID NO: 471 - DNA, 440 bp, artificial
  synthetic
gacgaacgctggcggcatgcctaacacatgcaagtcgaacggagaaagttcaacaccaagtatttcatccgctatagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcag

SEQ ID NO: 472 - DNA, 349 bp, artificial
  synthetic
gatgaacgctggcggcgtgcttaacacatgcaagtcgaacggagtgcctttgaaagaggattcgtccaattgataaggttacttagtggcggacgggtgagtaacgcgtgaggaacctgccttggagtggggaataacacagtgaaaattgtgctaataccgcataatgcagttgggccgcatggctctgactgccaaagatttatcgctctgagatggcctcgcgtctgattagctagttggtggggtaacggcccaccaaggcgacgatcagtagccggactgagaggttggccggccacattgggactgagacacggcccagactcctacggaaggcagcagggtt

SEQ ID NO: 473 - DNA, 344 bp, artificial
  synthetic
gatgaacgctggcggcgtgcttaacacatgcaagtcgaacgaagcacagaaagcagattacttcggtttgaagctttctgtgactgagtggcggacgggtgagtaacgcgtggataacctgcctcacacagggggataacagttagaaatgactgctaataccgcataagcgcacagtctcgcatgggacagtgtgaaaaactgaggtggtgtgagatggatccgcgtctgattaggcagttggcggggtaacggcccaccaaaccaacgatcagtagccggcctgagagggtgaacggccacattgggactgagacacggcccaaactcctacggaaggcagc

SEQ ID NO: 474 - DNA, 342 bp, artificial
  synthetic
gatgaacgctagcggcaggcttaacacatgcaagtcgaagggcatcggggagagtgcttgcactctctgccggcgactggcgcacgggtgagtaacacgtatgcaacctgccctccacagggggacaaccttccgaaagggaggctaatcccgcgtatattccctgggggcatccctgggggaggaaagggttaccggtggaggatgggcatgcggcgcattaggcagtaggcggggtaacggcccacctaaccgacgatgcgtaggggttctgagaggaaggtcccccacactggtactgagacacggaccagactcctacggaaggcagcagggttggtt

SEQ ID NO: 475 - DNA, 336 bp, artificial
  synthetic
gacgaacgctggcggcacgcctaacacatgcaagtcgaacggagaagagagcttcggctcttggatcagtggcggacgggtgagtaacacgtgagcaacctgcctttcagagggggacaacagttggaaacgactgctaataccgcataatgtatacgaatggcatcatttgtataccaaaggagcaatccgctgaaagatgggctcgcgtctgattagatagttggtgaggtaacggctcaccaagtcgacgatcagtagccggactgagaggttgaacggccacattgggactgagacacggcccagactcctacggaaggcagcagggttggt

SEQ ID NO: 476 - DNA, 338 bp, artificial
  synthetic
gatgaacgctggcggcgtgcctaatacatgcaagtcgaacgcttgcttgaggacttgtcttcaagcgggagtggcgaacgggtgagtaatacataagcaatctgcccatcggcctgggataacagttggaaacgactgctaataccggataggtgatgaagaggcatctcttgatcattaaagttgggatacaacacggatggatgagcttatggcgtattagctagtaggtgaggtaacggctcacctaggcgatgatacgtagccgacctgagagggtgaccggccacattgggactgagacacggcccaaactcctacggaaggcagcagggttg

SEQ ID NO: 477 - DNA, 305 bp, artificial
  synthetic
aacgaacgctggcggcatgcctaacacatgcaagtcgaacgagaccttcgggtctagtggcgcacgggtgcgtaacgcgtgggaatctgcccttgggttcggaataactgttagaaatgactgctaataccggatgatgacgtaagtccaaagatttatcgcccaaggatgagcccgcgtaggattagctagttggtgaggtaaaggctcaccaaggcgacgatccttagctggtctgagaggatgatcagccacactgggactgagacacggcccagactcctacgggaggcagcagggttggt

SEQ ID NO: 478 - DNA, 335 bp, artificial
  synthetic
gacgaacgctggcggcgtgcctaacacatgcaagtcgaacggacgaaggagcttcggctccctagttagtggcgaacgggtgagtaacgcgtgagcaacctgcctttcagagggggacaacagctggaaacggctgctaataccgcataacatgatttagccgcatgactggatcatcaaagatttatcgctgaaagatgggctcgcgtctgattagctagttggtggggtaaaggcccaccaaggcgacgatcagtagccggactgagaggttgaacggccacattgggactgagatacggcccagactcctacggaaggcagcagggttggtt

SEQ ID NO: 479 - DNA, 352 bp, artificial
  synthetic
gacgaacgctggcggcgcgcctaacacatgcaagtcgaacggagatcatttggtagaagttttcggatggacaccgagtgatcttagtggcgaacgggtgagtaacgcgtgagcaatctgcctcagagtgggggacaacagttggaaacgactgctaataccgcataagcccacagcaccgcatggtgcagggggaaaagatttattgctttgagatgagctcgcgtccaattagctagttggtgaggtaacggcccaccaaggcgacgattggtagccggactgagaggttgaacggccacattgggactgagatacggcccagactcctacggaaggcagcagggttggt

SEQ ID NO: 480 - DNA, 349 bp, artificial
  synthetic
gatgaacgctggcggcgtgcttaacacatgcaagtcgagcgaagcgatttaaatgagacttcggtggattttaaattgactgagcggcggacgggtgagtaacgcgtggataacctgcctcacacagggggataacagttagaaatgactgctaataccgcataagcgcacggtaccgcatggtacagtgtgaaaaactccggtggtgtgagatggatccgcgtctgattaggtagttggtgaggtaacggcccaccaagccgacgatcagtagccgacctgagagggtgaccggccacattgggactgagacacggcccaaactcctacggaaggcagcagggttggt

SEQ ID NO: 481 - DNA, 353 bp, artificial
  synthetic
gatgaacgctggcggcgtgcttaacacatgcaagtcgaacgaagcgatagagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacggaaggcagcagggttggt

SEQ ID NO: 482 - DNA, 347 bp, artificial
  synthetic
gatgaacgctggcggcgtgcctaacacatgcaagtcgagcgaagcacttgcgaatgatccttcgggtgattttgctggtgactgagcggcggacgggtgagtaacgcgtgggtaacctgcctcatacagggggataacagttagaaatgactgctaataccgcataagcgcacagtaccacatggtacggtgtgaaaaactccggtggtatgagatggacccgcgtctgattagctagttggtggggtaacggcccaccaaggcgacgatcagtagccgacctgagagggtgaccggccacattgggactgagacacggcccaaactcctacggaaggcagcagggt

SEQ ID NO: 483 - DNA, 336 bp, artificial
  synthetic
gacgaacgctggcggcgtgcctaacacatgcaagtcgagcgatgaagtttccttcgggaaatggattagcggcggacgggtgagtaacacgtgggtaacctgcctcatagtggggaatagcctttcgaaaggaagattaataccgcataagattgtagtttcgcatgaaacagcaattaaaggagcaattcgctatgagatggacccgcggcgcattagctagttggtgaggtaacggctcaccaaggccacgatgcgtagccgacctgagagggtgatcggccacattgggactgagacacggcccagactcctacggaaggcagcagggttggt

SEQ ID NO: 484 - DNA, 305 bp, artificial
  synthetic
aacgaacgctggcggcatgcctaatacatgcaagtcgaacgatcacttcggtgatagtggcgcacgggtgcgtaacgcgtgggaatctgcccttgggttcggaataacatctggaaacggatgctaataccggatgatgacgtaagtccaaagatttatcgcccagggatgagcccgcgtaggattagctagttggtggggtaaaggcctaccaaggcgacgatccttagctggtctgagaggatgatcagccacactgggactgagacacggcccagactcctacgggaggcagcagggttggt

SEQ ID NO: 485 - DNA, 341 bp, artificial
  synthetic
gatgaacgctagctacaggcttaacacatgcaagtcgaggggcagcatgactgaagcttgcttcagttgatggcgaccggcgtacgggtgcgtaacgcgtatccaacctgccctctactccggaacagcccgtcgaaaggcggattaatgccggatggtgtccacttggtgcatgccatggtggactaaaggtaacggtagaggatggggatgcgactgattaggtagacggcggggtaacggcccaccgtgccgacgatcagtaggggttctgagaggaaggtcccccacactggaactgagacacggtccagactcctacggaaggcagcagggttggt

SEQ ID NO: 486 - DNA, 344 bp, artificial
  synthetic
gatgaacgctggcggcgtgcctaatacatgcaagtagaacgctgaagcttggtgcttgcaccgagcggatgagttgcgaacgggtgagtaacgcgtaggtaacctgcctggtagcgggggataactattggaaacgatagctaataccgcataacagtagatattgcatgatatctgcttgaaaggggcaattgctccactaccagatggacctgcgttgtattagctagttggtgaggtaacggctcaccaaggcgacgatacatagccgacctgagagggtgatcggccacactgggactgagacacggcccagactcctacggaaggcagcagggttggtt

SEQ ID NO: 487 - DNA, 340 bp, artificial
  synthetic
gatgaacgctagctacaggcttaacacatgcaagtcgaggggcagcatgggattagcttgctaatcctgatggcgaccggcgcacgggtgcgtaacgcgtatccaacctgccctctactccggaacagcccgtcgaaaggcggattaatgccggatggtgtcacatgcccgcatgagtgtgtgactaaaggcaacggtagaggatggggatgcgactgattagctagttggcggggtaacggcccaccaaggctacgatcagtaggggttctgagaggaaggtcccccacattggaactgagacacggtccaaactcctacgggaggcagcagggttggt

SEQ ID NO: 488 - DNA, 343 bp, artificial
  synthetic
gatgaacgctggcggcgtgcttaacacatgcaagtcgaacgaagcggtttagcggaagttttcggatggaagttaaactgactgagtggcggacgggtgagtaacgcgtgggtaacctgcctcacacagggggacaacagctagaaatggctgctaataccgcataagcgcacagcttcgcatgaagcagtgtgaaaaactccggtggtgtgagatggacccgcgtctgattagctagttggtgaggtaacggctcaccaaggcgacgatcagtagccgacctgagagggtgaccggccacattgggactgagacacggcccaaactcctacgggaggcagca

SEQ ID NO: 489 - DNA, 336 bp, artificial
  synthetic
gacgaacgctggcggcgtgcctaacacatgcaagtcgaacggggttgagtggttcggtattcaacttagtggcgaacgggtgagtaacgcgtgaagaacctgcctttcagagggggacaacagttggaaacgactgctaataccgcataagcccacgggtcggcatcgaccagagggaaaaggagtgatccgctttgagatggcctcgcgtccgattagctagttggtgaggtaacggcccaccaaggcgacgatcggtagccggactgagaggttgaacggctacattgggactgagacacggcccagactcctacggaaggcagcagggttggt

SEQ ID NO: 490 - DNA, 335 bp, artificial
  synthetic
gacgaacgctggcggcgtgcctaacacatgcaagtcgaacggaagttagagcttcggttttaactttagtggcgaacgggtgagtaacgcgtgaggaacctgcctttcagtgggggacaacagttggaaacgactgctaataccgcatgacacttttgggagacatctcctggaagtcaaagctttatgtgctgaaagatggcctcgcgtctgattagctagttggtgaggtaacggctcaccaaggcgacgatcagtagccggtctgagaggatgaacggccacattgggactgagatacggcccagactcctacggaaggcagcagggttggt

SEQ ID NO: 491 - DNA, 351 bp, artificial
  synthetic
gatgaacgctggcggcgtgcttaacacatgcaagtcgaacggagtgctcatgacagaggattcgtccaatggagtgagttacttagtggcggacgggtgagtaacgcgtgagtaacctgccttggagtggggaataacaggtggaaacatctgctaataccgcatgatgcagttgggtcgcatggctctgactgccaaagatttatcgctctgagatggactcgcgtctgattagctggttggcggggtaacggcccaccaaggcgacgatcagtagccggactgagaggttggccggccacattgggactgagacacggcccagactcctacggaaggcagcagggttgg

SEQ ID NO: 492 - DNA, 366 bp, artificial
  synthetic
gacgaacgctggcggcgtgcctaatacatgcaagtcgagcgagtctgccttgaagatcggagtgcttgcactctgtgaaacaagatacaggctagcggcggacgggtgagtaacacgtgggtaacctgcccaagagatcgggataacacctggaaacagatgctaataccggataacaacagatgatgcctatcaactgtttaaaagatggttctgctatcactcttggatggacctgcggtgcattagctagttggtagggtaacggcctaccaaggcgatgatgcatagccgagttgagagactgatcggccacattgggactgagacacggcccaaactcctacgggaggcagcagggttggt

SEQ ID NO: 493 - DNA, 227 bp, artificial
  synthetic
ccccgcgaagagacgctcctcttgagggcacctgtccctctgtttgcatcccttggtgagggaccaagcgtgtgcggagggaggctgggaagcacagcctggctgtgtgtccactataaggtattcccgtggaaggcaaagcacagcgttaacagagtgctggccagctctgtcccacagttccctgtaccttggcacgcagcagcacgggaggcagcagggttggt

SEQ ID NO: 494 - DNA, 229 bp, artificial
  synthetic
tcatttcgggaacctttcccgctccagcacacacaactcagccaggccccagtggagctcaaagtggctcatgagccagggagctccctgaaactatcctctttcttgcttggccttggcgatgctgatgctgctggcgccccatggtcttcatccatgagtgaccatccaaatagggccagtctcccagcccctctaatggccctcttgggaaggcagcagggttggt

SEQ ID NO: 495 - DNA, 336 bp, artificial
  synthetic
gatgaacgctagcgacaggcttaacacatgcaagtcgaggggcagcgagggagtggcaacacttctgtcggcgaccggcgcacgggtgagtaacgcgtatgcaacctgcccttcacagggggataaccgggagaaattccgactaataccgcatacgtcctccgggggcatccccgggggatgaaagaattatcggtgaaggatgggcatgcgtgatattaggtagttggcggggcaacggcccaccaagcccacgatatctaggggttctgagaggaaggtcccccacattggtactgagacacggaccaaactcctacgggaggcagcagggtt

SEQ ID NO: 496 - DNA, 341 bp, artificial
  synthetic
gatgaacgctagcgacaggcttaacacatgcaagtcgaggggcagcgggggagcagcaatgctcccgccggcgaccggcgcacgggtgagtaacacgtatggaacctgcccgcagcagggggataagcggaagaaattccgtctaataccgcgtaacaccgcggaggggcatccctcggcggttaaagattcatcggctgcggatggccatgcggcgcattagctagtcggcggggtaacggcccaccgaggcgacgatgcgtaggggttctgagaggaaggtcccccacactggtactgagacacggaccagactcctacggaaggcagcagggttggtt

SEQ ID NO: 497 - DNA, 341 bp, artificial
  synthetic
gataaacgctggcggcgcacataagacatgcaagtcgaacggacttaaccgaaagtttacttttggagcggttagtggcggactggtgagtaacacgtaagcaacctgcctatcagaggggaacaacagttagaaatgactgctaataccgcatatacctaagtaccacatggtgcaatagggaaaggagcaatccgctgatagatgggcttgcgtctgattagatagttggtgtggtaacggcacaccaagtcgacgatcagtagccggactgagaggttgaacggccacattgggactgagatacggcccagactcctacggaaggcagcagggttggt

SEQ ID NO: 498 - DNA, 331 bp, artificial
  synthetic
gatgaacgctggcggcgtgcttaacacatgcaagtcgaacgatgatgcccacttgtgggtggattagtggcgaacgggtgagtaacacgtgagtaacctgcccttgactctgggataagcctgggaaactgggtctaataccggatatgaccttccatcgcatggtggttggtggaaagcttttgtggttttggatggactcgcggcctatcagcttgttggtgaggtaatggcttaccaaggcgacgacgggtagccggcctgagagggtgaccggccacactgggactgagacacggcccagactcctacgggaggcagcagggttggt

SEQ ID NO: 499 - DNA, 307 bp, artificial
  synthetic
agcgaacgctggcggcaggcttaacacatgcaagtcgagcgggcaccttcgggtgtcagcggcagacgggtgagtaacacgtgggaacgtacccttcggttcggaataacgctgggaaactagcgctaataccggatacgcccttatggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagggttggt

SEQ ID NO: 500 - DNA, 338 bp, artificial
  synthetic
gatgaacgctagctacaggcttaacacatgcaagtcgaggggcagcacagtgtagcaatacatgggtggcgaccggcgcacgggtgcgtaacgcgtatccaaccttcccgatactcttggatagccttccgaaagggagattaatacaagatggtgtttcaattccgcatgttattgaaactaaagatttatcggtatcggatggggatgcgtgacattagatagtaggcggggtaacggcccacctagtctacgatgtctaggggttctgagaggaaggtcccccacactggaactgagacacggtccagactcctacggaaggcagcagggttggt

SEQ ID NO: 501 - DNA, 334 bp, artificial
  synthetic
gacgaacgctggcggcgtgcctaacacatgcaagtcgagcgagtggagttcttcggaacaaagctagcggcggacgggtgagtaacacgtgggcaacctgcctcatagaggggaatagccttccgaaagggagattaataccgcataagattgtagcttcgcatgaagtagcaattaaaggagcaatccgctatgagatgggcccgcggcgcattagctagttggtgaggtaacggctcaccaaggcgacgatgcgtagccgacctgagagggtgatcggccacattgggactgagacacggcccagactcctacgggaggcagcagggttgtt

SEQ ID NO: 502 - DNA, 335 bp, artificial
  synthetic
gatgaacgctggcggcgtgcctaacacatgcaagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggaaatggggtcgcggcccattaggtagtaggcggggtaacggcccacctagcccgcgatgggtagccgggttgagagaccgaccggccacattgggactgagatacggcccagggggctacggaaggcagcagggttggtt

SEQ ID NO: 503 - DNA, 351 bp, artificial
  synthetic
gatgaacgctggcggcgtgcttaacacatgcaagtcgagcgaagcacttaagtttgattcttcggatgaagacttttgtgactgagcggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctagttggtggggtaacggcccaccaaggcgacgatccatagccggcctgagagggtgaacggccacattgggactgagacacggcccagactcctacgggaggcagcagggttggt

SEQ ID NO: 504 - DNA, 353 bp, artificial
  synthetic
gatgaacgctggcggcgtgcctaacacatgcaagtcgaacgaagcaatcttaatgaagttttcggatggatttgagattgacttagtggcggacgggtgagtaacgcgtgggtaacctgccttgtacagggggataacagctggaaacgactgctaataccgcatgatactttcgaggggcatcccttgaaagtcaaagctttatgtgctgaaagatggcttcgcgtctgattagctagttggtggggtaacggcccaccaaggcgacgatcagtagccggtctgagaggatgaacggccacattgggactgagatacggcccagactcctacgggaggcagcagggttggtt

SEQ ID NO: 505 - DNA, 343 bp, artificial
  synthetic
gacgaacgctggcggcgtgcctaatacatgcaagtagaacgctgaaggaggagcttgctctttccggatgagttgcgaacgggtgagtaacgcgtaggtaacctgcctggtagcgggggataactattggaaacgatagctaataccgcataacagtagatattgcatgatatctgcttgaaaggtgcaattgcaccactaccagatggacctgcgttgtattagctagttggtgaggtaacggctcaccaaggcgacgatacatagccgacctgagagggtgatcggccacactgggactgagacacggcccagactcctacgggaggcagcagggttggtt

SEQ ID NO: 506 - DNA, 348 bp, artificial
  synthetic
gatgaacgctggcggcgtgcttaacacatgcaagtcgagcgaagcacctaagaaagattcttcggatgaattcttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcagggtt

SEQ ID NO: 507 - DNA, 326 bp, artificial
  synthetic
gacgaacgctggcggcgtgcctaacacatgcaagtcgagcgatgaagttccttcgggaacggattagcggcggacgggtgagtaacacgtgggcaacctgccttatagaggggaatagccttccgaaaggaagattaataccgcataagattgtagcttcgcatgaagtagcaattaaaggagcaatccgctataagatgggcccgcggcgcattagctagttggtgaggtaacggctcaccaaggcgacgatgcgtagccgacctgagagggtgatcggccacattgggactgagacacggcccagactcctacggaaggcagca

SEQ ID NO: 508 - DNA, 345 bp, artificial
  synthetic
attgaacgctggcggaacgctttacacatgcaagtcgaacggtaacagcgaggaaagcttgcttttttcggctgacgagtggcgaacgggtgagtaatacatcggaacgtgtccgctcgtgggggacaaccatccgaaaggatggctaataccgcatgagttctacggaagaaagagggggacctgcttgcaggcctctcgcgagcggagcggccgatgactgattagccagttggtgaggtaacggctcaccaaagcaacgatcagtagctggtctgagaggacgaccagccacactgggactgagacacggcccagactcctacggaaggcagcagggttggt

SEQ ID NO: 509 - DNA, 347 bp, artificial
  synthetic
gatgaacgctggcggcgtgcttaacacatgcaagtcgaacgggaaacttttcattgaagcttcggcagatttggtctgtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatcagtagccgacctgagagggtgaccggccacattgggactgagacacggcccaaactcctacgggaggcagcagggtt

SEQ ID NO: 510 - DNA, 337 bp, artificial
  synthetic
gacgaacgctggcggcgtgcctaacacatgcaagtcgagcgaggaatgtcggatagcttgctatttgatatttctagcggcggacgggtgagtaacgcgtgagcaacctgcctttatcagggggataacgcatcgaaagatgtgctaataccgcgtaagaccacagcctcacatggggcaggggtcaaaggagcaatccggataaagatgggctcgcgtccgattagctagttggtgagataacagcccaccaaggcgacgatcggtagccgacctgagagggtgatcggccacattggaactgagagacggtccagactcctacggaaggcagcag

SEQ ID NO: 511 - DNA, 348 bp, artificial
  synthetic
gatgaacgctggcggcgtgcttaacacatgcaagtcgaacggagcacccctgaaggagttttcggacaactgaagggaatgcttagtggcggacgggtgagtaacgcgtgagtaacctgccttggagtggggaataacagttggaaacagctgctaataccgcatgatgcagttgagtcgcatggctctgactgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagggt

SEQ ID NO: 512 - DNA, 348 bp, artificial
  synthetic
gatgaacgctagcgacaggcttaacacatgcaagtcgaggggcagcgggggatgcgttcgcgcattctgccggcgaccggcgcacgggtgagtaacacgtatgcaacctgccccgtccagggggataatcggcggaaacgccgtctaataccgcgtatatcggtaccgggcatccgggattgaagaaagggccttagggtccgggacgggatgggcatgcggcgcattaggaagttggcggtgtaacggaccaccaatccgtcgatgcgtaggggttctgagaggaaggcccccccacactggtactgagacacggaccagactcctacggaaggcagcagggttggt

SEQ ID NO: 513 - DNA, 351 bp, artificial
  synthetic
gacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacggaaggcagcagggttggt

SEQ ID NO: 514 - DNA, 342 bp, artificial
  synthetic
gatgaacgccggcggtgtgcctaatacatgcaagtcgtacgcactggcccaactgattgacgttggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagca

SEQ ID NO: 515 - DNA, 303 bp, artificial
  synthetic
aacgaacgctggcggcaggcttaacacatgcaagtcgaacgccccgcaaggggagtggcagacgggtgagtaacgcgtgggaacgtaccctttactacggaataactcagggaaacttgtgctaataccgtatgtgcccttcgggggaaagatttatcggtaaaggatcggcccgcgttggattagctagttggtggggtaaaggcctaccaaggcgacgatccatagctggtctgagaggatgatcagccacattgggactgagacacggcccaaactcctacggaaggcagcagggttggt

SEQ ID NO: 516 - DNA, 347 bp, artificial
  synthetic
gatgaacgctggcggcgtgcttaacacatgcaagtcgagcgaagcgcttaagtttgattcttcggatgaagagttttgtgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagggt

SEQ ID NO: 517 - DNA, 335 bp, artificial
  synthetic
gataaacgctggcggcatgcctaatacatgcaagtcgaacggaatcaggcttcggtctgattcagtggcgaacgggtgaggaacacgtagggaacctgcccgcagccgggggatacgctttggaaacgaagtctaaaaccccataggagccattcaggcatctgaaaggcttgaaagtaacaactgttacggcggcggatggacctgcggtgcattagttagttggcggggcaacggcccaccaagacgatgatgcatagccggcctgagagggcggacggccacactgggactgagacacggcccagactcctacggaaggcagcagggttggt

SEQ ID NO: 518 - DNA, 353 bp, artificial
  synthetic
gatgaacgctggcggcgtgcttaatacatgcaagtcgaacgaagcaccttggacagaatccttcgggaggaagaccattgtgactgagtggcggacgggtgagtaacgcgtgggtaacctgccttgtacagggggataacagttggaaacgactgctaataccgcataagcgcacagtaccgcatggtacggtgtgaaaaactccggtggtacaagatggacccgcgtctgattagctggttggtgaggtaacggcccaccaaggcgacgatcagtagccgacttgagagagtgatcggccacattgggactgagacacggcccaaactcctacggaaggcagcagggttggt

SEQ ID NO: 519 - DNA, 332 bp, artificial
  synthetic
gatgaacgctagcgggaggcctaacacatgcaagccgagcggtatttgttcttcggaacagagagagcggcgcacgggtgcggaacacgtgtgcaacctgcctttatctgggggatagcctttcgaaaggaagattaataccccataatatattgagtggcatcatttgatattgaaaactccggtggatagagatgggcacgcgcaagattagatagttggtgaggtaacggctcaccaagtcaatgatctttagggggcctgagagggtgatcctccacactggtactgagacacggaccagactcctacgggaggcagcagggttggtt

SEQ ID NO: 520 - DNA, 351 bp, artificial
  synthetic
gatgaacgctggcggcgtgcttaacacatgcaagtcgaacggagttaacctctgtcaaagcttcggcagagacttggttaacttagtggcggacgggtgagtaacgcgtggataacctgccatatacagggggataacacttagaaataggtgctaataccgcataagcgcacagtttcgcatgaaacggtgtgaaaaactccggtggtatatgatggatccgcgtctgattagctagttggtgaggtaacggcccaccaaggcaacgatcagtagccggcctgagagggtgaacggccacattgggactgagacacggcccaaactcctacgggaggcagcagggttggt

SEQ ID NO: 521 - DNA, 331 bp, artificial
  synthetic
gacgaacgctggcggcgtgcttcatacatgcaagtcgaacgagaatctctagcttgctagagaggacagtggcggacgggtgagtaatgtgtagagaatctgcccttgagagggggacaacagagggaaacttctgctaataccccatatgagataagctgaaatgcttatcttgaaaactccggtgctcaaggatgagtctgcatctgattagctagttgggggtgtaatggaccaccaaggcgacgatcagtagctggtttgagaggatgatcagccacaatgggactgagacacggcccatactcctacggaaggcagcagggttggt

SEQ ID NO: 522 - DNA, 333 bp, artificial
  synthetic
gacgaacgctggcggcgtgcctaacacatgcaagtcgaacgggacttgccccttcggggacaagtttagtggcggacgggtgagtaacgcgtgagcaacctgcctttcagtgggggacaacagttggaaacgactgctaataccgcataacacttattaggggcatctctgataagtcaaagatttattgctgaaagatgggctcgcgtctgattagctagttggtggggtaacggcccaccaaggcgacgatcagtagccggactgagaggttgaacggccacattgggactgagatacggcccagactcctacggaaggcagcagggttgg

SEQ ID NO: 523 - DNA, 348 bp, artificial
  synthetic
gatgaacgctagcgacaggcttaacacatgcaagtcgaggggcagcggggaggaaagcttgctttcctccgccggcgaccggcgcacgggtgagtaacacgtatgcaacctgccctcggcagggggataatccggagaaatccggtctaataccgcgtggcacccctgaggggcatcccttgggggttaaaggaagcgattccggccgaggatgggcatgcgtcgcattaggcagttggcggtgtaacggaccaccaaaccgacgatgcgtaggggttctgagaggaaggtcccccacactggtactgagacacggaccagactcctacggaaggcagcagggttggt

SEQ ID NO: 524 - DNA, 342 bp, artificial
  synthetic
gatgaacgctagctacaggcttaacacatgcaagtcgaggggcagcatgttggttgcttgcaaccaacgatggcgaccggcgcacgggtgagtaacgcgtatccaaccttccgcatactcgggaatagcctttcgaaagaaagattaatgcccgatggtttcccgaatccgcatgagcgcgggaataaagattcatcggtatgcgatggggatgcgtcccattagcttgttggcggggtaacggcccaccaaggctacgatgggtaggggttctgagaggaaggtcccccacattggaactgagacacggtccaaactcctacggaaggcagcagggttggt

SEQ ID NO: 525 - DNA, 343 bp, artificial
  synthetic
gataaacgctggcggcgcacataagacatgcaagtcgaacggacttaactcattcttttagattgagagcggttagtggcggactggtgagtaacacgtaagcaacctgcctatcagaggggaataacaacgagaaatcgttgctaataccgcataagctagtagcatcgcatgatgtagctagaaaaggagcaatccgctgatagatgggcttgcgtctgattagctagttggtggggtaacggcctaccaaggcgacgatcagtagccggactgagaggttgaacggccacattgggactgagatacggcccagactcctacggaaggcagcagggttggt

SEQ ID NO: 526 - DNA, 342 bp, artificial
  synthetic
gacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataatagcagttgttgcatgacaactgtttgaaaggtgcaattgcaccactaccagatggacctgcgttgtattagctagttggtggggtaacggctcaccaaggcgacgatacatagccgacctgagagggtgatcggccacactgggactgagacacggcccagactcctacgggaggcagcagggttggtt

SEQ ID NO: 527 - DNA, 360 bp, artificial
  synthetic
gacaaacgctggcggcatgcctaacacatgcaagtcgaacggacggaggcttgagatctcttcggagtgaccgagcccgagttagtggcggatgggtgagtaacgcgtggggaacctaccttttagtggggaataatcgttggaaacgacgactaataccgcatacagtgtccggatcgcatgatccggataaaaaagacggcctttgtgctgtcgctaagagatggacccgcgtctgattagctagttggtaaggtaacggcttaccaaggcgacgatcagtagccggcctgagagggtgaacggccacattgggactgagacacggcccaaactcctacggaaggcagcagggttggt

SEQ ID NO: 528 - DNA, 352 bp, artificial
  synthetic
gatgaacgctggcggcgtgcttaacacatgcaagtcgaacgaagcactttaccacgattccttcgggatgacggtttagtgactgagtggcggacgggtgagtaacgcgtggggaacctgccccataccgggggataacagccggaaacggctgctaataccgcataagcgcacagtaccgcatggtacggtgtgaaaaactccggtggtatgggatggacccgcgtctgattagccagttggcggggtaacggcccaccaaagcgacgatcagtagccggcctgagagggcgaccggccacattgggactgagacacggcccagactcctacggaaggcagcagggttggt

SEQ ID NO: 529 - DNA, 355 bp, artificial
  synthetic
gatgaacgctggcggcgtgcctaacacatgcaagtcgaacgggtgcgcgccgaggaggccggttataccagccgaaacggtgcgcatgagtggcggacgggtgagtaacgcgtgggcaacctgccgtatacagggggataacacccggaaacgggtgctaataccgcataagcgcacgagtgccgcatggcacggtgtgaaaaactccggtggtatacgatgggcccgcgtccgattagctggttggcggggcagcggcccaccaaggcgacgatcggtagccggcctgagagggcggacggccacattgggactgagacacggcccaaactcctacggaaggcagcagggtttt

SEQ ID NO: 530 - DNA, 346 bp, artificial
  synthetic
gatgaacgctggcggcgtgcctaacacatgcaagtcgaacgaagcggatttgatgaagttttcggatgaattcaaatctgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacctgcgtctgattagctagttggtgaggtaacggcccaccaaggcgacgatcagtagccgacctgagagggtgaccggccacattgggactgagacacggcccaaactcctacgggaggcagcaggg

SEQ ID NO: 531 - DNA, 335 bp, artificial
  synthetic
gatgaacgctggcggcgtgcctaacacatgcaagtcgaacgaagcaccttgaagttttcggacggattggtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacctgcgtctgattagctagttggtgaggtaacggcccaccaaggcgacgatcagtagccgacctgagagggtgaccggccacattgggactgagacacggcccaaactcctacgggaggcagca

SEQ ID NO: 532 - DNA, 331 bp, artificial
  synthetic
gacgaacgctggcggcgtgcctaacacatgcaagtcgaacggagttacccttcggggtagcttagtggcgaacgggtgagtaacgcgtgaagaacctgcctttcagtgggggacaacagttggaaacgactgctaataccgcataatgtcatttgggggcatccccgaatgaccaaagatttattgctgaaagatggcttcgcgtctgattagctagttggtgaggtaacggctcaccaaggcgacgatcagtagccggtctgagaggatgaacggccacattgggactgagatacggcccagactcctacggaaggcagcagggttggtt

SEQ ID NO: 533 - DNA, 304 bp, artificial
  synthetic
aacgaacgctggcggcatgcctaacacatgcaagtcgaacgatgctttcgggcatagtggcgcacgggtgcgtaacgcgtgggaatctgcccttgggtctgggataacagttggaaacgactgctaataccggatgatatcgcgagatcaaagatttatcgcccgaggatgagcccgcgtaggattagctagttggtggggtaaaggcctaccaaggcgacgatccttagctggtctgagaggatgatcagccacactgggactgagacacggcccagactcctacgggaggcagcagggttgg

SEQ ID NO: 534 - DNA, 353 bp, artificial
  synthetic
gatgaacgctggcggcgtgcttaacacatgcaagtcgaacgaagcacttggaattgatttcttcggattgattttccaagtgactgagtggcggacgggtgagtaacgcgtgggcaacctgcctcatacagggggataacagttagaaatggctgctaataccgcataagcgcacagtaccgcatggtacggtgtgaaaaactccggtggtatgagatggacccgcgtctgattagctagttggtaaggtaacggcttaccaaggcgacgatcagtagccgacctgagagggtgatcggccacattgggactgagacacggcccaaactcctacgggaggcagcagggttggt

SEQ ID NO: 535 - DNA, 339 bp, artificial
  synthetic
gatgaacgctggcggcgtgcctaacacatgcaagtcgaacgggggcagattgaaacctagtgatatctgcccgagtggcggacgggtgagtaacgcgtggacaacctgccgcatgcagggggataccggctggaaacagccgctaataccgcatatgcgcacggcgccgcatggcgcagtgcggaaagggagcgatcccggcatgcgatgggtccgcgtccgattagcttgttggcggggcagcggcccaccaaggcgacgatcggtagccggcctgagagggcggacggccacattgggactgagacacggcccagactcctacggaaggcagcaggg

SEQ ID NO: 536 - DNA, 347 bp, artificial
  synthetic
gacgaacgctggcggcgtgcttaacacatgcaagtcgaacgagaatctgtggatcgaggattcgtccaagtgaagcagaggacagtggcggacgggtgagtaacgcgtgaggaacctgcctttcagagggggacaacagttggaaacgactgctaataccgcatgatacatttgggtggcatcatctgaatgtcaaagatttatcgctgaaagatggcctcgcgtctgattagctggttggtgaggtaacggcccaccaaggcgacgatcagtagccggactgagaggttgaccggccacattgggactgagatacggcccagactcctacgggaggcagcagggtt

SEQ ID NO: 537 - DNA, 305 bp, artificial
  synthetic
aatgaacgctggcggcatgcctaacacatgcaagtcgaacgaaggcttcggccttagtggcgcacgggtgcgtaacgcgtgggaatctgccccttggttcggaataacagttggaaacgactgctaataccggatgatgacgtaagtccaaagatttatcgccgagggatgagcccgcgtaggattagctagttggtgtggtaaaggcgcaccaaggcgacgatccttagctggtctgagaggatgatcagccacactgggactgagacacggcccagactcctacggaaggcagcagggttggt

SEQ ID NO: 538 - DNA, 352 bp, artificial
  synthetic
gacgaacgctggcggcgtgcttaacacatgcaagtcgaacggaccgagttgatagcttgctattgatgaggttagtggcaaacgggtgagtaacgcgtaggcaacctgcccttcagatggggacaacacctcgaaaggggtgctaataccgaatgacgtttcctggtcgcatgacctggaaaccaaaggccgggcaaccggtcactgaaggatgggcctgcgtctgattagctagttggtggggtaacggcccaccaaggcgacgatcagtagccggtctgagaggatgaacggccacattgggactgagacacggcccaaactcctacgggaggcagcagggttggttttt

SEQ ID NO: 539 - DNA, 341 bp, artificial
  synthetic
gacgaacgctggcggcgcgcctaacacatgcaagtcgaacggagctgagaggagcttgcttttcttagcttagtggcgaacgggtgagtaacgcgtgagtaacctgccctggagtgggggacaacagttggaaacgactgctaataccgcataagcccacggtgccgcatggcactgcgggaaaaggatttattcgctctaggatggactcgcgtccaattagctagttggtgaggtaacggcccaccaaggcgacgattggtagccggactgagaggttgaacggccacattgggactgagacacggcccagactcctacgggaggcagcagggttggtt

SEQ ID NO: 540 - DNA, 331 bp, artificial
  synthetic
gacgaacgctggcggcgtgcttaacacatgcaagtcgtacggtaaggccctttcgggggtacacgagtggcgaacgggtgagtaacacgtgagtaacctgcccacaactttgggataacgctaggaaactggtgctaatactggatatgtgctcctgctgcatggtgggggttggaaagctccggcggttgtggatggactcgcggcctatcagcttgttggtggggtagtggcctaccaaggcggcgacgggtagccggcctgagagggtgaccggccacattgggactgagatacggcccagactcctacggaaggcagcagggttggt

SEQ ID NO: 541 - DNA, 353 bp, artificial
  synthetic
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagcgcacagtaccgcatggtacagtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagggttggt

SEQ ID NO: 542 - DNA, 333 bp, artificial
  synthetic
gatgaacgctggcggcgtgcttaacacatgcaagtcgaacgatgaagcccagcttgctgggtggattagtggcgaacgggtgagtaacacgtgagtaacctgcccttgactctgggataagcctgggaaactgggtctaataccggataggaccgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagggttggt

SEQ ID NO: 543 - DNA, 374 bp, artificial
  synthetic
gacgaacgctggcggcgtgcctaacacatgcaagtcgaacggagttaaattcgacacccgagtatccggccgggaggcggggtgctgggggttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatggactcgcgtccgattagccagttggcggggtaacggcccaccaaagcgacgatcggtagccggactgagaggttgaacggccacattgggactgagacacggcccagactcctacggaaggcagcagggttggtt

SEQ ID NO: 544 - DNA, 344 bp, artificial
  synthetic
gatgaacgctagcgacaggcttaacacatgcaagtcgaggggcagcgggggcgcagcaatgcgcctgccggcgaccggcgcacgggtgagtaacacgtatgcaacctgcccgccgcaggggtataaccgggggaaaccccgactaatcccgcatgacaccccgtggaggcatctcctcggggtcaaaggagcgatccggcggcggatgggcatgcgtcgcattagctagtcggcggggtaacggcccaccgaggcgacgatgcgtaggggttctgagaggaaggcccccccacactggtactgagacacggaccagactcctacggaaggcagcagggttggtt

SEQ ID NO: 545 - DNA, 342 bp, artificial
  synthetic
gatgaacgctagctacaggcttaacacatgcaagtcgaggggcagcatggtcttagcttgctaaggccgatggcgaccggcgcacgggtgagtaacacgtatccaacctgccgacaacactgggatagcctttcgaaagaaagattaataccggatggcatagttttcccgcatgggatgattattaaagaatttcggttgtcgatggggatgcgttccattaggcagttggcggggtaacggcccaccaaaccaacgatggataggggttctgagaggaaggtcccccacattggaactgagacacggtccaaactcctacggaaggcagcagggttggtt

SEQ ID NO: 546 - DNA, 343 bp, artificial
  synthetic
gatgaacgctggcggcgtgcttaacacatgcaagtcgaacgaagcaccttatttgatttcttcggaatgaagatttggtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacggaaggcagca

SEQ ID NO: 547 - DNA, 350 bp, artificial
  synthetic
atgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcagaggtaaaaactccggtggtatgagatggacccgcgtctgattaggtagttggtggggtaacggcccaccaagccgacgatcagtagccgacctgagagggtgaccggccacattgggactgagacacggcccagactcctacggaaggcagcagggatggt

SEQ ID NO: 548 - DNA, 353 bp, artificial
  synthetic
gatgaacgctggcggcgtgcttaacacatgcaagtcgaacgaagcactttatttgatttcttcggaactgaagattttgtgactgagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacggaaggcagcagggttggtt

SEQ ID NO: 549 - DNA, 348 bp, artificial
  synthetic
gatgaacgctggcggcgtgcttaacacatgcaagtcgaacgaagcactttacttgatctcttcggagtgattgttctgtgactgagtggcggacgggtgagtaacgcgtgggtaacctgccttgtacagggggataacagttggaaacgactgctaataccgcataagcgcacaggaccgcatggtctggtgtgaaaaactccggtggtataagatggacccgcgtctgattagcttgttggtggggcaacggcctaccaaggcgacgatcagtagccgacctgagagggtgaccggccacattgggactgagacacggcccaaactcctacgggaggcagcagggtt

SEQ ID NO: 550 - DNA, 347 bp, artificial
  synthetic
gacgaacgctggcggcgtgcttaacacatgcaagtcgaacggagtttcttggatcaagacttcggtcaagtgaattgaaacttagtggcggactggtgagtaacgcgtgaggaacctgcctttcagagggggacaacagttggaaacgactgctaataccgcatgatgcgtttgggtcgcatggctcgaacgccaaagattttatcgctgaaagatggcctcgcgtctgattagctagttggtgaggtaacggcccaccaaggcgacgatcagtagccggactgagaggttgaccggccacattgggactgagatacggcccagactcctacgggaggcagcagggt

SEQ ID NO: 551 - DNA, 350 bp, artificial
  synthetic
gacgaacgctggcggcatgcctaacacatgcaagtcgaacgggaccgtacggatcggaggcttcggccaaagaactgtacatgtctagtggcggacgggtgagtaacgcgtgagtaacctgccttcaagagggggacaacatttggaaacagatgctaataccgcatatgcccacagtgccgagtggcacaggggggaaagatttatcgcttgaagatggactcgcgtcccattagttagttggcggggtaacggcccaccaagaccgcgatgggtagccggactgagaggttgaacggccacactgggactgagacacggcccagactcctacgggaggcagcagggtt

SEQ ID NO: 552 - DNA, 348 bp, artificial
  synthetic
gatgaacgctggcggcgtgcttaacacatgcaagtcgagcgaagcaccttgacggatttcttcggattgaagccttggtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaacggcccaccaaggcgacgatcagtagccggcctgagagggtgaacggccacattgggactgagacacggcccaaactcctacgggaggcagcagggtt

SEQ ID NO: 553 - DNA, 338 bp, artificial
  synthetic
gatgaacgctagcggcaggcttaacacatgcaagtcgaggggcagcggggtgtagcaatacactgccggcgaccggcgcacgggtgcgtaacgcgtatgcaacctacccataacaggggcataacactgagaaattggtactaattccccataacattcgaagaggcatctctttgggttgaaaactccggtggttatggatgggcatgcgttgtattagctagttggtgaggtaacggctcaccaaggcgacgatacatagggggactgagaggttaaccccccacattggtactgagacacggaccaaactcctacggaaggcagcagggttggtt

SEQ ID NO: 554 - DNA, 347 bp, artificial
  synthetic
gatgaacgctggcggcgtgcttaacacatgcaagtcgaacgaagcacttaagattgattcttcggatgaagtcttttgtgactgagtggcggacgggtgagtaacgcgtgggtaacctgcctcatacagggggataacagttagaaatgactgctaataccgcataagcgcacggtgtcgcatgacacagtgtgaaaaactccggtggtatgagatggacccgcgtctgattagctagttggcgaggtaacggcccaccaaggcgacgatcagtagccgacctgagagggtgatcggccacattgggactgagacacggcccaaactcctacgggaggcagcagggt

SEQ ID NO: 555 - DNA, 351 bp, artificial
  synthetic
gatgaacgctggcggcgtgcttaacacatgcaagtcgaacgaagcacttaagtttgattcttcggatgaagacttttgtgactgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacggaaggcagcagggttggt

SEQ ID NO: 556 - DNA, 338 bp, artificial
  synthetic
gatgaacgctagctacaggcttaacacatgcaagtcgaggggcagcatggtcttagcttgctaaggccgatggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacggaaggcagcagggtt

SEQ ID NO: 557 - DNA, 351 bp, artificial
  synthetic
gacgaacgctggcggcgtgcttaacacatgcaagtcgaacggggcgcttatgatggagatttcggtcaacggattaagttgcctagtggcggacgggtgagtaacgcgtgagtaacctgcctttcagagggggacaacagttggaaacgactgctaataccgcataatatatatggaccgcatgatctgtatatcaaagatttatcgctgaaagatggactcgcgtctgattagctagttggtgaggtaacggcccaccaaggcgacgatcagtagccggactgagaggttgaacggccacattgggactgagatacggcccagactcctacgggaggcagcagggttggt

SEQ ID NO: 558 - DNA, 350 bp, artificial
  synthetic
gatgaacgctggcggcgtgcttaacacatgcaagtcgaacgaagcaccttgatttgattcttcggatgaagatcttggtgactgagtggcggacgggtgagtaacgcgtgaggaacctgcctcaaagagggggacaacagttggaaacgactgctaataccgcataagcccacaggtcggcatcgaccagagggaaaaggagcaatccgctttgagatggcctcgcgtccgattagctagttggtgaggtaacggcccaccaaggcgacgatcggtagccggactgagaggttgaacggccacattgggactgagacacggcccagactcctacggaaggcagcagggtt

SEQ ID NO: 559 - DNA, 325 bp, artificial
  synthetic
gatgaacgctggcggcgtgcttaacacatgcaagtcgaacgaggctcttcggagccgagtggcggacgggtgagtaacgcgtgggtaacctgccctatacaggggaataactgtgagaaatcacagctaatgccgcataagcgcacagtaccgcatggtacggtgtgaaaagctccggcggtataggatggacccgcgtctgattaggtagttggtggggtaacggcctaccaagccgacgatcagtagccgacctgagagggtgaccggccacattgggactgagacacggcccagactcctacgggaggcagcagggttggtt

SEQ ID NO: 560 - DNA, 353 bp, artificial
  synthetic
gacgaacgctggcggcgtgcttaacacatgcaagtcgaacggagtgctcatgacagaggtttcggccaatggattgagttacttagtggcggactggtgagtaacgcgtgaggaacctgcctttcagagggggacaacagttggaaacgactgctaataccgcatgacacatcgaaggggcatccctttgatgtcaaagattttatcgctgaaagatggcctcgcgtctgattagctagttggtgaggtaacggcccaccaaggcgacgatcagtagccggactgagaggttgaccggccacattgggactgagatacggcccagactcctacgggaggcagcagggttggtt

SEQ ID NO: 561 - DNA, 345 bp, artificial
  synthetic
gatgaacgctggcggcatgcctaatacatgcaagtcgaacgaagctgattggaagcttgcttctgaaaggcttagtggcgaacgggtgagtaacacgtagggaacctgcccagatcacggggataacggttggaaacgacagctaagaccggataggtgatgacgaggcatcttgtcatcatgaaaagagctacggctctggagctggatggacctgcggcgcattagctagttggtgaggtaacggcccaccaaggcaatgatgcgtagccggcctgagagggcgaacggccacattgggactgagacacggcccaaactcctacgggaggcagcagggttggt

SEQ ID NO: 562 - DNA, 343 bp, artificial
  synthetic
gatgaacgctagcggcaggcttaacacatgcaagtcgaggggcagcgggaggaggtagcaataccaccttgccggcgaccggcggaagggtgcgtaacgcgtgagcaacctgcccgtcactggggaataaccgttggaaacgacgactaataccccatagttctggagggaggcatctcccatcaggtaaagagattcggtgacggatgggctcgcgtgacattagctagttggtagggtaacggcctaccaaggcgacgatgtctaggggttctgagaggaaggtcccccacactggaactgagacacggtccagactcctacgggaggcagcagggttggt

SEQ ID NO: 563 - DNA, 346 bp, artificial
  synthetic
gatgaacgctggcggcgtgcttaacacatgcaagtcgagcgaagcagttaagaagattcttcggatgattcttaactgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacggaaggcagcagggtt

SEQ ID NO: 564 - DNA, 339 bp, artificial
  synthetic
attgaacgctggcggcatgctttacacatgcaagtcgaacggtaacaggccttcgggtgctgacgagtggcgaacgggtgagtaatgcatcggaacgtgcccagaggtgggggataacgcagcgaaagctgtgctaataccgcatgtgatctgaggatgaaagcgggggaccaagcagcaatgtttggcctcgcgcctctggagcggccgatgtcagattaggtagttggtggggtaaaggcctaccaagccgacgatctgtagctggtctgagaggacgaccagccacactgggactgagacacggcccagactcctacggaaggcagcagggttggt

SEQ ID NO: 565 - DNA, 343 bp, artificial
  synthetic
gacgaacgctggcggcgtgcctaatacatgcaagtagaacgctgaggtttggtgtttacactagactgatgagttgcgaacgggtgagtaacgcgtaggtaacctgcctcatagcgggggataactattggaaacgatagctaataccgcataagagtaattaacacatgttagttatttaaaaggagcaattgcttcactgtgagatggacctgcgttgtattagctagttggtgaggtaaaggctcaccaaggcgacgatacatagccgacctgagagggtgatcggccacactgggactgagacacggcccagactcctacggaaggcagcagggttggt

SEQ ID NO: 566 - DNA, 338 bp, artificial
  synthetic
gatgaacgctagcgacaggcctaacacatgcaagtcgaggggcatcggggagtggcaacactccgccggcgaccggcgcacgggtgagtaacgcgtatgcaacctgcccgcaccagggggataaccgggagaaatcccgtctaataccgcgtaacgccttgtgggggcatccccatgaggccaaagggtttccgggagcggatgggcatgcgtgacattagctagttggcggggtaacggcccaccaaggcgacgatgtctaggggttctgagaggaaggtcccccacattggtactgagacacggaccaaactcctacggaaggcagcagggttggt

SEQ ID NO: 567 - DNA, 333 bp, artificial
  synthetic
gatgaacgctagcgggaggcctaacacatgcaagccgagcggtagaaagtagcttgctacttttgagagcggcgtacgggtgcgtaacacgtgtgcaacctgcctttatctggggaatagcctttcgaaaggaagattaatgctccataacatattgaatggcatcatttaatattgaaagctccggcggatagagatgggcacgcgcaagattagttagttggtgaggtaacggctcaccaaggcgatgatctttagggggcctgagagggtgatcccccacactggtactgagacacggaccagactcctacgggaggcagcagggttggt

SEQ ID NO: 568 - DNA, 364 bp, artificial
  synthetic
gacgaacgctggcggcatgcctaacacatgcaagtcgaacggagtttgttattagaagttcttcggaatggaagataataaacttagtggcggacgggtgagtaacgcgtggataacctgcctttttgtggggaacaacttcgagaaatcggagctaataccgcatgagcttataaagccgcatggcattataaggaaagatggcctctgaacatgctatcgcaaaaagatggatccgcgtctgattagctagttggtaaggtagcggcttaccaaggcgacgatcagtagccggcctgagagggtgaacggccacattgggactgagacacggcccaaactcctacgggaggcagcagggttt

SEQ ID NO: 569 - DNA, 350 bp, artificial
  synthetic
gacgaacgctggcggcgtgcttaacacatgcaagtcgaacgaagcttgattacggattcttcggatgaagtatgatatgactgagtggcggacgggtgagtaacgcgtgagtaacctgcccttcagagggggatagcgtttggaaacgaacggtaataccgcataatgtattttgaccgcatgatcgaaataccaaagatttatcgctgaaggatggactcgcgtctgattaggtagttggtggggtaacggcctaccaagccgacgatcagtagccggactgagaggttgatcggccacattgggactgagacacggcccagactcctacggaaggcagcagggttggt

SEQ ID NO: 570 - DNA, 338 bp, artificial
  synthetic
gatgaacgctagctacaggcttaacacatgcaagtcgaggggcagcatggtcttagcttgctaaggctgatggcgaccggcgcacgggtgagtaacacgtatccaacctgccgataactcggggatagcctttcgaaagaaagattaatacccgatggcataattagaccgcatggtcttattattaaagaatttcggttatcgatggggatgcgttccattaggcagttggtgaggtaacggctcaccaaaccttcgatggataggggttctgagaggaaggtcccccacattggaactgagacacggtccaaactcctacggaaggcagcagggtt

SEQ ID NO: 571 - DNA, 332 bp, artificial
  synthetic
gacgaacgctggcggcgtgcctaacacatgcaagtcgaacggaagttagagcttcggttttaactttagtggcgaacgggtgagtaacgcgtgaagaacctgcctttcagtgggggacaacagttggaaacgactgctaataccgcatgacgcttcttgggggcatccccgagaagtcaaagctttatgtgctgaaagatggcttcgcgtctgattagctagatggcggggtaacggcccaccatggcgacgatcagtagccggtctgagaggatgaacggccacattgggactgagatacggcccagactcctacggaaggcagcagggtt

SEQ ID NO: 572 - DNA, 351 bp, artificial
  synthetic
gacgaacgctggcggcgtgcttaacacatgcaagtcgaacggagcacccttgattgaggtttcggccaaatgataggaatgcttagtggcggactggtgagtaacgcgtgaggaacctgcctttcagagggggacaacagttggaaacgactgctaataccgcatgacacatagaggtcacatgacctttatgtcaaagatttatcgctgaaagatggcctcgcgtctgattagctagttggtgaggtaacggcccaccaaggcgacgatcagtagccggactgagaggttgaccggccacattgggactgagatacggcccagactcctacgggaggcagcagggttggt

SEQ ID NO: 573 - DNA, 335 bp, artificial
  synthetic
attgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagggttg

SEQ ID NO: 574 - DNA, 334 bp, artificial
  synthetic
gatgaacgctggcggcatgcctaatacatgcaagtcgaacgaaccgcttttataggcggagagtggcgaacgggtgagtaacacgtagggaacctacccatgcgagggggacaacttctggaaacggaagctaataccgaataaggaaatggaaggcatcttcgatttcttaaaggaggcgtaagccttgcgcaaggatggacctgcggtgcattagctggttggtaaggtaacggcttaccaaggcgatgatgcatagccgagttgagagactgatcggccacaatggaactgagacacggtccatactcctacggaaggcagcagggttggt

SEQ ID NO: 575 - DNA, 337 bp, artificial
  synthetic
gacgaacgctggcggcgtgcctaacacatgcaagtcgagcgaatgaagttccttcgggaacggatttagcggcggacgggtgagtaacacgtgggcaacctgcctcatagaggggaatagccttccgaaagggagattaataccgcataagattgtagtaccgcatggtacagcaattaaaggagcaatccgctatgagatgggcccgcggcgcattagctagttggtgaggtaacggctcaccaaggcgacgatgcgtagccgacctgagagggtgatcggccacattgggactgagacacggcccagactcctacggaaggcagcagggttggtt

SEQ ID NO: 576 - DNA, 348 bp, artificial
  synthetic
gatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattagctagttggtgaggtaacggcccaccaaggcgacgatcagtagccgacctgagagggtgaccggccacattgggactgagacacggcccaaactcctacggaaggcagcagggtt

SEQ ID NO: 577 - DNA, 353 bp, artificial
  synthetic
gatgaacgctggcggcgtgcttaacacatgcaagtcgaacgaagcactttatttgatttccttcgggactgaagattttgtgactgagtggcggacgggtgagtaacgcgtgggtaacctgccccataccgggggataacagctggaaacggctgctaataccgcataagcgcacagtaccgcatggtacggtgtgaaaaactccggtggtatgggatggacccgcgtctgattagccagttggcagggtaacggcctaccaaagcgacgatcagtagccgacctgagagggtgaccggccacattgggactgagacacggcccaaactcctacgggaggcagcagggttggt

SEQ ID NO: 578 - DNA, 341 bp, artificial
  synthetic
attgaacgctggcggcgtgcttaacacatgcaagtcgaacgagaaagttccttcgggaatgagtagagtggcgcacgggtgagtaacgcgtggataatctaccggggagtggggaataacagttggaaacggctgctaataccgcatacgctgcatatatgtctatgcaggaaagggggcctctgcatatgcttccgcttttcgatgagtccgcgtcccattagcttgttggcggggtaacggcccaccaaggcgacgatgggtagctggtctgagaggatgaccagccacactgggactggaacacggcccagactcctacggaaggcagcagggttggt

SEQ ID NO: 579 - DNA, 337 bp, artificial
  synthetic
attgaacgctggcggcaggcctaacacatgcaagtcggacggtagcacagaggagcttgctccttgggtgacgagtggcggacgggtgagtaatgtctggggatctgcccgatagagggggataaccactggaaacggtggctaataccgcataacgtcgcaagaccaaagtgggggaccttcgggcctcacaccatcggatgtgcccagatgggattagctagtaggtggggtaacggctcacctaggcgacgatccctagctggtctgagaggatgaccagccacactggaactgagacacggtccagactcctacggaaggcagcagggttggt

SEQ ID NO: 580 - DNA, 365 bp, artificial
  synthetic
gacgaacgctggcggcgtgcctaacacatgcaagtcgaacggagttaaacgctgaaattgagattagcttgctaaaggatttttcttgtttaacttagtggcggacgggtgagtaacgcgtgagtaacctgccttacagagggggacaacagttggaaacgactgctaataccgcataatgtctaaccgaggcatctcggatagaccaaaggagcaatccgctgtaagatggactcgcgtccaattagatagttggtggggtaacggcccaccaagtcgacgattggtagccggactgagaggttgaacggccacattgggactgagacacggcccagactcctacggaaggcagcagggttggt

SEQ ID NO: 581 - DNA, 347 bp, artificial
  synthetic
gatgaacgctggcggcgtgcttaacacatgcaagtcgaacgagaaccattggatcgaggattcgtccaagtgaaggtggggaaagtggcggacgggtgagtaacgcgtgagcaatctgccttggagtggggaataacggctggaaacagccgctaataccgcatgatacagctgggaggcatctccctggctgtcaaagatttatcgctctgagatgagctcgcgtctgattagctagttggcggggtaacggcccaccaaggcgacgatcagtagccggactgagaggttggccggccacattgggactgagacacggcccagactcctacgggaggcagcagggt

SEQ ID NO: 582 - DNA, 352 bp, artificial
  synthetic
gacgaacgctggcggcgtgcttaacacatgcaagtcgaacggagcgcctatgaaggagatttcggtcaacggaataggttgcttagtggcggacgggtgagtaacgcgtgaggaacctgcctttcagagggggacaacagttggaaacgactgctaataccgcataacacataggtgtcgcatggcatttatgtcaaagatttatcgctgaaagatggcctcgcgtctgattagctagttggtgaggtaacggctcaccaaggcgacgatcagtagccggactgagaggttggccggccacattgggactgagatacggcccagactcctacggaaggcagcagggttggtt

SEQ ID NO: 583 - DNA, 350 bp, artificial
  synthetic
gacgaacgctggcggcgtgcttaacacatgcaagtcgaacgggcccgctcttgtttttgggggcgggttgagtggcgaacgggtgagtatcacgtgagtaacctgccctcctctcctggataaccgcttgaaagggcggctaatacggggtggtccggttggtccgcatgggccggctgggatagtttcatcttcgggtgtttcggtgggggatgggctcgcggcctatcagcttgttggtggggtgatggcccaccaaggcggtgacgggtagccggcctgagagggtgggcggccacactgggactgagacacggcccagactcctacgggaggcagcagggttggtt

SEQ ID NO: 584 - DNA, 329 bp, artificial
  synthetic
gataaacgctggcggcatgcctaatacatgcaagtggaacgatttacttcggtaagtcgtcgcgaacgggtgagtaacacgtagataacctgccttatagggggggataaccattggaaacgatggataataccgcataaatacctactaggcatctagtagagtagaaaggagcaattgcttcgctatgagatggatctgcggcgcattagctagttggtgaggtaacggcccaccaaggcgacgatccgtagccggcctgagagggtgaacggccacattgggactgagacacggcccagactcctacgggaggcagcagggttttt

SEQ ID NO: 585 - DNA, 344 bp, artificial
  synthetic
gatgaacgctagcggcaggcttaacacatgcaagtcgcggggcagcggggaggaagcttgcttcctccgccggcgaccggcgcacgggtgagtaacacgtatgcaacctgccctcgtcagggggacaacccgccgaaaggcgggctaatcccgcgtatatgtctttggggcatcctgaagacaggaaaggtttcggccggacgaggatgggcatgcggcgcattaggcagttggcggggtaacggcccaccaaaccgacgatgcgtaggggttctgagaggaaggtcccccacactggtactgagacacggaccagactcctacggaaggcagcagggttggtt

SEQ ID NO: 586 - DNA, 342 bp, artificial
  synthetic
gatgaacgctggcggcgtgcctaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacggaaggcagcagggttggt

SEQ ID NO: 587 - DNA, 328 bp, artificial
  synthetic
gacgaacgctggcggcgtgcttcattcatgcaagtcgaacgagaatctttagcttgctagagaggaaagtggcggacgggtgagtaatatgtagagaatctgccctagagagggggacaacagttggaaacggctgctaataccccatatgagcgtatctgaaatggtattcttgaaaactccggtgctctaggatgagtctgcatctgattagctagttgggggtgtaatggaccaccaaggcgacgatcagtagctggtttgagaggatgatcagccacaatgggactgagacacggcccatactcctacgggaggcagcagggtt

SEQ ID NO: 588 - DNA, 330 bp, artificial
  synthetic
attgaacgctggcggcaggcttaatacatgcaagtcgaacggtaacagtaagagagtttactcttttagctgacgagtggcggacgggtgagtaatacctggggagctgcctggatgagggggataccttctggaaacggaagctaataccgcataaaccctgaggggaaaagggagcaatcccgcattcagatgcacccaggagggattagctagttggtggggtaaaggcctaccaaggcgatgatctctagctggtctgagaggatgaccagccacactggaactgagacacggtccagactcctacggaaggcagcagggttggtt

SEQ ID NO: 589 - DNA, 346 bp, artificial
  synthetic
gatgaacgctggcggcgtgcctaacacatgcaagtcgaacgaagcaccttacttgattcttcggatgaaggtttggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacggaaggcagcagggtt

SEQ ID NO: 590 - DNA, 342 bp, artificial
  synthetic
gacgaacgctggcggcgtgcctaatacatgcaagtcgagcgaatcttgaggtgcttgcacctcttggttagcggcggacgggtgagtaacacgtgggcaacctgcctgtaagactgggataacttcgggaaaccggagctaataccggataatccttttcctctcatgaggaaaagctgaaagtcggtttacgctgacacttacagatgggcccgcggcgcattagctagttggtgaggtaacggctcaccaaggcgacgatgcgtagccgacctgagagggtgatcggccacactgggactgagacacggcccagactcctacggaaggcagcagggttgg

SEQ ID NO: 591 - DNA, 348 bp, artificial
  synthetic
gatgaacgctagctacaggcttaacacatgcaagtcgaggggaaacgacatcgaaagcttgcttttgatgggcgtcgaccggcgcacgggtgagtaacgcgtatccaacctgcccatcacttgggaataaccttgcgaaagtaagactaatacccaatgatatccatagaagacatctgaaatggattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagggttggttttt

SEQ ID NO: 592 - DNA, 349 bp, artificial
  synthetic
gacgaacgctggcggcgcgcctaacacatgcaagtcgaacggagtgttttcacggaagttttcggatggaagtggttacacttagtggcggacgggtgagtaacacgtgagcaacctgcctttcagagggggataacagttggaaacgactgctaataccgcatgatattaccgggtcacatggcctggcaatcaaaggagcaatccgctgaaagatgggctcgcgtccgattagccagttggcggggtaatggcccaccaaagcgacgatcggtagccggactgagaggttgaacggccacattgggactgagacacggcccagactcctacggaaggcagcagggtt

SEQ ID NO: 593 - DNA, 342 bp, artificial
  synthetic
gatgaacgctagctacaggcttaacacatgcaagtcgaggggcatcgggaagaaagcttgctttctttgctggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacggaaggcagcagggttggt

SEQ ID NO: 594 - DNA, 344 bp, artificial
  synthetic
gatgaacgctagcgacaggcttaacacatgcaagtcgaggggcagcggggcggaagcttgctttcgccgccggcgaccggcgcacgggtgagtaacacgtatgcaacctgccctccacagggggataatcgggagaaatcccgtctaataccgcataacgccaccaacgggcatccgtaggtggccaaaggagcgatccggtggaggctgggcatgcgccgcattagccagttggcggggtaacggcccaccaaggcgacgatgcgtaggggttctgagaggaaggtcccccacactggtactgagacacggaccagactcctacggaaggcagcagggttggt

SEQ ID NO: 595 - DNA, 343 bp, artificial
  synthetic
gatgaacgctggcggcgtgcttaacacatgcaagtcgagcgaagcacttatgattgacttttcggaggatttcattagtgactgagcggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtttgattagctagttggtgaggtaacggcccaccaaggcgacgatcaatagccgacctgagagggtgaccggccacattgggactgagacacggcccaaactcctacggaaggcagca

SEQ ID NO: 596 - DNA, 335 bp, artificial
  synthetic
ggcgaacgctggcggcgtgcctaacacatgcaagtcgaacggagttaagcccttcgggacttaacttagtggcgaacgggtgagtaacgcgtgaggaacctgcctttcagtgggggacaacagttggaaacgactgctaataccgcatgatactttttgggggcatccctggaaagtcaaagatttattgctgaaagatggcctcgcgtctgattagctggttggcggggtaacggcccaccaaggcgacgatcagtagccggtctgagaggatgaacggccacattgggactgagatacggcccagactcctacgggaggcagcagggttggtt

SEQ ID NO: 597 - DNA, 337 bp, artificial
  synthetic
attgaacgctggcggcaggcttaacacatgcaagtcgagcggtagcacaggggagcttgctccccgggtgacgagcggcggacgggtgagtaatgtctgggaaactgcctgatggagggggataactactggaaacggtagctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagggttggt

SEQ ID NO: 598 - DNA, 331 bp, artificial
  synthetic
attgaacgctggcggcaggcctaacacatgcaagtcgagcggtaacacagggagcttgctcctgggtgacgagcggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacggaaggcagcagggt

SEQ ID NO: 599 - DNA, 340 bp, artificial
  synthetic
gacgaacgctggcggcgcgcctaacacatgcaagtcgaacggagtcaagaggagcttgcttttcttgacttagtggcgaacgggtgagtaacgcgtgagtaacctgccctggagtgggggacaacagttggaaacgactgctaataccgcataagcccacggcccggcatcgggctgagggaaaaggatttattcgcttcaggatggactcgcgtccaattagctagttggtgaggtaacggcccaccaaggcgacgattggtagccggactgagaggttgaacggccacattgggactgagacacggcccagactcctacggaaggcagcagggttggt

SEQ ID NO: 600 - DNA, 333 bp, artificial
  synthetic
gacgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacaggtcggcatcgaccagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcaggg

SEQ ID NO: 601 - DNA, 350 bp, artificial
  synthetic
gatgaacgctggcggcgtgcttaacacatgcaagtcgaacgaagcactttgatcgatttcttcggattgaaattttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggtggggtaacggcctaccaaggcgacgatcagtagccgacctgagagggtgaccggccacattgggactgagacacggcccagactcctacggaaggcagcagggaatt

SEQ ID NO: 602 - DNA, 342 bp, artificial
  synthetic
gatgaacgctagctacaggcttaacacatgcaagtcgaggggcatcaggaagaaagcttgctttctttgctggcgaccggcgcacgggtgagtaacacgtatccaacctgccgatgactcggggatagcctttcgaaagaaagattaatacccgatggtatatctgaaaggcatctttcagctattaaagaatttcggtcattgatggggatgcgttccattaggttgttggcggggtaacggcccaccaagccatcgatggataggggttctgagaggaaggtcccccacattggaactgagacacggtccaaactcctacgggaggcagcagggttggtt

SEQ ID NO: 603 - DNA, 349 bp, artificial
  synthetic
gatgaacgctggcggcgtgcttaacacatgcaagtcgaacgggaaatatttcattgagacttcggtggatttgatctatttctagtggcggacgggtgagtaacgcgtgggtaacctgccttatacagggggataacagtcagaaatggctgctaataccgcataagcgcacagtaccgcatggtccggtgtgaaaaactccggtggtatgagatggacccgcgtctgattagctagttggtggggtaacggcccaccaaggcgacgatcagtagccgacctgagagggtgaccggccacattgggactgagacacggcccaaactcctacggaaggcagcagggttgg

SEQ ID NO: 604 - DNA, 338 bp, artificial
  synthetic
gatgaacgctagcggcaggcttaacacatgcaagtcgaggggcagcgagattgaagcttgcttcaattgtcggcgaccggcggacgggtgcgtaacgcgtatgcaacctacccataacagggggataacactgagaaattggtactaataccccataacattccgagtggcatcacttggggttgaaagctgcggtggttatggatgggcatgcgttgtattagctagttggtgaggtaacggctcaccaaggcgacgatacatagggggactgagaggttaaccccccacattggtactgagacacggaccaaactcctacggaaggcagcagggtt

SEQ ID NO: 605 - DNA, 350 bp, artificial
  synthetic
gatgaacgctggcggcgtgcctaatacatgcaagtcgagcgagatgttagcgcatgaaccttcgggggattatgctaacggacagcggcggacgggtgagtaacgcgtaggcaacctgcccctgacagagggatagccattggaaacgatgattaaaacctcatgacaccgtagaagcacatgcttcatcggtcaaagatttatcggtcagggatgggcctgcgtctgattaactagttggtgaggtaacggctcaccaaggtgacgatcagtagccgacctgagagggtgatcggccacattggaactgagacacggtccaaacttctacggaaggcagcagggttggt

SEQ ID NO: 606 - DNA, 349 bp, artificial
  synthetic
gatgaacgctggcggcgtgcttaacacatgcaagtcgagcgaagcacttaagtttgattcttcggatgaagacttttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacggaaggcagcagggttg

SEQ ID NO: 607 - DNA, 348 bp, artificial
  synthetic
gacgaacgctggcggcgtgcttaacacatgcaagtcgaacggagcacccttgaaagagatttcggtcaatggataggaatgcttagtggcggacgggtgagtaacgcgtgaggaacctgcctttcagagggggacaacagctggaaacgactgctaataccgcatgacgcatcgaagtcgcatggctttgatgtcaaagatttatcgctggaagatggcctcgcgtctgattagctagttggtgaggtaacggcccaccaaggcgacgatcagtagccggcctgagagggtggacggccacattgggactgagacacggaccagactcctacggaaggcagcagggtt

SEQ ID NO: 608 - DNA, 349 bp, artificial
  synthetic
gatgaacgctggcggcgtgcttaacacatgcaagtcgaacgaagcacttttatttgatttcttcggaatgaagattttggtgactgagtggcggacgggtgagtaacgcgtgggtaacctgcctcacacagggggataacagttagaaatagctgctaataccgcataagcgcacggttccgcatggaacagtgtgaaaaactccggtggtgtgagatggacccgcgtctgattagccagttggcggggtaacggcccaccaaagcgacgatcagtagccggcctgagagggtgaacggccacattgggactgagacacggcccaaactcctacggaaggcagcagggt

SEQ ID NO: 609 - DNA, 339 bp, artificial
  synthetic
gataaacgctagcggcaggcctaacacatgcaagtcgaggggcagcggaagaagtagcttgctacttctgccggcgaccggcgcacgggtgcgtaacgcgtatgcaacctacctttaacagggggataatccgaagaaatttggtctaataccccataatatttcagaaggcatcttttgaggttgaaaactccggtggttaaagatgggcatgcgttgtattagctagatggtgaggtaacggctcaccattgcgatgatacatagggggactgagaggttttccccccacactggtactgagacacggaccagactcctacgggaggcagcagggtt

SEQ ID NO: 610 - DNA, 322 bp, artificial
  synthetic
agtgaacgctggcggcgtgcctaatacatgcaagtcgaacgatgaatcttctagcttgctagaagtggattagtggcgcacgggtgagtaatgcataggttatgtgccctttagtctgggatagccactggaaacggtgattaatactggatactccctacgggggaaagtttttcgctaaaggatcagcctatgtcctatcagcttgttggtgaggtaatggctcaccaaggctatgacgggtatccggcctgagagggtgatcggacacactggaactgagacacggtccagactcctacggaaggcagcagggttggtt

SEQ ID NO: 611 - DNA, 326 bp, artificial
  synthetic
gatgaacgctagcggcaggcctaatacatgcaagtcgaacgggtgcagcaatgtactagtggcgcacgggtgcgtaacacgtaaccaacctacccagaactgggggatagcccgccgaaaggcggattaataccgcataagccgtgtgagtggcatcacgtacacggtaaagatttattggttttggatggggttgcgggtcattagctagttggtacggtaacggcgtaccaaggcgacgatgactaggggagctgagaggctggtccccccacacgggcactgagatacgggcccgactcctacggaaggcagcagggttggtt

SEQ ID NO: 612 - DNA, 348 bp, artificial
  synthetic
atgaacgctggcggcgtgcctaacacatgcaagtcgaacgaagcactttaacttgatttcttcggaatgaaggtcttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacggaaggcagcagggtt

SEQ ID NO: 613 - DNA, 343 bp, artificial
  synthetic
gatgaacgctggcggcgtgcttaacacatgcaagtcgaacgaagcacttatctttgattcttcggatgaagaggtttgtgactgagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactgagacacggcccaaactcctacggaaggcagca

SEQ ID NO: 614 - DNA, 350 bp, artificial
  synthetic
gatgaacgctggcggcgtgcctaacacatgcaagtcgaacgaagcacctaactttgattctttcgggatgaagagttttgtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagctggttggcgaggtaacggctcaccaaggcgacgatcagtagccggcctgagagggtgaacggccacattgggactgagacacggcccaaactcctacgggaggcagcagggtt

SEQ ID NO: 615 - DNA, 340 bp, artificial
  synthetic
gatgaacgctagcggcaggcttaacacatgcaagtcgaagggcagcgtggggagtgcttgcactccctgacggcgactggcgcacgggtgagtaacacgtatgcaacctgccctccacagggggacaaccttccgaaagggaggctaatcccgcgtatatgtcttcggggcatcccggagacaggaaaggtttcggccggtggaggatgggcatgcggcgcattagctagttggcggggcaacggcccaccaaggcgacgatgcgtaggggttctgagaggaaggtcccccacactggtactgagacacggaccagactcctacggaaggcagcagggtt

SEQ ID NO: 616 - DNA, 340 bp, artificial
  synthetic
gatgaacgctagcggcaggcttaacacatgcaagtcgaggggcagcatttgggtagcaatacctgagatggcgaccggcgcacgggtgcgtaacgcgtatgcaacctacccataacaggggcataacactgagaaattggtactaattccccataacattcgaagaggcatcttttcgggttgaaaactccggtggttatggatgggcatgcgttgtattagctggttggtgaggtaacggctcaccaaggcgacgatacatagggggactgagaggttaaccccccacattggtactgagacacggaccaaactcctacggaaggcagcagggttggtt

SEQ ID NO: 617 - DNA, 345 bp, artificial
  synthetic
gatgaacgctggcggcgtgcttaacacatgcaagtcgaacgaagcactttaacttgatttcttcggaatgattgtttttgtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagctagttggtggggtaacggcccaccaaggcgacgatcagtagccgacctgagagggtgaccggccacattgggactgagacacggcccaaactcctacgggaggcagca

SEQ ID NO: 618 - DNA, 334 bp, artificial
  synthetic
gacgaacgctggcggcgtgcttaacacatgcaagtcgaacggaagtaaagatttcggtttttactttagtggcgaacgggtgagtaacgcgtgaggaacctgcctttcagtgggggacaacagttggaaacgactgctaataccgcatgatactattgagtggcatcacttgatagtcaaagatttattgctgaaagatggcctcgcgtctgattagctagttggtggggtaacggcccaccaaggcgacgatcagtagccggactgagaggttgaccggccacattgggactgagatacggcccagactcctacggaaggcagcagggttggt

SEQ ID NO: 619 - DNA, 334 bp, artificial
  synthetic
gacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacagcaccgcatggtgcagagggaaaaggagcaatccgctttgagatggcctcgcgtccgattagctagttggtgaggtaacggcccaccaaggcgacgatcagtagccggcctgagagggtgaacggccacattgggactgagacacggcccagactcctacggaaggcagcagggt

SEQ ID NO: 620 - DNA, 348 bp, artificial
  synthetic
gatgaacgctggcggcgtgcttaacacatgcaagtcgaacgaagcactttaattgatttcttcggaatgaagattttgtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagctagttggtggggtaacggcccaccaaggcgacgatcagtagccgacctgagagggtgaccggccacattgggactgagacacggcccaaactcctacggaaggcagcagggtt

SEQ ID NO: 621 - DNA, 349 bp, artificial
  synthetic
ggatgaacgctggcggcgtgcttaacacatgcaagtcgaacgaagcacttaagaacaattcttcggaggcgttcttttgtgactgagtggcggacgggtgagtaacgcgtgggtaacctgccttacactgggggataacacttagaaataggtgctaataccgcataagcgcacgagaccgcatggtctagtgtgaaaaactccggtggtgtaagatggacccgcgtctgattagctagttggcggggtaacggcccaccaaggcgacgatcagtagccggcctgagagggtggacggccacattgggactgagacacggcccaaactcctacggaaggcagcagggtt

SEQ ID NO: 622 - DNA, 346 bp, artificial
  synthetic
gatgaacgctggcggcgtgcttaacacatgcaagtcgaacgaagcaccttaatttgattcttcggatgaagatttttgtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcaggg

SEQ ID NO: 623 - DNA, 347 bp, artificial
  synthetic
atgaacgctggcggcgtgcttaacacatgcaagtcgaacgaagcactttaattgatttcttcggaatgaagattttgtgactgagtggcggacgggtgagtaacgcgtgggtaacctgcctcatacagggggataacagttagaaatgactgctaataccgcataagcgcacagtaccgcatggtacagtgtgaaaaactccggtggtatgagatggacccgcgtctgattagctagttggtggggtaacggcccaccaaggcgacgatcagtagccgacctgagagggtgaccggccacattgggactgagacacggcccaaactcctacgggaggcagcagggtt

(19) United States
(12) Patent Application Publication
(10) Pub. No.: US 2014/0171339 A1
(43) Pub. Date: Jun. 19, 2014

(54) METHODS AND KITS FOR DETECTING ADENOMAS, COLORECTAL CANCER, AND USES THEREOF

(71) Applicants: Temitope Keku, Chapel Hill, NC (US); Anthony Fodor, Charlotte, NC (US); Nina Sanapareddy, Basking Ridge, NJ (US)

(72) Inventors: Temitope Keku, Chapel Hill, NC (US); Anthony Fodor, Charlotte, NC (US); Nina Sanapareddy, Basking Ridge, NJ (US)

(73) Assignee: The University of North Carolina at Chapel Hill, Chapel Hill, NC (US)

(21) Appl. No.: 14/124,443

(22) PCT Filed: Jun. 6, 2012
(86) PCT No.: PCT/US12/41020

§ 371 Date: Dec. 6, 2013

Related U.S. Application Data
(60) Provisional application No. 61/493,770, filed on Jun. 6, 2011.

(51) Int. Cl. C12Q 1/68

(52) U.S. Cl. CPC C12Q 1/6886
USPC 506/9; 435/39; 435/6.11; 435/6.12; 435/7.92

(57) ABSTRACT

This invention is directed to a novel method to detect adenomas and colorectal cancer (CRC) using a bacterial signature. Included in the invention are methods of (a) determining an individual's risk developing adenomas or CRC; (b) determine whether or not a patient should have a colonoscopy; (c) differential diagnosis; (d) staging; (e) selecting therapies; (f) monitoring therapies; (g) patient surveillance; and (h) drug screening. Kits and reagents for detecting adenomas and CRC and/or drug screening are also part of the invention.

### RELATED APPLICATION

This application claims the benefit of U.S. Prov. Patent Appl. No. 61/493,770, filed Jun. 6, 2011 entitled “Methods and Kits for Detecting Adenomas, Colorectal Cancer and Uses Thereof” naming Keku et al. as inventors with Atty. Dkt. No. UNC10007USV. The entire contents of which are hereby incorporated by reference including all text, tables, and drawings.

### STATEMENT REGARDING FEDERALLY SPONSORED RESEARCH OR DEVELOPMENT

This invention was made in part with government support under grant number RO1 CA 136887 awarded by the National Cancer Institute. The United States Government has certain rights in the invention.

### 1. FIELD OF THE INVENTION

This invention relates generally to the discovery of a novel method to detect adenomas and colorectal cancer (“CRC”) using a microbial signature. Included in the invention are methods of (a) determining an individual's risk developing adenomas or CRC; (b) determine whether or not a patient should have a colonoscopy; (c) differential diagnosis; (d) staging; (e) selecting therapies; (f) monitoring therapies; (g) patient surveillance; and (h) drug screening. Kits and reagents for detecting adenomas and CRC and/or drug screening are also part of the invention.

### 2. BACKGROUND OF THE INVENTION

2.1. Colorectal Cancer (“CRC”)

CRC is categorized by the American Cancer Society (“ACS”) as a cancer which originates in the colon or rectum. In the United States CRC for men and women combined is the second most common cause of cancer death. In 2011 the ACS estimates that there will be about 101,700 new cases of colon cancer and 39,510 new cases of rectal cancer in the United States alone. CRC will cause an estimated 49,380 deaths. More than 95% of CRC cases are adenocarcinomas. American Cancer Society Detailed Guide: Colorectal Cancer (“ACS Guide CRC”), Mar. 2, 2011 http://www.cancer.org/Cancer/ColonandRectumCancer/DetailedGuide.

The majority (˜90%) of CRC cases arise sporadically from benign adenomatous polyps. Lance P. Recent developments in colorectal cancer. J R Coll Physicians Lond 31:483-7 (1997). The risk of developing CRC varies markedly within populations and geographical regions and, as not all adenomas ultimately progress to cancer, there is a strong indication that other factors are crucial to malignant transformation. Moore, W. E. & Moore, L. H. Intestinal floras of populations that have a high risk of colon cancer. Appl Environ Microbiol 61, 3202-3207 (1995). Although age, tobacco and alcohol consumption, lack of physical activity, and body weight are considered important risk factors for CRC (Cope, G. F. et al., Alcohol consumption in patients with colorectal adenomatous polyps. Gut 32, 70-72 (1991)), the most significant risk factor appears to be diet. Bingham, S. A. Diet and colorectal cancer prevention. Biochem Soc Trans 28, 12-16 (2000). Another routinely cited critical factor in CRC development is the role of host microbiota. Moore & Moore (1995).

Adenomas originate in the glandular epithelium and have a dysplastic morphology. Fearon, E. R. Annu. Rev. Pathol. Mech. Dis. 6: 479-507 (2011). Some of these adenomas mature into large polyps, undergo abnormal growth and development, and ultimately progress into CRC. M. L. Davila & A. D. Davila, Screening for Colon and Rectal Cancer, in Colon and Rectal Cancer 55-56 (Peter S. Edelstein ed., 2000). This progression would appear to take at least 10 years in most patients, rendering it a readily treatable form of cancer if diagnosed early and the CRC is localized. Davila at 56; Walter J. Burdette, Cancer: Etiology, Diagnosis, and Treatment 125 (1998).

A number of hereditary and nonhereditary conditions have also been linked to a heightened risk of developing CRC, including familial adenomatous polyposis (“FAP”), hereditary nonpolyposis CRC (Lynch syndrome or HNPCC), a personal and/or family history of CRC or adenomatous polyps, inflammatory bowel disease, diabetes mellitus, and obesity. Davila at 47; Henry T. Lynch & Jane F. Lynch, Hereditary Nonpolyposis Colorectal Cancer (Lynch Syndromes), in Colon and Rectal Cancer 67-68 (Peter S. Edelstein ed., 2000).

Environmental/dietary factors associated with an increased risk of CRC include diets high in red or processed meats, physical inactivity, obesity, smoking, excessive alcohol consumption and type 2 diabetes. ACS Guide CRC. Conversely, environmental/dietary factors associated with a reduced risk of CRC include a diet high in fruits and vegetables and increased physical activity. Folate, vitamin D, and calcium supplements may lower CRC risk also. Similarly, aspirin or other non-steroidal anti-inflammatory drugs (“NSAIDs”) have been associated with lower CRC risk. ACS Guide CRC.

2.2. CRC Molecular Biology

Researchers have spent many years studying the molecular biology associated with CRC. Approximately 15-30% of CRC instances have a major hereditary component, the remainder are due to somatic, or acquired defects. Fearon at 480. The genetic changes fall into several categories. For oncogenes they may be (i) mutations that activate or up-regulate; (ii) gene rearrangements that alter function; or (iii) gene rearrangements leading to upregulation and/or unregulated gene expression. For tumor suppressor genes the changes may be (i) mutations that inactivate tumor suppressors; (ii) loss of heterozygosity (LOH) destroying or eliminating entirely tumor suppressors; or (iii) epigenetic silencing such as methylation that reduce or shut down expression. Fearon at 480.

Defects in the tumor suppressor gene, adenomatous polyposis coli (“APC”), are present in the majority of CRC cases. APC defects are present also in >90% of the cases of FAP. Fearon at 481. Other major factors in the multi-step development of CRC are point mutations in oncogenes KRAS and BRAF; gene amplification of EGFR; and either mutations or allele loss for the tumor suppressor gene p53. Additional point mutations implicated are found in NRAS, PIK3CA, CDK8, CMYC, CCNE1, CTNNB1, NEU (HER2) and MYB. Other tumor suppressor genes implicated in the cascade are FBXW7, PTEN, SMAD4, SMAD2, SMAD3, TGFβIIR, TCF7L2, ACVR2 and BAX. Fearon at 488.

As discussed above, epigenetic silencing by DNA methylation also accounts for the lost of tumor suppressor genes. A strong association between microsatellite instability (“MSI”) and CpG island methylation has been well characterized in sporadic CRC with high MSI but not in those of hereditary origin. In one experiment, DNA methylation of MLH1, CDKN2A, MGMT, THBS1, RARB, APC, and p14ARF genes has been shown in 80%, 55%, 23%, 23%, 58%, 35%, and 50% of 40 sporadic CRCs with high MSI, respectively. Yamamoto, H. et al. Genes Chromosomes Cancer 33: 322-325 (2002); and Kim, K. M. et al. Oncogene. 12; 21(35): 5441-9 (2002). Others have reported hypermethylation and transcriptional silencing of secreted Frizzled-related proteins (“SFRPs”) and putative tumor suppressor, hypermethylated in cancer 1 (“HIC1”). Fearon at 496.

2.3. CRC Detection

Because CRC is often treatable when detected at an early, localized stage, current guidelines recommend screening tests should be a part of routine care for all adults starting at age 50. The current tests may be divided into two types: fecal tests and structural examination tests. Examples of fecal tests are (i) the fecal occult blood test (“FOBT”); (ii) the fecal immunochemical test (“FIT”); and (iii) the stool DNA (“sDNA”) test. Structural examination tests are (i) colonoscopy; (ii) flexible sigmoidoscopy; (iii) double-contrast barium enema (“DCBE”); (iv) CT colonography (virtual colonoscopy); and (v) capsule endoscopy.

These tests have advantages and disadvantages. Current fecal tests suffer from issues of accuracy, precision, inter- and intra-individual variability, and compliance due to patient's being uncomfortable with sample collection. If a fecal test is positive, a patient will be referred for a colonoscopy for a thorough examination and intervention (removal of adenomas) if necessary. The structural examination tests require both purging of a patient's bowels and pumping air into the colon to aid visualization. Each of the tests is described in greater detail below.

2.3.1. Fecal Blood Tests

Both the FOBT and FIT screen for CRC by detecting the amount of blood in the stool. The tests are based on the premise that neoplastic tissue, particularly malignant tissue, bleeds more than typical mucosa, with the amount of bleeding increasing with polyp size and cancer stage. Davila at 56-57. Multiple testing is recommended because of intermittent bleeding. While fecal blood tests may detect some early stage tumors in the lower colon, they are unable to detect (i) CRC in the upper colon because any blood will be metabolized and/or (ii) smaller adenomatous polyps, thus creating false negatives. Any gastro-intestinal bleeding due to hemorrhoids, fissures, inflammatory disorders (ulcerative colitis, Crohn's disease), infectious diseases, even long distance running, will create false positives. Beg et al. Occult Gastro-Intestinal Bleeding: Detection, Interpretation and Evaluation. J Indian Acad Clin Med 3(2) 153-158 (2002).

2.3.2. Fecal Occult Blood Test (“FOBT”)

FOBTs are guaiac-based and measure the peroxidase activity of heme or hemoglobin. They are inexpensive and relatively easy to administer. Commercially available products are HemeOccult® II, and HemeOccult® Sensa® (Beckman-Coulter Inc., Los Angeles, Calif.). In addition to the false positives and false negatives mentioned above, certain foods with peroxidase activity (uncooked fruits and vegetables, red meat) also create false positives.

2.3.3. Fecal Immunochemistry Test (“FIT”)

FIT is generally more accurate than FOBT. Rather than FOBT's chemical reaction to detect heme from blood, FIT uses antibodies to detect blood related proteins such as hemoglobin. Commercially available products are InSure® (Enterix Inc., a Quest Diagnostics company, Lyndhurst, N.J.); Hemoccult®-ICT (Beckman Coulter, Inc.); MonoHaem (Chemicon International, Inc., Temecula, Calif.); OC Auto Micro 80 (Polymedco, Cortland Manor. NY); and Magstream 1000/Hem SP (Fujirebio Inc. Tokyo, Japan). In addition to the issues from false positives or false negatives associated with blood in stools and/or metabolism, any metabolic denaturing or digestion of globin proteins or post-collection sample handling that denatures globin epitopes will create false negatives for the FIT.

2.3.4. Stool DNA (“sDNA”) Test

The sDNA test measures a variety of DNA markers measured in a lab from a stool sample collected by the patient. Current sDNA tests, available from Exact Sciences Corp. (Madison, Wis.), measure mutations in K-ras, APC, P53 genes; BAT-26 (an MSI marker); a marker for DNA integrity; and methylation of the vimentin gene. Levin et al. Screening and Surveillance for the Early Detection of Colorectal Cancer and Adenomatos Polyps. CA Cancer J Clinicians 58(3) 130-160 (2008). While some guidelines recommend sDNA testing other guidelines are more conservative and do not recommend sDNA testing. In one study a version of the sDNA test was superior to FOBT, but it still only detected 15% of the advanced adenomas. Imperiale et al. Fecal DNA versus fecal occult blood for colorectal-cancer screening in an average-risk population. N Engl J Med 351:2704-2714 (2004).

2.3.5. Colonoscopy and Sigmoidoscopy

Colonoscopy allows direct visualization of the bowel, and enables one to detect, biopsy, and remove adenomatous polyps. Davila at 59-61. Colonoscopy is the “gold standard” diagnostic for colon cancer. Despite these advantages, there are downsides. In addition to the patient discomfort discussed above, colonoscopy is a relatively expensive procedure and there are risks of possible bowel perforation and hemorrhaging. Davila at 59-60. Moreover, the skill and experience of doctors vary and some studies have reported missing 6-12% of large adenomas (=10 mm) and failing to detect cancer in 5% of the cases. Levin et al. at 145.

Flexible sigmoidoscopy, by definition, is limited to the sigmoid colon. A sigmoidscope is about 60 cm long (˜2 feet). Thus, a doctor can only examine the rectum and the lower half of the colon. Sigmoidoscopy requires the same preparation and invasiveness as colonoscopy, with those drawbacks. For the portions examined, it has the advantages of the colonoscopy. However, flexible sigmoidoscopy does only half the job.

2.3.6. Double-Contrast Barium Enema and CT Colonography

Double-contrast barium enema (“DCBE”) is also referred to as air-contrast enema. It requires the same prep as a colonoscopy to purge the patient's colon and the patient's colon is imaged using X-rays with a barium contrast agent. While it is recommended by most guidelines, DCBE suffers from two shortcomings One, patient discomfort during the prep and examination and two, if something suspicious is seen, it does not provide the opportunity for a biopsy or polypectomy. Thus, if there is a positive test result, the patient will need a colonoscopy follow up. CT colonography also known as a virtual colonoscopy uses a computed tomography (CT or CAT) scan to image the rectum and colon. Though it requires a colon preparation, it is minimally invasive and gaining acceptance. Unfortunately, like the DCBE, a positive test will require a colonoscopy to investigate and intervene if necessary.

2.3.7. Capsule Endoscopy

Capsule endoscopy involves the ingestion of a small capsule with video cameras at each end. Lieberman. Progress and Challenges in Colorectal Cancer Screening and Surveillance. Gastroenterology 138: 2115-2126 (2010). As it passes through the colon images are transmitted and recorded. Some studies have reported detection of 73% of the advanced adenomas and 74% of the CRC cases. Lieberman at 2119. The shortcomings are similar to DCBE or CT colonography because it requires similar patient preparation and positive results require a subsequent colonoscopy. In addition, insufficient battery life and inadequate imaging in periods of rapid motility are disadvantages for the current generation capsule endoscopy products.

2.4. CRC Staging

Once CRC has been diagnosed, treatment decisions are typically made using the stage of cancer progression. A number of techniques are employed to stage the cancer (some of which are also used to screen for colon cancer), including pathologic examination of resected colon, sigmoidoscopy, colonoscopy, and various imaging techniques. AJCC Cancer Staging Handbook, 143-164, Edge et al. eds., 7^{th} ed. 2011). Proximal lymph node evaluation, sentinel node evaluation, chest/abdominal/pelvic CT, MRI scans, positron emission tomography (“PET”) scans, liver functionality tests (for liver metastases), and blood tests (complete blood count (“CBC”), carcinoembryonic antigen (“CEA”), CA 19-9) are employed to determine the stage. NCCN Clinical Practice Guidelines in Oncology: Colon Cancer Version 3.2011, Feb. 25, 2011 http://www.nccn.org/professionals/physician_gls/pdf/colon.pdf.

Several classification systems have been devised to stage the extent of CRC, including the Dukes' system and the more detailed International Union against Cancer-American Joint Committee on Cancer TNM staging system. Burdette at 126-27. The TNM system, which is used for either clinical or pathological staging, is divided into four stages, each of which evaluates the extent of cancer growth with respect to primary tumor (T), regional lymph nodes (N), and distant metastasis (M). Fleming at 84-85. The system focuses on the extent of tumor invasion into the intestinal wall; invasion of adjacent structures; the number of regional lymph nodes that have been affected; and whether distant metastasis has occurred. Fleming at 81.

Stage 0 is characterized by in situ carcinoma (Tis), in which the cancer cells are located inside the glandular basement membrane (intraepithelial) or lamina propria (intramucosal). In this stage, the cancer has not spread to the regional lymph nodes (N0), and there is no distant metastasis (M0). In stage I, there is still no spread of the cancer to the regional lymph nodes and no distant metastasis, but the tumor has invaded the submucosa (T1) or has progressed further to invade the muscularis propria (T2). Stage II also involves no spread of the cancer to the regional lymph nodes and no distant metastasis, but the tumor has invaded the subserosa, or the nonperitonealized pericolic or perirectal tissues (T3), or has progressed to invade other organs or structures, and/or has perforated the visceral peritoneum (T4). Stage III is characterized by any of the T substages, no distant metastasis, and either spread to 1 to 3 regional lymph nodes (N1) or spread to four or more regional lymph nodes (N2). Lastly, stage IV involves any of the T or N substages, as well as distant metastasis (M1a or M1b). Physicians will also assign a grade, that is, characterize CRC based on the appearance of the cells ranging from G1 (well-differentiated, almost normal) to G4 (undifferentiated, very abnormal) where a high grade is an indication of a poor prognosis. ACS Guide CRC; Fleming at 84-85; Burdette at 127.

2.5. CRC Therapy

For the treatment of CRC, surgical resection results in a cure for roughly 50% of patients. Chemotherapy and irradiation maybe used both preoperatively (neoadjuvant) and postoperatively (adjuvant) in treating CRC. Chemotherapeutic agents, particularly 5-fluorouracil (5-FU), are powerful weapons in treating CRC. Other agents include oxaliplatin (Eloxatin®), irinotecan (Camptosar®), leucovorin, capecitabine (Xeloda®), bevacizumab (Avastin®), cetuximab (Erbitux®), and panitumumab (Vectibix®). These drugs are frequently combined. Common combinations are FOLFOX (5-FU, leucovorin, oxaliplatin); FOLFIRI (5-FU, leucovorin, irinotecan); and FOLFOXIRI (5-FU, leucovorin, irinotecan, oxaliplatin). Bevacizumab is a targeted therapeutic, specifically a monoclonal antibody that binds to vascular endothelial growth factor (VEGF) to prevent formation of blood vessels around the tumor. Cetuximab and panitumumab are monoclonal antibodies that target epidermal growth factor receptor (EGFR).

Many patients will develop a recurrence of CRC following surgical resection, particularly in the first 2 or 3 years. Accordingly, CRC patients must be closely monitored to determine response to therapy and to detect persistent or recurrent disease and metastasis.

From the foregoing, it is clear that improved procedures used for detecting, diagnosing, monitoring, staging, prognosticating, and preventing the recurrence of CRC are of critical importance to the outcome of the patient. Moreover, current procedures, while helpful in each of these analyses, are limited by their specificity, sensitivity, invasiveness, and/or cost effectiveness. As such, minimally invasive, highly specific and sensitive procedures would be highly desirable. Accordingly, there is a great need for more sensitive and accurate methods for predicting whether a person is likely to develop CRC, for diagnosing CRC, for monitoring the progression of the disease, for staging CRC, for determining whether CRC has metastasized, and for imaging CRC.

### 3. SUMMARY OF THE INVENTION

In particular non-limiting embodiments, this disclosure is directed to a method for detecting colorectal adenoma in a patient which comprises: (a) obtaining a suitable patient sample; (b) measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (c) comparing the patient sample levels with levels associated with a control sample, wherein elevated levels are indicative of whether or not colorectal adenoma is present or absent in the patient.

The disclosure is also directed to a kit for detecting colorectal adenoma in a patient sample which comprises: (a) a means for measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (b) instructions for comparing the patient sample levels with levels associated with healthy patient controls. In the kit elevated levels are indicative of whether or not colorectal adenoma is present or absent in the patient.

The disclosure is also directed to a method of identifying a compound that prevents or treats colorectal adenomas, the method comprising the steps of: (a) contacting a tissue or an animal model with a compound; (b) measuring a level of four or more bacteria selected from group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (c) determining a functional effect of the compound on the bacteria levels.

### 4. BRIEF DESCRIPTION OF THE FIGURES

FIG. 1: Richness (left panel) and evenness (right panel) for the Operational Taxonomic Units (“OTUs”) observed for cases (n=33) vs. controls (n=38). OTUs were created with the program AbundantOTU x. Ye, Y. Identification and Quantification of Abundant Species from Pyrosequences of 16S rRNA by Consensus Alignment. Proc BIBM 153-157 (2010). The x-axis is proportional to the number of subjects in each category. By the Wilcoxon test, cases had a significantly higher richness (p=0.0061) than controls, but there was no significant difference in evenness (p=0.36).

FIG. 2: Maximum likelihood tree generated from the 371 OTUs in which the OTU was observed in at least 25% of the patients studied. The tree was generated using the RaxXML EPA server (http://i12k-exelixis3.informatik.tu-muenchen.de/raxml) (see methods). Branches are colored based on RDP Phylum level assignments. Black branches represent OTUs significantly different between cases and controls within each Phylum (at 10% False Discovery Rate (“FDR”)).

FIG. 3: Richness (left panel) and evenness (right panel) at the phylum level in cases (n=33) vs. controls (n=38). By the Wilcoxon test, cases had a significantly higher richness (p=0.0041) than controls, but there was no significant difference in evenness (p=0.75).

FIG. 4: Richness (left panel) and evenness (right panel) at the genus level, in cases (n=33) vs. controls (n=38). By the Wilcoxon test, cases had a significantly higher richness (p=0.0013) than controls, but there was no significant difference in evenness (p=0.56).

FIG. 5: Principal Component Analysis (PCoA) PCoA generated from Fast UniFrac analysis on the tree displayed in FIG. 2. (Cases-squares; controls-circles).

FIG. 6: Regressions between q-PCR results and results from pyrosequencing data for genera Helicobacter, Acidovorax and Cloacibacterium. Reasonable correlations were obtained using the two methods; by linear regression: Acidovorax R=0.6, p<0.001; Cloacibacterium R=0.61, p<0.001 and Helicobacter R=0.56, p<0.0001.

FIG. 7: Rank-abundance curve in which the x-axis is the log abundance rank of the top 371 OTUs and the y-axis is the average log normalized sequence count across all samples. The OTU is marked by squares if the difference between cases and controls is significant at 10% FDR and by open circles if the difference is not significant at 10% FDR.

FIG. 8: Richness (left panel) and evenness (right panel) at the OTU level, in Normal (n=27) vs. Overweight (n=25) vs. Obese (n=18) Body Mass Index (“BMI”) categories. No significant difference was seen by the Kruskal-Wallis test in richness (p=0.21) or evenness (p=0.42) between the 3 categories.

FIG. 9: Richness (left panel) and evenness (right panel) at the OTU level, in Low-Risk (n=25) vs. Medium-Risk (n=16) vs. High-Risk (n=30) Waist-to-hip ratio (“WHR”) categories. No significant difference was seen by the Kruskal-Wallis test in richness (p=0.26) or evenness (p=0.76) between the 3 categories.

FIG. 10: Regressions on log-normalized abundance of OTU16 (top ranking OTU based on regression p-Value) vs. BMI of all samples. Note that after correction for multiple hypothesis testing, this regression is not significant at a 10% FDR threshold (see Table 6).

FIG. 11: Regressions on log-normalized abundance of OTU4 (top ranking OTU based on regression p-Value) vs. WHR of all samples. Note that after correction for multiple hypothesis testing, this regression is not significant at a 10% FDR threshold (see Table 7).

FIGS. 12-1-12-7: Maximum likelihood tree generated from the top 371 OTUs using RaxXML EPA server. FIG. 12-1 Proteobacteria; FIG. 12-2 Bacteriodes; FIG. 12-3-12-6 Firmicutes; FIG. 12-7 Other. In bold associated with the black axes are the OTUs significantly different. Leaf nodes are labeled with the Ribosomal Database Project (RDP) Classifier call of the consensus sequence at 80%. Wang, Q., Garrity, G. M., Tiedje, J. M. & Cole, J. R. Naive Bayesian classifier for rapid assignment of rRNA sequences into the new bacterial taxonomy. Appl Environ Microbiol 73, 5261-5267 (2007). Branches are black if the OTU was significantly different between cases and controls and gray if not significant (at 10% FDR).

FIG. 13: Abundance of Fusobacterium in rectal mucosal biopsies from adenoma cases and non-adenoma controls. qPCR results show that Fusobacterium is more abundant in cases than controls.

FIG. 14: Correlations between Fusobacterium abundance and local cytokine gene expression in adenoma cases and non-adenoma controls. Results suggest a significant positive correlation between Fusobacterium abundance and local inflammation in cases but not controls. The correlations were significant for IL-10 (r=0.44, p=0.01) and TNF-α (r=0.33, p=0.06).

FIG. 15: Log Abundance of Fusobacterium in matched normal colon and colorectal cancer tissue. Fusobacterium abundance was evaluated in DNA samples from normal colon and tumor tissue by qPCR using Fusobacterium-specific primers. Results suggest that Fusobacterium is increased in colon cancer tissue compared to normal tissue (ttest p=0.0005).

FIG. 16: Hierarchical clustering of bacterial community profiles in rectal swabs and rectal biopsies. Bray-Curtis similarities were used to construct a dendrogram composed of the samples provided by the participants (1-11). Each participant is represented twice: rectal swab (light gray triangles) and rectal biopsy (dark gray triangles).

FIG. 17: Distribution of Terminal-restriction fragments (T-RFs) in rectal swabs and rectal biopsies. Bars represent the average abundance of each T-RF grouped by biopsies (dark gray) or swabs (light gray). Asterisks represent T-RFs that are significantly different (p<0.05) between rectal biopsies and rectal swabs as assessed by t-test.

FIG. 18: Measures of T-RF Diversity in rectal swabs and rectal biopsies. Bars represent average diversity as estimated by T-RF richness (p=0.014), evenness (p=0.058) and Shannon's diversity (p=0.04). Calculated standard error is represented atop each bar graph. Statistical significance (*) was calculated by t-test.

FIG. 19: Quantitative PCR of Bacterial 16S RNA Gene of (FIG. 19A) Lactobacillus spp., (FIG. 19B) Eubacteria, (FIG. 19C) Bacteroides spp., (FIG. 19D) E. coli, (FIG. 19E) Clostridium spp. and (FIG. 19F) Bifidobacterium spp. in rectal swabs and rectal biopsies. A significant increase in Lactobacillus spp. (p=0.04) and Eubacterium spp. (p=0.011) was observed in rectal swabs compared to rectal biopsies (*).

FIG. 20: Hierarchical Clustering of bacterial communities in rectal swabs and rectal biopsies by adenoma status. Bray-Curtis similarities were used to construct dendrograms composed of the samples provided by the participants (1-11). Each participant is represented twice: for the rectal swab (light gray triangles) and rectal biopsy (dark triangles). FIG. 20A: adenoma cases FIG. 20B: non-adenoma controls. Significance values were calculated from Analysis of Similarity (ANOSIM).

FIG. 21: Pair-wise comparisons of bacterial community composition based on Bray-Curtis similarities; swabs (top row); biopsies (left column).

### 5. DETAILED DESCRIPTION OF THE INVENTION

This disclosure is directed to a method for detecting colorectal adenoma in a patient which comprises: (a) obtaining a suitable patient sample; (b) measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (c) comparing the patient sample levels with levels associated with a control sample, wherein elevated levels are indicative of whether or not colorectal adenoma is present or absent in the patient.

In some embodiments, the bacteria are selected from the group consisting of Acidovorax, Acinetobacter, Aquabacterium, Azonexus, Cloacibacterium, Dechloromonas, Delftia, Fusobacterium, Helicobacter, Lactobacillus, Lactococcus, Leuconostoc, Sphingobium, Stenotrophomonas, Succinivibrio, Turicibacter, and Weissella. The Fusobacterium may be F. nucleatum. The method may further comprising measuring levels of Bacteroides, Bifidobacteriaceae, Dorea, or Streptococcus, wherein decreased levels of Bacteroides, Bifidobacteriaceae, Dorea, or Streptococcus, are indicative of whether or not adenoma is present or absent in the patient. In one aspect of the disclosure, 8, 12, 15, 20 or 30 bacteria are measured. In another aspect, the bacteria are measured using the Operational Taxonomic Units (OTUs), such as those exemplified in Table 3. The specific OTUs correspond to the consensus sequences in the sequence listing, e.g., OTU72, Aquabacterium corresponds to consensus sequence #72 in U.S. Prov. Patent Appl. No. 61/493,770, which is SEQ ID No. 82 in the sequence listing. Similarly, OTU1 corresponds to SEQ ID No. 11, OTU100 to SEQ ID No. 110, OTU110 to SEQ ID No. 120, OTU353 to SEQ ID No. 363 . . . OTU613 to SEQ ID No. 623. One of ordinary skill could readily use the OTUs of interest and the sequence listing to find the name and additional details for any individual bacterial genus and species of interest or combinations or sets of bacteria to select patients likely to have adenomas. The sequences in the sequence listing may readily be entered into databases such as the SEQ MATCH section of the Ribosomal Database project (http://rdp.cme.msu.edu/index.jsp) or BLAST search in the 16S ribosomal RNA database of the National Center for Biotechnology Information (NCBI)(http://blast.ncbi.nlm.nih.gov/Blast.cgi).

Examples of OTUs/SEQ ID Nos. (#) of particular interest in combination for the claimed invention include up-regulation of OTU11(#21), OTU36(#46), OTU59(#69), OTU67(#77), OTU86(#96), OTU91(#101), OTU124(#134), OTU133(#143), OTU159(#169), OTU186(#196), OTU197(#207), OTU242(#252), OTU313 (#323), OTU322(#332), OTU330(#340), OTU353 (#463), OTU370(#380), OTU442(#452), OTU491 (#501), OTU501(#511) and down-regulation of OTU8 (#18), OTU66(#76), OTU169(#179).

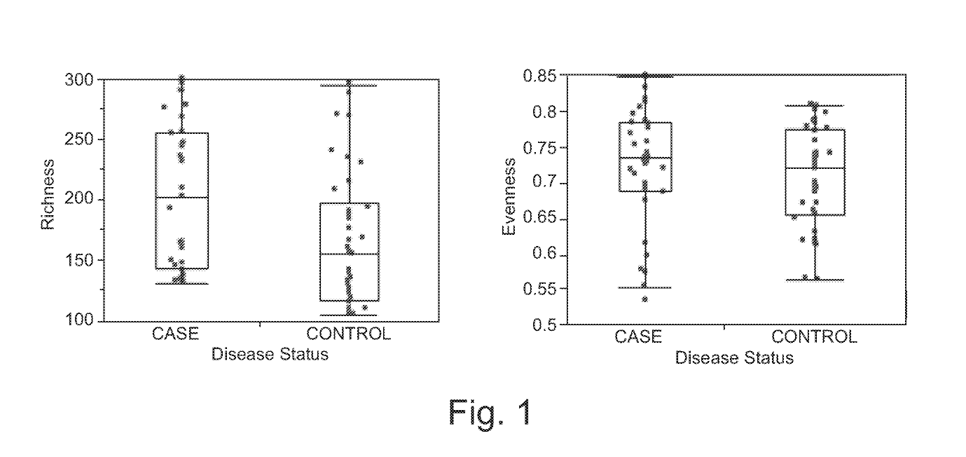

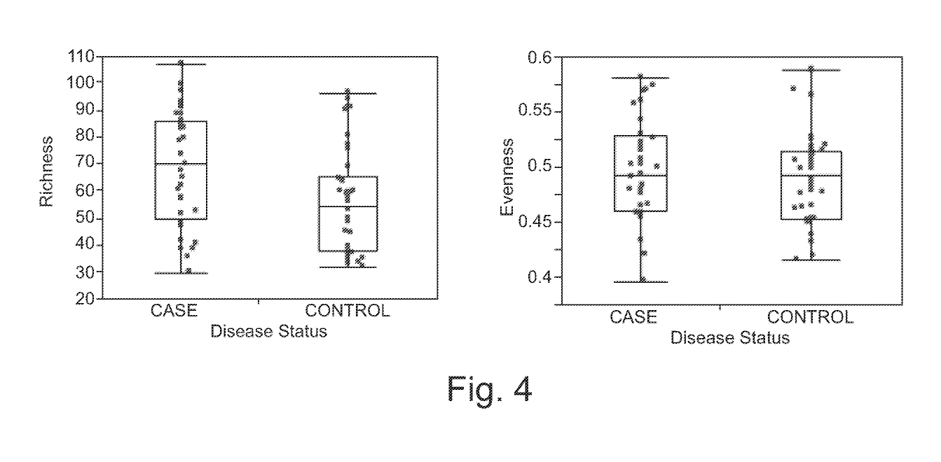

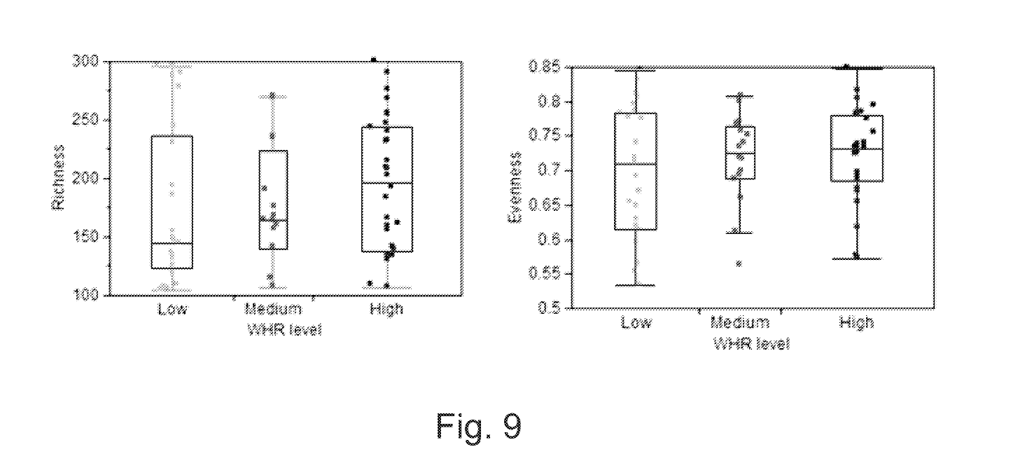

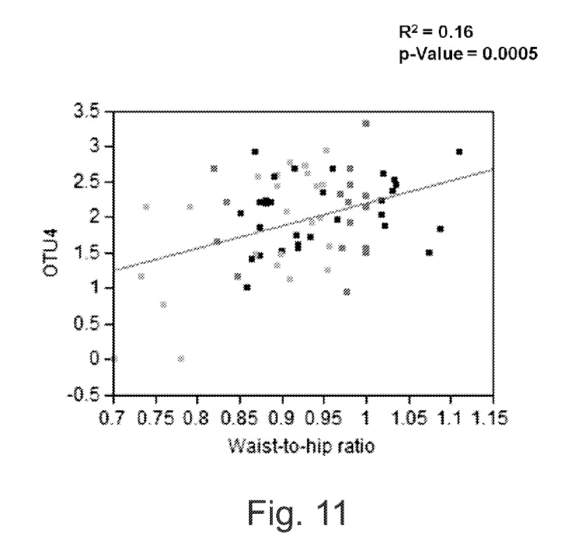

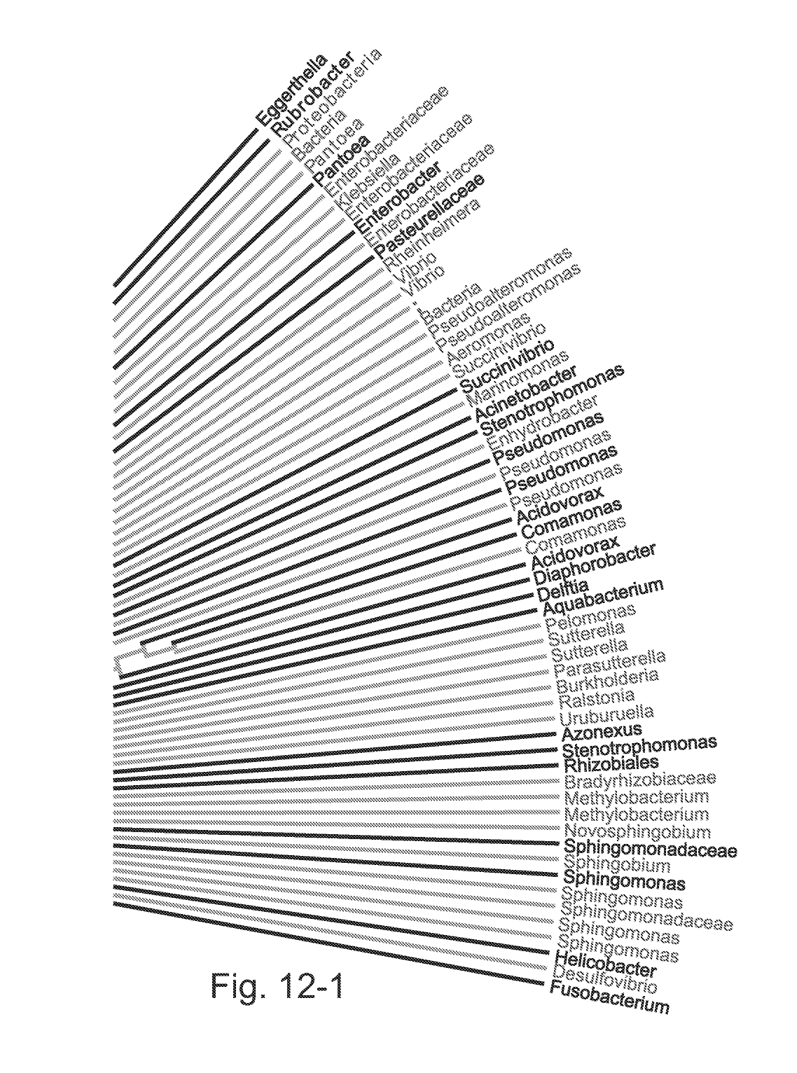

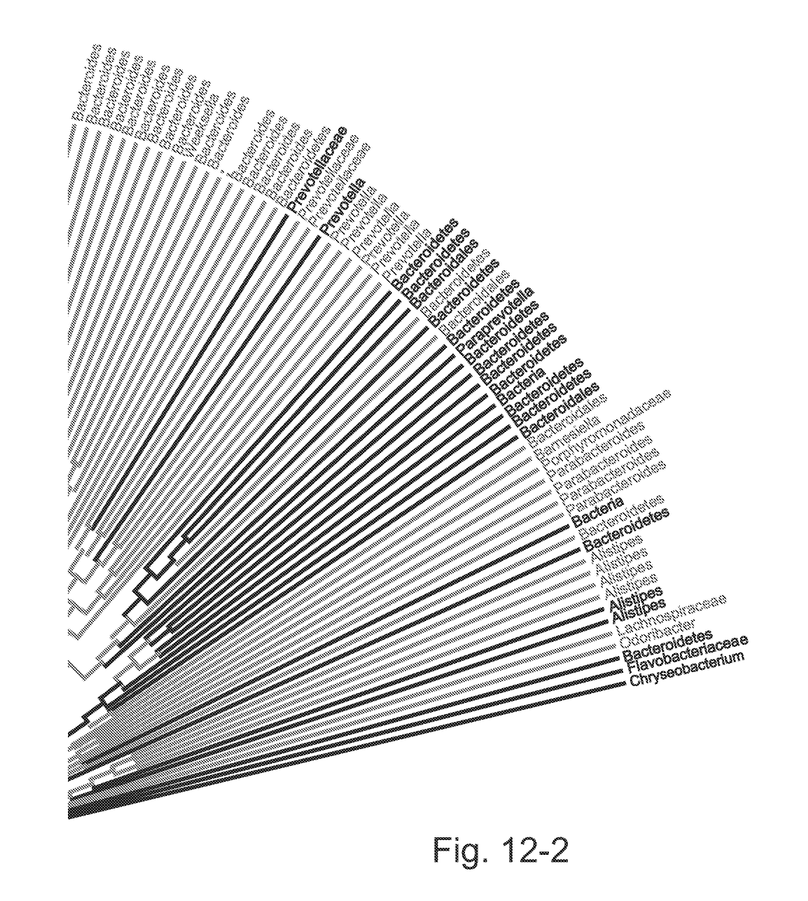

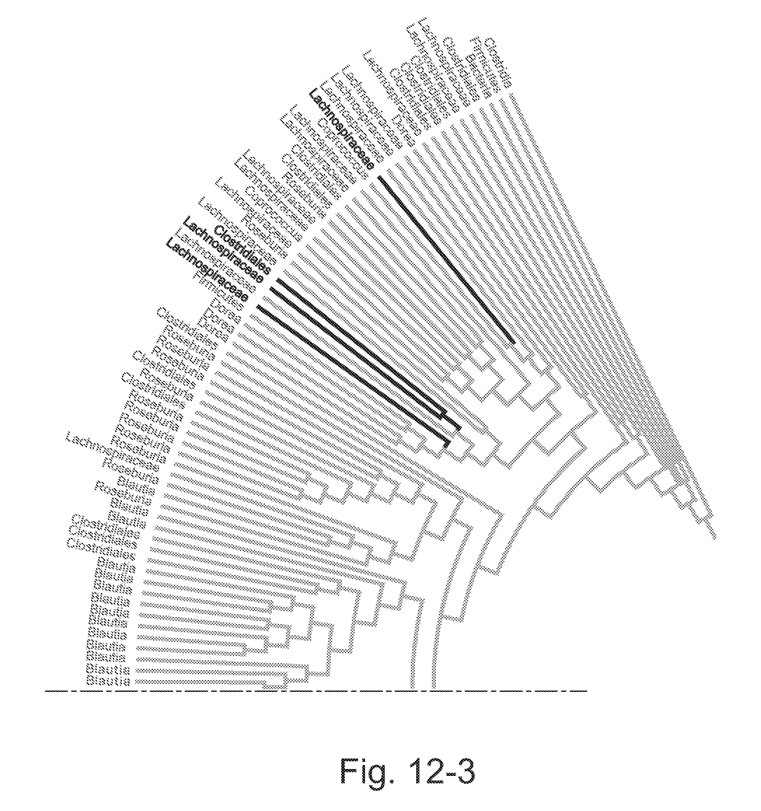

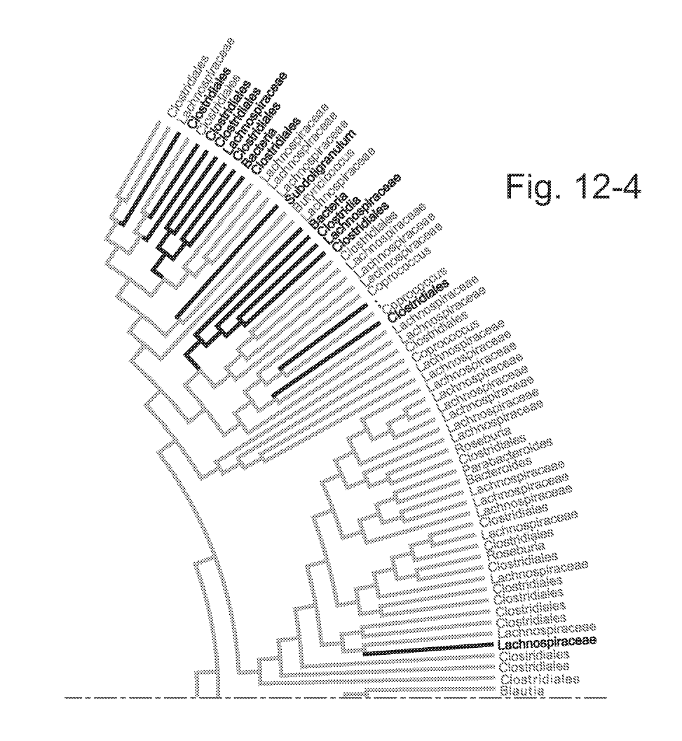

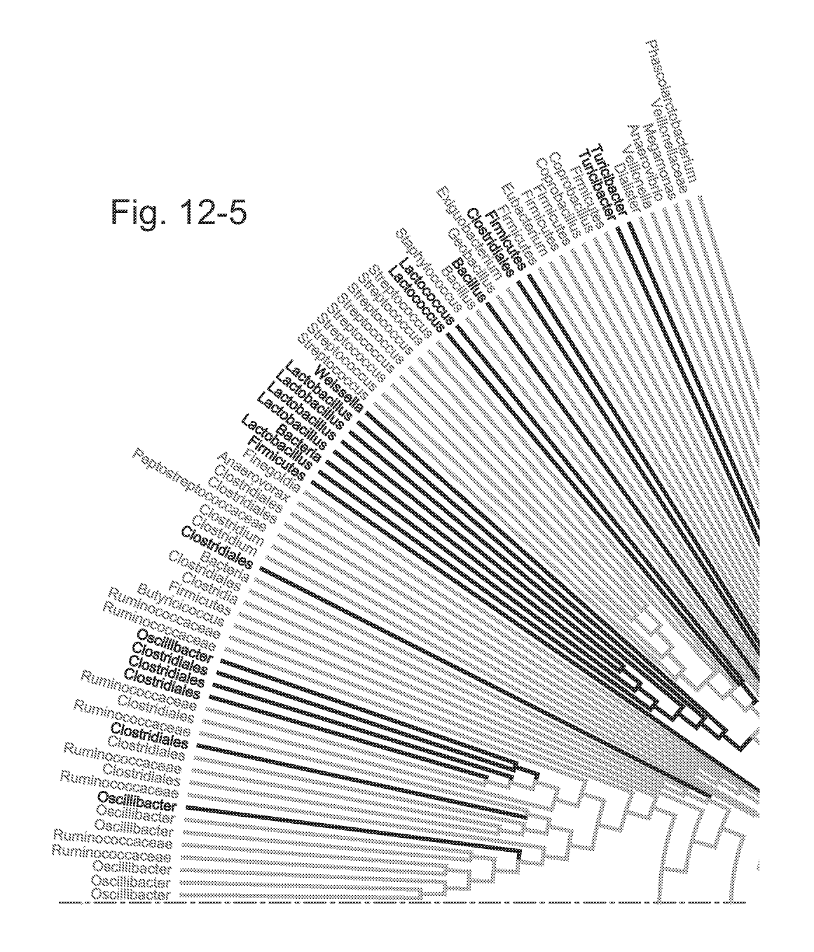

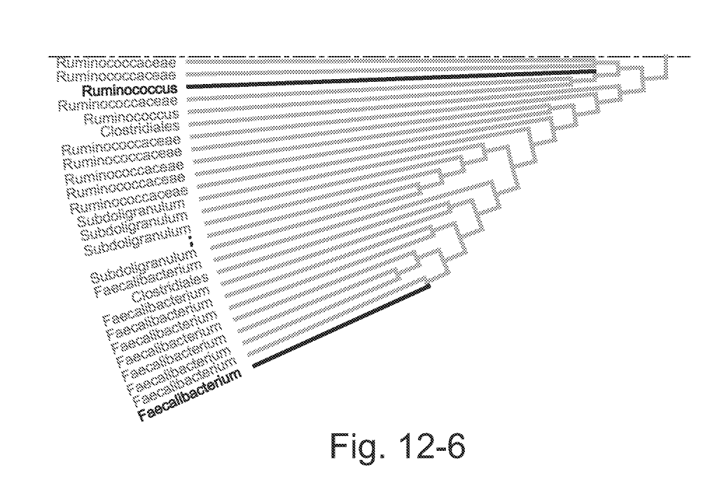

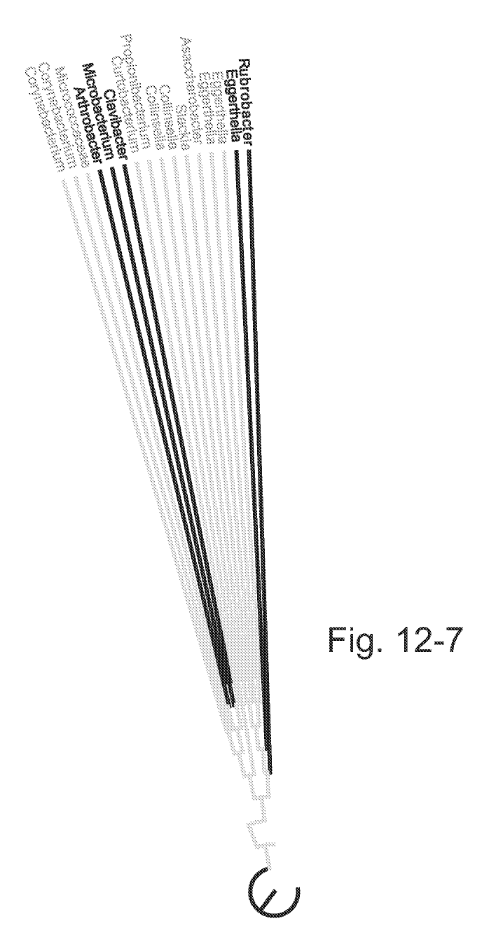

Alternatively, bacteria may be selected such that 2 or more bacteria are from the phyla, Proteobacteria; 2 or more bacteria are from the phyla Bacteriodetes; and 2 or more bacteria are from the phyla Firmicutes. One of ordinary skill could select multiple bacteria from different phyla or similar phyla that are different between cases and controls using groupings in FIG. 12-1-12-7.

The bacteria levels may be measured using bacterial nucleic acids such as 16S rRNA genes. They may also be measured using terminal restriction fragment length polymorphism (“T-RFLP”), fluorescence in-situ hybridization (“FISH”), polymerase chain reaction (“PCR”), pyrosequencing, or microarray.

The bacteria in the patient sample are cultured prior to measuring the levels. The bacteria levels may also be measured using antibodies. In some aspects of the disclosure, the patient sample may be a fecal sample. Alternatively, the patient sample is a biopsy sample such as a mucosa biopsy sample. The patient sample may also be a sample obtained by a rectal swab. The colorectal adenoma may be an adenocarcinoma.

The disclosure is also directed to a method for determining whether or not a patient should have a colonoscopy or a method for monitoring a patient for colorectal adenoma recurrence using the steps described above.

The disclosure is also directed to a kit for detecting colorectal adenoma in a patient sample which comprises: (a) a means for measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (b) instructions for comparing the patient sample levels with levels associated with healthy patient controls. In the kit elevated levels are indicative of whether or not colorectal adenoma is present or absent in the patient.

The disclosure is also directed to a kit comprising: (a) a reagent selected from a group consisting of: (i) nucleic acid probes capable of specifically hybridizing with nucleic acids from five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; (ii) a pair of nucleic acid primers capable of PCR amplification of five or more said bacteria; and (iii) four or more antibodies specific for said bacteria; and (b) instructions for use in measuring levels in a tissue sample from a patient suspected of having colorectal adenoma.

The disclosure is also directed to a method of identifying a compound that prevents or treats colorectal adenomas, the method comprising the steps of: (a) contacting a tissue or an animal model with a compound; (b) measuring a level of four or more bacteria selected from group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and (c) determining a functional effect of the compound on the bacteria levels. Thus by determining functional effects, one of ordinary skill may identify a compound that prevents or treats colorectal adenomas.

Also included in the methods and kits disclosed above are methods further comprising measuring analytes in a fecal test such as FOBT, FIT, or sDNA test. The methods disclosed above are complementary and may be used in combination with structural tests such as colonoscopy, flexible sigmoidoscopy, DCBE, CT colonography or capsule endoscopy. For CRC staging one may use the methods or kits described above in combination with pathologic examination of a colon biopsy, proximal lymph node evaluation, sentinel node evaluation, chest/abdominal/pelvic CT, MRI scans, positron emission tomography (“PET”) scans, liver functionality tests (for liver metastases), and blood tests (complete blood count (“CBC”), carcinoembryonic antigen (“CEA”), CA 19-9).

### 5.1. DEFINITIONS

The term “adenoma” refers to a growth of epithelial cells of glandular origin which may be benign or malignant. They are also referred to as adenomatous polyps. Adenomas may be peduculated (large head with a narrow stalk) or sessile (broad based). They may be classified as tubular adenomas, tubulovillous adenomas, villous adenomas, and flat adenomas. The adenoma may be an adenocarcinoma. The adenoma may be an adenoma from a human patient which may be a large adenoma>10 cm, a small adenoma<5 cm, or an adenoma between 0.5 cm and 15 cm in length.

The terms “nucleic acid” and “nucleic acid molecule” may be used interchangeably throughout the disclosure. The terms refer to nucleic acids of any composition from, such as DNA (e.g., complementary DNA (“cDNA”), genomic DNA (“gDNA”) and the like), ribosomal DNA (“rDNA”), RNA (e.g., messager RNA (“mRNA”), short inhibitory RNA (“siRNA”), ribosomal RNA (“rRNA”), transfer RNA (“tRNA”), microRNA, and the like), and/or DNA or RNA analogs (e.g., containing base analogs, sugar analogs and/or a non-native backbone and the like), RNA/DNA hybrids and polyamide nucleic acids (“PNAs”), all of which can be in single- or double-stranded form, and unless otherwise limited, can encompass known analogs of natural nucleotides that can function in a similar manner as naturally occurring nucleotides. Examples of nucleic acids are SEQ ID Nos. 1-623.

A nucleic acid in some examples may be from a microorganism which may be cultured (Cannon et al., App Envir Microbiol 3878-3885 (2002); Eckburg et al., Sci 308 1635-1638 (2005); Moore and Moore 1995; or Anaerobe Laboratory Manual. Holdeman et al. eds. 1977, 4^{th} Ed. p. 1-156); uncultured (Jurgens et al., FEMS Microbiol Ecol. 34(1) 45-56 (2000); Palmer et al., Nuc Acids Res 34(1) e5 (2006); Palmer et al. PLoS Biol 5(7) e177 1556-1573 (2007); Scanlon et al., Envir. Micro. 10(3) 789-798 (2008); Zengler et al., Proc Nat Acad Sci 99(24) 15681-15686 (2002), the contents of which are hereby incorporated by reference in their entireties. A nucleic acid may be a small subunit (“SSU”) rDNA, 16S, or 23S rRNA fragment or full-length rRNA sequence. It may be a nucleic acid encoding a 16S variable region such as V1, V2, V3, V4, V5, V6, V7, V8, V9, or a combination thereof. In some examples, the V2, V3, or V6 regions may be used. A nucleic acid may also be a ribosomal intergenic spacer (“RIS”) or internal transcribed spacer (“ITS”) fragment. It may be a sequence found using microarray or FISH analysis.

A template nucleic acid in some embodiments may be specific for a single bacteria taxa or a nucleic acid capable of binding to a variety of taxa. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses methylated forms, conservatively modified variants thereof (e.g., degenerate codon substitutions), alleles, orthologs, single nucleotide polymorphisms (“SNPs”), and complementary sequences as well as the sequence explicitly indicated. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has, or has the possibility of occurring. For example a location containing CpG is a methylation site wherein the cytosine may or may not be methylated.

As used herein, a “CpG site” or “methylation site” is a nucleotide within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated. An example of a methylated nucleic acid associated with CRC is vimentin. Shirahata et al., Anticancer Res. 30(12) 5015-5018 (2010).

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., Genome Research, 14, 247-266 (2004)). Others have defined a CpG island less stringently as a sequence at least 200 nucleotides in length, having a greater than 50% GC content, and an OCF/ECF ratio greater than 0.6 (Takai et al., Proc. Natl. Acad. Sci. USA, 99, 3740-3745 (2002)).

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (“PCR”), to amplify a nucleotide sequence based on the polynucleotide sequence corresponding to a particular genomic sequence, e.g., one specific for a particular bacteria. At least one of the PCR primers for amplification of a polynucleotide sequence is sequence-specific for the sequence.

The term “template” refers to any nucleic acid molecule that can be used for amplification in the technology. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes, but is not limited to, polymerase chain reaction, self-sustained sequence reaction, ligase chain reaction, rapid amplification of cDNA ends, polymerase chain reaction and ligase chain reaction, Q-β replicase amplification, strand displacement amplification, rolling circle amplification, or splice overlap extension polymerase chain reaction. In some embodiments, a single molecule of nucleic acid may be amplified.

The term “sensitivity” as used herein refers to the number of true positives divided by the number of true positives plus the number of false negatives, where sensitivity (“sens”) may be within the range of 0<sens<1. Ideally, method embodiments herein have the number of false negatives equaling zero or close to equaling zero, so that no subject is wrongly identified as not having adenoma when they indeed have adenoma. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having adenoma when they do not in fact have adenoma. Hence, a method that has both sensitivity and specificity equaling one, or 100%, is preferred.

The phrase “functional effects” in the context of assays for testing means compounds that modulate a phenotype or a gene associated with adenoma either in vitro, in cell culture, in tissue samples, or in vivo. This may also be a chemical or phenotypic effect such as altered bacterial profiles in vivo, e.g., changing from a high risk of adenoma or CRC bacterial profile to a low risk profile; altered expression of genes associated with adenoma or CRC; altered transcriptional activity of a gene hyper- or hypomethylated in adenoma; or altered activities and the downstream effects of proteins encoded by these genes. A functional effect may include transcriptional activation or repression, the ability of cells to proliferate, expression in cells during adenoma progression, and other characteristics of colorectal cells. “Functional effects” include in vitro, in vivo, and ex vivo activities. By “determining the functional effect” is meant assaying for a compound that increases or decreases the transcription of genes or the translation of proteins that are indirectly or directly under the influence of a gene hyper- or hypomethylated in adenoma or adenocarcinoma. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. Validation of the functional effect of a compound on adenoma occurrence or progression can also be performed using assays known to those of skill in the art such as studies using Min (multiple intestinal neoplasia) mice. Alternatively, a colon tissue may be maintained in culture. Bareiss et al., Histochem Cell Biol 129 795-804 (2008). The functional effects can be evaluated by many means known to those skilled in the art, e.g., microscopy for quantitative or qualitative measures of alterations in morphological features, measurement of changes in RNA or protein levels for other genes associated with bacteria differentially expressed in adenoma, measurement of RNA stability, identification of downstream or reporter gene expression (CAT, luciferase, β-gal, GFP, and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of the expression of genes hyper- or hypomethylated in adenoma, mutations associated with adenoma, or the translation proteins encoded thereby Inhibitors, activators, or modulators also include naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., (1)(a) the mRNA expression, or (b) proteins expressed by genes hyper- or hypomethylated in adenoma in vitro, in cells, or cell extracts; (2) applying putative modulator compounds; and (3) determining the functional effects on activity, as described above.

Assays comprising in vivo measurement of bacterial profiles associated with a high risk of adenoma or CRC; or genes hyper- or hypomethylated in adenoma are treated with a potential activator, inhibitor, or modulator are compared to control assays without the inhibitor, activator, or modulator to examine the extent of inhibition. Controls (untreated) are assigned a relative activity value of 100% Inhibition of a bacterial profile, or methylation, expression, or proteins encoded by genes hyper- or hypomethylated in adenoma is achieved when the activity value relative to the control is about 80%, preferably 50%, more preferably 25-0%. Activation of a bacterial profile or methylation, expression, or proteins encoded by genes hyper- or hypomethylated in adenoma is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide, small organic molecule, polysaccharide, peptide, circular peptide, lipid, fatty acid, siRNA, polynucleotide, oligonucleotide, etc., to be tested for the capacity to directly or indirectly modulate associated with adenoma. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (“HTS”) methods are employed for such an analysis. The compound may be “small organic molecule” that is an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### 5.2. SAMPLES

The sample may be from a patient suspected of having adenoma or from a patient diagnosed with CRC. The biological sample may also be from a subject with an ambiguous diagnosis in order to clarify the diagnosis. The sample may be obtained for the purpose of differential diagnosis, e.g., to confirm the diagnosis. The sample may also be obtained for the purpose of prognosis, i.e., determining the course of the disease and selecting primary treatment options. Tumor staging and grading are examples of prognosis. The sample may also be evaluated to select or monitor therapy, selecting likely responders in advance from non-responders or monitoring response in the course of therapy. In addition, the sample may be evaluated as part of post-treatment ongoing surveillance of patients who have had adenoma or CRC.

Biological samples may be obtained using any of a number of methods in the art. Examples of biological samples comprising bacteria include those obtained from excised biopsies, such as punch biopsies, shave biopsies, fine needle aspirates (“FNA”), or surgical excisions; or biopsy from non-cutaneous tissues such as lymph node tissue, mucosa, conjuctiva, or uvea, other embodiments. Representative biopsy techniques include, but are not limited to, mucosal biopsy, excisional biopsy, incisional biopsy, needle biopsy, surgical biopsy. A diagnosis or prognosis made by endoscopy or fluoroscopy can require a “core-needle biopsy” of the tumor mass, or a “fine-needle aspiration biopsy” which generally contains a suspension of cells from within the tumor mass.

A sample may also be a sample from a muscosal surface, such as a fecal or rectal swab sample, a blood and blood fractions or products (e.g., serum, plasma, platelets, red blood cells, white blood cells, circulating tumor cells isolated from blood, free DNA isolated from blood, and the like), sputum, lymph and tongue tissue, cultured cells, e.g., primary cultures, explants, and transformed cells, stool, urine, etc. A sample is typically obtained from a eukaryotic organism, most preferably a mammal such as a primate e.g., chimpanzee or human; cow; dog; cat; a rodent, e.g., guinea pig; rat; mouse; rabbit. Example 6.3 below shows rectal swab sample collection and and analysis.

Sample handling for bacterial analysis in stool samples is described in Wu et al. Sampling and pyrosequencing methods for characterizing bacterial communities in the human gut using 16S sequence tags. BMC Microbiology 10: 206 (2010), the contents of which is hereby incorporated by reference in its entirety. Commercially available kits include QIAamp DNA Stool Minikit (Cat#51504, Qiagen, Valencia, Calif.), PSP Spin Stool DNA Plus Kit (Cat#10381102, Invitek, Berlin, Germany), MoBio PowerSoil DNA Isolation Kit (Cat#12888-05, Mo Bio Laboratories, Carlsbad, Calif.).

A sample can be treated with a fixative such as Carnoy's fixative and embedded in paraffin (“FFPE”) and sectioned for use in the methods of the invention. Alternatively, fresh or frozen tissue may be used. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Typically, biological samples, once obtained, are harvested and processed prior to hybridization using standard methods known in the art. Such processing typically includes fixation in chloroform-acetic acid-alcohol based solution such as Carnoy's fixative and protease treatment.

5.2.1. Nucleic Acid Sequence Amplification and Detection

In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the invention may use any nucleic acid amplification or detection methods known to one skilled in the art, such as those described in U.S. Pat. No. 5,525,462 (Takarada et al.); U.S. Pat. No. 6,114,117 (Hepp et al.); U.S. Pat. No. 6,127,120 (Graham et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

In some embodiments, the nucleic acids are amplified by PCR amplification using methodologies known to one skilled in the art. One skilled in the art will recognize, however, that amplification can be accomplished by any known method, such as polymerase chain reaction (PCR), ligase chain reaction (LCR), Qβ-replicase amplification, rolling circle amplification, transcription amplification, self-sustained sequence replication, nucleic acid sequence-based amplification (NASBA), each of which provides sufficient amplification. Branched-DNA technology may also be used to qualitatively demonstrate the presence of a sequence of the technology or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. Machines for pyrosequencing and methylation specific reagents are available from Qiagen, Inc. (Valencia, Calif.). An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., J. Biotech. 102, 117-124 (2003)). Such a system can be used to exponentially amplify amplification products generated by a process described herein, e.g., by ligating a heterologous nucleic acid to the first amplification product generated by a process described herein.

Amplified sequences may also be measured using the Agilent 2100 Bioanalyzer to quantify amplified PCR products prior to pooling and pyrosequencing, or invasive cleavage reactions such as the Invader® technology (Zou et al., Association of Clinical Chemistry (AACC) poster presentation on Jul. 28, 2010, “Sensitive Quantification of Methylated Markers with a Novel Methylation Specific Technology,” available at www.exactsciences.com; and U.S. Pat. No. 7,011,944 (Prudent et al.) which are incorporated herein by reference in their entirety).

5.2.2. High Throughput and Single Molecule Sequencing Technology

Suitable next generation nucleic acid sequencing and detection technologies are widely available. Examples include the 454 Life Sciences platform (Roche, Branford, Conn.) (Margulies et al. Nature, 437, 376-380 (2005)); Illumina's Genome Analyzer, GoldenGate Methylation Assay, or Infinium Methylation Assays (Illumina, San Diego, Calif.; Bibkova et al., 2006, Genome Res. 16, 383-393; U.S. Pat. Nos. 6,306,597 and 7,598,035 (Macevicz); U.S. Pat. No. 7,232,656 (Balasubramanian et al.)); or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); or the Helicos True Single Molecule DNA sequencing technology (Harris et al., 2008 Science, 320, 106-109; U.S. Pat. Nos. 7,037,687 and 7,645,596 (Williams et al.); U.S. Pat. No. 7,169,560 (Lapidus et al.); U.S. Pat. No. 7,769,400 (Harris)), the single molecule, real-time (SMRT™) technology of Pacific Biosciences, and sequencing (Soni and Meller, Clin. Chem. 53, 1996-2001 (2007)) which are incorporated herein by reference in their entirety. These systems allow the sequencing of many nucleic acid molecules isolated from a specimen at high orders of multiplexing in a parallel fashion (Dear, Brief Funct. Genomic Proteomic, 1(4), 397-416 (2003) and McCaughan and Dear, J. Pathol., 220, 297-306 (2010)). Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (“TIRM”). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully. Bailey et al. recently reported a highly sensitive (15 pg methylated DNA) method using quantum dots to detect methylation status using fluorescence resonance energy transfer (MS-qFRET)(Bailey et al. Genome Res. 19(8), 1455-1461 (2009), which is incorporated herein by reference in its entirety).

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslaysky et al., PNAS 100(7): 3960-3964 (2003); U.S. Pat. No. 7,297,518 (Quake et al.) which are incorporated herein by reference in their entirety). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

The technology may be practiced with digital PCR. Digital PCR was developed by Kalinina and colleagues (Kalinina et al., Nucleic Acids Res. 25; 1999-2004 (1997)) and further developed by Vogelstein and Kinzler, Proc. Natl. Acad. Sci. U.S.A. 96; 9236-9241 (1999)). The application of digital PCR is described by Cantor et al. (PCT Pub. Nos. WO 2005/023091A2 (Cantor et al.); WO 2007/092473 A2, (Quake et al.)), which are hereby incorporated by reference in their entirety. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in PCT Pub. No. WO 2009/091934 (Cantor).

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected.

A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

The invention encompasses any method known in the art for enhancing the sensitivity of the detectable signal in such assays, including, but not limited to, the use of cyclic probe technology (Bakkaoui et al., 1996, BioTechniques 20: 240-8, which is incorporated herein by reference in its entirety); and the use of branched probes (Urdea et al., 1993, Clin. Chem. 39, 725-6; which is incorporated herein by reference in its entirety). The hybridization complexes are detected according to well-known techniques in the art.

Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

5.2.3. Mass Spectroscopic Detection Methods

Another method for analyzing bacteria in samples is mass spectrometry. The assay can also be done in multiplex. Mass spectrometry is a particularly effective method for the detection of specific polypeptides or polynucleotides associated with bacteria. See for example, Identification of Microorganisms by Mass Spectrometry, Ed. Wilkons and Lay, Wiley-Interscience, 2006; U.S. Pat. No. 7,070,739 (Anderson and Anderson); U.S. Pat. No. 6,177,266 (Krishnamurthy and Ross); PCT Pub Nos. WO 2010/062354 A1 (Hyman et al.); WO 2008/058024 A2 (Eckstein and Eckstein); WO 2001/079523 A2 (Pineda and Lin); European Patent Pub. No. EP 1437673 B1 (Kallow et al.); U.S. Patent Pub. No. US 2005/0142584 A1 (Willson et al.); which are hereby incorporated by reference in their entirety.

5.2.4. Fluorescence In Situ Hybridization (FISH)

In some examples, the invention may further encompass detecting and/or quantitating using fluorescence in situ hybridization (FISH) in a sample, preferably a tissue sample, obtained from a subject in accordance with the methods of the invention. FISH is a common methodology used in the art, especially in the detection of specific chromosomal aberrations in tumor cells, for example, to aid in diagnosis and tumor staging. As applied in the methods of the invention, it can be used to detect types and levels of bacteria. For reviews of FISH methodology, see, e.g., Harmsen et al., Appl Environ Microbiol 68 2982-2990 (2002); Kalliomaki et al., J Allerg Clin Immunol 107 129-134 (2001); Tkachuk et al., Genet. Anal. Tech. Appl. 8: 67-74 (1991); Trask et al., Trends Genet. 7 (5): 149-154 (1991); and Weier et al., Expert Rev. Mol. Diagn. 2 (2): 109-119 (2002); U.S. Pat. No. 6,174,681 (Halling et al.); all of which are incorporated herein by reference in their entirety. Example 6.2 below shows FISH staining for Fusobacterium.

In alternative embodiments, the invention encompasses use of bacteria specific gene expression and/or antibody assays either in situ, i.e., directly upon tissue sections (fixed and/or frozen) of patient tissue obtained from biopsies or resections, such that no nucleic acid purification is necessary; or based on extracted and/or amplified nucleic acids. Targets for such assays are disclosed in Haqq et al., Proc. Nat. Acad. Sci. USA, 102(17), 6092-6097 (2005); Riker et al., BMC Med. Genomics, 1, 13, pub. 28 Apr. 2008; Hoek et al., Can. Res. 64, 5270-5282 (2004); PCT Pub. Nos. WO 2008/030986 and WO 2009/111661 (Kashani-Sabet & Haqq); U.S. Pat. No. 7,247,426 (Yakhini et al.), all of which are incorporated herein by reference in their entirety. For in situ procedures see, e.g., Nuovo, G. J., 1992, PCR In Situ Hybridization: Protocols And Applications, Raven Press, N.Y., which is incorporated herein by reference in its entirety.

5.2.5. Microarrays

In some examples, DNA microarrays may used. Methods for making nucleic acid microarrays are known to the skilled artisan and are described, for example, in Lockhart et al., Nat. Biotech. 14, 1675-1680 (1996) Schena et al., Proc. Natl. Acad. Sci. USA, 93, 10614-10619 (1996), U.S. Pat. No. 5,837,832 (Chee et al.) and PCT Pub. No. WO 00/56934 (Englert et al.), herein incorporated by reference. Microarrays specific for gut microbes have been described, for example, Paliy et al. Appl Environ Microbiol 75 3572-3579 (2009); Palmer et al. (2006); and Palmer et al. (2007), herein incorporated by reference. Additional examples of microarray analysis for bacteria include Al-Khaldi et al. Nutrition 20 32-38 (2004); Apte and Singh Methods Mol Biol 402:329-346 (2007); Cleven et al. J Clin Microbiol 44(7) 2389-2397(2006); Dols et al. Am J Obstet Gyn 204(4) 305.e1-305.e7 (April 2011); Franke-Whittle et al. Application of COMPOCHIP Microarray to Investigate the Bacterial Communities of Different Composts. Microbial Ecol 57(3) 510-521 (2009); Huyghe et al. Appl Environ Microbiol 74(6):1876-85 (2008); Jarvinen et al. BMC Microbiol 9 161 (2009); Liu et al. Exp Biol Med 230(8) 587-591 (2005); Mao et al. Digestion 78 131-138 (2008); Pathak et al. Appl Microbiol Biotechnol 90(5) 1739-1754 (2011); Reyes-Lopez et al. Fingerprinting of prokaryotic 16S rRNA genes using oligodeoxyribonucleotide microarrays and virtual hybridization. Nucleic Acids Res 31:779-789 (2003); Thomassen et al. Custom Design and Analysis of High-Density Oligonucleotide Bacterial Tiling Microarrays PLoS ONE 4(6): e5943. doi:10.1371/journal.pone.0005943 (2009); Tissari et al. Lancet 375 224-230 (2010); PCT Publ. Nos. WO 2008/130394 (Andersen & Desantis) and WO 2010/151842 (Andersen et al.); herein incorporated by reference. To produce a nucleic acid microarray, oligonucleotides may be synthesized or bound to the surface of a substrate using a chemical coupling procedure and an ink jet application apparatus, as described U.S. Pat. No. 6,015,880 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

5.2.6. Antibody Staining/Detection

In some embodiments, the invention may encompass detecting and/or quantitating using antibodies either alone or in conjunction with measurement of bacterial nucleic acid levels. Antibodies are already used in current practice in the classification and/or diagnosis of bacteria.

Antibody reagents can be used in assays to detect expression levels of in patient samples using any of a number of immunoassays known to those skilled in the art Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., 1996, Curr. Opin. Biotechnol., 7, 60-65. The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated Immunoassays can also be used in conjunction with laser induced fluorescence. See, e.g., Schmalzing et al., 1997, Electrophoresis, 18, 2184-2193; Bao, 1997, J. Chromatogr. B. Biomed. Sci., 699, 463-480. Liposome immunoassays, such as flow-injection liposome immunoassays and liposome immunosensors, are also suitable for use in the present invention. See, e.g., Rongen et al., 1997, J. Immunol. Methods, 204, 105-133. In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.) and can be performed using a Behring Nephelometer Analyzer (Fink et al., 1989, J. Clin. Chem. Clin. Biochem., 27, 261-276).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 ¹²⁵I can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-/3-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot. The antibodies may be in an array one or more antibodies, single or double stranded nucleic acids, proteins, peptides or fragments thereof, amino acid probes, or phage display libraries. Many protein/antibody arrays are described in the art. These include, for example, arrays produced by Ciphergen Biosystems (Fremont, Calif.), Packard BioScience Company (Meriden Conn.), Zyomyx (Hayward, Calif.) and Phylos (Lexington, Mass.). Examples of such arrays are described in the following patents: U.S. Pat. No. 6,225,047 (Hutchens and Yip); U.S. Pat. No. 6,537,749 (Kuimelis and Wagner); and U.S. Pat. No. 6,329,209 (Wagner et al.), all of which are incorporated herein by reference in their entirety.

5.2.7. Fingerprinting Methods

In some examples, fingerprinting methods such as denaturing gradient gel electrophoresis (DGGE) or terminal restriction fragment length polymorphism (T-RFLP) may be used. DGGE studies the electrophoretic migration patterns of PCR amplicons of bacterial sequences such as the V6-V8 regions of the 16S rRNA gene. Differences in the DGGE patterns can be used to identify the bacterial communities. In T-RFLP analysis, a bacterial gene is amplified by PCR, such as the 16S rRNA gene and digested with a series of restriction endonucleases. Based on the sequence of the 16S gene, fragments of differing lengths will be generated. Those restriction fragments will give rise to a distinctive pattern in a capillary sequencer or gel electrophoresis. For DGGE, see Zoetendal et al., Appl Environ Microbiol 68 3401-3407 (2002), for T-RFLP, see Li et al., J Microbiol Methods 68 303-311 (2007); Osborn et al., Environ Microbiol 2 39-50 (2000); and Shen, X. J., et al. Gut Microbes 1, 138-147 (2010), incorporated herein by reference.

### 5.3. COMPOSITIONS AND KITS

The invention provides compositions and kits for detecting and/or measuring types and levels of bacteria using DNA assays, antibodies specific for the polypeptides or nucleic acids specific for the polynucleotides. Kits for carrying out the diagnostic assays of the invention typically include, a suitable container means, (i) a probe that comprises an antibody or nucleic acid sequence that specifically binds to the marker polypeptides or polynucleotides of the invention; (ii) a label for detecting the presence of the probe; and (iii) instructions for how to measure the type and level of a particular bacteria (or polypeptide or polynucleotide). The kits may include several antibodies or polynucleotide sequences encoding polypeptides of the invention, e.g., a first antibody and/or second and/or third and/or additional antibodies that recognize a protein associated with a particular bacteria. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.4. IN VIVO IMAGING

The various markers of the invention also provide reagents for in vivo imaging such as, for instance, the imaging of adenoma specific bacteria using labeled reagents that detect (i) nucleic acids associated with particular bacteria, (ii) a polypeptides associated with a particular bacteria. In vivo imaging techniques may be used, for example, as guides for surgical resection or to detect the distant spread of CRC. For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720 (Harada et al.); U.S. Pat. No. 5,227,487 (Haugland and Whitaker); and U.S. Pat. No. 5,543,295 (Bronstein et al.). Other fluorescent labels suitable for use in the practice of this invention include a fluorescein dye. Typical fluorescein dyes include, but are not limited to, 5-carboxyfluorescein, fluorescein-5-isothiocyanate, and 6-carboxyfluorescein; examples of other fluorescein dyes can be found, for example, in U.S. Pat. No. 4,439,356 (Khanna and Colvin); U.S. Pat. No. 5,066,580 (Lee), U.S. Pat. No. 5,750,409 (Hermann et al.); and U.S. Pat. No. 6,008,379 (Benson et al.). The kits may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. No. 5,936,087 (Benson et al.), U.S. Pat. No. 6,025,505 (Lee et al.); U.S. Pat. No. 6,080,852 (Lee et al.). The kits may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

### 5.5. METHODS TO IDENTIFY COMPOUNDS

A variety of methods may be used to identify compounds that modulate the growth of adenomas and prevent or treat adenocarcinoma progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on bacteria associated with adenoma can be determined. The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate the expression patterns of bacteria differentially detected in adenoma. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175 (Rutter and Santi), Furka, Int. J. Pept. Prot. Res., 37:487-493 (1991); and Houghton et al., Nature, 354:84-88 (1991)). Other chemistries for generating chemical diversity libraries can also be used. Such chemistries include, but are not limited to: U.S. Pat. No. 6,075,121 (Bartlett et al.) peptoids; U.S. Pat. No. 6,060,596 (Lerner et al.) encoded peptides; U.S. Pat. No. 5,858,670 (Lam et al.) random bio-oligomers; U.S. Pat. No. 5,288,514 (Ellman) benzodiazepines; U.S. Pat. No. 5,539,083 (Cook et al.) peptide nucleic acid libraries; U.S. Pat. No. 5,593,853 (Chen and Radmer) carbohydrate libraries; U.S. Pat. No. 5,569,588 (Ashby and Rine) isoprenoids; U.S. Pat. No. 5,549,974 (Holmes) thiazolidinones and metathiazanones; U.S. Pat. No. 5,525,735 (Takarada et al.) and U.S. Pat. No. 5,519,134 (Acevado and Hebert) pyrrolidines; U.S. Pat. No. 5,506,337 (Summerton and Weller) morpholino compounds; U.S. Pat. No. 5,288,514 (Ellman) benzodiazepines; diversomers such as hydantoins, benzodiazepines and dipeptides (Hobbs et al., 1993, Proc. Nat. Acad. Sci. USA, 90, 6909-6913), vinylogous polypeptides (Hagihara et al., 1992, J. Amer. Chem. Soc., 114, 6568), nonpeptidal peptidomimetics with glucose scaffolding (Hirschmann et al., 1992, J. Amer. Chem. Soc., 114, 9217-9218), analogous organic syntheses of small compound libraries (Chen et al., 1994, J. Amer. Chem. Soc., 116:2661 (1994)), oligocarbamates (Cho et al., 1993, Science, 261, 1303 (1993)), and/or peptidyl phosphonates (Campbell et al., 1994, J. Org. Chem., 59:658), nucleic acid libraries (see Ausubel, Berger and Sambrook, all supra); antibody libraries (see, e.g., Vaughn et al., 1996, Nat. Biotech., 14(3):309-314, carbohydrate libraries, e.g., Liang et al., 1996, Science, 274:1520-1522, small organic molecule libraries (see, e.g., benzodiazepines, Baum, 1993, C&EN, January 18, page 33. Devices for the preparation of combinatorial libraries are commercially available (see, e.g., 357 MPS, 390 MPS, Advanced Chem Tech, Louisville Ky., Symphony, Rainin, Woburn, Mass., 433 A Applied Biosystems, Foster City, Calif., 9050 Plus, Millipore, Bedford, Mass.). In addition, numerous combinatorial libraries are themselves commercially available (see, e.g., ComGenex (Princeton, N.J.), Asinex (Moscow, RU), Tripos, Inc. (St. Louis, Mo.), ChemStar, Ltd., (Moscow, RU), 3D Pharmaceuticals (Exton, Pa.), Martek Biosciences (Columbia, Md.), etc.).

Methylation modifiers are known and have been the basis for several approved drugs. Major classes of enzymes are DNA methyl transferases (DNMTs), histone deacetylases (HDACs), histone methyl transferases (HMTs), and histone acetylases (HATs). DNMT inhibitors azacitidine (Vidaza®) and decitabine have been approved for myelodysplastic syndromes (for a review see Musolino et al., Eur. J. Haematol. 84, 463-473 (2010); Issa, Hematol. Oncol. Clin. North Am. 24(2), 317-330 (2010); Howell et al., Cancer Control, 16(3) 200-218 (2009); which are hereby incorporated by reference in their entirety). HDAC inhibitor, vorinostat (Zolinza®, SAHA) has been approved by FDA for treating cutaneous T-cell lymphoma (CTCL) for patients with progressive, persistent, or recurrent disease (Marks and Breslow, Nat. Biotech. 25(1), 84-90 (2007)). Specific examples of compound libraries include: DNA methyl transferase (DNMT) inhibitor libraries available from Chem Div (San Diego, Calif.); cyclic peptides (Nauman et al., ChemBioChem 9, 194-197 (2008)); natural product DNMT libraries (Medina-Franco et al., Mol. Divers., 15(2):293-304 (2010)); HDAC inhibitors from a cyclic α3β-tetrapeptide library (Olsen and Ghadiri, J. Med. Chem. 52(23), 7836-7846 (2009)); HDAC inhibitors from chlamydocin (Nishino et al., Amer. Peptide Symp. 9(7), 393-394 (2006)).

### 5.6. METHODS OF INHIBITION USING NUCLEIC ACIDS

A variety of nucleic acids, such as antisense nucleic acids, siRNAs or ribozymes, may be used to inhibit the function of the markers of this invention. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

The following Examples further illustrate the invention and are not intended to limit the scope of the invention.

### 6. EXAMPLES

### 6.1. Microbial Signature Associated with Adenoma and CRC

454 titanium pyrosequencing of the V1-V2 region of the 16S rRNA gene was used to characterize adherent bacterial communities from mucosal biopsies of 33 adenoma subjects and 38 non-adenoma subjects. 87 taxa (including known pathogens) were found that had significantly higher relative abundances in cases vs. controls while only 5 taxa were more abundant in control samples. In addition adenoma samples had a pronounced increase in average microbial richness suggesting that conditions associated with colorectal adenomas create an environment in which potentially pathogenic microbes can flourish. Intriguingly, the magnitude of the differences between adenoma case and control in the gut microbiota was more pronounced than differences in the microbiota associated with patient obesity. Because the microbial signature associated with colorectal adenomas is generally distinct from microbial signatures associated with known risk factors such as increased body mass index (BMI), these results suggest that detection gut microbiota has potential utility as a diagnostic tool indicating the presence of adenomas.

One aim of this study was to use high throughput pyrosequencing approaches to explore the microbiome of the distal gut in individuals who have colorectal adenomas compared to a control group of individuals without adenomas. Associations of the microbiota with Body Mass Index (BMI) and Waist-to-Hip Ratio (WHR), which are known risk factors for colorectal cancer, were also evaluated. Caan, B. J., et al. Body size and the risk of colon cancer in a large case-control study. Int J Obes Relat Metab Disord 22, 178-184 (1998).

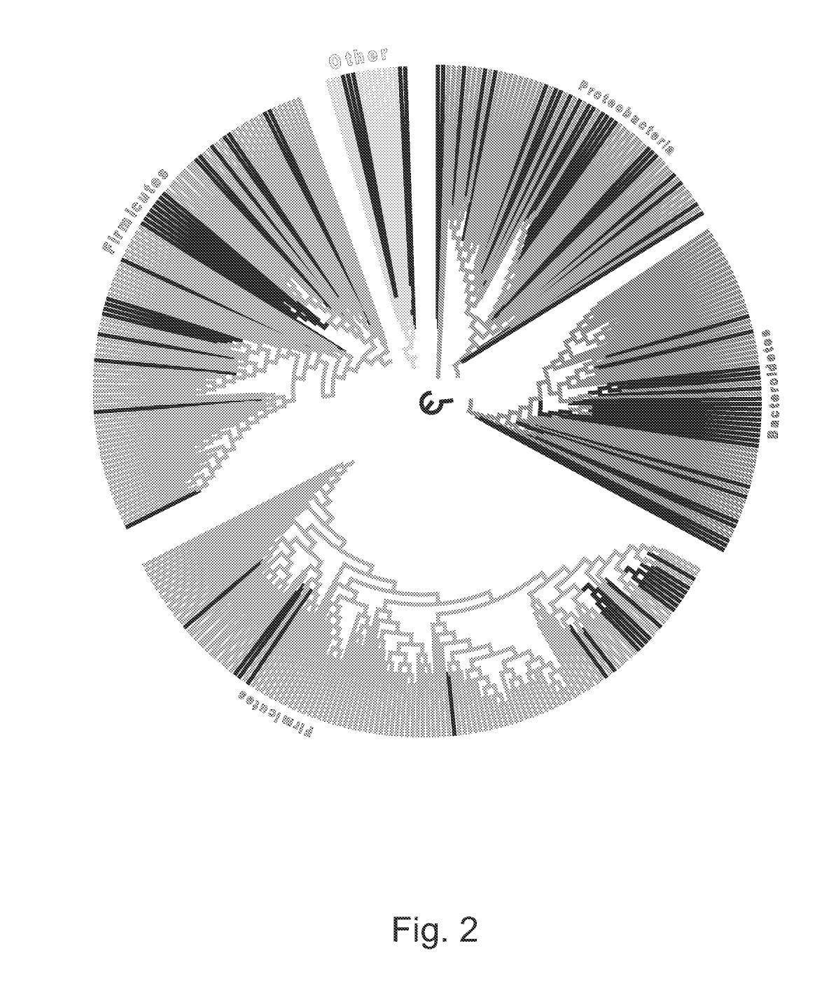

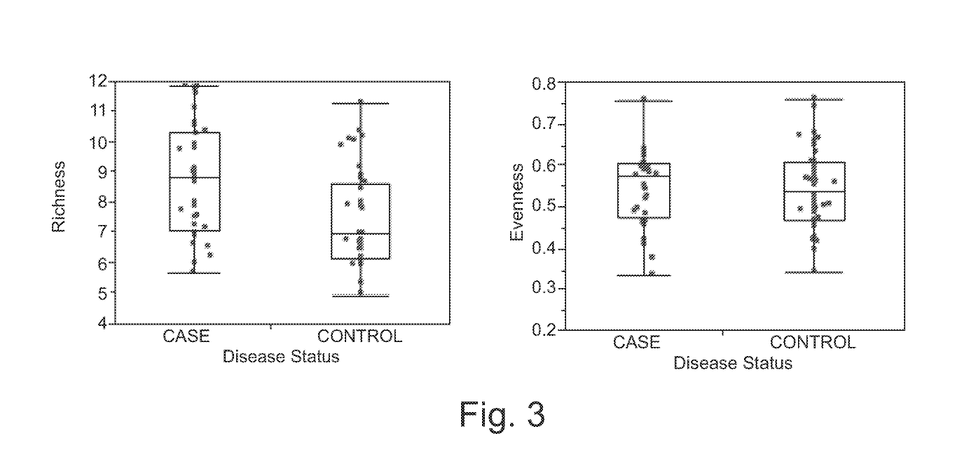

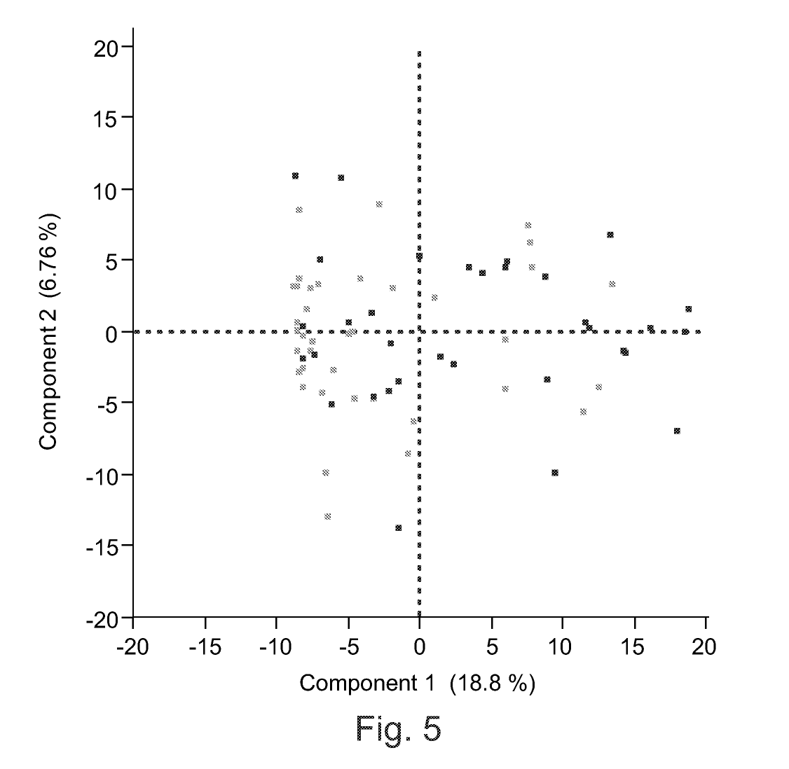

To evaluate associations between the gut microbiota and the presence of adenomas, mucosal biopsies were collected from the same region (˜10-12 cm regions from the anal verge) from 33 adenoma subjects and 38 controls. One analyses looked at global signatures of the entire microbial community. At the phylum, genus and Operational Taxonomic Unit (OTU) levels significant differences were found in richness (i.e. the number of taxa present in a sample), but no differences in evenness (i.e. how evenly distributed taxa are within a sample), between cases and controls (FIGS. 1, 3 & 4). In order to see whether case samples cluster separately from control samples, UniFrac was used to cluster the sequences based on their placement in the phylogenetic tree shown in FIG. 2. Lozupone C, Knight R. UniFrac: a new phylogenetic method for comparing microbial communities. Appl Environ Microbiol 71:8228-35 (2005). Running 100 permutations on the abundance weighted tree using the UniFrac significance test resulted in a p-value of 0.02 suggesting a marginally significant separation between cases and controls when considering all of the nodes of the phylogenetic tree. Similarly, weak clustering was seen when principle co-ordinate analysis (PCoA) was used on the same tree using FastUnifrac (FIG. 5).

Many individual bacterial taxa were different between cases and controls. By examining the results of the Ribosomal Database Project (“RDP”) classification algorithm at the phylum level at a 10% false discovery rate (“FDR”) threshold cases had higher relative abundance of TM7, Cyanobacteria and Verrucomicrobia compared to controls (Table 1). Wang Q, Garrity G M, Tiedje J M, Cole J R. Naive Bayesian classifier for rapid assignment of rRNA sequences into the new bacterial taxonomy. Appl Environ Microbiol 2007; 73:5261-7.

Table 1:

Wilcoxon-tests on log-normalized abundances of all phyla in cases (33 subjects) vs. controls (38 subjects). Only phyla which have at least 1 sequence assigned to them in 25% of the samples are shown. The direction of change shows the relative abundance in cases compared to controls. Wilcoxon p-Values were corrected for multiple testing using (n*p)/R where n=total number of taxa tested, p=raw p-Value and R=sorted Rank of the taxon. Benjamini, Y. & Hochberg, Y. A Practical and Powerful Approach to Multiple Testing. J Royal Statistical Soc Series B (Methodological) Vol. 57, 12 (1995).

TABLE 1

| | Wilcoxon | | | |
|---|---|---|---|---|
| Phylum Name | p-Value | Rank | (n*p)/R | Direction |
| TM7 | 0.00020 | 1 | 0.00180 | Up |
| Cyanobacteria* | 0.00220 | 2 | 0.00990 | Up |
| Verrucomicrobia | 0.00610 | 3 | 0.01830 | Up |
| Firmicutes | 0.04740 | 4 | 0.10665 | Down |
| Acidobacteria | 0.06010 | 5 | 0.10818 | Up |
| Fusobacteria | 0.17740 | 6 | 0.26610 | Up |
| Proteobacteria | 0.18110 | 7 | 0.23284 | Up |
| Actinobacteria | 0.31030 | 8 | 0.34909 | Up |
| Bacteroidetes | 0.83560 | 9 | 0.83560 | Up |

*While the sequences classified to Cyanobacteria may in fact originate from plastids or from non-Cyanobacteria, other human and animal gut studies have also observed sequences classified to Cyanobacteria. Ley, R. E., et al. Obesity alters gut microbial ecology. Proc Natl Acad Sci USA 102, 11070-11075 (2005).

At the genus level, the relative abundance levels of 24 genera including Acidovorax, Aquabacterium, Cloacibacterium, Helicobacter, Lactococcus, Lactobacillus and Pseudomonas were higher in case vs. control (Table 2).

Table 2:

Wilcoxon-tests on log-normalized abundances of genera in cases (33 subjects) vs. controls (38 subjects). Only genera which have at least 1 sequence assigned to them in 25% of the samples are shown. The direction of change shows the relative abundance in cases compared to controls. Wilcoxon p-Values were corrected for multiple testing using (n*p)/R where n=total number of taxa tested, p=raw p-Value and R=sorted Rank of the taxon. Benjamini & Hochberg (1995).

TABLE 2

| | Wilcoxon | | | |
|---|---|---|---|---|
| Genus | p-Value | Rank | (n*p)/R | Direction |
| Helicobacter | 0.00003 | 1 | 0.00290 | Up |
| Aquabacterium | 0.00005 | 2 | 0.00270 | Up |
| Weissella | 0.00026 | 3 | 0.00870 | Up |
| Lactococcus | 0.00070 | 4 | 0.01748 | Up |
| Acidovorax | 0.00083 | 5 | 0.01666 | Up |
| Turicibacter | 0.00128 | 6 | 0.02138 | Up |
| Lactobacillus | 0.00134 | 7 | 0.01917 | Up |
| Sphingobium | 0.00137 | 8 | 0.01715 | Up |
| Cloacibacterium | 0.00145 | 9 | 0.01611 | Up |
| Stenotrophomonas | 0.00171 | 10 | 0.01709 | Up |
| Succinivibrio | 0.00261 | 11 | 0.02374 | Up |
| Azonexus | 0.00324 | 12 | 0.02702 | Up |
| Leuconostoc | 0.00326 | 13 | 0.02504 | Up |
| Delftia | 0.00385 | 14 | 0.02752 | Up |
| Dechloromonas | 0.00401 | 15 | 0.02673 | Up |
| Akkermansia | 0.00595 | 16 | 0.03717 | Up |
| Bryantella | 0.00682 | 17 | 0.04012 | Up |
| Acinetobacter | 0.00711 | 18 | 0.03947 | Up |
| Agrobacterium | 0.00882 | 19 | 0.04643 | Up |
| Streptococcus | 0.01006 | 20 | 0.05028 | Down |
| Bacillaceae_1 | 0.01384 | 21 | 0.06590 | Up |
| Allobaculum | 0.01408 | 22 | 0.06400 | Up |
| Serratia | 0.01620 | 23 | 0.07044 | Up |
| Rubrobacterineae | 0.01729 | 24 | 0.07206 | Up |
| Chryseobacterium | 0.01947 | 25 | 0.07788 | Up |
| Micrococcineae | 0.01948 | 26 | 0.07493 | Up |
| Pantoea | 0.02126 | 27 | 0.07873 | Up |
| Gp2 | 0.02315 | 28 | 0.08267 | Up |
| Pseudomonas | 0.02367 | 29 | 0.08161 | Up |
| Exiguobacterium | 0.02493 | 30 | 0.08310 | Up |
| Gp1 | 0.02806 | 31 | 0.09051 | Up |
| Pseudoxanthomonas | 0.04403 | 32 | 0.13759 | Up |
| Dorea | 0.04758 | 33 | 0.14418 | Down |
| Novosphingobium | 0.04910 | 34 | 0.14441 | Up |
| Sutterella | 0.05041 | 35 | 0.14403 | Up |
| Bifidobacteriaceae | 0.05077 | 36 | 0.14102 | Down |
| Chryseomonas | 0.05792 | 37 | 0.15654 | Up |
| Comamonas | 0.07497 | 38 | 0.19730 | Up |
| Carnobacteriaceae_1 | 0.07831 | 39 | 0.20080 | Up |
| Alistipes | 0.08070 | 40 | 0.20175 | Up |
| Bacteroides | 0.09360 | 41 | 0.22829 | Down |
| Staphylococcus | 0.10208 | 42 | 0.24304 | Up |
| Variovorax | 0.10572 | 43 | 0.24585 | Up |
| Flavimonas | 0.11058 | 44 | 0.25131 | Up |
| Shinella | 0.12952 | 45 | 0.28783 | Up |
| Syntrophococcus | 0.13651 | 46 | 0.29676 | Up |
| Methylobacterium | 0.13766 | 47 | 0.29290 | Up |
| Roseburia | 0.15451 | 48 | 0.32189 | Up |
| Enterobacter | 0.15715 | 49 | 0.32072 | Up |
| Erwinia | 0.16696 | 50 | 0.33392 | Up |
| Rheinheimera | 0.17078 | 51 | 0.33486 | Down |
| Prevotella | 0.19727 | 52 | 0.37936 | Up |
| Succinispira | 0.20400 | 53 | 0.38491 | Up |
| Pedobacter | 0.23060 | 54 | 0.42704 | Up |
| Fusobacterium | 0.23880 | 55 | 0.43419 | Up |
| Sphingomonas | 0.25308 | 56 | 0.45192 | Up |
| Bradyrhizobium | 0.25361 | 57 | 0.44492 | Down |
| Propionibacterineae | 0.26446 | 58 | 0.45596 | Up |
| Burkholderia | 0.26620 | 59 | 0.45119 | Up |
| Veillonella | 0.28595 | 60 | 0.47659 | Down |
| Vibrio | 0.28683 | 61 | 0.47022 | Down |
| Papillibacter | 0.28810 | 62 | 0.46468 | Up |
| Marinomonas | 0.31275 | 63 | 0.49643 | Down |
| Bilophila | 0.40399 | 64 | 0.63123 | Up |
| Gemella | 0.40841 | 65 | 0.62832 | Up |
| Enhydrobacter | 0.44562 | 66 | 0.67518 | Up |
| Anaerococcus | 0.45866 | 67 | 0.68456 | Up |
| Pseudoalteromonas | 0.47369 | 68 | 0.69660 | Down |
| Finegoldia | 0.49275 | 69 | 0.71413 | Down |
| Haemophilias | 0.49499 | 70 | 0.70712 | Down |
| Butyrivibrio | 0.52466 | 71 | 0.73896 | Up |
| Coprococcus | 0.53663 | 72 | 0.74532 | Up |
| Clostridiaceae_1 | 0.57343 | 73 | 0.78553 | Up |
| Ruminococcaceae_Incertae_Sedis | 0.59101 | 74 | 0.79867 | Up |
| Paracoccus | 0.61333 | 75 | 0.81777 | Up |
| Anaerotruncus | 0.64579 | 76 | 0.84973 | Down |
| Parabacteroides | 0.64883 | 77 | 0.84264 | Up |
| Lachnospiraceae_Incertae_Sedis | 0.68417 | 78 | 0.87714 | Up |
| Citrobacter | 0.68862 | 79 | 0.87167 | Up |
| Coprobacillus | 0.69082 | 80 | 0.86352 | Down |
| Desulfovibrio | 0.71148 | 81 | 0.87837 | Down |
| Shigella | 0.72933 | 82 | 0.88943 | Down |
| Actinomycineae | 0.74703 | 83 | 0.90004 | Down |
| Uruburuella | 0.75252 | 84 | 0.89586 | Down |
| Corynebacterineae | 0.78329 | 85 | 0.92152 | Down |
| Megamonas | 0.84097 | 86 | 0.97787 | Down |
| Aeromonas | 0.85775 | 87 | 0.98592 | Down |
| Holdemania | 0.86825 | 88 | 0.98665 | Up |
| Subdoligranulum | 0.87174 | 89 | 0.97948 | Up |
| Coriobacterineae | 0.87710 | 90 | 0.97456 | Down |
| Ralstonia | 0.88637 | 91 | 0.97403 | Up |
| Erysipelotrichaceae_Incertae_Sedis | 0.89520 | 92 | 0.97304 | Up |
| Allomonas | 0.91827 | 93 | 0.98739 | Down |
| Peptostreptococcaceae_Incertae_Sedis | 0.93100 | 94 | 0.99043 | Up |
| Brevundimonas | 0.94692 | 95 | 0.99676 | Down |
| Carnobacteriaceae_2 | 0.94786 | 96 | 0.98736 | Up |
| Anaerovorax | 0.96308 | 97 | 0.99286 | Down |
| Faecalibacterium | 0.97701 | 98 | 0.99695 | Up |
| Ruminococcus | 0.98616 | 99 | 0.99612 | Up |
| Dialister | 0.99025 | 100 | 0.99025 | Up |

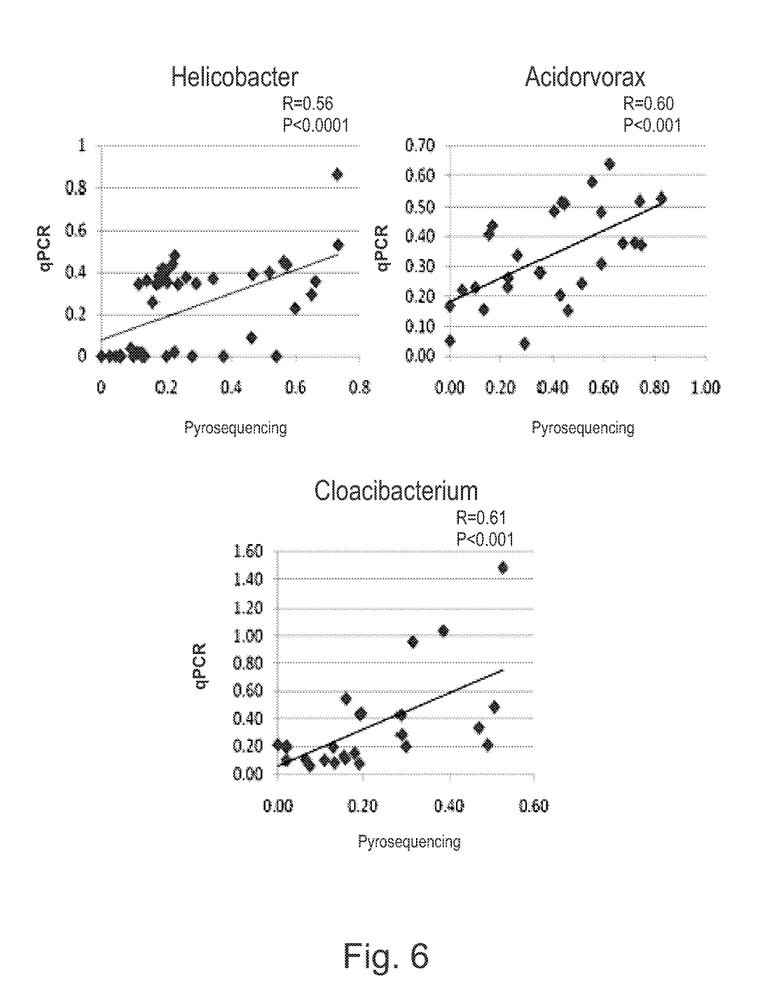

Remarkably, only one genus, Streptococcus, had a higher relative abundance in the control group. In other words, Streptococcus was down-regulated in the cases with a statistical significance of p<0.05. In order to validate these pyrosequencing results, qPCR assays were prepared for a subset of observed genera that were significantly different in their relative abundances between cases and controls (i.e., Helicobacter spp, Acidovorax spp and Cloacibacteria spp.). The two methods correlated as expected (FIG. 6), validating the pyrosequencing results.

Operational Taxonomic Units (OTUs), which are clusters of sequences in which the average percent identity of all of the sequences within a cluster is >=97%, were analyzed. At the OTU level at a 10% false discovery rate threshold 87 OTUs were found with significantly higher relative abundance in cases vs. controls and only 5 OTUs higher in controls (Table 3).

Table 3:

Wilcoxon-tests on log-normalized abundances of OTUs (97%) in cases (33 subjects) vs. controls (38 subjects). Only OTUs which have at least 1 sequence assigned to them in 25% of the samples are shown. RDP classification of consensus sequences at genus level shown. Wilcoxon p-Values were corrected for multiple testing using (n*p)/R where n=total number of taxa tested, p=raw p-Value and R=sorted Rank of the taxon. Benjamini & Hochberg (1995).

TABLE 3

| OTU | Wilcoxon | | | | |
|---|---|---|---|---|---|
| name | p-Value | Rank | (n*p)/R | Dir. | RDP Assignment |
| OTU72 | 0.000084 | 1 | 0.031257 | Up | Aquabacterium |
| OTU226 | 0.000085 | 2 | 0.015686 | Up | Rikenella |
| OTU200 | 0.000087 | 3 | 0.010705 | Up | Helicobacter |
| OTU432 | 0.000111 | 4 | 0.010297 | Up | Paludibacter |
| OTU285 | 0.000137 | 5 | 0.010167 | Up | Butyrivibrio |
| OTU157 | 0.000139 | 6 | 0.008578 | Up | Marinilabilia |
| OTU240 | 0.000318 | 7 | 0.016856 | Up | Weissella |
| OTU370 | 0.000384 | 8 | 0.017786 | Up | Lactobacillus |
| OTU284 | 0.000424 | 9 | 0.017486 | Down | Rubritepida |
| OTU22 | 0.00043 | 10 | 0.015937 | Up | Acidovorax |
| OTU96 | 0.000484 | 11 | 0.016326 | Up | Diaphorobacter |
| OTU119 | 0.000579 | 12 | 0.017915 | Up | Lachnobacterium |
| OTU213 | 0.000679 | 13 | 0.019378 | Up | Lactococcus |
| OTU73 | 0.000703 | 14 | 0.018642 | Up | Lactococcus |
| OTU306 | 0.000821 | 15 | 0.020303 | Down | Oligotropha |
| OTU373 | 0.000896 | 16 | 0.020772 | Up | Sporobacter |
| OTU501 | 0.000947 | 17 | 0.020667 | Up | Ruminococcaceae Incertae Sedis |
| OTU37 | 0.001006 | 18 | 0.020743 | Up | Cloacibacterium |
| OTU109 | 0.001008 | 19 | 0.019674 | Up | Turicibacter |
| OTU100 | 0.001258 | 20 | 0.023329 | Up | Xylanibacter |
| OTU122 | 0.001335 | 21 | 0.023579 | Up | Prevotella |
| OTU46 | 0.001398 | 22 | 0.023569 | Up | Bacillaceae 1 |
| OTU525 | 0.001497 | 23 | 0.024146 | Up | Catonella |
| OTU70 | 0.001582 | 24 | 0.02446 | Up | Sphingobium |
| OTU91 | 0.001641 | 25 | 0.024351 | Up | Lactobacillus |
| OTU75 | 0.001703 | 26 | 0.024306 | Up | Stenotrophomonas |
| OTU328 | 0.00179 | 27 | 0.02459 | Up | Parasporobacterium |
| OTU309 | 0.002063 | 28 | 0.027333 | Up | Paludibacter |
| OTU230 | 0.002084 | 29 | 0.026658 | Up | Butyrivibrio |
| OTU371 | 0.002129 | 30 | 0.02633 | Up | Comamonas |
| OTU177 | 0.002213 | 31 | 0.026484 | Up | Butyrivibrio |
| OTU136 | 0.002304 | 32 | 0.026712 | Up | Micrococcineae |
| OTU357 | 0.002384 | 33 | 0.026803 | Up | Coprococcus |
| OTU387 | 0.002449 | 34 | 0.026723 | Up | Coprococcus |
| OTU124 | 0.002547 | 35 | 0.026996 | Up | Lactobacillus |
| OTU38 | 0.002829 | 36 | 0.029152 | Up | Pseudomonas |
| OTU56 | 0.002884 | 37 | 0.028914 | Up | Delftia |
| OTU202 | 0.002913 | 38 | 0.028437 | Up | Lachnospiraceae Incertae Sedis |
| OTU133 | 0.002963 | 39 | 0.028182 | Up | Faecalibacterium |
| OTU242 | 0.003059 | 40 | 0.028371 | Up | Coriobacterineae |
| OTU189 | 0.00349 | 41 | 0.031576 | Up | Acidovorax |
| OTU439 | 0.003755 | 42 | 0.033171 | Down | Algibacter |
| OTU265 | 0.003802 | 43 | 0.032805 | Up | Sphingomonas |
| OTU139 | 0.003893 | 44 | 0.032827 | Up | Azonexus |
| OTU95 | 0.004005 | 45 | 0.03302 | Up | Ruminococcus |
| OTU23 | 0.004051 | 46 | 0.032674 | Up | Lachnospiraceae Incertae Sedis |
| OTU59 | 0.004084 | 47 | 0.032241 | Up | Acinetobacter |
| OTU502 | 0.004279 | 48 | 0.033077 | Up | Paludibacter |
| OTU64 | 0.004323 | 49 | 0.032735 | Up | Erwinia |
| OTU454 | 0.004669 | 50 | 0.034641 | Up | Paludibacter |
| OTU286 | 0.005422 | 51 | 0.039446 | Up | Hallella |
| OTU464 | 0.005427 | 52 | 0.038721 | Up | Marinilabilia |
| OTU161 | 0.006285 | 53 | 0.043997 | Up | Prevotella |
| OTU423 | 0.007065 | 54 | 0.048543 | Up | Parasporobacterium |
| OTU53 | 0.007612 | 55 | 0.051345 | Up | Succinivibrio |
| OTU239 | 0.007843 | 56 | 0.051957 | Up | Succinispira |
| OTU319 | 0.008701 | 57 | 0.056633 | Up | Agrobacterium |
| OTU193 | 0.008755 | 58 | 0.056004 | Up | Xylanibacter |
| OTU61 | 0.009098 | 59 | 0.057207 | Up | Papillibacter |
| OTU365 | 0.009827 | 60 | 0.060762 | Up | Succinispira |
| OTU437 | 0.010114 | 61 | 0.061514 | Up | Marinilabilia |
| OTU225 | 0.010608 | 62 | 0.063477 | Up | Prevotella |
| OTU366 | 0.01081 | 63 | 0.063657 | Up | Coprococcus |
| OTU92 | 0.01095 | 64 | 0.063478 | Up | Rubrobacterineae |
| OTU463 | 0.01103 | 65 | 0.062958 | Up | Lachnospiraceae Incertae Sedis |
| OTU97 | 0.011294 | 66 | 0.063484 | Up | Pseudomonas |
| OTU21 | 0.011865 | 67 | 0.065699 | Up | Finegoldia |
| OTU149 | 0.012682 | 68 | 0.069192 | Down | Haemophilias |
| OTU241 | 0.013048 | 69 | 0.070156 | Up | Chryseobacterium |
| OTU250 | 0.013254 | 70 | 0.070246 | Up | Paludibacter |
| OTU210 | 0.013651 | 71 | 0.071332 | Up | Allobaculum |
| OTU347 | 0.013893 | 72 | 0.071586 | Down | Vitellibacter |
| OTU191 | 0.014678 | 73 | 0.074597 | Up | Subdoligranulum |
| OTU404 | 0.014845 | 74 | 0.074425 | Up | Hallella |
| OTU396 | 0.014935 | 75 | 0.073878 | Up | Coprococcus |
| OTU345 | 0.01502 | 76 | 0.073319 | Up | Butyrivibrio |
| OTU401 | 0.015426 | 77 | 0.074324 | Up | Alistipes |
| OTU67 | 0.015821 | 78 | 0.075251 | Up | Lactobacillus |
| OTU407 | 0.016533 | 79 | 0.077644 | Up | Turicibacter |
| OTU313 | 0.016785 | 80 | 0.077842 | Up | Enterobacter |
| OTU353 | 0.017139 | 81 | 0.0785 | Up | Dorea |
| OTU418 | 0.019841 | 82 | 0.08977 | Up | Stenotrophomonas |
| OTU393 | 0.020465 | 83 | 0.091478 | Up | Micrococcineae |
| OTU120 | 0.020843 | 84 | 0.092056 | Up | Micrococcineae |
| OTU413 | 0.021269 | 85 | 0.092833 | Up | Subdoligranulum |
| OTU341 | 0.021427 | 86 | 0.092433 | Up | Prevotella |
| OTU93 | 0.021869 | 87 | 0.093258 | Up | Alistipes |
| OTU186 | 0.022338 | 88 | 0.094173 | Up | Faecalibacterium |
| OTU79 | 0.022545 | 89 | 0.093981 | Up | Lachnospiraceae Incertae Sedis |
| OTU197 | 0.023847 | 90 | 0.098304 | Up | Lactobacillus |
| OTU219 | 0.024265 | 91 | 0.098928 | Up | Rikenella |
| OTU86 | 0.02429 | 92 | 0.097951 | Up | Fusobacterium |
| OTU297 | 0.0273 | 93 | 0.108905 | Up | Bacillaceae 1 |
| OTU442 | 0.02802 | 94 | 0.110588 | Up | Roseburia |
| OTU389 | 0.028617 | 95 | 0.111759 | Up | Parabacteroides |
| OTU352 | 0.028801 | 96 | 0.111304 | Down | Saprospira |
| OTU49 | 0.031048 | 97 | 0.118749 | Up | Sutterella |
| OTU329 | 0.032674 | 98 | 0.123693 | Down | Methanohalobium |
| OTU176 | 0.033016 | 99 | 0.123727 | Up | Erwinia |
| OTU484 | 0.033734 | 100 | 0.125152 | Down | Effluviibacter |
| OTU569 | 0.033751 | 101 | 0.123975 | Up | Erwinia |
| OTU66 | 0.034683 | 102 | 0.126152 | Down | Streptococcus |
| OTU391 | 0.03501 | 103 | 0.126103 | Up | Aquiflexum |
| OTU356 | 0.036933 | 104 | 0.131753 | Up | Novosphingobium |
| OTU11 | 0.041357 | 105 | 0.146129 | Up | Bacteroides |
| OTU330 | 0.04391 | 106 | 0.153686 | Up | Coriobacterineae |
| OTU361 | 0.04391 | 107 | 0.152249 | Up | Succinivibrio |
| OTU113 | 0.044104 | 108 | 0.151507 | Up | Rikenella |
| OTU45 | 0.04423 | 109 | 0.150544 | Down | Xenohaliotis |
| OTU471 | 0.045642 | 110 | 0.153937 | Up | Lachnospiraceae Incertae Sedis |
| OTU247 | 0.047313 | 111 | 0.158135 | Up | Xylanibacter |
| OTU283 | 0.050651 | 112 | 0.16778 | Up | Anaerophaga |
| OTU128 | 0.055374 | 113 | 0.181802 | Up | Prevotella |
| OTU270 | 0.056309 | 114 | 0.183252 | Up | Succinispira |
| OTU57 | 0.061822 | 115 | 0.199442 | Down | Lachnospiraceae Incertae Sedis |
| OTU77 | 0.06775 | 116 | 0.216684 | Up | Coprococcus |
| OTU138 | 0.068101 | 117 | 0.215945 | Down | Simkania |
| OTU491 | 0.068451 | 118 | 0.215214 | Up | Clostridiaceae 1 |
| OTU169 | 0.069264 | 119 | 0.215941 | Down | Streptococcus |
| OTU207 | 0.070648 | 120 | 0.218419 | Up | Succinispira |
| OTU237 | 0.072858 | 121 | 0.223392 | Up | Prevotella |
| OTU499 | 0.075097 | 122 | 0.22837 | Down | Lachnospiraceae Incertae Sedis |
| OTU14 | 0.07526 | 123 | 0.227004 | Up | Erysipelotrichaceae Incertae Sedis |
| OTU417 | 0.07743 | 124 | 0.231665 | Up | Lachnobacterium |
| OTU111 | 0.080236 | 125 | 0.23814 | Up | Peptostreptococcaceae Incertae Sedis |
| OTU322 | 0.080575 | 126 | 0.237249 | Up | Roseburia |
| OTU244 | 0.081081 | 127 | 0.236857 | Up | Prevotella |
| OTU350 | 0.083008 | 128 | 0.240595 | Up | Coprococcus |
| OTU159 | 0.084952 | 129 | 0.244319 | Up | Faecalibacterium |
| OTU224 | 0.088054 | 130 | 0.251292 | Up | Prevotella |
| OTU338 | 0.09269 | 131 | 0.262503 | Up | Micrococcineae |
| OTU376 | 0.093281 | 132 | 0.262177 | Up | Methylobacterium |
| OTU254 | 0.093506 | 133 | 0.260833 | Down | Lachnospiraceae Incertae Sedis |
| OTU36 | 0.094305 | 134 | 0.261099 | Up | Bacteroides |
| OTU8 | 0.095901 | 135 | 0.263551 | Down | Dorea |
| OTU326 | 0.096151 | 136 | 0.262295 | Down | Lachnospiraceae Incertae Sedis |
| OTU282 | 0.104442 | 137 | 0.282832 | Down | Streptococcus |
| OTU264 | 0.107146 | 138 | 0.288052 | Up | Comamonas |
| OTU26 | 0.11087 | 139 | 0.29592 | Down | Dorea |
| OTU137 | 0.1132 | 140 | 0.299979 | Up | Prevotella |
| OTU222 | 0.116058 | 141 | 0.305373 | Up | Prevotella |
| OTU85 | 0.117436 | 142 | 0.306821 | Up | Bacteroides |
| OTU397 | 0.12782 | 143 | 0.331617 | Up | Peptostreptococcaceae Incertae Sedis |
| OTU167 | 0.129522 | 144 | 0.333699 | Up | Allobaculum |
| OTU420 | 0.13338 | 145 | 0.341269 | Up | Dorea |
| OTU474 | 0.13338 | 146 | 0.338931 | Up | Sphingobium |
| OTU29 | 0.137289 | 147 | 0.346491 | Down | Lachnospiraceae Incertae Sedis |
| OTU144 | 0.138737 | 148 | 0.347779 | Down | Dorea |
| OTU172 | 0.140932 | 149 | 0.350912 | Down | Marinilabilia |
| OTU409 | 0.141562 | 150 | 0.350129 | Up | Alkalilimnicola |
| OTU68 | 0.145429 | 151 | 0.357313 | Up | Dorea |
| OTU216 | 0.146992 | 152 | 0.358776 | Up | Sphingomonas |
| OTU421 | 0.150949 | 153 | 0.366028 | Down | Streptococcus |
| OTU476 | 0.157687 | 154 | 0.379882 | Down | Streptococcus |
| OTU519 | 0.159874 | 155 | 0.382665 | Up | Catonella |
| OTU143 | 0.160715 | 156 | 0.382213 | Down | Lachnospiraceae Incertae Sedis |
| OTU275 | 0.160841 | 157 | 0.380078 | Up | Lachnospiraceae Incertae Sedis |
| OTU206 | 0.161316 | 158 | 0.378785 | Up | Paludibacter |
| OTU419 | 0.161556 | 159 | 0.376965 | Up | Micrococcineae |
| OTU1 | 0.163025 | 160 | 0.378015 | Down | Bacteroides |
| OTU248 | 0.16912 | 161 | 0.389711 | Up | Lachnospiraceae Incertae Sedis |
| OTU134 | 0.169695 | 162 | 0.388622 | Down | Ruminococcaceae Incertae Sedis |
| OTU141 | 0.174538 | 163 | 0.397262 | Up | Faecalibacterium |
| OTU368 | 0.176676 | 164 | 0.399676 | Up | Ruminococcaceae Incertae Sedis |
| OTU205 | 0.17885 | 165 | 0.402142 | Up | Erysipelotrichaceae Incertae Sedis |
| OTU300 | 0.17925 | 166 | 0.400614 | Down | Lachnospiraceae Incertae Sedis |
| OTU152 | 0.183253 | 167 | 0.407108 | Down | Faecalibacterium |
| OTU82 | 0.189641 | 168 | 0.418791 | Up | Roseburia |
| OTU28 | 0.194628 | 169 | 0.427261 | Down | Bacteroides |
| OTU299 | 0.195265 | 170 | 0.426137 | Up | Lachnospiraceae Incertae Sedis |
| OTU135 | 0.19551 | 171 | 0.424178 | Up | Clostridiaceae 1 |
| OTU267 | 0.197149 | 172 | 0.425246 | Up | Parabacteroides |
| OTU249 | 0.197702 | 173 | 0.423974 | Up | Faecalibacterium |
| OTU334 | 0.205736 | 174 | 0.438667 | Up | Citrobacter |
| OTU34 | 0.206355 | 175 | 0.437473 | Down | Dorea |
| OTU192 | 0.212037 | 176 | 0.446964 | Up | Sphingomonas |
| OTU153 | 0.213057 | 177 | 0.446576 | Up | Roseburia |
| OTU266 | 0.214087 | 178 | 0.446215 | Down | Bacteroides |
| OTU87 | 0.215609 | 179 | 0.446876 | Up | Propionibacterineae |
| OTU235 | 0.224633 | 180 | 0.462994 | Up | Desulfovibrio |
| OTU50 | 0.226155 | 181 | 0.463556 | Up | Sutterella |
| OTU33 | 0.229786 | 182 | 0.468411 | Down | Lachnospiraceae Incertae Sedis |
| OTU90 | 0.231703 | 183 | 0.469737 | Up | Lachnospiraceae Incertae Sedis |
| OTU204 | 0.231703 | 184 | 0.467184 | Up | Dialister |
| OTU395 | 0.236361 | 185 | 0.474 | Up | Subdoligranulum |
| OTU317 | 0.237329 | 186 | 0.473383 | Up | Prevotella |
| OTU203 | 0.238017 | 187 | 0.472215 | Down | Rheinheimera |
| OTU165 | 0.23893 | 188 | 0.471505 | Up | Alistipes |
| OTU303 | 0.245272 | 189 | 0.481459 | Down | Faecalibacterium |
| OTU15 | 0.246531 | 190 | 0.481385 | Up | Roseburia |
| OTU127 | 0.246632 | 191 | 0.479061 | Down | Lachnospiraceae Incertae Sedis |
| OTU412 | 0.248001 | 192 | 0.47921 | Up | Sphingomonas |
| OTU178 | 0.250803 | 193 | 0.482114 | Up | Lachnospiraceae Incertae Sedis |
| OTU195 | 0.252465 | 194 | 0.482808 | Down | Pseudoalteromonas |
| OTU162 | 0.255823 | 195 | 0.486719 | Down | Veillonella |
| OTU154 | 0.260826 | 196 | 0.493707 | Down | Faecalibacterium |
| OTU190 | 0.260891 | 197 | 0.491324 | Up | Ruminococcaceae Incertae Sedis |
| OTU74 | 0.263322 | 198 | 0.493397 | Up | Ruminococcus |
| OTU425 | 0.264265 | 199 | 0.492674 | Up | Enhydrobacter |
| OTU118 | 0.26768 | 200 | 0.496547 | Up | Burkholderia |
| OTU83 | 0.268729 | 201 | 0.496012 | Down | Dorea |
| OTU188 | 0.269309 | 202 | 0.494622 | Down | Lachnospiraceae Incertae Sedis |
| OTU156 | 0.275877 | 203 | 0.504188 | Up | Lachnospiraceae Incertae Sedis |
| OTU146 | 0.277131 | 204 | 0.503998 | Down | Vibrio |
| OTU84 | 0.277838 | 205 | 0.50282 | Down | Marinomonas |
| OTU3 | 0.286165 | 206 | 0.515375 | Down | Lachnospiraceae Incertae Sedis |
| OTU170 | 0.2869 | 207 | 0.514203 | Down | Bacteroides |
| OTU5 | 0.293459 | 208 | 0.52343 | Up | Sphingomonas |
| OTU19 | 0.296777 | 209 | 0.526814 | Up | Syntrophococcus |
| OTU142 | 0.301855 | 210 | 0.533278 | Down | Lachnospiraceae Incertae Sedis |
| OTU307 | 0.303841 | 211 | 0.534242 | Up | Megamonas |
| OTU360 | 0.310287 | 212 | 0.543003 | Down | Faecalibacterium |
| OTU227 | 0.314679 | 213 | 0.548103 | Down | Lachnospiraceae Incertae Sedis |
| OTU145 | 0.31593 | 214 | 0.54771 | Up | Afipia |
| OTU453 | 0.318042 | 215 | 0.548807 | Up | Faecalibacterium |
| OTU296 | 0.326377 | 216 | 0.560583 | Up | Papillibacter |
| OTU166 | 0.328441 | 217 | 0.561529 | Down | Lachnospiraceae Incertae Sedis |
| OTU7 | 0.330993 | 218 | 0.563296 | Up | Bacteroides |
| OTU256 | 0.33172 | 219 | 0.561955 | Up | Anaerotruncus |
| OTU274 | 0.333905 | 220 | 0.563085 | Down | Lachnospiraceae Incertae Sedis |
| OTU65 | 0.334251 | 221 | 0.561118 | Up | Lachnospiraceae Incertae Sedis |
| OTU327 | 0.337489 | 222 | 0.564002 | Up | Pelomonas |
| OTU168 | 0.342414 | 223 | 0.569666 | Down | Roseburia |
| OTU89 | 0.347493 | 224 | 0.575535 | Up | Bacteroides |
| OTU71 | 0.353559 | 225 | 0.582979 | Up | Lachnospiraceae Incertae Sedis |
| OTU47 | 0.353621 | 226 | 0.580501 | Down | Succinispira |
| OTU349 | 0.371504 | 227 | 0.607171 | Up | Syntrophococcus |
| OTU495 | 0.372554 | 228 | 0.606217 | Down | Streptococcus |
| OTU304 | 0.375615 | 229 | 0.608529 | Down | Faecalibacterium |
| OTU181 | 0.376974 | 230 | 0.608075 | Up | Bacteroides |
| OTU199 | 0.379331 | 231 | 0.609229 | Up | Acetanaerobacterium |
| OTU44 | 0.383199 | 232 | 0.612788 | Up | Lachnospiraceae Incertae Sedis |
| OTU183 | 0.383518 | 233 | 0.610665 | Down | Bacteroides |
| OTU364 | 0.384954 | 234 | 0.610333 | Up | Exiguobacterium |
| OTU6 | 0.403239 | 235 | 0.636604 | Down | Lachnospiraceae Incertae Sedis |
| OTU553 | 0.403416 | 236 | 0.634184 | Up | Syntrophococcus |
| OTU88 | 0.409553 | 237 | 0.641115 | Down | Streptococcus |
| OTU268 | 0.412992 | 238 | 0.643782 | Up | Staphylococcus |
| OTU198 | 0.417755 | 239 | 0.648482 | Up | Lachnospiraceae Incertae Sedis |
| OTU160 | 0.428286 | 240 | 0.662059 | Down | Lachnospiraceae Incertae Sedis |
| OTU315 | 0.440228 | 241 | 0.677696 | Down | Coriobacterineae |
| OTU20 | 0.44566 | 242 | 0.683222 | Down | Lachnospiraceae Incertae Sedis |
| OTU354 | 0.450531 | 243 | 0.687848 | Up | Anaerotruncus |
| OTU179 | 0.450803 | 244 | 0.685442 | Up | Ruminococcaceae Incertae Sedis |
| OTU76 | 0.454998 | 245 | 0.688997 | Down | Lachnobacterium |
| OTU374 | 0.455869 | 246 | 0.687509 | Down | Lachnospiraceae Incertae Sedis |
| OTU4 | 0.464125 | 247 | 0.697128 | Up | Lachnospiraceae Incertae Sedis |
| OTU24 | 0.466828 | 248 | 0.69836 | Up | Lachnospiraceae Incertae Sedis |
| OTU173 | 0.473245 | 249 | 0.705117 | Down | Anaerotruncus |
| OTU54 | 0.476242 | 250 | 0.706743 | Up | Lachnospiraceae Incertae Sedis |
| OTU288 | 0.477369 | 251 | 0.705593 | Up | Ruminococcaceae Incertae Sedis |
| OTU229 | 0.478121 | 252 | 0.703901 | Down | Coriobacterineae |
| OTU367 | 0.484431 | 253 | 0.710371 | Up | Pseudomonas |
| OTU233 | 0.495265 | 254 | 0.723399 | Up | Syntrophococcus |
| OTU359 | 0.499339 | 255 | 0.72649 | Up | Faecalibacterium |
| OTU452 | 0.505628 | 256 | 0.732766 | Down | Butyrivibrio |
| OTU455 | 0.508508 | 257 | 0.734071 | Down | Finegoldia |
| OTU41 | 0.508672 | 258 | 0.731462 | Down | Subdoligranulum |
| OTU62 | 0.508801 | 259 | 0.728823 | Down | Ruminococcus |
| OTU400 | 0.515068 | 260 | 0.734962 | Up | Bryantella |
| OTU42 | 0.519408 | 261 | 0.738315 | Up | Prevotella |
| OTU470 | 0.521033 | 262 | 0.737799 | Down | Lachnospiraceae Incertae Sedis |
| OTU422 | 0.524664 | 263 | 0.740116 | Up | Peptococcaceae 1 |
| OTU566 | 0.531236 | 264 | 0.746548 | Down | Dorea |
| OTU214 | 0.531345 | 265 | 0.743883 | Down | Roseburia |
| OTU375 | 0.534803 | 266 | 0.74591 | Up | Pseudomonas |
| OTU456 | 0.541252 | 267 | 0.752076 | Down | Anaerovorax |
| OTU538 | 0.541252 | 268 | 0.74927 | Down | Lachnospiraceae Incertae Sedis |
| OTU272 | 0.543323 | 269 | 0.749342 | Down | Sporobacter |
| OTU182 | 0.544691 | 270 | 0.748446 | Down | Lachnospiraceae Incertae Sedis |
| OTU260 | 0.549257 | 271 | 0.751935 | Down | Erysipelotrichaceae Incertae Sedis |
| OTU406 | 0.551284 | 272 | 0.751935 | Up | Bacteroides |
| OTU17 | 0.554959 | 273 | 0.754175 | Down | Escherichia |
| OTU123 | 0.562088 | 274 | 0.761075 | Up | Papillibacter |
| OTU58 | 0.577186 | 275 | 0.778677 | Down | Peptostreptococcaceae Incertae Sedis |
| OTU380 | 0.597757 | 276 | 0.803507 | Down | Sporobacter |
| OTU372 | 0.598207 | 277 | 0.801208 | Up | Allomonas |
| OTU460 | 0.598207 | 278 | 0.798326 | Up | Lachnospiraceae Incertae Sedis |
| OTU164 | 0.598254 | 279 | 0.795527 | Down | Faecalibacterium |
| OTU9 | 0.606837 | 280 | 0.804058 | Up | Bacteroides |
| OTU493 | 0.611938 | 281 | 0.807932 | Down | Lachnospiraceae Incertae Sedis |
| OTU411 | 0.61495 | 282 | 0.80903 | Up | Faecalibacterium |
| OTU506 | 0.61495 | 283 | 0.806172 | Up | Syntrophococcus |
| OTU104 | 0.620801 | 284 | 0.810976 | Down | Syntrophococcus |
| OTU184 | 0.621999 | 285 | 0.80969 | Down | Lachnospiraceae Incertae Sedis |
| OTU60 | 0.622167 | 286 | 0.807077 | Up | Subdoligranulum |
| OTU196 | 0.627379 | 287 | 0.811003 | Down | Bacteroides |
| OTU305 | 0.635906 | 288 | 0.819171 | Down | Lachnospiraceae Incertae Sedis |
| OTU408 | 0.636907 | 289 | 0.817621 | Up | Bryantella |
| OTU217 | 0.637392 | 290 | 0.815422 | Up | Prevotella |
| OTU27 | 0.644638 | 291 | 0.821858 | Up | Lachnospiraceae Incertae Sedis |
| OTU117 | 0.644751 | 292 | 0.819187 | Down | Naxibacter |
| OTU238 | 0.648684 | 293 | 0.821372 | Down | Lachnospiraceae Incertae Sedis |
| OTU129 | 0.649316 | 294 | 0.819374 | Down | Roseburia |
| OTU148 | 0.651838 | 295 | 0.819769 | Down | Lachnospiraceae Incertae Sedis |
| OTU343 | 0.668166 | 296 | 0.837465 | Up | Lachnobacterium |
| OTU429 | 0.668166 | 297 | 0.834645 | Down | Dorea |
| OTU363 | 0.670411 | 298 | 0.834639 | Up | Faecalibacterium |
| OTU140 | 0.671784 | 299 | 0.833551 | Up | Faecalibacterium |
| OTU52 | 0.672431 | 300 | 0.831573 | Up | Lachnospiraceae Incertae Sedis |
| OTU378 | 0.689349 | 301 | 0.849663 | Down | Bacillaceae 1 |
| OTU508 | 0.689557 | 302 | 0.847104 | Down | Lachnospiraceae Incertae Sedis |
| OTU10 | 0.689926 | 303 | 0.844761 | Up | Coprobacillus |
| OTU32 | 0.690686 | 304 | 0.84291 | Down | Erysipelotrichaceae Incertae Sedis |
| OTU80 | 0.698714 | 305 | 0.849911 | Down | Lachnospiraceae Incertae Sedis |
| OTU110 | 0.712924 | 306 | 0.864363 | Up | Lachnospiraceae Incertae Sedis |
| OTU106 | 0.715991 | 307 | 0.865253 | Down | Lachnospiraceae Incertae Sedis |
| OTU379 | 0.716925 | 308 | 0.863568 | Up | Roseburia |
| OTU171 | 0.716992 | 309 | 0.860854 | Down | Bacteroides |
| OTU30 | 0.725113 | 310 | 0.867797 | Up | Bryantella |
| OTU324 | 0.738903 | 311 | 0.881456 | Up | Faecalibacterium |
| OTU311 | 0.740828 | 312 | 0.880921 | Up | Lachnospiraceae Incertae Sedis |
| OTU101 | 0.745441 | 313 | 0.883574 | Down | Pseudoalteromonas |
| OTU287 | 0.751988 | 314 | 0.888496 | Down | Anaerovorax |
| OTU212 | 0.757145 | 315 | 0.891749 | Down | Coprobacillus |
| OTU55 | 0.767222 | 316 | 0.900757 | Up | Parabacteroides |
| OTU392 | 0.768645 | 317 | 0.899582 | Up | Lachnospiraceae Incertae Sedis |
| OTU114 | 0.768686 | 318 | 0.8968 | Up | Megamonas |
| OTU243 | 0.772843 | 319 | 0.898824 | Up | Anaerotruncus |
| OTU108 | 0.77323 | 320 | 0.896464 | Up | Lachnospiraceae Incertae Sedis |
| OTU231 | 0.775025 | 321 | 0.895745 | Up | Anaerotruncus |
| OTU316 | 0.775025 | 322 | 0.892964 | Up | Alistipes |
| OTU403 | 0.784314 | 323 | 0.900868 | Up | Methylobacterium |
| OTU131 | 0.784488 | 324 | 0.898287 | Up | Lachnospiraceae Incertae Sedis |
| OTU103 | 0.789604 | 325 | 0.901363 | Up | Roseburia |
| OTU105 | 0.793064 | 326 | 0.902536 | Up | Bacteroides |
| OTU155 | 0.800433 | 327 | 0.908137 | Down | Roseburia |
| OTU107 | 0.811899 | 328 | 0.918337 | Down | Ruminococcus |
| OTU269 | 0.815747 | 329 | 0.919885 | Down | Butyrivibrio |
| OTU312 | 0.819071 | 330 | 0.920834 | Down | Coriobacterineae |
| OTU18 | 0.822123 | 331 | 0.921474 | Up | Faecalibacterium |
| OTU115 | 0.825146 | 332 | 0.922076 | Down | Roseburia |
| OTU126 | 0.825636 | 333 | 0.919852 | Down | Aeromonas |
| OTU40 | 0.830942 | 334 | 0.922993 | Up | Lachnospiraceae Incertae Sedis |
| OTU12 | 0.832163 | 335 | 0.921589 | Up | Bryantella |
| OTU416 | 0.838341 | 336 | 0.925668 | Up | Lachnospiraceae Incertae Sedis |
| OTU102 | 0.839205 | 337 | 0.923873 | Down | Lachnospiraceae Incertae Sedis |
| OTU130 | 0.847691 | 338 | 0.930453 | Up | Lachnospiraceae Incertae Sedis |
| OTU51 | 0.849066 | 339 | 0.929213 | Down | Klebsiella |
| OTU187 | 0.853675 | 340 | 0.93151 | Down | Erysipelotrichaceae Incertae Sedis |
| OTU492 | 0.860391 | 341 | 0.936085 | Down | Coriobacterineae |
| OTU158 | 0.870215 | 342 | 0.944005 | Down | Bacteroides |
| OTU43 | 0.871472 | 343 | 0.942613 | Down | Lachnospiraceae Incertae Sedis |
| OTU445 | 0.874152 | 344 | 0.942763 | Down | Corynebacterineae |
| OTU424 | 0.874975 | 345 | 0.940915 | Down | Streptococcus |
| OTU35 | 0.885406 | 346 | 0.949381 | Down | Bryantella |
| OTU358 | 0.886366 | 347 | 0.947671 | Up | Roseburia |
| OTU39 | 0.889892 | 348 | 0.948707 | Down | Coriobacterineae |
| OTU291 | 0.890838 | 349 | 0.946994 | Up | Syntrophococcus |
| OTU292 | 0.892843 | 350 | 0.946414 | Down | Alistipes |
| OTU94 | 0.894124 | 351 | 0.945072 | Down | Anaerotruncus |
| OTU31 | 0.903421 | 352 | 0.952185 | Up | Coprococcus |
| OTU399 | 0.913216 | 353 | 0.959782 | Down | Ralstonia |
| OTU253 | 0.914073 | 354 | 0.957969 | Down | Uruburuella |
| OTU69 | 0.921491 | 355 | 0.963023 | Down | Lachnospiraceae Incertae Sedis |
| OTU547 | 0.921893 | 356 | 0.960737 | Up | Subdoligranulum |
| OTU25 | 0.931086 | 357 | 0.967599 | Up | Parabacteroides |
| OTU277 | 0.933541 | 358 | 0.967441 | Down | Lachnospiraceae Incertae Sedis |
| OTU293 | 0.935543 | 359 | 0.966814 | Down | Lachnospiraceae Incertae Sedis |
| OTU98 | 0.93936 | 360 | 0.968063 | Up | Lachnospiraceae Incertae Sedis |
| OTU194 | 0.949283 | 361 | 0.975579 | Down | Alistipes |
| OTU344 | 0.961288 | 362 | 0.985187 | Down | Carnobacteriaceae 1 |
| OTU48 | 0.967805 | 363 | 0.989134 | Down | Bacteroides |
| OTU132 | 0.972304 | 364 | 0.991002 | Down | Parabacteroides |
| OTU355 | 0.973371 | 365 | 0.989371 | Down | Corynebacterineae |
| OTU458 | 0.984021 | 366 | 0.997463 | Up | Roseburia |
| OTU180 | 0.98511 | 367 | 0.995847 | Down | Roseburia |
| OTU151 | 0.985591 | 368 | 0.993626 | Down | Subdoligranulum |
| OTU16 | 0.986197 | 369 | 0.991542 | Down | Lachnospiraceae Incertae Sedis |
| OTU2 | 0.986203 | 370 | 0.988868 | Up | Faecalibacterium |
| OTU150 | 0.995379 | 371 | 0.995379 | Up | Ruminococcaceae Incertae Sedis |

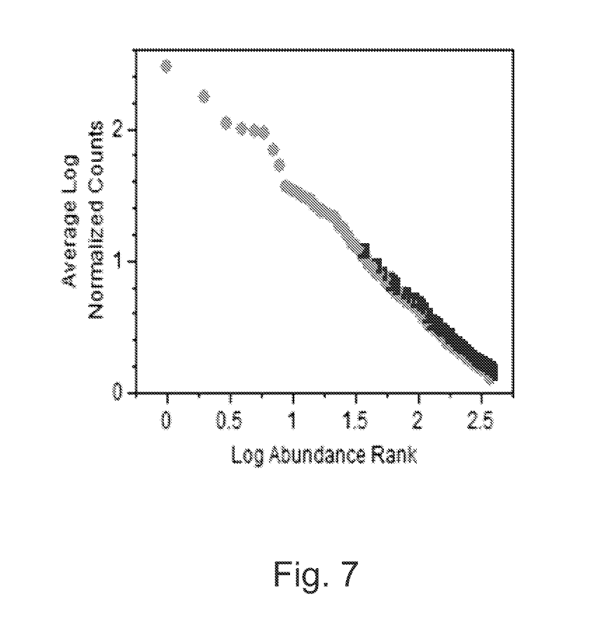

When the RDP classification algorithm was used to classify the consensus sequence for each of the 92 significantly different OTUs, bacteria with higher relative abundance in cases were mostly members of the phyla Firmicutes (42.6%), Bacteroidetes (25.5%) and Proteobacteria (24.5%) (FIG. 2 & FIG. 12-1-12-7). A rank-abundance curve demonstrates that the OTU differences between cases and controls (significant at 10% FDR) are entirely in low abundance taxa (FIG. 7). This observation explains why there are differences between case and control in richness (FIG. 1), which depends on the total number of taxa observed, but not evenness, which is more sensitive to changes in high-abundance taxa.

Since obesity is a risk-factor for development of colorectal cancer, and changes in the human microbiome have been associated with obesity, the relationship between the relative abundance levels of the individual taxa and the risk factors, BMI and Waist-to-Hip Ratio (WHR) was evaluated. Turnbaugh, P. J., et al. A core gut microbiome in obese and lean twins. Nature 457, 480-484 (2009); Zhang, H., et al. Human gut microbiota in obesity and after gastric bypass. Proc Natl Acad Sci USA 106, 2365-2370 (2009). Subjects were classified into one of three BMI categories; Normal (BMI<25), Overweight (BMI=25-29) and Obese (BMI 30 and above) and three WHR levels; low, medium and high based on accepted thresholds (http://www.bmi-calculator.net/waist-to-hip-ratio-calculator/waist-to-hip-ratio-chart.php). For each OTU, the non-parametric Kruskal-Wallis test was performed between the three groups for BMI and WHR. There were no OTUs that showed significant differences between the various BMI and WHR risk factor categories even if a false discovery rate threshold as high as <200% (Tables 4 & 5).

Table 4:

Kruskal-Wallis tests on log-normalized abundances of OTUs (97%) in BMI categories Normal (<25) vs. Overweight (26-30) vs. Obese (>30). RDP classification of consensus sequences at genus level shown. Only OTUs which have at least 1 sequence assigned to them in 25% of the samples are shown. Kruskal-Wallis p-Values were corrected for multiple testing using (n*p)/R where n=total number of taxa tested, p=raw p-Value and R=sorted Rank of the taxon. Benjamini & Hochberg (1995).

TABLE 4

| | KW | | (n * p)/ | |
|---|---|---|---|---|
| OTUname | p-Value | RANK | R | RDP Assignment |
| OTU153 | 0.0125 | 1 | 4.6375 | Roseburia |
| OTU306 | 0.0202 | 2 | 3.7471 | Oligotropha |
| OTU445 | 0.0252 | 3 | 3.1164 | Corynebacterineae |
| OTU4 | 0.0256 | 4 | 2.3744 | Lachnospiraceae Incertae Sedis |
| OTU538 | 0.0295 | 5 | 2.1889 | Lachnospiraceae Incertae Sedis |
| OTU439 | 0.037 | 6 | 2.28783 | Algibacter |
| OTU72 | 0.0371 | 7 | 1.9663 | Aquabacterium |
| OTU525 | 0.0374 | 8 | 1.73443 | Catonella |
| OTU75 | 0.0376 | 9 | 1.54996 | Stenotrophomonas |
| OTU110 | 0.0412 | 10 | 1.52852 | Lachnospiraceae Incertae Sedis |
| OTU98 | 0.0416 | 11 | 1.40305 | Lachnospiraceae Incertae Sedis |
| OTU277 | 0.0429 | 12 | 1.32633 | Lachnospiraceae Incertae Sedis |
| OTU28 | 0.0442 | 13 | 1.2614 | Bacteroides |
| OTU156 | 0.0452 | 14 | 1.1978 | Lachnospiraceae Incertae Sedis |
| OTU16 | 0.0517 | 15 | 1.27871 | Lachnospiraceae Incertae Sedis |
| OTU43 | 0.054 | 16 | 1.25213 | Lachnospiraceae Incertae Sedis |
| OTU27 | 0.0549 | 17 | 1.19811 | Lachnospiraceae Incertae Sedis |
| OTU470 | 0.0686 | 18 | 1.41392 | Lachnospiraceae Incertae Sedis |
| OTU39 | 0.0705 | 19 | 1.37661 | Coriobacterineae |
| OTU506 | 0.0736 | 20 | 1.36528 | Syntrophococcus |
| OTU157 | 0.0758 | 21 | 1.33913 | Marinilabilia |
| OTU9 | 0.0786 | 22 | 1.32548 | Bacteroides |
| OTU131 | 0.0788 | 23 | 1.27108 | Lachnospiraceae Incertae Sedis |
| OTU240 | 0.0798 | 24 | 1.23358 | Weissella |
| OTU566 | 0.0815 | 25 | 1.20946 | Dorea |
| OTU288 | 0.0848 | 26 | 1.21003 | Ruminococcaceae Incertae Sedis |
| OTU1 | 0.0869 | 27 | 1.19407 | Bacteroides |
| OTU341 | 0.0879 | 28 | 1.16468 | Prevotella |
| OTU326 | 0.0911 | 29 | 1.16545 | Lachnospiraceae Incertae Sedis |
| OTU380 | 0.0947 | 30 | 1.17112 | Sporobacter |
| OTU214 | 0.0954 | 31 | 1.14172 | Roseburia |
| OTU11 | 0.0984 | 32 | 1.14083 | Bacteroides |
| OTU172 | 0.0997 | 33 | 1.12087 | Marinilabilia |
| OTU173 | 0.1008 | 34 | 1.09991 | Anaerotruncus |
| OTU499 | 0.1021 | 35 | 1.08226 | Lachnospiraceae Incertae Sedis |
| OTU7 | 0.1026 | 36 | 1.05735 | Bacteroides |
| OTU357 | 0.1084 | 37 | 1.08693 | Coprococcus |
| OTU356 | 0.1086 | 38 | 1.06028 | Novosphingobium |
| OTU248 | 0.1124 | 39 | 1.06924 | Lachnospiraceae Incertae Sedis |
| OTU328 | 0.1146 | 40 | 1.06292 | Parasporobacterium |
| OTU56 | 0.119 | 41 | 1.0768 | Delftia |
| OTU96 | 0.1197 | 42 | 1.05735 | Diaphorobacter |
| OTU372 | 0.1223 | 43 | 1.05519 | Allomonas |
| OTU241 | 0.1272 | 44 | 1.07253 | Chryseobacterium |
| OTU371 | 0.1295 | 45 | 1.06766 | Comamonas |
| OTU305 | 0.1297 | 46 | 1.04606 | Lachnospiraceae Incertae Sedis |
| OTU47 | 0.1317 | 47 | 1.03959 | Succinispira |
| OTU204 | 0.1363 | 48 | 1.05349 | Dialister |
| OTU59 | 0.1363 | 49 | 1.03199 | Acinetobacter |
| OTU138 | 0.147 | 50 | 1.09074 | Simkania |
| OTU519 | 0.1476 | 51 | 1.07372 | Catonella |
| OTU197 | 0.1479 | 52 | 1.05521 | Lactobacillus |
| OTU132 | 0.1487 | 53 | 1.0409 | Parabacteroides |
| OTU79 | 0.1491 | 54 | 1.02437 | Lachnospiraceae Incertae Sedis |
| OTU370 | 0.1519 | 55 | 1.02463 | Lactobacillus |
| OTU97 | 0.152 | 56 | 1.007 | Pseudomonas |
| OTU501 | 0.1567 | 57 | 1.01992 | Ruminococcaceae Incertae Sedis |
| OTU329 | 0.1616 | 58 | 1.03368 | Methanohalobium |
| OTU266 | 0.1618 | 59 | 1.01742 | Bacteroides |
| OTU464 | 0.1618 | 60 | 1.00046 | Marinilabilia |
| OTU338 | 0.1692 | 61 | 1.02907 | Micrococcineae |
| OTU304 | 0.1731 | 62 | 1.03581 | Faecalibacterium |
| OTU374 | 0.1784 | 63 | 1.05058 | Lachnospiraceae Incertae Sedis |
| OTU411 | 0.1827 | 64 | 1.05909 | Faecalibacterium |
| OTU139 | 0.1839 | 65 | 1.04964 | Azonexus |
| OTU399 | 0.1849 | 66 | 1.03936 | Ralstonia |
| OTU40 | 0.1864 | 67 | 1.03216 | Lachnospiraceae Incertae Sedis |
| OTU200 | 0.1891 | 68 | 1.03171 | Helicobacter |
| OTU12 | 0.1918 | 69 | 1.03127 | Bryantella |
| OTU432 | 0.1919 | 70 | 1.01707 | Paludibacter |
| OTU452 | 0.1938 | 71 | 1.01267 | Butyrivibrio |
| OTU86 | 0.1953 | 72 | 1.00634 | Fusobacterium |
| OTU547 | 0.1959 | 73 | 0.9956 | Subdoligranulum |
| OTU51 | 0.1975 | 74 | 0.99017 | Klebsiella |
| OTU148 | 0.1994 | 75 | 0.98637 | Lachnospiraceae Incertae Sedis |
| OTU391 | 0.2026 | 76 | 0.98901 | Aquiflexum |
| OTU120 | 0.2027 | 77 | 0.97665 | Micrococcineae |
| OTU367 | 0.2053 | 78 | 0.97649 | Pseudomonas |
| OTU287 | 0.2077 | 79 | 0.9754 | Anaerovorax |
| OTU412 | 0.2092 | 80 | 0.97017 | Sphingomonas |
| OTU502 | 0.2095 | 81 | 0.95956 | Paludibacter |
| OTU319 | 0.2113 | 82 | 0.956 | Agrobacterium |
| OTU23 | 0.215 | 83 | 0.96102 | Lachnospiraceae Incertae Sedis |
| OTU269 | 0.2155 | 84 | 0.95179 | Butyrivibrio |
| OTU177 | 0.2167 | 85 | 0.94583 | Butyrivibrio |
| OTU437 | 0.2182 | 86 | 0.9413 | Marinilabilia |
| OTU136 | 0.2206 | 87 | 0.94072 | Micrococcineae |
| OTU182 | 0.2221 | 88 | 0.93635 | Lachnospiraceae Incertae Sedis |
| OTU243 | 0.223 | 89 | 0.92958 | Anaerotruncus |
| OTU14 | 0.2291 | 90 | 0.9444 | Erysipelotrichaceae Incertae |
| | | | | Sedis |
| OTU283 | 0.2296 | 91 | 0.93606 | Anaerophaga |
| OTU421 | 0.2297 | 92 | 0.92629 | Streptococcus |
| OTU238 | 0.2308 | 93 | 0.92072 | Lachnospiraceae Incertae Sedis |
| OTU442 | 0.2308 | 94 | 0.91092 | Roseburia |
| OTU492 | 0.2332 | 95 | 0.91071 | Coriobacterineae |
| OTU29 | 0.235 | 96 | 0.90818 | Lachnospiraceae Incertae Sedis |
| OTU406 | 0.2368 | 97 | 0.9057 | Bacteroides |
| OTU265 | 0.2376 | 98 | 0.89949 | Sphingomonas |
| OTU90 | 0.2431 | 99 | 0.91101 | Lachnospiraceae Incertae Sedis |
| OTU38 | 0.2507 | 100 | 0.9301 | Pseudomonas |
| OTU32 | 0.251 | 101 | 0.92199 | Erysipelotrichaceae Incertae |
| | | | | Sedis |
| OTU458 | 0.2529 | 102 | 0.91986 | Roseburia |
| OTU474 | 0.2555 | 103 | 0.9203 | Sphingobium |
| OTU569 | 0.259 | 104 | 0.92393 | Erwinia |
| OTU101 | 0.2611 | 105 | 0.92255 | Pseudoalteromonas |
| OTU162 | 0.2672 | 106 | 0.9352 | Veillonella |
| OTU22 | 0.2693 | 107 | 0.93374 | Acidovorax |
| OTU37 | 0.2702 | 108 | 0.92819 | Cloacibacterium |
| OTU416 | 0.2715 | 109 | 0.9241 | Lachnospiraceae Incertae Sedis |
| OTU80 | 0.273 | 110 | 0.92075 | Lachnospiraceae Incertae Sedis |
| OTU392 | 0.2753 | 111 | 0.92015 | Lachnospiraceae Incertae Sedis |
| OTU87 | 0.2765 | 112 | 0.91591 | Propionibacterineae |
| OTU161 | 0.2781 | 113 | 0.91305 | Prevotella |
| OTU109 | 0.2825 | 114 | 0.91936 | Turicibacter |
| OTU297 | 0.2949 | 115 | 0.95137 | Bacillaceae 1 |
| OTU216 | 0.3 | 116 | 0.95948 | Sphingomonas |
| OTU127 | 0.3011 | 117 | 0.95477 | Lachnospiraceae Incertae Sedis |
| OTU256 | 0.3017 | 118 | 0.94857 | Anaerotruncus |
| OTU195 | 0.3058 | 119 | 0.95338 | Pseudoalteromonas |
| OTU119 | 0.3065 | 120 | 0.9476 | Lachnobacterium |
| OTU239 | 0.3065 | 121 | 0.93976 | Succinispira |
| OTU183 | 0.3107 | 122 | 0.94483 | Bacteroides |
| OTU146 | 0.3111 | 123 | 0.93836 | Vibrio |
| OTU70 | 0.3138 | 124 | 0.93887 | Sphingobium |
| OTU300 | 0.3145 | 125 | 0.93344 | Lachnospiraceae Incertae Sedis |
| OTU354 | 0.3245 | 126 | 0.95547 | Anaerotruncus |
| OTU128 | 0.3258 | 127 | 0.95175 | Prevotella |
| OTU345 | 0.3295 | 128 | 0.95504 | Butyrivibrio |
| OTU144 | 0.3315 | 129 | 0.95338 | Dorea |
| OTU133 | 0.3389 | 130 | 0.96717 | Faecalibacterium |
| OTU393 | 0.3441 | 131 | 0.97451 | Micrococcineae |
| OTU401 | 0.3465 | 132 | 0.97388 | Alistipes |
| OTU226 | 0.3468 | 133 | 0.96739 | Rikenella |
| OTU313 | 0.347 | 134 | 0.96072 | Enterobacter |
| OTU454 | 0.3474 | 135 | 0.95471 | Paludibacter |
| OTU6 | 0.3478 | 136 | 0.94878 | Lachnospiraceae Incertae Sedis |
| OTU118 | 0.3482 | 137 | 0.94294 | Burkholderia |
| OTU176 | 0.3533 | 138 | 0.94981 | Erwinia |
| OTU397 | 0.357 | 139 | 0.95286 | Peptostreptococcaceae Incertae |
| | | | | Sedis |
| OTU180 | 0.3577 | 140 | 0.94791 | Roseburia |
| OTU168 | 0.3627 | 141 | 0.95434 | Roseburia |
| OTU419 | 0.3647 | 142 | 0.95284 | Micrococcineae |
| OTU50 | 0.3647 | 143 | 0.94618 | Sutterella |
| OTU34 | 0.3652 | 144 | 0.9409 | Dorea |
| OTU71 | 0.3653 | 145 | 0.93466 | Lachnospiraceae Incertae Sedis |
| OTU64 | 0.3681 | 146 | 0.93538 | Erwinia |
| OTU159 | 0.375 | 147 | 0.94643 | Faecalibacterium |
| OTU199 | 0.376 | 148 | 0.94254 | Acetanaerobacterium |
| OTU88 | 0.3762 | 149 | 0.93671 | Streptococcus |
| OTU178 | 0.3777 | 150 | 0.93418 | Lachnospiraceae Incertae Sedis |
| OTU352 | 0.3778 | 151 | 0.92824 | Saprospira |
| OTU237 | 0.381 | 152 | 0.92994 | Prevotella |
| OTU210 | 0.3815 | 153 | 0.92508 | Allobaculum |
| OTU225 | 0.3842 | 154 | 0.92557 | Prevotella |
| OTU74 | 0.3866 | 155 | 0.92535 | Ruminococcus |
| OTU334 | 0.3908 | 156 | 0.9294 | Citrobacter |
| OTU192 | 0.3917 | 157 | 0.92561 | Sphingomonas |
| OTU158 | 0.3954 | 158 | 0.92844 | Bacteroides |
| OTU353 | 0.396 | 159 | 0.924 | Dorea |
| OTU229 | 0.4 | 160 | 0.9275 | Coriobacterineae |
| OTU193 | 0.4004 | 161 | 0.92266 | Xylanibacter |
| OTU230 | 0.4021 | 162 | 0.92086 | Butyrivibrio |
| OTU57 | 0.4051 | 163 | 0.92204 | Lachnospiraceae Incertae Sedis |
| OTU19 | 0.409 | 164 | 0.92524 | Syntrophococcus |
| OTU363 | 0.4092 | 165 | 0.92008 | Faecalibacterium |
| OTU65 | 0.4105 | 166 | 0.91744 | Lachnospiraceae Incertae Sedis |
| OTU145 | 0.4157 | 167 | 0.9235 | Afipia |
| OTU270 | 0.4187 | 168 | 0.92463 | Succinispira |
| OTU84 | 0.4201 | 169 | 0.92223 | Marinomonas |
| OTU100 | 0.4225 | 170 | 0.92204 | Xylanibacter |
| OTU366 | 0.4227 | 171 | 0.91709 | Coprococcus |
| OTU403 | 0.4238 | 172 | 0.91413 | Methylobacterium |
| OTU267 | 0.4253 | 173 | 0.91206 | Parabacteroides |
| OTU170 | 0.4256 | 174 | 0.90746 | Bacteroides |
| OTU423 | 0.43 | 175 | 0.9116 | Parasporobacterium |
| OTU268 | 0.4307 | 176 | 0.9079 | Staphylococcus |
| OTU365 | 0.4311 | 177 | 0.90361 | Succinispira |
| OTU181 | 0.4312 | 178 | 0.89874 | Bacteroides |
| OTU364 | 0.4323 | 179 | 0.896 | Exiguobacterium |
| OTU491 | 0.4335 | 180 | 0.89349 | Clostridiaceae 1 |
| OTU105 | 0.4364 | 181 | 0.8945 | Bacteroides |
| OTU5 | 0.4368 | 182 | 0.8904 | Sphingomonas |
| OTU322 | 0.4414 | 183 | 0.89486 | Roseburia |
| OTU224 | 0.4432 | 184 | 0.89363 | Prevotella |
| OTU213 | 0.4468 | 185 | 0.89602 | Lactococcus |
| OTU343 | 0.4495 | 186 | 0.89658 | Lachnobacterium |
| OTU26 | 0.4516 | 187 | 0.89596 | Dorea |
| OTU49 | 0.4579 | 188 | 0.90362 | Sutterella |
| OTU186 | 0.4584 | 189 | 0.89982 | Faecalibacterium |
| OTU45 | 0.4603 | 190 | 0.8988 | Xenohaliotis |
| OTU344 | 0.4722 | 191 | 0.91721 | Carnobacteriaceae 1 |
| OTU114 | 0.4744 | 192 | 0.91668 | Megamonas |
| OTU194 | 0.478 | 193 | 0.91885 | Alistipes |
| OTU249 | 0.4809 | 194 | 0.91966 | Faecalibacterium |
| OTU73 | 0.4888 | 195 | 0.92997 | Lactococcus |
| OTU122 | 0.4898 | 196 | 0.92712 | Prevotella |
| OTU307 | 0.4912 | 197 | 0.92505 | Megamonas |
| OTU124 | 0.5009 | 198 | 0.93856 | Lactobacillus |
| OTU187 | 0.5039 | 199 | 0.93943 | Erysipelotrichaceae Incertae |
| | | | | Sedis |
| OTU235 | 0.5047 | 200 | 0.93622 | Desulfovibrio |
| OTU149 | 0.5059 | 201 | 0.93378 | Haemophilus |
| OTU309 | 0.5061 | 202 | 0.92952 | Paludibacter |
| OTU143 | 0.5074 | 203 | 0.92732 | Lachnospiraceae Incertae Sedis |
| OTU31 | 0.5076 | 204 | 0.92314 | Coprococcus |
| OTU30 | 0.5115 | 205 | 0.92569 | Bryantella |
| OTU151 | 0.5116 | 206 | 0.92138 | Subdoligranulum |
| OTU425 | 0.5166 | 207 | 0.92589 | Enhydrobacter |
| OTU41 | 0.5176 | 208 | 0.92322 | Subdoligranulum |
| OTU291 | 0.5193 | 209 | 0.92182 | Syntrophococcus |
| OTU82 | 0.5226 | 210 | 0.92326 | Roseburia |
| OTU206 | 0.5229 | 211 | 0.91941 | Paludibacter |
| OTU160 | 0.5232 | 212 | 0.9156 | Lachnospiraceae Incertae Sedis |
| OTU135 | 0.5243 | 213 | 0.91322 | Clostridiaceae 1 |
| OTU418 | 0.5253 | 214 | 0.91068 | Stenotrophomonas |
| OTU152 | 0.5303 | 215 | 0.91508 | Faecalibacterium |
| OTU46 | 0.5305 | 216 | 0.91118 | Bacillaceae 1 |
| OTU76 | 0.5306 | 217 | 0.90715 | Lachnobacterium |
| OTU89 | 0.5315 | 218 | 0.90453 | Bacteroides |
| OTU330 | 0.532 | 219 | 0.90124 | Coriobacterineae |
| OTU471 | 0.535 | 220 | 0.9022 | Lachnospiraceae Incertae Sedis |
| OTU171 | 0.5368 | 221 | 0.90114 | Bacteroides |
| OTU103 | 0.5438 | 222 | 0.90878 | Roseburia |
| OTU244 | 0.5447 | 223 | 0.9062 | Prevotella |
| OTU358 | 0.5453 | 224 | 0.90315 | Roseburia |
| OTU453 | 0.5461 | 225 | 0.90046 | Faecalibacterium |
| OTU111 | 0.5483 | 226 | 0.90009 | Peptostreptococcaceae Incertae |
| | | | | Sedis |
| OTU189 | 0.5493 | 227 | 0.89775 | Acidovorax |
| OTU24 | 0.55 | 228 | 0.89496 | Lachnospiraceae Incertae Sedis |
| OTU376 | 0.5502 | 229 | 0.89137 | Methylobacterium |
| OTU203 | 0.5533 | 230 | 0.8925 | Rheinheimera |
| OTU455 | 0.5625 | 231 | 0.90341 | Finegoldia |
| OTU484 | 0.5693 | 232 | 0.91039 | Effluviibacter |
| OTU350 | 0.5747 | 233 | 0.91508 | Coprococcus |
| OTU35 | 0.5757 | 234 | 0.91276 | Bryantella |
| OTU69 | 0.5784 | 235 | 0.91313 | Lachnospiraceae Incertae Sedis |
| OTU91 | 0.5813 | 236 | 0.91382 | Lactobacillus |
| OTU66 | 0.5835 | 237 | 0.91341 | Streptococcus |
| OTU463 | 0.5846 | 238 | 0.91129 | Lachnospiraceae Incertae Sedis |
| OTU387 | 0.58818 | 239 | 0.91303 | Coprococcus |
| OTU378 | 0.589 | 240 | 0.9105 | Bacillaceae 1 |
| OTU126 | 0.5937 | 241 | 0.91395 | Aeromonas |
| OTU373 | 0.5949 | 242 | 0.91202 | Sporobacter |
| OTU169 | 0.595 | 243 | 0.90842 | Streptococcus |
| OTU233 | 0.5959 | 244 | 0.90606 | Syntrophococcus |
| OTU284 | 0.5973 | 245 | 0.90448 | Rubritepida |
| OTU108 | 0.6038 | 246 | 0.91061 | Lachnospiraceae Incertae Sedis |
| OTU247 | 0.6044 | 247 | 0.90782 | Xylanibacter |
| OTU130 | 0.6073 | 248 | 0.9085 | Lachnospiraceae Incertae Sedis |
| OTU165 | 0.6145 | 249 | 0.91558 | Alistipes |
| OTU327 | 0.615 | 250 | 0.91266 | Pelomonas |
| OTU106 | 0.6165 | 251 | 0.91124 | Lachnospiraceae Incertae Sedis |
| OTU420 | 0.6168 | 252 | 0.90807 | Dorea |
| OTU207 | 0.6187 | 253 | 0.90726 | Succinispira |
| OTU324 | 0.6203 | 254 | 0.90603 | Faecalibacterium |
| OTU275 | 0.6213 | 255 | 0.90393 | Lachnospiraceae Incertae Sedis |
| OTU347 | 0.6235 | 256 | 0.90359 | Vitellibacter |
| OTU198 | 0.6266 | 257 | 0.90455 | Lachnospiraceae Incertae Sedis |
| OTU493 | 0.6268 | 258 | 0.90133 | Lachnospiraceae Incertae Sedis |
| OTU60 | 0.6291 | 259 | 0.90114 | Subdoligranulum |
| OTU164 | 0.6307 | 260 | 0.89996 | Faecalibacterium |
| OTU85 | 0.6349 | 261 | 0.90248 | Bacteroides |
| OTU155 | 0.6395 | 262 | 0.90555 | Roseburia |
| OTU188 | 0.6396 | 263 | 0.90225 | Lachnospiraceae Incertae Sedis |
| OTU117 | 0.6399 | 264 | 0.89925 | Naxibacter |
| OTU404 | 0.6453 | 265 | 0.90342 | Hallella |
| OTU53 | 0.6509 | 266 | 0.90783 | Succinivibrio |
| OTU67 | 0.6584 | 267 | 0.91486 | Lactobacillus |
| OTU134 | 0.6601 | 268 | 0.9138 | Ruminococcaceae Incertae Sedis |
| OTU286 | 0.6604 | 269 | 0.91081 | Hallella |
| OTU476 | 0.6642 | 270 | 0.91266 | Streptococcus |
| OTU508 | 0.6654 | 271 | 0.91094 | Lachnospiraceae Incertae Sedis |
| OTU361 | 0.6727 | 272 | 0.91754 | Succinivibrio |
| OTU274 | 0.681 | 273 | 0.92546 | Lachnospiraceae Incertae Sedis |
| OTU113 | 0.6855 | 274 | 0.92818 | Rikenella |
| OTU212 | 0.6881 | 275 | 0.92831 | Coprobacillus |
| OTU52 | 0.69227 | 276 | 0.93055 | Lachnospiraceae Incertae Sedis |
| OTU299 | 0.6954 | 277 | 0.93138 | Lachnospiraceae Incertae Sedis |
| OTU315 | 0.6976 | 278 | 0.93097 | Coriobacterineae |
| OTU429 | 0.6982 | 279 | 0.92843 | Dorea |
| OTU107 | 0.6991 | 280 | 0.92631 | Ruminococcus |
| OTU42 | 0.7035 | 281 | 0.92882 | Prevotella |
| OTU20 | 0.7054 | 282 | 0.92803 | Lachnospiraceae Incertae Sedis |
| OTU15 | 0.7074 | 283 | 0.92737 | Roseburia |
| OTU285 | 0.7114 | 284 | 0.92933 | Butyrivibrio |
| OTU102 | 0.7156 | 285 | 0.93154 | Lachnospiraceae Incertae Sedis |
| OTU375 | 0.7256 | 286 | 0.94125 | Pseudomonas |
| OTU389 | 0.7273 | 287 | 0.94017 | Parabacteroides |
| OTU202 | 0.7275 | 288 | 0.93716 | Lachnospiraceae Incertae Sedis |
| OTU222 | 0.7295 | 289 | 0.93649 | Prevotella |
| OTU395 | 0.7357 | 290 | 0.94119 | Subdoligranulum |
| OTU250 | 0.7363 | 291 | 0.93872 | Paludibacter |
| OTU115 | 0.7405 | 292 | 0.94084 | Roseburia |
| OTU21 | 0.7508 | 293 | 0.95067 | Finegoldia |
| OTU33 | 0.7525 | 294 | 0.94958 | Lachnospiraceae Incertae Sedis |
| OTU360 | 0.7528 | 295 | 0.94674 | Faecalibacterium |
| OTU231 | 0.7545 | 296 | 0.94567 | Anaerotruncus |
| OTU292 | 0.7554 | 297 | 0.94361 | Alistipes |
| OTU242 | 0.7656 | 298 | 0.95315 | Coriobacterineae |
| OTU311 | 0.7664 | 299 | 0.95095 | Lachnospiraceae Incertae Sedis |
| OTU205 | 0.7694 | 300 | 0.95149 | Erysipelotrichaceae Incertae |
| | | | | Sedis |
| OTU217 | 0.7694 | 301 | 0.94833 | Prevotella |
| OTU140 | 0.77 | 302 | 0.94593 | Faecalibacterium |
| OTU317 | 0.7757 | 303 | 0.94978 | Prevotella |
| OTU190 | 0.7768 | 304 | 0.948 | Ruminococcaceae Incertae Sedis |
| OTU282 | 0.7852 | 305 | 0.95511 | Streptococcus |
| OTU312 | 0.7899 | 306 | 0.95769 | Coriobacterineae |
| OTU303 | 0.798 | 307 | 0.96436 | Faecalibacterium |
| OTU296 | 0.8006 | 308 | 0.96436 | Papillibacter |
| OTU150 | 0.8055 | 309 | 0.96712 | Ruminococcaceae Incertae Sedis |
| OTU184 | 0.8057 | 310 | 0.96424 | Lachnospiraceae Incertae Sedis |
| OTU104 | 0.8059 | 311 | 0.96138 | Syntrophococcus |
| OTU154 | 0.808 | 312 | 0.96079 | Faecalibacterium |
| OTU553 | 0.8125 | 313 | 0.96306 | Syntrophococcus |
| OTU254 | 0.8131 | 314 | 0.9607 | Lachnospiraceae Incertae Sedis |
| OTU359 | 0.8214 | 315 | 0.96743 | Faecalibacterium |
| OTU166 | 0.8253 | 316 | 0.96894 | Lachnospiraceae Incertae Sedis |
| OTU142 | 0.8254 | 317 | 0.966 | Lachnospiraceae Incertae Sedis |
| OTU417 | 0.8299 | 318 | 0.96822 | Lachnobacterium |
| OTU10 | 0.833 | 319 | 0.96879 | Coprobacillus |
| OTU18 | 0.837 | 320 | 0.9704 | Faecalibacterium |
| OTU68 | 0.8376 | 321 | 0.96807 | Dorea |
| OTU3 | 0.8382 | 322 | 0.96575 | Lachnospiraceae Incertae Sedis |
| OTU407 | 0.839 | 323 | 0.96368 | Turicibacter |
| OTU495 | 0.8404 | 324 | 0.96231 | Streptococcus |
| OTU61 | 0.8405 | 325 | 0.95946 | Papillibacter |
| OTU17 | 0.846 | 326 | 0.96278 | Escherichia |
| OTU83 | 0.8462 | 327 | 0.96006 | Dorea |
| OTU54 | 0.8468 | 328 | 0.95781 | Lachnospiraceae Incertae Sedis |
| OTU409 | 0.848 | 329 | 0.95626 | Alkalilimnicola |
| OTU25 | 0.8491 | 330 | 0.95459 | Parabacteroides |
| OTU253 | 0.8496 | 331 | 0.95227 | Uruburuella |
| OTU355 | 0.8553 | 332 | 0.95577 | Corynebacterineae |
| OTU264 | 0.8585 | 333 | 0.95647 | Comamonas |
| OTU129 | 0.8632 | 334 | 0.95882 | Roseburia |
| OTU94 | 0.8638 | 335 | 0.95663 | Anaerotruncus |
| OTU227 | 0.868 | 336 | 0.95842 | Lachnospiraceae Incertae Sedis |
| OTU413 | 0.8732 | 337 | 0.9613 | Subdoligranulum |
| OTU8 | 0.8757 | 338 | 0.9612 | Dorea |
| OTU92 | 0.8801 | 339 | 0.96318 | Rubrobacterineae |
| OTU36 | 0.8815 | 340 | 0.96187 | Bacteroides |
| OTU191 | 0.8823 | 341 | 0.95992 | Subdoligranulum |
| OTU422 | 0.8834 | 342 | 0.95831 | Peptococcaceae 1 |
| OTU396 | 0.8849 | 343 | 0.95714 | Coprococcus |
| OTU167 | 0.8882 | 344 | 0.95791 | Allobaculum |
| OTU93 | 0.895 | 345 | 0.96245 | Alistipes |
| OTU408 | 0.8976 | 346 | 0.96246 | Bryantella |
| OTU260 | 0.9 | 347 | 0.96225 | Erysipelotrichaceae Incertae |
| | | | | Sedis |
| OTU2 | 0.9165 | 348 | 0.97707 | Faecalibacterium |
| OTU456 | 0.9187 | 349 | 0.97661 | Anaerovorax |
| OTU293 | 0.9214 | 350 | 0.97668 | Lachnospiraceae Incertae Sedis |
| OTU219 | 0.9222 | 351 | 0.97475 | Rikenella |
| OTU349 | 0.9245 | 352 | 0.9744 | Syntrophococcus |
| OTU460 | 0.9246 | 353 | 0.97175 | Lachnospiraceae Incertae Sedis |
| OTU95 | 0.9326 | 354 | 0.97739 | Ruminococcus |
| OTU48 | 0.9459 | 355 | 0.98853 | Bacteroides |
| OTU55 | 0.9609 | 356 | 1.00139 | Parabacteroides |
| OTU196 | 0.9689 | 357 | 1.0069 | Bacteroides |
| OTU368 | 0.9705 | 358 | 1.00574 | Ruminococcaceae Incertae Sedis |
| OTU424 | 0.9713 | 359 | 1.00377 | Streptococcus |
| OTU137 | 0.9718 | 360 | 1.00149 | Prevotella |
| OTU123 | 0.9789 | 361 | 1.00602 | Papillibacter |
| OTU316 | 0.9789 | 362 | 1.00324 | Alistipes |
| OTU62 | 0.9824 | 363 | 1.00405 | Ruminococcus |
| OTU272 | 0.9832 | 364 | 1.00211 | Sporobacter |
| OTU379 | 0.9862 | 365 | 1.00241 | Roseburia |
| OTU44 | 0.9892 | 366 | 1.00271 | Lachnospiraceae Incertae Sedis |
| OTU141 | 0.9895 | 367 | 1.00028 | Faecalibacterium |
| OTU58 | 0.9913 | 368 | 0.99938 | Peptostreptococcaceae Incertae |
| | | | | Sedis |
| OTU400 | 0.9926 | 369 | 0.99798 | Bryantella |
| OTU179 | 0.9933 | 370 | 0.99598 | Ruminococcaceae Incertae Sedis |
| OTU77 | 0.9993 | 371 | 0.9993 | Coprococcus |

TABLE 5

| OTUname | KW_p-Value | RANK | (n * p)/R | RDP Assignment |
|---|---|---|---|---|
| OTU299 | 0.0059 | 1 | 2.1889 | Lachnospiraceae Incertae Sedis |
| OTU538 | 0.0068 | 2 | 1.2614 | Lachnospiraceae Incertae Sedis |
| OTU306 | 0.0149 | 3 | 1.84263 | Oligotropha |
| OTU569 | 0.0174 | 4 | 1.61385 | Erwinia |
| OTU387 | 0.022 | 5 | 1.6324 | Coprococcus |
| OTU349 | 0.0265 | 6 | 1.63858 | Syntrophococcus |
| OTU8 | 0.0268 | 7 | 1.4204 | Dorea |
| OTU419 | 0.0338 | 8 | 1.56748 | Micrococcineae |
| OTU484 | 0.0349 | 9 | 1.43866 | Effluviibacter |
| OTU19 | 0.0404 | 10 | 1.49884 | Syntrophococcus |
| OTU464 | 0.0406 | 11 | 1.36933 | Marinilabilia |
| OTU156 | 0.0414 | 12 | 1.27995 | Lachnospiraceae Incertae Sedis |
| OTU248 | 0.0432 | 13 | 1.23286 | Lachnospiraceae Incertae Sedis |
| OTU48 | 0.046 | 14 | 1.219 | Bacteroides |
| OTU210 | 0.0463 | 15 | 1.14515 | Allobaculum |
| OTU172 | 0.048 | 16 | 1.113 | Marinilabilia |
| OTU93 | 0.0497 | 17 | 1.08463 | Alistipes |
| OTU373 | 0.0556 | 18 | 1.14598 | Sporobacter |
| OTU168 | 0.0571 | 19 | 1.11495 | Roseburia |
| OTU250 | 0.0588 | 20 | 1.09074 | Paludibacter |
| OTU375 | 0.0613 | 21 | 1.08297 | Pseudomonas |
| OTU291 | 0.0616 | 22 | 1.0388 | Syntrophococcus |
| OTU35 | 0.0698 | 23 | 1.1259 | Bryantella |
| OTU357 | 0.0708 | 24 | 1.09445 | Coprococcus |
| OTU439 | 0.071 | 25 | 1.05364 | Algibacter |
| OTU110 | 0.0715 | 26 | 1.02025 | Lachnospiraceae Incertae Sedis |
| OTU525 | 0.0717 | 27 | 0.98521 | Catonella |
| OTU67 | 0.0736 | 28 | 0.9752 | Lactobacillus |
| OTU5 | 0.0741 | 29 | 0.94797 | Sphingomonas |
| OTU96 | 0.0766 | 30 | 0.94729 | Diaphorobacter |
| OTU493 | 0.0787 | 31 | 0.94186 | Lachnospiraceae Incertae Sedis |
| OTU566 | 0.0835 | 32 | 0.96808 | Dorea |
| OTU84 | 0.0839 | 33 | 0.94324 | Marinomonas |
| OTU34 | 0.0849 | 34 | 0.92641 | Dorea |
| OTU399 | 0.0853 | 35 | 0.90418 | Ralstonia |
| OTU366 | 0.0882 | 36 | 0.90895 | Coprococcus |
| OTU142 | 0.0913 | 37 | 0.91547 | Lachnospiraceae Incertae Sedis |
| OTU95 | 0.0916 | 38 | 0.89431 | Ruminococcus |
| OTU360 | 0.0918 | 39 | 0.87328 | Faecalibacterium |
| OTU45 | 0.0918 | 40 | 0.85145 | Xenohaliotis |
| OTU508 | 0.0926 | 41 | 0.83792 | Lachnospiraceae Incertae Sedis |
| OTU329 | 0.0961 | 42 | 0.84888 | Methanohalobium |
| OTU151 | 0.0962 | 43 | 0.83 | Subdoligranulum |
| OTU501 | 0.0979 | 44 | 0.82548 | Ruminococcaceae Incertae Sedis |
| OTU244 | 0.1002 | 45 | 0.82609 | Prevotella |
| OTU315 | 0.1064 | 46 | 0.85814 | Coriobacterineae |
| OTU553 | 0.1072 | 47 | 0.8462 | Syntrophococcus |
| OTU230 | 0.1095 | 48 | 0.84634 | Butyrivibrio |
| OTU316 | 0.1102 | 49 | 0.83437 | Alistipes |
| OTU197 | 0.1107 | 50 | 0.82139 | Lactobacillus |
| OTU104 | 0.1147 | 51 | 0.83439 | Syntrophococcus |
| OTU191 | 0.1181 | 52 | 0.8426 | Subdoligranulum |
| OTU161 | 0.1184 | 53 | 0.8288 | Prevotella |
| OTU243 | 0.1184 | 54 | 0.81345 | Anaerotruncus |
| OTU62 | 0.1192 | 55 | 0.80406 | Ruminococcus |
| OTU23 | 0.1193 | 56 | 0.79036 | Lachnospiraceae Incertae Sedis |
| OTU205 | 0.1197 | 57 | 0.7791 | Erysipelotrichaceae Incertae Sedis |
| OTU106 | 0.125 | 58 | 0.79957 | Lachnospiraceae Incertae Sedis |
| OTU224 | 0.1271 | 59 | 0.79922 | Prevotella |
| OTU74 | 0.131 | 60 | 0.81002 | Ruminococcus |
| OTU372 | 0.1312 | 61 | 0.79795 | Allomonas |
| OTU470 | 0.1338 | 62 | 0.80064 | Lachnospiraceae Incertae Sedis |
| OTU160 | 0.1368 | 63 | 0.8056 | Lachnospiraceae Incertae Sedis |
| OTU404 | 0.1385 | 64 | 0.80287 | Hallella |
| OTU190 | 0.1394 | 65 | 0.79565 | Ruminococcaceae Incertae Sedis |
| OTU432 | 0.1402 | 66 | 0.78809 | Paludibacter |
| OTU471 | 0.1412 | 67 | 0.78187 | Lachnospiraceae Incertae Sedis |
| OTU28 | 0.144 | 68 | 0.78565 | Bacteroides |
| OTU233 | 0.145 | 69 | 0.77964 | Syntrophococcus |
| OTU41 | 0.1468 | 70 | 0.77804 | Subdoligranulum |
| OTU365 | 0.1534 | 71 | 0.80157 | Succinispira |
| OTU395 | 0.1557 | 72 | 0.80229 | Subdoligranulum |
| OTU305 | 0.1573 | 73 | 0.79943 | Lachnospiraceae Incertae Sedis |
| OTU30 | 0.1594 | 74 | 0.79915 | Bryantella |
| OTU154 | 0.1597 | 75 | 0.78998 | Faecalibacterium |
| OTU46 | 0.1602 | 76 | 0.78203 | Bacillaceae 1 |
| OTU100 | 0.1611 | 77 | 0.77621 | Xylanibacter |
| OTU254 | 0.1671 | 78 | 0.7948 | Lachnospiraceae Incertae Sedis |
| OTU200 | 0.1725 | 79 | 0.81009 | Helicobacter |
| OTU421 | 0.1763 | 80 | 0.81759 | Streptococcus |
| OTU277 | 0.1773 | 81 | 0.81208 | Lachnospiraceae Incertae Sedis |
| OTU239 | 0.1778 | 82 | 0.80444 | Succinispira |
| OTU1 | 0.1808 | 83 | 0.80815 | Bacteroides |
| OTU68 | 0.1814 | 84 | 0.80118 | Dorea |
| OTU72 | 0.1816 | 85 | 0.79263 | Aquabacterium |
| OTU495 | 0.1891 | 86 | 0.81577 | Streptococcus |
| OTU275 | 0.1938 | 87 | 0.82643 | Lachnospiraceae Incertae Sedis |
| OTU370 | 0.1946 | 88 | 0.82042 | Lactobacillus |
| OTU284 | 0.1958 | 89 | 0.8162 | Rubritepida |
| OTU195 | 0.1959 | 90 | 0.80754 | Pseudoalteromonas |
| OTU91 | 0.1979 | 91 | 0.80682 | Lactobacillus |
| OTU82 | 0.198 | 92 | 0.79846 | Roseburia |
| OTU378 | 0.1982 | 93 | 0.79067 | Bacillaceae 1 |
| OTU206 | 0.2061 | 94 | 0.81344 | Paludibacter |
| OTU317 | 0.2063 | 95 | 0.80566 | Prevotella |
| OTU165 | 0.2065 | 96 | 0.79804 | Alistipes |
| OTU113 | 0.2074 | 97 | 0.79325 | Rikenella |
| OTU130 | 0.2101 | 98 | 0.79538 | Lachnospiraceae Incertae Sedis |
| OTU138 | 0.2157 | 99 | 0.80833 | Acidovorax |
| OTU22 | 0.2166 | 100 | 0.80359 | Coriobacterineae |
| OTU492 | 0.2189 | 101 | 0.80408 | Lactococcus |
| OTU73 | 0.2211 | 102 | 0.8042 | Prevotella |
| OTU137 | 0.225 | 103 | 0.81044 | Afipia |
| OTU145 | 0.23 | 104 | 0.82048 | Erwinia |
| OTU64 | 0.2302 | 105 | 0.81337 | Streptococcus |
| OTU282 | 0.2306 | 106 | 0.8071 | Prevotella |
| OTU42 | 0.231 | 107 | 0.80094 | Enhydrobacter |
| OTU425 | 0.2351 | 108 | 0.80761 | Cloacibacterium |
| OTU37 | 0.2366 | 109 | 0.80531 | Papillibacter |
| OTU61 | 0.2382 | 110 | 0.80338 | Roseburia |
| OTU180 | 0.2389 | 111 | 0.79849 | Streptococcus |
| OTU169 | 0.2395 | 112 | 0.79334 | Micrococcineae |
| OTU136 | 0.2416 | 113 | 0.79322 | Faecalibacterium |
| OTU304 | 0.2444 | 114 | 0.79537 | Lachnospiraceae Incertae Sedis |
| OTU188 | 0.2467 | 115 | 0.79588 | Coprobacillus |
| OTU10 | 0.2477 | 116 | 0.79221 | Prevotella |
| OTU128 | 0.2568 | 117 | 0.8143 | Dorea |
| OTU420 | 0.2582 | 118 | 0.8118 | Paludibacter |
| OTU454 | 0.2585 | 119 | 0.80591 | Uruburuella |
| OTU253 | 0.2599 | 120 | 0.80352 | Bacteroides |
| OTU406 | 0.2601 | 121 | 0.7975 | Bacteroides |
| OTU7 | 0.2613 | 122 | 0.79461 | Weissella |
| OTU240 | 0.2614 | 123 | 0.78845 | Coriobacterineae |
| OTU312 | 0.2621 | 124 | 0.78419 | Acinetobacter |
| OTU59 | 0.2645 | 125 | 0.78504 | Acidovorax |
| OTU189 | 0.2663 | 126 | 0.78411 | Rubrobacterineae |
| OTU92 | 0.2691 | 127 | 0.78611 | Xylanibacter |
| OTU193 | 0.2737 | 128 | 0.7933 | Streptococcus |
| OTU424 | 0.2749 | 129 | 0.7906 | Papillibacter |
| OTU123 | 0.2753 | 130 | 0.78566 | Ruminococcaceae Incertae Sedis |
| OTU368 | 0.2773 | 131 | 0.78533 | Faecalibacterium |
| OTU18 | 0.2803 | 132 | 0.78781 | Bryantella |
| OTU12 | 0.2818 | 133 | 0.78607 | Sphingomonas |
| OTU192 | 0.284 | 134 | 0.7863 | Succinispira |
| OTU207 | 0.284 | 135 | 0.78047 | Lachnospiraceae Incertae Sedis |
| OTU416 | 0.2856 | 136 | 0.7791 | Allobaculum |
| OTU167 | 0.2875 | 137 | 0.77856 | Lachnospiraceae Incertae Sedis |
| OTU98 | 0.2908 | 138 | 0.78179 | Faecalibacterium |
| OTU249 | 0.2916 | 139 | 0.7783 | Lachnospiraceae Incertae Sedis |
| OTU300 | 0.2948 | 140 | 0.78122 | Roseburia |
| OTU214 | 0.2976 | 141 | 0.78305 | Klebsiella |
| OTU51 | 0.299 | 142 | 0.78119 | Streptococcus |
| OTU476 | 0.3015 | 143 | 0.78221 | Marinilabilia |
| OTU437 | 0.3067 | 144 | 0.79018 | Faecalibacterium |
| OTU453 | 0.3096 | 145 | 0.79215 | Paludibacter |
| OTU309 | 0.3132 | 146 | 0.79587 | Sporobacter |
| OTU380 | 0.321 | 147 | 0.81014 | Pseudomonas |
| OTU367 | 0.3238 | 148 | 0.81169 | Faecalibacterium |
| OTU133 | 0.3241 | 149 | 0.80699 | Prevotella |
| OTU225 | 0.3246 | 150 | 0.80284 | Vitellibacter |
| OTU347 | 0.3294 | 151 | 0.80932 | Propionibacterineae |
| OTU87 | 0.3324 | 152 | 0.81132 | Coprococcus |
| OTU350 | 0.3391 | 153 | 0.82226 | Streptococcus |
| OTU66 | 0.3455 | 154 | 0.83234 | Pelomonas |
| OTU327 | 0.3464 | 155 | 0.82913 | Exiguobacterium |
| OTU364 | 0.3494 | 156 | 0.83094 | Lachnospiraceae Incertae Sedis |
| OTU127 | 0.3529 | 157 | 0.83392 | Finegoldia |
| OTU21 | 0.3576 | 158 | 0.83968 | Rikenella |
| OTU226 | 0.3623 | 159 | 0.84537 | Ruminococcaceae Incertae Sedis |
| OTU150 | 0.3626 | 160 | 0.84078 | Lachnospiraceae Incertae Sedis |
| OTU71 | 0.3626 | 161 | 0.83556 | Bacteroides |
| OTU183 | 0.364 | 162 | 0.8336 | Corynebacterineae |
| OTU445 | 0.3681 | 163 | 0.83782 | Lactococcus |
| OTU213 | 0.369 | 164 | 0.83475 | Anaerotruncus |
| OTU231 | 0.3705 | 165 | 0.83306 | Lachnobacterium |
| OTU119 | 0.3712 | 166 | 0.82961 | Lachnospiraceae Incertae Sedis |
| OTU460 | 0.3766 | 167 | 0.83664 | Chryseobacterium |
| OTU241 | 0.3767 | 168 | 0.83188 | Sphingomonas |
| OTU412 | 0.3778 | 169 | 0.82937 | Carnobacteriaceae 1 |
| OTU344 | 0.3792 | 170 | 0.82755 | Vibrio |
| OTU146 | 0.3819 | 171 | 0.82857 | Megamonas |
| OTU114 | 0.3867 | 172 | 0.8341 | Micrococcineae |
| OTU393 | 0.3888 | 173 | 0.83378 | Lachnobacterium |
| OTU417 | 0.3916 | 174 | 0.83496 | Lachnospiraceae Incertae Sedis |
| OTU131 | 0.3917 | 175 | 0.8304 | Saprospira |
| OTU352 | 0.3921 | 176 | 0.82653 | Roseburia |
| OTU358 | 0.3996 | 177 | 0.83758 | Lachnospiraceae Incertae Sedis |
| OTU227 | 0.4027 | 178 | 0.83934 | Succinivibrio |
| OTU53 | 0.4074 | 179 | 0.84439 | Bacteroides |
| OTU36 | 0.4117 | 180 | 0.84856 | Coriobacterineae |
| OTU39 | 0.4129 | 181 | 0.84633 | Pseudomonas |
| OTU97 | 0.4193 | 182 | 0.85473 | Bacteroides |
| OTU89 | 0.4203 | 183 | 0.85208 | Faecalibacterium |
| OTU186 | 0.4216 | 184 | 0.85007 | Streptococcus |
| OTU88 | 0.4223 | 185 | 0.84688 | Anaerophaga |
| OTU283 | 0.4327 | 186 | 0.86307 | Lachnospiraceae Incertae Sedis |
| OTU16 | 0.4394 | 187 | 0.87175 | Faecalibacterium |
| OTU324 | 0.44 | 188 | 0.8683 | Coprobacillus |
| OTU212 | 0.4402 | 189 | 0.8641 | Succinivibrio |
| OTU361 | 0.4418 | 190 | 0.86267 | Butyrivibrio |
| OTU177 | 0.4429 | 191 | 0.86029 | Roseburia |
| OTU379 | 0.4443 | 192 | 0.85852 | Lachnospiraceae Incertae Sedis |
| OTU3 | 0.4476 | 193 | 0.86041 | Agrobacterium |
| OTU319 | 0.4476 | 194 | 0.85598 | Coriobacterineae |
| OTU229 | 0.4528 | 195 | 0.86148 | Lachnospiraceae Incertae Sedis |
| OTU202 | 0.4564 | 196 | 0.8639 | Lachnospiraceae Incertae Sedis |
| OTU311 | 0.461 | 197 | 0.86818 | Sphingomonas |
| OTU265 | 0.4622 | 198 | 0.86604 | Aquiflexum |
| OTU391 | 0.4654 | 199 | 0.86766 | Peptostreptococcaceae Incertae Sedis |
| OTU397 | 0.4706 | 200 | 0.87296 | Prevotella |
| OTU222 | 0.4779 | 201 | 0.88209 | Lachnospiraceae Incertae Sedis |
| OTU40 | 0.4816 | 202 | 0.88452 | Bacteroides |
| OTU196 | 0.4846 | 203 | 0.88565 | Lachnospiraceae Incertae Sedis |
| OTU24 | 0.4884 | 204 | 0.88822 | Bryantella |
| OTU408 | 0.4951 | 205 | 0.89601 | Roseburia |
| OTU153 | 0.4971 | 206 | 0.89526 | Fusobacterium |
| OTU86 | 0.5011 | 207 | 0.89811 | Lachnospiraceae Incertae Sedis |
| OTU326 | 0.5018 | 208 | 0.89504 | Clostridiaceae 1 |
| OTU491 | 0.5047 | 209 | 0.8959 | Bacteroides |
| OTU171 | 0.5061 | 210 | 0.89411 | Citrobacter |
| OTU334 | 0.5071 | 211 | 0.89163 | Alistipes |
| OTU194 | 0.508 | 212 | 0.889 | Aeromonas |
| OTU126 | 0.5122 | 213 | 0.89214 | Prevotella |
| OTU237 | 0.5138 | 214 | 0.89075 | Dorea |
| OTU26 | 0.5169 | 215 | 0.89195 | Subdoligranulum |
| OTU60 | 0.517 | 216 | 0.888 | Lachnospiraceae Incertae Sedis |
| OTU52 | 0.5335 | 217 | 0.91211 | Ruminococcus |
| OTU107 | 0.5352 | 218 | 0.91082 | Catonella |
| OTU519 | 0.5367 | 219 | 0.9092 | Faecalibacterium |
| OTU140 | 0.5398 | 220 | 0.9103 | Papillibacter |
| OTU296 | 0.5432 | 221 | 0.91189 | Sutterella |
| OTU49 | 0.548 | 222 | 0.9158 | Lachnobacterium |
| OTU343 | 0.5663 | 223 | 0.94214 | Lactobacillus |
| OTU124 | 0.5814 | 224 | 0.96294 | Ruminococcaceae Incertae Sedis |
| OTU288 | 0.5881 | 225 | 0.96971 | Marinilabilia |
| OTU157 | 0.5897 | 226 | 0.96805 | Megamonas |
| OTU307 | 0.5901 | 227 | 0.96444 | Bacteroides |
| OTU266 | 0.5921 | 228 | 0.96346 | Finegoldia |
| OTU455 | 0.5928 | 229 | 0.96039 | Bacteroides |
| OTU11 | 0.5944 | 230 | 0.95879 | Anaerotruncus |
| OTU94 | 0.6022 | 231 | 0.96717 | Turicibacter |
| OTU109 | 0.6054 | 232 | 0.96812 | Bacteroides |
| OTU85 | 0.6056 | 233 | 0.96428 | Roseburia |
| OTU115 | 0.6061 | 234 | 0.96095 | Butyrivibrio |
| OTU452 | 0.6141 | 235 | 0.96949 | Xylanibacter |
| OTU247 | 0.6152 | 236 | 0.96712 | Faecalibacterium |
| OTU359 | 0.6155 | 237 | 0.9635 | Bacteroides |
| OTU170 | 0.6263 | 238 | 0.97629 | Prevotella |
| OTU341 | 0.6266 | 239 | 0.97267 | Lachnospiraceae Incertae Sedis |
| OTU392 | 0.6282 | 240 | 0.97109 | Faecalibacterium |
| OTU164 | 0.6284 | 241 | 0.96737 | Lachnospiraceae Incertae Sedis |
| OTU57 | 0.631 | 242 | 0.96736 | Lachnospiraceae Incertae Sedis |
| OTU166 | 0.6318 | 243 | 0.9646 | Rikenella |
| OTU219 | 0.6379 | 244 | 0.96992 | Parabacteroides |
| OTU389 | 0.6418 | 245 | 0.97187 | Clostridiaceae 1 |
| OTU135 | 0.6419 | 246 | 0.96807 | Haemophilus |
| OTU149 | 0.6421 | 247 | 0.96445 | Alkalilimnicola |
| OTU409 | 0.6428 | 248 | 0.96161 | Lachnospiraceae Incertae Sedis |
| OTU102 | 0.643 | 249 | 0.95804 | Peptostreptococcaceae Incertae Sedis |
| OTU58 | 0.644 | 250 | 0.9557 | Burkholderia |
| OTU118 | 0.6467 | 251 | 0.95588 | Parabacteroides |
| OTU55 | 0.6552 | 252 | 0.9646 | Parasporobacterium |
| OTU328 | 0.6559 | 253 | 0.96181 | Lachnospiraceae Incertae Sedis |
| OTU238 | 0.6571 | 254 | 0.95978 | Stenotrophomonas |
| OTU75 | 0.6579 | 255 | 0.95718 | Dorea |
| OTU429 | 0.6587 | 256 | 0.9546 | Peptococcaceae 1 |
| OTU422 | 0.6675 | 257 | 0.96359 | Prevotella |
| OTU122 | 0.6782 | 258 | 0.97524 | Rheinheimera |
| OTU203 | 0.6874 | 259 | 0.98465 | Stenotrophomonas |
| OTU418 | 0.6879 | 260 | 0.98158 | Lachnospiraceae Incertae Sedis |
| OTU463 | 0.6882 | 261 | 0.97825 | Prevotella |
| OTU217 | 0.6897 | 262 | 0.97664 | Ruminococcaceae Incertae Sedis |
| OTU179 | 0.69 | 263 | 0.97335 | Dorea |
| OTU353 | 0.6943 | 264 | 0.9757 | Lachnospiraceae Incertae Sedis |
| OTU20 | 0.6949 | 265 | 0.97286 | Lachnospiraceae Incertae Sedis |
| OTU6 | 0.6964 | 266 | 0.97129 | Anaerovorax |
| OTU456 | 0.6974 | 267 | 0.96905 | Bacteroides |
| OTU158 | 0.6984 | 268 | 0.96681 | Alistipes |
| OTU292 | 0.6998 | 269 | 0.96515 | Lachnospiraceae Incertae Sedis |
| OTU65 | 0.7036 | 270 | 0.9668 | Butyrivibrio |
| OTU345 | 0.7042 | 271 | 0.96405 | Lachnospiraceae Incertae Sedis |
| OTU69 | 0.7079 | 272 | 0.96555 | Parabacteroides |
| OTU267 | 0.7093 | 273 | 0.96392 | Sphingobium |
| OTU474 | 0.7138 | 274 | 0.9665 | Lachnospiraceae Incertae Sedis |
| OTU184 | 0.7144 | 275 | 0.96379 | Syntrophococcus |
| OTU506 | 0.7161 | 276 | 0.96258 | Lachnospiraceae Incertae Sedis |
| OTU44 | 0.7174 | 277 | 0.96085 | Roseburia |
| OTU15 | 0.7254 | 278 | 0.96807 | Bacteroides |
| OTU105 | 0.7299 | 279 | 0.97058 | Lachnospiraceae Incertae Sedis |
| OTU374 | 0.7312 | 280 | 0.96884 | Butyrivibrio |
| OTU285 | 0.7314 | 281 | 0.96566 | Methylobacterium |
| OTU376 | 0.732 | 282 | 0.96302 | Anaerotruncus |
| OTU256 | 0.7326 | 283 | 0.9604 | Lachnospiraceae Incertae Sedis |
| OTU27 | 0.7346 | 284 | 0.95964 | Parasporobacterium |
| OTU423 | 0.7388 | 285 | 0.96174 | Anaerovorax |
| OTU287 | 0.7472 | 286 | 0.96927 | Paludibacter |
| OTU502 | 0.7498 | 287 | 0.96925 | Lachnospiraceae Incertae Sedis |
| OTU274 | 0.7517 | 288 | 0.96834 | Lachnospiraceae Incertae Sedis |
| OTU293 | 0.7548 | 289 | 0.96896 | Pseudoalteromonas |
| OTU101 | 0.7558 | 290 | 0.9669 | Faecalibacterium |
| OTU141 | 0.761 | 291 | 0.97021 | Roseburia |
| OTU129 | 0.7628 | 292 | 0.96917 | Comamonas |
| OTU264 | 0.7667 | 293 | 0.9708 | Coprococcus |
| OTU77 | 0.7678 | 294 | 0.96889 | Lachnospiraceae Incertae Sedis |
| OTU182 | 0.7731 | 295 | 0.97227 | Corynebacterineae |
| OTU355 | 0.7757 | 296 | 0.97225 | Lachnospiraceae Incertae Sedis |
| OTU90 | 0.777 | 297 | 0.9706 | Lachnospiraceae Incertae Sedis |
| OTU29 | 0.7788 | 298 | 0.96958 | Lachnospiraceae Incertae Sedis |
| OTU178 | 0.7861 | 299 | 0.97539 | Veillonella |
| OTU162 | 0.7889 | 300 | 0.97561 | Dorea |
| OTU83 | 0.7948 | 301 | 0.97964 | Parabacteroides |
| OTU25 | 0.7955 | 302 | 0.97725 | Acetanaerobacterium |
| OTU199 | 0.7962 | 303 | 0.97489 | Dialister |
| OTU204 | 0.808 | 304 | 0.98608 | Anaerotruncus |
| OTU354 | 0.8095 | 305 | 0.98467 | Lachnospiraceae Incertae Sedis |
| OTU143 | 0.8198 | 306 | 0.99394 | Roseburia |
| OTU458 | 0.8218 | 307 | 0.99312 | Erysipelotrichaceae Incertae Sedis |
| OTU187 | 0.8256 | 308 | 0.99447 | Lachnospiraceae Incertae Sedis |
| OTU54 | 0.8309 | 309 | 0.99762 | Hallella |
| OTU286 | 0.8311 | 310 | 0.99464 | Comamonas |
| OTU371 | 0.8371 | 311 | 0.9986 | Lachnospiraceae Incertae Sedis |
| OTU4 | 0.8391 | 312 | 0.99778 | Micrococcineae |
| OTU120 | 0.8398 | 313 | 0.99542 | Alistipes |
| OTU401 | 0.8408 | 314 | 0.99343 | Peptostreptococcaceae Incertae Sedis |
| OTU111 | 0.8414 | 315 | 0.99098 | Sutterella |
| OTU50 | 0.8421 | 316 | 0.98867 | Pseudomonas |
| OTU38 | 0.8472 | 317 | 0.99152 | Micrococcineae |
| OTU338 | 0.8506 | 318 | 0.99237 | Lachnospiraceae Incertae Sedis |
| OTU80 | 0.8517 | 319 | 0.99054 | Erysipelotrichaceae Incertae Sedis |
| OTU260 | 0.8519 | 320 | 0.98767 | Erysipelotrichaceae Incertae Sedis |
| OTU32 | 0.8541 | 321 | 0.98714 | Lachnobacterium |
| OTU76 | 0.8553 | 322 | 0.98545 | Delftia |
| OTU56 | 0.8691 | 323 | 0.99825 | Enterobacter |
| OTU313 | 0.8702 | 324 | 0.99643 | Faecalibacterium |
| OTU411 | 0.871 | 325 | 0.99428 | Succinispira |
| OTU47 | 0.8731 | 326 | 0.99362 | Azonexus |
| OTU139 | 0.8742 | 327 | 0.99183 | Roseburia |
| OTU103 | 0.8747 | 328 | 0.98937 | Lachnospiraceae Incertae Sedis |
| OTU198 | 0.8811 | 329 | 0.99358 | Sphingobium |
| OTU70 | 0.8829 | 330 | 0.99259 | Faecalibacterium |
| OTU303 | 0.8873 | 331 | 0.99453 | Novosphingobium |
| OTU356 | 0.8948 | 332 | 0.99991 | Turicibacter |
| OTU407 | 0.8955 | 333 | 0.99769 | Parabacteroides |
| OTU132 | 0.8999 | 334 | 0.99959 | Lachnospiraceae Incertae Sedis |
| OTU79 | 0.9073 | 335 | 1.0048 | Subdoligranulum |
| OTU413 | 0.9088 | 336 | 1.00347 | Sporobacter |
| OTU272 | 0.9089 | 337 | 1.0006 | Subdoligranulum |
| OTU547 | 0.9101 | 338 | 0.99896 | Erwinia |
| OTU176 | 0.9119 | 339 | 0.99798 | Coriobacterineae |
| OTU330 | 0.913 | 340 | 0.99624 | Faecalibacterium |
| OTU363 | 0.9162 | 341 | 0.9968 | Coprococcus |
| OTU396 | 0.9174 | 342 | 0.99519 | Anaerotruncus |
| OTU173 | 0.9183 | 343 | 0.99326 | Staphylococcus |
| OTU268 | 0.9239 | 344 | 0.99642 | Lachnospiraceae Incertae Sedis |
| OTU108 | 0.926 | 345 | 0.99579 | Escherichia |
| OTU17 | 0.9269 | 346 | 0.99387 | Bacteroides |
| OTU9 | 0.9287 | 347 | 0.99293 | Erysipelotrichaceae Incertae Sedis |
| OTU14 | 0.9289 | 348 | 0.99029 | Lachnospiraceae Incertae Sedis |
| OTU148 | 0.9313 | 349 | 0.99001 | Roseburia |
| OTU155 | 0.9313 | 350 | 0.98718 | Butyrivibrio |
| OTU269 | 0.9376 | 351 | 0.99102 | Coprococcus |
| OTU31 | 0.9397 | 352 | 0.99042 | Lachnospiraceae Incertae Sedis |
| OTU499 | 0.9451 | 353 | 0.99329 | Lachnospiraceae Incertae Sedis |
| OTU33 | 0.9497 | 354 | 0.99531 | Roseburia |
| OTU322 | 0.9515 | 355 | 0.99438 | Desulfovibrio |
| OTU235 | 0.9547 | 356 | 0.99493 | Sphingomonas |
| OTU216 | 0.9582 | 357 | 0.99578 | Naxibacter |
| OTU117 | 0.9598 | 358 | 0.99465 | Faecalibacterium |
| OTU2 | 0.9697 | 359 | 1.00211 | Faecalibacterium |
| OTU152 | 0.9698 | 360 | 0.99943 | Lachnospiraceae Incertae Sedis |
| OTU43 | 0.9713 | 361 | 0.99821 | Succinispira |
| OTU270 | 0.9719 | 362 | 0.99606 | Bacteroides |
| OTU181 | 0.9731 | 363 | 0.99455 | Ruminococcaceae Incertae Sedis |
| OTU134 | 0.9734 | 364 | 0.99212 | Faecalibacterium |
| OTU159 | 0.9739 | 365 | 0.98991 | Dorea |
| OTU144 | 0.9784 | 366 | 0.99177 | Bacillaceae 1 |
| OTU297 | 0.9809 | 367 | 0.99159 | Methylobacterium |
| OTU403 | 0.9815 | 368 | 0.9895 | Coriobacterineae |
| OTU242 | 0.9892 | 369 | 0.99456 | Roseburia |
| OTU442 | 0.9918 | 370 | 0.99448 | Bryantella |
| OTU400 | 0.9995 | 371 | 0.9995 | Simkania |

Table 5:

KruskalWallis-tests on log-normalized abundances of OTUs (97%) in WHR levels low, medium and high. Only OTUs which have at least 1 sequence assigned to them in 25% of the samples are shown. RDP classification of consensus sequences at genus level shown. Kruskal-Wallis p-Values were corrected for multiple testing using (n*p)/R where n=total number of taxa tested, p=raw p-Value and R=sorted rank of the taxon. Benjamini & Hochberg (1995).

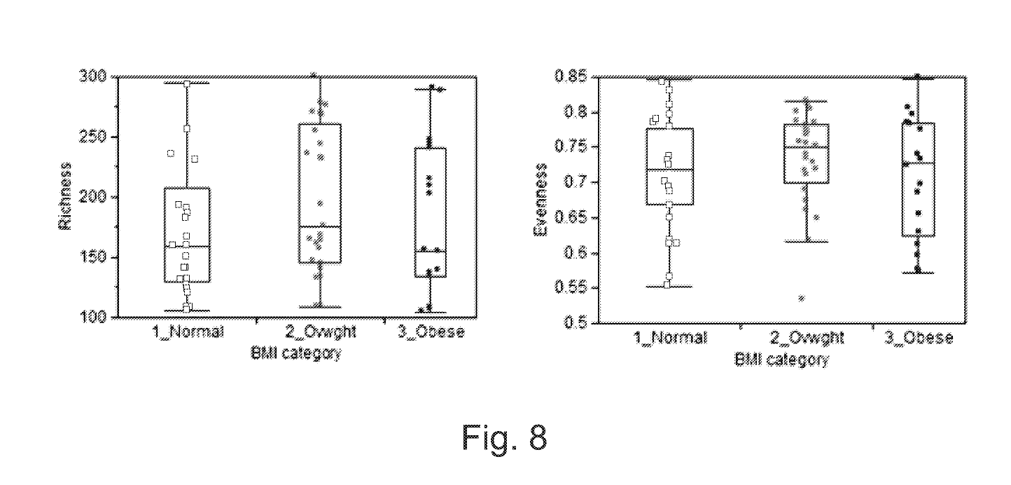

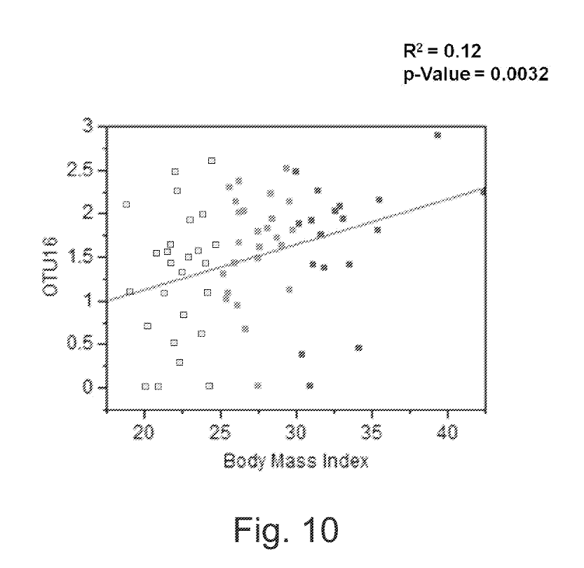

Likewise, there were no significant differences in the diversity measures, richness and evenness, between the various risk factor categories (FIGS. 8 & 9). Finally, regressions between BMI values and WHR values against each taxa at the OTU level also showed no significant association between the OTUs with either BMI or WHR at an FDR threshold of <10% (FIGS. 10 & 11, Tables 6 & 7). Subjects were classified into one of three BMI categories; Normal (<25), Overweight (25-29) and Obese (30 and above) and three WHR levels; low, medium and high based on the accepted thresholds in the medical field (http://www.bmi-calculator.net/waist-to-hip-ratio-calculator/waist-to-hip-ratio-chart.php). For each OTU, the non-parametric Kruskal-Wallis test was performed between the three groups for BMI and WHR. Results indicate that there were no OTUs that showed significant differences between the various BMI and WHR risk factor categories even if a false discovery rate threshold was set as high as <600% (Tables 4 & 5).

Table 6:

Regressions on log-normalized abundances of OTUs (97%) vs BMIs of all samples with RDP classifications of consensus sequences at genus level shown. Only OTUs which have at least 1 sequence assigned to them in 25% of the samples are shown. Regression p-Values were corrected for multiple testing using (n*p)/R where n=total number of taxa tested, p =raw p-Value and R=sorted rank of the taxon. Benjamini & Hochberg (1995).

TABLE 6

| OtuName | R2 | p-Value | RANK | (n * p)/R | RDP assignment |
|---|---|---|---|---|---|
| OTU16 | 0.12079 | 0.00320 | 1 | 1.18672 | Lachnospiraceae Incertae Sedis |
| OTU492 | 0.08200 | 0.01624 | 2 | 3.01333 | Coriobacterineae |
| OTU39 | 0.07881 | 0.01857 | 3 | 2.29692 | Coriobacterineae |
| OTU306 | 0.07825 | 0.01901 | 4 | 1.76333 | Oligotropha |
| OTU40 | 0.07472 | 0.02204 | 5 | 1.63559 | Lachnospiraceae Incertae Sedis |
| OTU43 | 0.07415 | 0.02257 | 6 | 1.39583 | Lachnospiraceae Incertae Sedis |
| OTU305 | 0.07331 | 0.02339 | 7 | 1.23956 | Lachnospiraceae Incertae Sedis |
| OTU357 | 0.07070 | 0.02609 | 8 | 1.20976 | Coprococcus |
| OTU4 | 0.06895 | 0.02808 | 9 | 1.15764 | Lachnospiraceae Incertae Sedis |
| OTU138 | 0.06863 | 0.02846 | 10 | 1.05595 | Simkania |
| OTU277 | 0.06168 | 0.03817 | 11 | 1.28733 | Lachnospiraceae Incertae Sedis |
| OTU237 | 0.05815 | 0.04432 | 12 | 1.37034 | Prevotella |
| OTU131 | 0.05790 | 0.04479 | 13 | 1.27825 | Lachnospiraceae Incertae Sedis |
| OTU372 | 0.05470 | 0.05141 | 14 | 1.36242 | Allomonas |
| OTU329 | 0.05378 | 0.05339 | 15 | 1.32046 | Methanohalobium |
| OTU105 | 0.05349 | 0.05406 | 16 | 1.25351 | Bacteroides |
| OTU172 | 0.05309 | 0.05498 | 17 | 1.19992 | Marinilabilia |
| OTU370 | 0.05290 | 0.05540 | 18 | 1.14185 | Lactobacillus |
| OTU397 | 0.05190 | 0.05789 | 19 | 1.13039 | Peptostreptococcaceae Incertae Sedis |
| OTU27 | 0.05132 | 0.05932 | 20 | 1.10034 | Lachnospiraceae Incertae Sedis |
| OTU67 | 0.05116 | 0.05973 | 21 | 1.05515 | Lactobacillus |
| OTU439 | 0.05040 | 0.06178 | 22 | 1.0418 | Algibacter |
| OTU110 | 0.04969 | 0.06362 | 23 | 1.02621 | Lachnospiraceae Incertae Sedis |
| OTU210 | 0.04921 | 0.06494 | 24 | 1.00386 | Allobaculum |
| OTU380 | 0.04900 | 0.06547 | 25 | 0.9715 | Sporobacter |
| OTU401 | 0.04780 | 0.06903 | 26 | 0.98507 | Alistipes |
| OTU204 | 0.04685 | 0.07191 | 27 | 0.98812 | Dialister |
| OTU288 | 0.04564 | 0.07576 | 28 | 1.00382 | Ruminococcaceae Incertae Sedis |
| OTU66 | 0.04482 | 0.07851 | 29 | 1.00441 | Streptococcus |
| OTU432 | 0.04450 | 0.07967 | 30 | 0.98528 | Paludibacter |
| OTU72 | 0.04432 | 0.08022 | 31 | 0.96009 | Aquabacterium |
| OTU151 | 0.04226 | 0.08778 | 32 | 1.01767 | Subdoligranulum |
| OTU167 | 0.04143 | 0.09100 | 33 | 1.02308 | Allobaculum |
| OTU80 | 0.04059 | 0.09443 | 34 | 1.03038 | Lachnospiraceae Incertae Sedis |
| OTU153 | 0.04043 | 0.09509 | 35 | 1.00798 | Roseburia |
| OTU146 | 0.03945 | 0.09927 | 36 | 1.02302 | Vibrio |
| OTU95 | 0.03897 | 0.10141 | 37 | 1.01683 | Ruminococcus |
| OTU420 | 0.03810 | 0.10547 | 38 | 1.02974 | Dorea |
| OTU547 | 0.03780 | 0.10677 | 39 | 1.01571 | Subdoligranulum |
| OTU352 | 0.03760 | 0.10776 | 40 | 0.99945 | Saprospira |
| OTU164 | 0.03704 | 0.11044 | 41 | 0.99931 | Faecalibacterium |
| OTU26 | 0.03681 | 0.11160 | 42 | 0.98578 | Dorea |
| OTU180 | 0.03632 | 0.11402 | 43 | 0.98373 | Roseburia |
| OTU373 | 0.03570 | 0.11708 | 44 | 0.98718 | Sporobacter |
| OTU23 | 0.03559 | 0.11780 | 45 | 0.97118 | Lachnospiraceae Incertae Sedis |
| OTU230 | 0.03428 | 0.12490 | 46 | 1.00738 | Butyrivibrio |
| OTU350 | 0.03420 | 0.12520 | 47 | 0.98831 | Coprococcus |
| OTU88 | 0.03418 | 0.12545 | 48 | 0.96966 | Streptococcus |
| OTU241 | 0.03414 | 0.12570 | 49 | 0.95172 | Chryseobacterium |
| OTU309 | 0.03300 | 0.13230 | 50 | 0.98164 | Paludibacter |
| OTU154 | 0.03088 | 0.14566 | 51 | 1.05962 | Faecalibacterium |
| OTU499 | 0.03070 | 0.14702 | 52 | 1.04891 | Lachnospiraceae Incertae Sedis |
| OTU21 | 0.03053 | 0.14799 | 53 | 1.03595 | Finegoldia |
| OTU452 | 0.03010 | 0.15062 | 54 | 1.03479 | Butyrivibrio |
| OTU399 | 0.02990 | 0.15230 | 55 | 1.02734 | Ralstonia |
| OTU96 | 0.02898 | 0.15887 | 56 | 1.05251 | Diaphorobacter |
| OTU195 | 0.02838 | 0.16331 | 57 | 1.06294 | Pseudoalteromonas |
| OTU186 | 0.02821 | 0.16461 | 58 | 1.05293 | Faecalibacterium |
| OTU470 | 0.02760 | 0.16933 | 59 | 1.06475 | Lachnospiraceae Incertae Sedis |
| OTU84 | 0.02759 | 0.16939 | 60 | 1.04742 | Marinomonas |
| OTU229 | 0.02747 | 0.17030 | 61 | 1.03575 | Coriobacterineae |
| OTU566 | 0.02738 | 0.17105 | 62 | 1.02355 | Dorea |
| OTU98 | 0.02716 | 0.17278 | 63 | 1.01746 | Lachnospiraceae Incertae Sedis |
| OTU104 | 0.02705 | 0.17369 | 64 | 1.00683 | Syntrophococcus |
| OTU111 | 0.02684 | 0.17532 | 65 | 1.00067 | Peptostreptococcaceae Incertae Sedis |
| OTU59 | 0.02682 | 0.17553 | 66 | 0.98668 | Acinetobacter |
| OTU267 | 0.02664 | 0.17697 | 67 | 0.97997 | Parabacteroides |
| OTU157 | 0.02651 | 0.17809 | 68 | 0.97165 | Marinilabilia |
| OTU182 | 0.02499 | 0.19123 | 69 | 1.02819 | Lachnospiraceae Incertae Sedis |
| OTU231 | 0.02456 | 0.19512 | 70 | 1.03411 | Anaerotruncus |
| OTU30 | 0.02451 | 0.19561 | 71 | 1.02215 | Bryantella |
| OTU214 | 0.02440 | 0.19663 | 72 | 1.0132 | Roseburia |
| OTU538 | 0.02330 | 0.20675 | 73 | 1.05076 | Lachnospiraceae Incertae Sedis |
| OTU464 | 0.02320 | 0.20799 | 74 | 1.04277 | Marinilabilia |
| OTU356 | 0.02290 | 0.21102 | 75 | 1.04383 | Novosphingobium |
| OTU376 | 0.02220 | 0.21838 | 76 | 1.06602 | Methylobacterium |
| OTU3 | 0.02217 | 0.21861 | 77 | 1.05332 | Lachnospiraceae Incertae Sedis |
| OTU416 | 0.02120 | 0.22887 | 78 | 1.0886 | Lachnospiraceae Incertae Sedis |
| OTU358 | 0.02080 | 0.23330 | 79 | 1.09562 | Roseburia |
| OTU197 | 0.02052 | 0.23674 | 80 | 1.0979 | Lactobacillus |
| OTU200 | 0.02050 | 0.23707 | 81 | 1.08584 | Helicobacter |
| OTU495 | 0.02040 | 0.23841 | 82 | 1.07867 | Streptococcus |
| OTU65 | 0.01999 | 0.24295 | 83 | 1.08596 | Lachnospiraceae Incertae Sedis |
| OTU454 | 0.02000 | 0.24329 | 84 | 1.07452 | Paludibacter |
| OTU425 | 0.01990 | 0.24367 | 85 | 1.06355 | Enhydrobacter |
| OTU46 | 0.01953 | 0.24861 | 86 | 1.07251 | Bacillaceae 1 |
| OTU155 | 0.01951 | 0.24887 | 87 | 1.06126 | Roseburia |
| OTU240 | 0.01947 | 0.24930 | 88 | 1.05105 | Weissella |
| OTU266 | 0.01923 | 0.25225 | 89 | 1.05153 | Bacteroides |
| OTU463 | 0.01920 | 0.25304 | 90 | 1.04308 | Lachnospiraceae Incertae Sedis |
| OTU107 | 0.01902 | 0.25492 | 91 | 1.03928 | Ruminococcus |
| OTU101 | 0.01890 | 0.25641 | 92 | 1.03401 | Pseudoalteromonas |
| OTU102 | 0.01859 | 0.26038 | 93 | 1.03872 | Lachnospiraceae Incertae Sedis |
| OTU82 | 0.01851 | 0.26140 | 94 | 1.03169 | Roseburia |
| OTU115 | 0.01843 | 0.26242 | 95 | 1.02482 | Roseburia |
| OTU51 | 0.01794 | 0.26901 | 96 | 1.0396 | Klebsiella |
| OTU392 | 0.01770 | 0.27267 | 97 | 1.04288 | Lachnospiraceae Incertae Sedis |
| OTU198 | 0.01753 | 0.27460 | 98 | 1.03955 | Lachnospiraceae Incertae Sedis |
| OTU334 | 0.01747 | 0.27545 | 99 | 1.03225 | Citrobacter |
| OTU423 | 0.01720 | 0.27857 | 100 | 1.03349 | Parasporobacterium |
| OTU371 | 0.01710 | 0.28002 | 101 | 1.02858 | Comamonas |
| OTU365 | 0.01710 | 0.28007 | 102 | 1.01868 | Succinispira |
| OTU367 | 0.01670 | 0.28614 | 103 | 1.03066 | Pseudomonas |
| OTU378 | 0.01660 | 0.28836 | 104 | 1.02867 | Bacillaceae 1 |
| OTU12 | 0.01642 | 0.29042 | 105 | 1.02615 | Bryantella |
| OTU47 | 0.01639 | 0.29086 | 106 | 1.01801 | Succinispira |
| OTU124 | 0.01633 | 0.29173 | 107 | 1.01152 | Lactobacillus |
| OTU212 | 0.01631 | 0.29201 | 108 | 1.00313 | Coprobacillus |
| OTU203 | 0.01613 | 0.29472 | 109 | 1.00314 | Rheinheimera |
| OTU456 | 0.01590 | 0.29808 | 110 | 1.00533 | Anaerovorax |
| OTU19 | 0.01563 | 0.30240 | 111 | 1.01072 | Syntrophococcus |
| OTU268 | 0.01537 | 0.30653 | 112 | 1.01537 | Staphylococcus |
| OTU60 | 0.01513 | 0.31036 | 113 | 1.01896 | Subdoligranulum |
| OTU50 | 0.01506 | 0.31153 | 114 | 1.01382 | Sutterella |
| OTU75 | 0.01487 | 0.31460 | 115 | 1.01494 | Stenotrophomonas |
| OTU192 | 0.01447 | 0.32129 | 116 | 1.02757 | Sphingomonas |
| OTU36 | 0.01438 | 0.32279 | 117 | 1.02354 | Bacteroides |
| OTU389 | 0.01430 | 0.32348 | 118 | 1.01705 | Parabacteroides |
| OTU28 | 0.01423 | 0.32534 | 119 | 1.01429 | Bacteroides |
| OTU6 | 0.01415 | 0.32671 | 120 | 1.01009 | Lachnospiraceae Incertae Sedis |
| OTU292 | 0.01378 | 0.33313 | 121 | 1.0214 | Alistipes |
| OTU282 | 0.01372 | 0.33422 | 122 | 1.01634 | Streptococcus |
| OTU194 | 0.01359 | 0.33650 | 123 | 1.01497 | Alistipes |
| OTU15 | 0.01342 | 0.33965 | 124 | 1.01622 | Roseburia |
| OTU37 | 0.01340 | 0.33987 | 125 | 1.00874 | Cloacibacterium |
| OTU300 | 0.01337 | 0.34042 | 126 | 1.00234 | Lachnospiraceae Incertae Sedis |
| OTU165 | 0.01333 | 0.34119 | 127 | 0.9967 | Alistipes |
| OTU188 | 0.01329 | 0.34201 | 128 | 0.99129 | Lachnospiraceae Incertae Sedis |
| OTU156 | 0.01310 | 0.34551 | 129 | 0.99369 | Lachnospiraceae Incertae Sedis |
| OTU304 | 0.01300 | 0.34727 | 130 | 0.99105 | Faecalibacterium |
| OTU299 | 0.01299 | 0.34741 | 131 | 0.98388 | Lachnospiraceae Incertae Sedis |
| OTU406 | 0.01300 | 0.34761 | 132 | 0.97701 | Bacteroides |
| OTU177 | 0.01289 | 0.34929 | 133 | 0.97433 | Butyrivibrio |
| OTU553 | 0.01251 | 0.35656 | 134 | 0.98718 | Syntrophococcus |
| OTU190 | 0.01250 | 0.35680 | 135 | 0.98053 | Ruminococcaceae Incertae Sedis |
| OTU429 | 0.01210 | 0.36396 | 136 | 0.99285 | Dorea |
| OTU149 | 0.01212 | 0.36424 | 137 | 0.98637 | Haemophilus |
| OTU24 | 0.01209 | 0.36477 | 138 | 0.98066 | Lachnospiraceae Incertae Sedis |
| OTU42 | 0.01196 | 0.36740 | 139 | 0.98061 | Prevotella |
| OTU136 | 0.01194 | 0.36780 | 140 | 0.97468 | Micrococcineae |
| OTU286 | 0.01183 | 0.37015 | 141 | 0.97395 | Hallella |
| OTU33 | 0.01131 | 0.38093 | 142 | 0.99523 | Lachnospiraceae Incertae Sedis |
| OTU455 | 0.01130 | 0.38152 | 143 | 0.98982 | Finegoldia |
| OTU418 | 0.01100 | 0.38698 | 144 | 0.997 | Stenotrophomonas |
| OTU91 | 0.01089 | 0.38984 | 145 | 0.99745 | Lactobacillus |
| OTU256 | 0.01057 | 0.39700 | 146 | 1.00883 | Anaerotruncus |
| OTU41 | 0.01030 | 0.40320 | 147 | 1.0176 | Subdoligranulum |
| OTU126 | 0.01009 | 0.40791 | 148 | 1.02254 | Aeromonas |
| OTU134 | 0.01007 | 0.40846 | 149 | 1.01703 | Ruminococcaceae Incertae Sedis |
| OTU396 | 0.00984 | 0.41387 | 150 | 1.02364 | Coprococcus |
| OTU244 | 0.00967 | 0.41805 | 151 | 1.02712 | Prevotella |
| OTU403 | 0.00966 | 0.41823 | 152 | 1.02081 | Methylobacterium |
| OTU344 | 0.00957 | 0.42046 | 153 | 1.01954 | Carnobacteriaceae 1 |
| OTU17 | 0.00947 | 0.42293 | 154 | 1.01888 | Escherichia |
| OTU491 | 0.00942 | 0.42407 | 155 | 1.01503 | Clostridiaceae 1 |
| OTU44 | 0.00929 | 0.42739 | 156 | 1.01641 | Lachnospiraceae Incertae Sedis |
| OTU29 | 0.00920 | 0.42964 | 157 | 1.01526 | Lachnospiraceae Incertae Sedis |
| OTU79 | 0.00897 | 0.43556 | 158 | 1.02274 | Lachnospiraceae Incertae Sedis |
| OTU284 | 0.00891 | 0.43701 | 159 | 1.01969 | Rubritepida |
| OTU324 | 0.00890 | 0.43714 | 160 | 1.01362 | Faecalibacterium |
| OTU366 | 0.00888 | 0.43768 | 161 | 1.00857 | Coprococcus |
| OTU248 | 0.00884 | 0.43878 | 162 | 1.00486 | Lachnospiraceae Incertae Sedis |
| OTU476 | 0.00881 | 0.43963 | 163 | 1.00062 | Streptococcus |
| OTU94 | 0.00876 | 0.44084 | 164 | 0.99725 | Anaerotruncus |
| OTU319 | 0.00861 | 0.44499 | 165 | 1.00054 | Agrobacterium |
| OTU87 | 0.00860 | 0.44510 | 166 | 0.99478 | Propionibacterineae |
| OTU11 | 0.00856 | 0.44623 | 167 | 0.99133 | Bacteroides |
| OTU404 | 0.00834 | 0.45223 | 168 | 0.99867 | Hallella |
| OTU45 | 0.00830 | 0.45326 | 169 | 0.99502 | Xenohaliotis |
| OTU61 | 0.00826 | 0.45441 | 170 | 0.99169 | Papillibacter |
| OTU283 | 0.00824 | 0.45488 | 171 | 0.9869 | Anaerophaga |
| OTU22 | 0.00814 | 0.45764 | 172 | 0.98711 | Acidovorax |
| OTU144 | 0.00814 | 0.45765 | 173 | 0.98144 | Dorea |
| OTU347 | 0.00805 | 0.46007 | 174 | 0.98094 | Vitellibacter |
| OTU285 | 0.00766 | 0.47129 | 175 | 0.99914 | Butyrivibrio |
| OTU424 | 0.00762 | 0.47244 | 176 | 0.99589 | Streptococcus |
| OTU189 | 0.00739 | 0.47908 | 177 | 1.00417 | Acidovorax |
| OTU417 | 0.00736 | 0.47998 | 178 | 1.0004 | Lachnobacterium |
| OTU34 | 0.00734 | 0.48061 | 179 | 0.99612 | Dorea |
| OTU525 | 0.00724 | 0.48367 | 180 | 0.99691 | Catonella |
| OTU7 | 0.00717 | 0.48574 | 181 | 0.99564 | Bacteroides |
| OTU32 | 0.00699 | 0.49123 | 182 | 1.00136 | Erysipelotrichaceae Incertae Sedis |
| OTU168 | 0.00696 | 0.49246 | 183 | 0.99838 | Roseburia |
| OTU265 | 0.00694 | 0.49309 | 184 | 0.99422 | Sphingomonas |
| OTU445 | 0.00686 | 0.49542 | 185 | 0.99352 | Corynebacterineae |
| OTU272 | 0.00661 | 0.50356 | 186 | 1.00441 | Sporobacter |
| OTU143 | 0.00640 | 0.51031 | 187 | 1.01243 | Lachnospiraceae Incertae Sedis |
| OTU31 | 0.00633 | 0.51268 | 188 | 1.01172 | Coprococcus |
| OTU48 | 0.00615 | 0.51875 | 189 | 1.01829 | Bacteroides |
| OTU184 | 0.00604 | 0.52262 | 190 | 1.02049 | Lachnospiraceae Incertae Sedis |
| OTU361 | 0.00599 | 0.52411 | 191 | 1.01804 | Succinivibrio |
| OTU243 | 0.00590 | 0.52745 | 192 | 1.01919 | Anaerotruncus |
| OTU159 | 0.00582 | 0.53006 | 193 | 1.01892 | Faecalibacterium |
| OTU400 | 0.00581 | 0.53056 | 194 | 1.01464 | Bryantella |
| OTU458 | 0.00574 | 0.53301 | 195 | 1.01409 | Roseburia |
| OTU253 | 0.00565 | 0.53639 | 196 | 1.01531 | Uruburuella |
| OTU74 | 0.00557 | 0.53901 | 197 | 1.01509 | Ruminococcus |
| OTU139 | 0.00546 | 0.54311 | 198 | 1.01765 | Azonexus |
| OTU199 | 0.00544 | 0.54396 | 199 | 1.01411 | Acetanaerobacterium |
| OTU364 | 0.00541 | 0.54523 | 200 | 1.0114 | Exiguobacterium |
| OTU129 | 0.00538 | 0.54619 | 201 | 1.00815 | Roseburia |
| OTU71 | 0.00534 | 0.54778 | 202 | 1.00608 | Lachnospiraceae Incertae Sedis |
| OTU317 | 0.00530 | 0.54939 | 203 | 1.00405 | Prevotella |
| OTU52 | 0.00529 | 0.54965 | 204 | 0.99961 | Lachnospiraceae Incertae Sedis |
| OTU53 | 0.00528 | 0.54981 | 205 | 0.99502 | Succinivibrio |
| OTU62 | 0.00497 | 0.56195 | 206 | 1.01205 | Ruminococcus |
| OTU9 | 0.00494 | 0.56331 | 207 | 1.00961 | Bacteroides |
| OTU311 | 0.00484 | 0.56729 | 208 | 1.01184 | Lachnospiraceae Incertae Sedis |
| OTU76 | 0.00483 | 0.56755 | 209 | 1.00747 | Lachnobacterium |
| OTU89 | 0.00483 | 0.56764 | 210 | 1.00282 | Bacteroides |
| OTU216 | 0.00471 | 0.57232 | 211 | 1.0063 | Sphingomonas |
| OTU58 | 0.00470 | 0.57286 | 212 | 1.00251 | Peptostreptococcaceae Incertae Sedis |
| OTU133 | 0.00469 | 0.57321 | 213 | 0.99841 | Faecalibacterium |
| OTU493 | 0.00435 | 0.58737 | 214 | 1.01829 | Lachnospiraceae Incertae Sedis |
| OTU327 | 0.00434 | 0.58810 | 215 | 1.01482 | Pelomonas |
| OTU49 | 0.00427 | 0.59075 | 216 | 1.01466 | Sutterella |
| OTU242 | 0.00427 | 0.59078 | 217 | 1.01005 | Coriobacterineae |
| OTU359 | 0.00427 | 0.59097 | 218 | 1.00573 | Faecalibacterium |
| OTU316 | 0.00424 | 0.59231 | 219 | 1.00341 | Alistipes |
| OTU73 | 0.00421 | 0.59368 | 220 | 1.00116 | Lactococcus |
| OTU2 | 0.00416 | 0.59600 | 221 | 1.00053 | Faecalibacterium |
| OTU484 | 0.00410 | 0.59856 | 222 | 1.00029 | Effluviibacter |
| OTU297 | 0.00408 | 0.59957 | 223 | 0.99749 | Bacillaceae 1 |
| OTU150 | 0.00406 | 0.60032 | 224 | 0.99428 | Ruminococcaceae Incertae Sedis |
| OTU239 | 0.00388 | 0.60851 | 225 | 1.00337 | Succinispira |
| OTU205 | 0.00376 | 0.61391 | 226 | 1.00778 | Erysipelotrichaceae Incertae Sedis |
| OTU38 | 0.00375 | 0.61436 | 227 | 1.00408 | Pseudomonas |
| OTU117 | 0.00370 | 0.61669 | 228 | 1.00347 | Naxibacter |
| OTU274 | 0.00366 | 0.61881 | 229 | 1.00253 | Lachnospiraceae Incertae Sedis |
| OTU341 | 0.00361 | 0.62128 | 230 | 1.00214 | Prevotella |
| OTU170 | 0.00359 | 0.62208 | 231 | 0.9991 | Bacteroides |
| OTU207 | 0.00358 | 0.62246 | 232 | 0.9954 | Succinispira |
| OTU90 | 0.00346 | 0.62846 | 233 | 1.00069 | Lachnospiraceae Incertae Sedis |
| OTU296 | 0.00337 | 0.63322 | 234 | 1.00396 | Papillibacter |
| OTU238 | 0.00333 | 0.63519 | 235 | 1.00279 | Lachnospiraceae Incertae Sedis |
| OTU227 | 0.00333 | 0.63529 | 236 | 0.9987 | Lachnospiraceae Incertae Sedis |
| OTU374 | 0.00321 | 0.64151 | 237 | 1.00423 | Lachnospiraceae Incertae Sedis |
| OTU114 | 0.00320 | 0.64157 | 238 | 1.0001 | Megamonas |
| OTU152 | 0.00316 | 0.64412 | 239 | 0.99986 | Faecalibacterium |
| OTU395 | 0.00315 | 0.64466 | 240 | 0.99653 | Subdoligranulum |
| OTU326 | 0.00296 | 0.65473 | 241 | 1.0079 | Lachnospiraceae Incertae Sedis |
| OTU226 | 0.00293 | 0.65630 | 242 | 1.00615 | Rikenella |
| OTU56 | 0.00271 | 0.66884 | 243 | 1.02115 | Delftia |
| OTU57 | 0.00270 | 0.66907 | 244 | 1.01731 | Lachnospiraceae Incertae Sedis |
| OTU249 | 0.00269 | 0.66999 | 245 | 1.01456 | Faecalibacterium |
| OTU187 | 0.00262 | 0.67379 | 246 | 1.01616 | Erysipelotrichaceae Incertae Sedis |
| OTU173 | 0.00255 | 0.67803 | 247 | 1.01842 | Anaerotruncus |
| OTU77 | 0.00255 | 0.67813 | 248 | 1.01446 | Coprococcus |
| OTU519 | 0.00254 | 0.67847 | 249 | 1.01089 | Catonella |
| OTU313 | 0.00252 | 0.67991 | 250 | 1.00899 | Enterobacter |
| OTU233 | 0.00249 | 0.68143 | 251 | 1.00722 | Syntrophococcus |
| OTU179 | 0.00241 | 0.68654 | 252 | 1.01074 | Ruminococcaceae Incertae Sedis |
| OTU506 | 0.00237 | 0.68930 | 253 | 1.01079 | Syntrophococcus |
| OTU103 | 0.00225 | 0.69653 | 254 | 1.01738 | Roseburia |
| OTU407 | 0.00223 | 0.69779 | 255 | 1.01521 | Turicibacter |
| OTU269 | 0.00222 | 0.69851 | 256 | 1.0123 | Butyrivibrio |
| OTU222 | 0.00220 | 0.69989 | 257 | 1.01035 | Prevotella |
| OTU193 | 0.00215 | 0.70341 | 258 | 1.01149 | Xylanibacter |
| OTU132 | 0.00199 | 0.71391 | 259 | 1.02263 | Parabacteroides |
| OTU411 | 0.00192 | 0.71867 | 260 | 1.02548 | Faecalibacterium |
| OTU109 | 0.00191 | 0.71934 | 261 | 1.02251 | Turicibacter |
| OTU181 | 0.00189 | 0.72104 | 262 | 1.02101 | Bacteroides |
| OTU413 | 0.00183 | 0.72484 | 263 | 1.0225 | Subdoligranulum |
| OTU508 | 0.00183 | 0.72503 | 264 | 1.01889 | Lachnospiraceae Incertae Sedis |
| OTU127 | 0.00172 | 0.73283 | 265 | 1.02596 | Lachnospiraceae Incertae Sedis |
| OTU219 | 0.00164 | 0.73945 | 266 | 1.03134 | Rikenella |
| OTU202 | 0.00152 | 0.74899 | 267 | 1.04073 | Lachnospiraceae Incertae Sedis |
| OTU158 | 0.00145 | 0.75455 | 268 | 1.04455 | Bacteroides |
| OTU113 | 0.00145 | 0.75468 | 269 | 1.04084 | Rikenella |
| OTU291 | 0.00143 | 0.75607 | 270 | 1.0389 | Syntrophococcus |
| OTU35 | 0.00138 | 0.75983 | 271 | 1.0402 | Bryantella |
| OTU69 | 0.00138 | 0.76032 | 272 | 1.03706 | Lachnospiraceae Incertae Sedis |
| OTU360 | 0.00138 | 0.76046 | 273 | 1.03345 | Faecalibacterium |
| OTU270 | 0.00137 | 0.76063 | 274 | 1.0299 | Succinispira |
| OTU569 | 0.00136 | 0.76170 | 275 | 1.0276 | Erwinia |
| OTU148 | 0.00121 | 0.77482 | 276 | 1.04151 | Lachnospiraceae Incertae Sedis |
| OTU206 | 0.00118 | 0.77735 | 277 | 1.04114 | Paludibacter |
| OTU338 | 0.00110 | 0.78478 | 278 | 1.04732 | Micrococcineae |
| OTU25 | 0.00110 | 0.78564 | 279 | 1.04471 | Parabacteroides |
| OTU108 | 0.00109 | 0.78588 | 280 | 1.04129 | Lachnospiraceae Incertae Sedis |
| OTU328 | 0.00104 | 0.79060 | 281 | 1.04382 | Parasporobacterium |
| OTU419 | 0.00104 | 0.79110 | 282 | 1.04078 | Micrococcineae |
| OTU225 | 0.00104 | 0.79121 | 283 | 1.03725 | Prevotella |
| OTU123 | 0.00104 | 0.79133 | 284 | 1.03375 | Papillibacter |
| OTU460 | 0.00098 | 0.79703 | 285 | 1.03754 | Lachnospiraceae Incertae Sedis |
| OTU70 | 0.00094 | 0.80105 | 286 | 1.03913 | Sphingobium |
| OTU1 | 0.00093 | 0.80167 | 287 | 1.0363 | Bacteroides |
| OTU387 | 0.00093 | 0.80206 | 288 | 1.03321 | Coprococcus |
| OTU345 | 0.00090 | 0.80526 | 289 | 1.03374 | Butyrivibrio |
| OTU137 | 0.00090 | 0.80547 | 290 | 1.03045 | Prevotella |
| OTU10 | 0.00089 | 0.80605 | 291 | 1.02764 | Coprobacillus |
| OTU312 | 0.00083 | 0.81254 | 292 | 1.03237 | Coriobacterineae |
| OTU307 | 0.00080 | 0.81611 | 293 | 1.03337 | Megamonas |
| OTU353 | 0.00079 | 0.81796 | 294 | 1.03218 | Dorea |
| OTU196 | 0.00078 | 0.81801 | 295 | 1.02875 | Bacteroides |
| OTU8 | 0.00078 | 0.81824 | 296 | 1.02556 | Dorea |
| OTU178 | 0.00072 | 0.82507 | 297 | 1.03064 | Lachnospiraceae Incertae Sedis |
| OTU106 | 0.00072 | 0.82581 | 298 | 1.02811 | Lachnospiraceae Incertae Sedis |
| OTU437 | 0.00071 | 0.82714 | 299 | 1.02632 | Marinilabilia |
| OTU393 | 0.00069 | 0.82865 | 300 | 1.02476 | Micrococcineae |
| OTU502 | 0.00067 | 0.83190 | 301 | 1.02536 | Paludibacter |
| OTU349 | 0.00066 | 0.83311 | 302 | 1.02345 | Syntrophococcus |
| OTU343 | 0.00065 | 0.83398 | 303 | 1.02115 | Lachnobacterium |
| OTU354 | 0.00064 | 0.83515 | 304 | 1.01921 | Anaerotruncus |
| OTU120 | 0.00064 | 0.83562 | 305 | 1.01644 | Micrococcineae |
| OTU368 | 0.00060 | 0.83993 | 306 | 1.01835 | Ruminococcaceae Incertae Sedis |
| OTU330 | 0.00060 | 0.84109 | 307 | 1.01643 | Coriobacterineae |
| OTU18 | 0.00058 | 0.84311 | 308 | 1.01557 | Faecalibacterium |
| OTU379 | 0.00055 | 0.84661 | 309 | 1.01647 | Roseburia |
| OTU355 | 0.00052 | 0.85194 | 310 | 1.01958 | Corynebacterineae |
| OTU169 | 0.00048 | 0.85685 | 311 | 1.02216 | Streptococcus |
| OTU217 | 0.00044 | 0.86299 | 312 | 1.02619 | Prevotella |
| OTU97 | 0.00044 | 0.86362 | 313 | 1.02365 | Pseudomonas |
| OTU315 | 0.00043 | 0.86508 | 314 | 1.02211 | Coriobacterineae |
| OTU453 | 0.00041 | 0.86851 | 315 | 1.02292 | Faecalibacterium |
| OTU293 | 0.00041 | 0.86858 | 316 | 1.01975 | Lachnospiraceae Incertae Sedis |
| OTU160 | 0.00039 | 0.87159 | 317 | 1.02006 | Lachnospiraceae Incertae Sedis |
| OTU93 | 0.00038 | 0.87290 | 318 | 1.01839 | Alistipes |
| OTU303 | 0.00037 | 0.87374 | 319 | 1.01617 | Faecalibacterium |
| OTU128 | 0.00036 | 0.87555 | 320 | 1.01509 | Prevotella |
| OTU86 | 0.00035 | 0.87754 | 321 | 1.01423 | Fusobacterium |
| OTU264 | 0.00035 | 0.87829 | 322 | 1.01195 | Comamonas |
| OTU171 | 0.00034 | 0.87891 | 323 | 1.00952 | Bacteroides |
| OTU100 | 0.00032 | 0.88369 | 324 | 1.01187 | Xylanibacter |
| OTU176 | 0.00032 | 0.88369 | 325 | 1.00877 | Erwinia |
| OTU235 | 0.00030 | 0.88760 | 326 | 1.01013 | Desulfovibrio |
| OTU142 | 0.00027 | 0.89298 | 327 | 1.01314 | Lachnospiraceae Incertae Sedis |
| OTU183 | 0.00025 | 0.89598 | 328 | 1.01344 | Bacteroides |
| OTU391 | 0.00024 | 0.89806 | 329 | 1.01271 | Aquiflexum |
| OTU85 | 0.00024 | 0.89815 | 330 | 1.00974 | Bacteroides |
| OTU224 | 0.00023 | 0.90135 | 331 | 1.01028 | Prevotella |
| OTU55 | 0.00023 | 0.90176 | 332 | 1.00769 | Parabacteroides |
| OTU166 | 0.00022 | 0.90242 | 333 | 1.00539 | Lachnospiraceae Incertae Sedis |
| OTU322 | 0.00021 | 0.90433 | 334 | 1.00451 | Roseburia |
| OTU14 | 0.00020 | 0.90785 | 335 | 1.0054 | Erysipelotrichaceae Incertae Sedis |
| OTU408 | 0.00019 | 0.90951 | 336 | 1.00425 | Bryantella |
| OTU54 | 0.00018 | 0.91151 | 337 | 1.00347 | Lachnospiraceae Incertae Sedis |
| OTU64 | 0.00017 | 0.91495 | 338 | 1.00428 | Erwinia |
| OTU83 | 0.00017 | 0.91541 | 339 | 1.00182 | Dorea |
| OTU68 | 0.00016 | 0.91804 | 340 | 1.00175 | Dorea |
| OTU5 | 0.00015 | 0.92125 | 341 | 1.0023 | Sphingomonas |
| OTU145 | 0.00014 | 0.92320 | 342 | 1.00149 | Afipia |
| OTU119 | 0.00014 | 0.92370 | 343 | 0.9991 | Lachnobacterium |
| OTU442 | 0.00011 | 0.93035 | 344 | 1.00337 | Roseburia |
| OTU412 | 0.00011 | 0.93055 | 345 | 1.00068 | Sphingomonas |
| OTU474 | 0.00011 | 0.93058 | 346 | 0.99781 | Sphingobium |
| OTU20 | 0.00011 | 0.93225 | 347 | 0.99673 | Lachnospiraceae Incertae Sedis |
| OTU254 | 0.00010 | 0.93343 | 348 | 0.99512 | Lachnospiraceae Incertae Sedis |
| OTU260 | 0.00010 | 0.93363 | 349 | 0.99248 | Erysipelotrichaceae Incertae Sedis |
| OTU287 | 0.00010 | 0.93561 | 350 | 0.99175 | Anaerovorax |
| OTU250 | 0.00009 | 0.93893 | 351 | 0.99243 | Paludibacter |
| OTU422 | 0.00009 | 0.93947 | 352 | 0.99018 | Peptococcaceae 1 |
| OTU140 | 0.00008 | 0.94086 | 353 | 0.98883 | Faecalibacterium |
| OTU421 | 0.00008 | 0.94289 | 354 | 0.98817 | Streptococcus |
| OTU161 | 0.00006 | 0.94925 | 355 | 0.99203 | Prevotella |
| OTU135 | 0.00006 | 0.94978 | 356 | 0.9898 | Clostridiaceae 1 |
| OTU375 | 0.00005 | 0.95255 | 357 | 0.98991 | Pseudomonas |
| OTU191 | 0.00005 | 0.95294 | 358 | 0.98754 | Subdoligranulum |
| OTU122 | 0.00004 | 0.95860 | 359 | 0.99064 | Prevotella |
| OTU162 | 0.00004 | 0.95894 | 360 | 0.98824 | Veillonella |
| OTU501 | 0.00004 | 0.95986 | 361 | 0.98645 | Ruminococcaceae Incertae Sedis |
| OTU275 | 0.00004 | 0.96063 | 362 | 0.98452 | Lachnospiraceae Incertae Sedis |
| OTU213 | 0.00004 | 0.96094 | 363 | 0.98212 | Lactococcus |
| OTU141 | 0.00003 | 0.96233 | 364 | 0.98084 | Faecalibacterium |
| OTU363 | 0.00003 | 0.96649 | 365 | 0.98238 | Faecalibacterium |
| OTU130 | 0.00002 | 0.97345 | 366 | 0.98675 | Lachnospiraceae Incertae Sedis |
| OTU409 | 0.00001 | 0.97609 | 367 | 0.98673 | Alkalilimnicola |
| OTU471 | 0.00001 | 0.97787 | 368 | 0.98584 | Lachnospiraceae Incertae Sedis |
| OTU247 | 0.00000 | 0.98560 | 369 | 0.99095 | Xylanibacter |
| OTU118 | 0.00000 | 0.99027 | 370 | 0.99295 | Burkholderia |
| OTU92 | 0.00000 | 0.99641 | 371 | 0.99641 | Rubrobacterineae |

Table 7: Regressions on log-normalized abundances of OTUs (97%) vs. WHRs of all samples with RDP classification of consensus sequences at genus level shown. Only OTUs which have at least 1 sequence assigned to them in 25% of the samples are shown. Regression p-Values were corrected for multiple testing using (n*p)/R where n=total number of taxa tested, p=raw p-Value and R=sorted rank of the taxon. Benjamini & Hochberg (1995).

TABLE 7

| OTUname | R2 | p-Value | Rank | (n*p)/R | RDP assignment |
|---|---|---|---|---|---|
| OTU4 | 0.16058 | 0.00053 | 1 | 0.19811 | Lachnospiraceae Incertae Sedis |
| OTU492 | 0.16000 | 0.00054 | 2 | 0.09998 | Coriobacterineae |
| OTU305 | 0.15413 | 0.00071 | 3 | 0.08756 | Lachnospiraceae Incertae Sedis |
| OTU79 | 0.09585 | 0.00861 | 4 | 0.79813 | Lachnospiraceae Incertae Sedis |
| OTU476 | 0.09510 | 0.00890 | 5 | 0.66061 | Streptococcus |
| OTU132 | 0.09057 | 0.01076 | 6 | 0.66561 | Parabacteroides |
| OTU123 | 0.09019 | 0.01094 | 7 | 0.57987 | Papillibacter |
| OTU31 | 0.07537 | 0.02050 | 8 | 0.95086 | Coprococcus |
| OTU249 | 0.07253 | 0.02314 | 9 | 0.9537 | Faecalibacterium |
| OTU416 | 0.06910 | 0.02679 | 10 | 0.99377 | Lachnospiraceae Incertae Sedis |
| OTU471 | 0.06680 | 0.02958 | 11 | 0.99774 | Lachnospiraceae Incertae Sedis |
| OTU3 | 0.06375 | 0.03364 | 12 | 1.04016 | Lachnospiraceae Incertae Sedis |
| OTU54 | 0.06336 | 0.03421 | 13 | 0.97625 | Lachnospiraceae Incertae Sedis |
| OTU36 | 0.06000 | 0.03952 | 14 | 1.0472 | Bacteroides |
| OTU282 | 0.05870 | 0.04177 | 15 | 1.03316 | Streptococcus |
| OTU162 | 0.05520 | 0.04858 | 16 | 1.12656 | Veillonella |
| OTU11 | 0.05483 | 0.04936 | 17 | 1.07724 | Bacteroides |
| OTU420 | 0.05420 | 0.05065 | 18 | 1.04393 | Dorea |
| OTU2 | 0.05334 | 0.05265 | 19 | 1.02803 | Faecalibacterium |
| OTU306 | 0.05307 | 0.05327 | 20 | 0.98819 | Oligotropha |
| OTU14 | 0.05298 | 0.05347 | 21 | 0.94458 | Erysipelotrichaceae Incertae Sedis |
| OTU122 | 0.04952 | 0.06214 | 22 | 1.04792 | Prevotella |
| OTU65 | 0.04587 | 0.07291 | 23 | 1.17604 | Lachnospiraceae Incertae Sedis |
| OTU242 | 0.04413 | 0.07870 | 24 | 1.21653 | Coriobacterineae |
| OTU199 | 0.04234 | 0.08517 | 25 | 1.26385 | Acetanaerobacterium |
| OTU330 | 0.04207 | 0.08618 | 26 | 1.22971 | Coriobacterineae |
| OTU239 | 0.04187 | 0.08696 | 27 | 1.19491 | Succinispira |
| OTU197 | 0.04077 | 0.09130 | 28 | 1.20971 | Lactobacillus |
| OTU229 | 0.03893 | 0.09909 | 29 | 1.26763 | Coriobacterineae |
| OTU149 | 0.03824 | 0.10219 | 30 | 1.26381 | Haemophilias |
| OTU28 | 0.03786 | 0.10396 | 31 | 1.24416 | Bacteroides |
| OTU49 | 0.03752 | 0.10553 | 32 | 1.2235 | Sutterella |
| OTU237 | 0.03741 | 0.10605 | 33 | 1.19224 | Prevotella |
| OTU29 | 0.03739 | 0.10616 | 34 | 1.15839 | Lachnospiraceae Incertae Sedis |
| OTU27 | 0.03664 | 0.10980 | 35 | 1.16391 | Lachnospiraceae Incertae Sedis |
| OTU74 | 0.03641 | 0.11095 | 36 | 1.14341 | Ruminococcus |
| OTU284 | 0.03627 | 0.11165 | 37 | 1.11954 | Rubritepida |
| OTU198 | 0.03622 | 0.11189 | 38 | 1.09235 | Lachnospiraceae Incertae Sedis |
| OTU329 | 0.03581 | 0.11399 | 39 | 1.08437 | Methanohalobium |
| OTU283 | 0.03545 | 0.11583 | 40 | 1.07435 | Anaerophaga |
| OTU72 | 0.03517 | 0.11730 | 41 | 1.06145 | Aquabacterium |
| OTU309 | 0.03504 | 0.11804 | 42 | 1.04269 | Paludibacter |
| OTU59 | 0.03413 | 0.12299 | 43 | 1.06115 | Acinetobacter |
| OTU470 | 0.03410 | 0.12300 | 44 | 1.03708 | Lachnospiraceae Incertae Sedis |
| OTU173 | 0.03391 | 0.12420 | 45 | 1.02394 | Anaerotruncus |
| OTU454 | 0.03280 | 0.13051 | 46 | 1.05262 | Paludibacter |
| OTU16 | 0.03271 | 0.13118 | 47 | 1.03546 | Lachnospiraceae Incertae Sedis |
| OTU356 | 0.03220 | 0.13429 | 48 | 1.03794 | Novosphingobium |
| OTU46 | 0.03150 | 0.13869 | 49 | 1.05007 | Bacillaceae 1 |
| OTU98 | 0.03113 | 0.14105 | 50 | 1.04662 | Lachnospiraceae Incertae Sedis |
| OTU288 | 0.03108 | 0.14138 | 51 | 1.02847 | Ruminococcaceae Incertae Sedis |
| OTU474 | 0.03040 | 0.14608 | 52 | 1.04224 | Sphingobium |
| OTU104 | 0.02913 | 0.15475 | 53 | 1.08326 | Syntrophococcus |
| OTU429 | 0.02890 | 0.15635 | 54 | 1.07418 | Dorea |
| OTU41 | 0.02856 | 0.15889 | 55 | 1.07178 | Subdoligranulum |
| OTU117 | 0.02834 | 0.16052 | 56 | 1.06347 | Naxibacter |
| OTU96 | 0.02828 | 0.16096 | 57 | 1.04767 | Diaphorobacter |
| OTU143 | 0.02795 | 0.16346 | 58 | 1.04555 | Lachnospiraceae Incertae Sedis |
| OTU367 | 0.02760 | 0.16620 | 59 | 1.04507 | Pseudomonas |
| OTU34 | 0.02734 | 0.16820 | 60 | 1.04003 | Dorea |
| OTU200 | 0.02721 | 0.16926 | 61 | 1.02946 | Helicobacter |
| OTU525 | 0.02660 | 0.17395 | 62 | 1.04092 | Catonella |
| OTU42 | 0.02657 | 0.17443 | 63 | 1.02721 | Prevotella |
| OTU376 | 0.02630 | 0.17634 | 64 | 1.02221 | Methylobacterium |
| OTU128 | 0.02590 | 0.18004 | 65 | 1.02761 | Prevotella |
| OTU368 | 0.02540 | 0.18463 | 66 | 1.03784 | Ruminococcaceae Incertae Sedis |
| OTU58 | 0.02536 | 0.18466 | 67 | 1.0225 | Peptostreptococcaceae Incertae Sedis |
| OTU349 | 0.02528 | 0.18537 | 68 | 1.01137 | Syntrophococcus |
| OTU268 | 0.02473 | 0.19030 | 69 | 1.02319 | Staphylococcus |
| OTU88 | 0.02472 | 0.19038 | 70 | 1.00902 | Streptococcus |
| OTU327 | 0.02412 | 0.19593 | 71 | 1.02381 | Pelomonas |
| OTU370 | 0.02370 | 0.19945 | 72 | 1.02772 | Lactobacillus |
| OTU134 | 0.02349 | 0.20191 | 73 | 1.02617 | Ruminococcaceae Incertae Sedis |
| OTU150 | 0.02343 | 0.20256 | 74 | 1.01552 | Ruminococcaceae Incertae Sedis |
| OTU203 | 0.02326 | 0.20419 | 75 | 1.01007 | Rheinheimera |
| OTU391 | 0.02320 | 0.20459 | 76 | 0.99874 | Aquiflexum |
| OTU363 | 0.02250 | 0.21188 | 77 | 1.02088 | Faecalibacterium |
| OTU413 | 0.02250 | 0.21201 | 78 | 1.00838 | Subdoligranulum |
| OTU231 | 0.02211 | 0.21589 | 79 | 1.01386 | Anaerotruncus |
| OTU66 | 0.02207 | 0.21626 | 80 | 1.00289 | Streptococcus |
| OTU350 | 0.02190 | 0.21793 | 81 | 0.99816 | Coprococcus |
| OTU269 | 0.02141 | 0.22340 | 82 | 1.01077 | Butyrivibrio |
| OTU131 | 0.02120 | 0.22564 | 83 | 1.0086 | Lachnospiraceae Incertae Sedis |
| OTU61 | 0.02022 | 0.23682 | 84 | 1.04596 | Papillibacter |
| OTU235 | 0.02020 | 0.23709 | 85 | 1.03484 | Desulfovibrio |
| OTU343 | 0.02019 | 0.23722 | 86 | 1.02337 | Lachnobacterium |
| OTU172 | 0.01971 | 0.24294 | 87 | 1.03601 | Marinilabilia |
| OTU299 | 0.01952 | 0.24515 | 88 | 1.03353 | Lachnospiraceae Incertae Sedis |
| OTU425 | 0.01920 | 0.24895 | 89 | 1.03778 | Enhydrobacter |
| OTU213 | 0.01908 | 0.25071 | 90 | 1.0335 | Lactococcus |
| OTU25 | 0.01902 | 0.25143 | 91 | 1.02507 | Parabacteroides |
| OTU140 | 0.01892 | 0.25267 | 92 | 1.01892 | Faecalibacterium |
| OTU403 | 0.01870 | 0.25498 | 93 | 1.01717 | Methylobacterium |
| OTU204 | 0.01831 | 0.26054 | 94 | 1.02831 | Dialister |
| OTU157 | 0.01811 | 0.26320 | 95 | 1.02788 | Marinilabilia |
| OTU359 | 0.01780 | 0.26799 | 96 | 1.03568 | Faecalibacterium |
| OTU214 | 0.01759 | 0.27025 | 97 | 1.03365 | Roseburia |
| OTU566 | 0.01752 | 0.27111 | 98 | 1.02633 | Dorea |
| OTU37 | 0.01740 | 0.27290 | 99 | 1.02267 | Cloacibacterium |
| OTU371 | 0.01740 | 0.27331 | 100 | 1.01397 | Comamonas |
| OTU18 | 0.01721 | 0.27546 | 101 | 1.01184 | Faecalibacterium |
| OTU146 | 0.01721 | 0.27553 | 102 | 1.00216 | Vibrio |
| OTU354 | 0.01710 | 0.27690 | 103 | 0.99738 | Anaerotruncus |
| OTU357 | 0.01690 | 0.27932 | 104 | 0.99642 | Coprococcus |
| OTU334 | 0.01680 | 0.28133 | 105 | 0.99405 | Citrobacter |
| OTU352 | 0.01630 | 0.28894 | 106 | 1.0113 | Saprospira |
| OTU274 | 0.01605 | 0.29249 | 107 | 1.01413 | Lachnospiraceae Incertae Sedis |
| OTU326 | 0.01598 | 0.29346 | 108 | 1.0081 | Lachnospiraceae Incertae Sedis |
| OTU1 | 0.01594 | 0.29407 | 109 | 1.00092 | Bacteroides |
| OTU191 | 0.01560 | 0.29941 | 110 | 1.00983 | Subdoligranulum |
| OTU40 | 0.01507 | 0.30780 | 111 | 1.02877 | Lachnospiraceae Incertae Sedis |
| OTU226 | 0.01504 | 0.30832 | 112 | 1.0213 | Rikenella |
| OTU48 | 0.01480 | 0.31210 | 113 | 1.02469 | Bacteroides |
| OTU39 | 0.01476 | 0.31278 | 114 | 1.0179 | Coriobacterineae |
| OTU364 | 0.01470 | 0.31323 | 115 | 1.0105 | Exiguobacterium |
| OTU178 | 0.01467 | 0.31438 | 116 | 1.00547 | Lachnospiraceae Incertae Sedis |
| OTU113 | 0.01446 | 0.31778 | 117 | 1.00765 | Rikenella |
| OTU32 | 0.01434 | 0.31990 | 118 | 1.0058 | Erysipelotrichaceae Incertae Sedis |
| OTU296 | 0.01416 | 0.32295 | 119 | 1.00685 | Papillibacter |
| OTU153 | 0.01415 | 0.32311 | 120 | 0.99894 | Roseburia |
| OTU502 | 0.01410 | 0.32410 | 121 | 0.99373 | Paludibacter |
| OTU324 | 0.01390 | 0.32745 | 122 | 0.99577 | Faecalibacterium |
| OTU110 | 0.01387 | 0.32801 | 123 | 0.98936 | Lachnospiraceae Incertae Sedis |
| OTU315 | 0.01382 | 0.32888 | 124 | 0.98397 | Coriobacterineae |
| OTU102 | 0.01344 | 0.33568 | 125 | 0.99631 | Lachnospiraceae Incertae Sedis |
| OTU193 | 0.01339 | 0.33664 | 126 | 0.99121 | Xylanibacter |
| OTU15 | 0.01337 | 0.33695 | 127 | 0.98432 | Roseburia |
| OTU103 | 0.01314 | 0.34116 | 128 | 0.98882 | Roseburia |
| OTU184 | 0.01280 | 0.34746 | 129 | 0.99928 | Lachnospiraceae Incertae Sedis |
| OTU169 | 0.01267 | 0.34993 | 130 | 0.99865 | Streptococcus |
| OTU23 | 0.01263 | 0.35081 | 131 | 0.99351 | Lachnospiraceae Incertae Sedis |
| OTU53 | 0.01249 | 0.35340 | 132 | 0.99326 | Succinivibrio |
| OTU247 | 0.01237 | 0.35585 | 133 | 0.99263 | Xylanibacter |
| OTU7 | 0.01232 | 0.35687 | 134 | 0.98806 | Bacteroides |
| OTU20 | 0.01229 | 0.35738 | 135 | 0.98213 | Lachnospiraceae Incertae Sedis |
| OTU77 | 0.01223 | 0.35855 | 136 | 0.97811 | Coprococcus |
| OTU358 | 0.01210 | 0.36096 | 137 | 0.97748 | Roseburia |
| OTU423 | 0.01200 | 0.36253 | 138 | 0.97464 | Parasporobacterium |
| OTU508 | 0.01190 | 0.36508 | 139 | 0.97443 | Lachnospiraceae Incertae Sedis |
| OTU322 | 0.01160 | 0.37141 | 140 | 0.98423 | Roseburia |
| OTU84 | 0.01152 | 0.37297 | 141 | 0.98135 | Marinomonas |
| OTU210 | 0.01152 | 0.37298 | 142 | 0.97447 | Allobaculum |
| OTU22 | 0.01147 | 0.37410 | 143 | 0.97058 | Acidovorax |
| OTU380 | 0.01120 | 0.37870 | 144 | 0.97568 | Sporobacter |
| OTU553 | 0.01109 | 0.38216 | 145 | 0.97781 | Syntrophococcus |
| OTU389 | 0.01090 | 0.38598 | 146 | 0.9808 | Parabacteroides |
| OTU392 | 0.01060 | 0.39195 | 147 | 0.98921 | Lachnospiraceae Incertae Sedis |
| OTU344 | 0.01063 | 0.39231 | 148 | 0.98343 | Carnobacteriaceae 1 |
| OTU506 | 0.01060 | 0.39366 | 149 | 0.98018 | Syntrophococcus |
| OTU177 | 0.01020 | 0.40194 | 150 | 0.99414 | Butyrivibrio |
| OTU399 | 0.01000 | 0.40554 | 151 | 0.99638 | Ralstonia |
| OTU300 | 0.00991 | 0.40888 | 152 | 0.99798 | Lachnospiraceae Incertae Sedis |
| OTU316 | 0.00972 | 0.41345 | 153 | 1.00255 | Alistipes |
| OTU456 | 0.00959 | 0.41661 | 154 | 1.00364 | Anaerovorax |
| OTU293 | 0.00946 | 0.41960 | 155 | 1.00433 | Lachnospiraceae Incertae Sedis |
| OTU21 | 0.00935 | 0.42250 | 156 | 1.0048 | Finegoldia |
| OTU361 | 0.00922 | 0.42574 | 157 | 1.00604 | Succinivibrio |
| OTU202 | 0.00914 | 0.42775 | 158 | 1.00439 | Lachnospiraceae Incertae Sedis |
| OTU366 | 0.00895 | 0.43267 | 159 | 1.00957 | Coprococcus |
| OTU35 | 0.00884 | 0.43540 | 160 | 1.00958 | Bryantella |
| OTU275 | 0.00833 | 0.44901 | 161 | 1.03468 | Lachnospiraceae Incertae Sedis |
| OTU126 | 0.00830 | 0.44997 | 162 | 1.03049 | Aeromonas |
| OTU189 | 0.00828 | 0.45054 | 163 | 1.02547 | Acidovorax |
| OTU158 | 0.00826 | 0.45096 | 164 | 1.02016 | Bacteroides |
| OTU43 | 0.00807 | 0.45634 | 165 | 1.02607 | Lachnospiraceae Incertae Sedis |
| OTU105 | 0.00801 | 0.45787 | 166 | 1.02332 | Bacteroides |
| OTU9 | 0.00797 | 0.45913 | 167 | 1.01997 | Bacteroides |
| OTU297 | 0.00745 | 0.47430 | 168 | 1.04742 | Bacillaceae 1 |
| OTU80 | 0.00741 | 0.47546 | 169 | 1.04376 | Lachnospiraceae Incertae Sedis |
| OTU277 | 0.00732 | 0.47801 | 170 | 1.04318 | Lachnospiraceae Incertae Sedis |
| OTU395 | 0.00727 | 0.47963 | 171 | 1.04061 | Subdoligranulum |
| OTU365 | 0.00727 | 0.47972 | 172 | 1.03475 | Succinispira |
| OTU67 | 0.00726 | 0.47982 | 173 | 1.02898 | Lactobacillus |
| OTU372 | 0.00714 | 0.48370 | 174 | 1.03133 | Allomonas |
| OTU419 | 0.00701 | 0.48759 | 175 | 1.03368 | Micrococcineae |
| OTU101 | 0.00697 | 0.48875 | 176 | 1.03027 | Pseudoalteromonas |
| OTU10 | 0.00692 | 0.49053 | 177 | 1.02818 | Coprobacillus |
| OTU154 | 0.00685 | 0.49266 | 178 | 1.02683 | Faecalibacterium |
| OTU93 | 0.00677 | 0.49515 | 179 | 1.02626 | Alistipes |
| OTU62 | 0.00672 | 0.49684 | 180 | 1.02404 | Ruminococcus |
| OTU404 | 0.00645 | 0.50544 | 181 | 1.03602 | Hallella |
| OTU406 | 0.00645 | 0.50564 | 182 | 1.03073 | Bacteroides |
| OTU241 | 0.00635 | 0.50892 | 183 | 1.03175 | Chryseobacterium |
| OTU151 | 0.00634 | 0.50932 | 184 | 1.02695 | Subdoligranulum |
| OTU307 | 0.00629 | 0.51093 | 185 | 1.02461 | Megamonas |
| OTU155 | 0.00621 | 0.51362 | 186 | 1.02448 | Roseburia |
| OTU264 | 0.00619 | 0.51413 | 187 | 1.02 | Comamonas |
| OTU124 | 0.00607 | 0.51856 | 188 | 1.02333 | Lactobacillus |
| OTU227 | 0.00595 | 0.52273 | 189 | 1.0261 | Lachnospiraceae Incertae Sedis |
| OTU12 | 0.00581 | 0.52761 | 190 | 1.03023 | Bryantella |
| OTU442 | 0.00580 | 0.52800 | 191 | 1.02559 | Roseburia |
| OTU187 | 0.00572 | 0.53082 | 192 | 1.0257 | Erysipelotrichaceae Incertae Sedis |
| OTU45 | 0.00570 | 0.53138 | 193 | 1.02145 | Xenohaliotis |
| OTU240 | 0.00562 | 0.53429 | 194 | 1.02176 | Weissella |
| OTU95 | 0.00533 | 0.54511 | 195 | 1.03711 | Ruminococcus |
| OTU87 | 0.00532 | 0.54540 | 196 | 1.03237 | Propionibacterineae |
| OTU129 | 0.00524 | 0.54849 | 197 | 1.03295 | Roseburia |
| OTU243 | 0.00519 | 0.55054 | 198 | 1.03156 | Anaerotruncus |
| OTU133 | 0.00517 | 0.55109 | 199 | 1.02741 | Faecalibacterium |
| OTU401 | 0.00516 | 0.55153 | 200 | 1.0231 | Alistipes |
| OTU421 | 0.00511 | 0.55354 | 201 | 1.02171 | Streptococcus |
| OTU152 | 0.00508 | 0.55466 | 202 | 1.01871 | Faecalibacterium |
| OTU253 | 0.00503 | 0.55669 | 203 | 1.0174 | Uruburuella |
| OTU171 | 0.00501 | 0.55767 | 204 | 1.01419 | Bacteroides |
| OTU109 | 0.00499 | 0.55827 | 205 | 1.01034 | Turicibacter |
| OTU445 | 0.00483 | 0.56471 | 206 | 1.01703 | Corynebacterineae |
| OTU137 | 0.00471 | 0.56939 | 207 | 1.0205 | Prevotella |
| OTU100 | 0.00458 | 0.57482 | 208 | 1.02527 | Xylanibacter |
| OTU130 | 0.00454 | 0.57648 | 209 | 1.02333 | Lachnospiraceae Incertae Sedis |
| OTU328 | 0.00454 | 0.57671 | 210 | 1.01885 | Parasporobacterium |
| OTU378 | 0.00452 | 0.57768 | 211 | 1.01573 | Bacillaceae 1 |
| OTU183 | 0.00442 | 0.58181 | 212 | 1.01816 | Bacteroides |
| OTU26 | 0.00438 | 0.58344 | 213 | 1.01623 | Dorea |
| OTU432 | 0.00438 | 0.58352 | 214 | 1.01161 | Paludibacter |
| OTU317 | 0.00426 | 0.58867 | 215 | 1.01579 | Prevotella |
| OTU256 | 0.00424 | 0.58935 | 216 | 1.01226 | Anaerotruncus |
| OTU353 | 0.00424 | 0.58952 | 217 | 1.00789 | Dorea |
| OTU114 | 0.00424 | 0.58957 | 218 | 1.00335 | Megamonas |
| OTU453 | 0.00421 | 0.59104 | 219 | 1.00126 | Faecalibacterium |
| OTU94 | 0.00411 | 0.59542 | 220 | 1.0041 | Anaerotruncus |
| OTU460 | 0.00405 | 0.59791 | 221 | 1.00373 | Lachnospiraceae Incertae Sedis |
| OTU194 | 0.00393 | 0.60353 | 222 | 1.0086 | Alistipes |
| OTU159 | 0.00384 | 0.60749 | 223 | 1.01066 | Faecalibacterium |
| OTU141 | 0.00369 | 0.61465 | 224 | 1.01802 | Faecalibacterium |
| OTU90 | 0.00369 | 0.61470 | 225 | 1.01357 | Lachnospiraceae Incertae Sedis |
| OTU217 | 0.00367 | 0.61566 | 226 | 1.01066 | Prevotella |
| OTU397 | 0.00363 | 0.61788 | 227 | 1.00983 | Peptostreptococcaceae Incertae Sedis |
| OTU374 | 0.00353 | 0.62263 | 228 | 1.01314 | Lachnospiraceae Incertae Sedis |
| OTU148 | 0.00345 | 0.62636 | 229 | 1.01476 | Lachnospiraceae Incertae Sedis |
| OTU19 | 0.00337 | 0.63044 | 230 | 1.01693 | Syntrophococcus |
| OTU422 | 0.00334 | 0.63195 | 231 | 1.01495 | Peptococcaceae 1 |
| OTU418 | 0.00309 | 0.64516 | 232 | 1.0317 | Stenotrophomonas |
| OTU33 | 0.00308 | 0.64573 | 233 | 1.02818 | Lachnospiraceae Incertae Sedis |
| OTU38 | 0.00307 | 0.64629 | 234 | 1.02467 | Pseudomonas |
| OTU75 | 0.00303 | 0.64841 | 235 | 1.02366 | Stenotrophomonas |
| OTU138 | 0.00287 | 0.65749 | 236 | 1.0336 | Simkania |
| OTU396 | 0.00276 | 0.66333 | 237 | 1.03838 | Coprococcus |
| OTU311 | 0.00274 | 0.66482 | 238 | 1.03634 | Lachnospiraceae Incertae Sedis |
| OTU73 | 0.00270 | 0.66679 | 239 | 1.03505 | Lactococcus |
| OTU455 | 0.00255 | 0.67552 | 240 | 1.04424 | Finegoldia |
| OTU407 | 0.00250 | 0.67877 | 241 | 1.04492 | Turicibacter |
| OTU238 | 0.00247 | 0.68035 | 242 | 1.04301 | Lachnospiraceae Incertae Sedis |
| OTU501 | 0.00245 | 0.68189 | 243 | 1.04107 | Ruminococcaceae Incertae Sedis |
| OTU6 | 0.00241 | 0.68430 | 244 | 1.04047 | Lachnospiraceae Incertae Sedis |
| OTU225 | 0.00236 | 0.68772 | 245 | 1.04141 | Prevotella |
| OTU347 | 0.00233 | 0.68910 | 246 | 1.03925 | Vitellibacter |
| OTU355 | 0.00229 | 0.69179 | 247 | 1.03909 | Corynebacterineae |
| OTU135 | 0.00229 | 0.69192 | 248 | 1.03508 | Clostridiaceae 1 |
| OTU8 | 0.00225 | 0.69454 | 249 | 1.03483 | Dorea |
| OTU417 | 0.00225 | 0.69474 | 250 | 1.031 | Lachnobacterium |
| OTU30 | 0.00217 | 0.69963 | 251 | 1.03412 | Bryantella |
| OTU484 | 0.00210 | 0.70453 | 252 | 1.03722 | Effluviibacter |
| OTU265 | 0.00199 | 0.71215 | 253 | 1.04431 | Sphingomonas |
| OTU24 | 0.00195 | 0.71462 | 254 | 1.04379 | Lachnospiraceae Incertae Sedis |
| OTU224 | 0.00194 | 0.71545 | 255 | 1.04092 | Prevotella |
| OTU219 | 0.00181 | 0.72457 | 256 | 1.05005 | Rikenella |
| OTU499 | 0.00174 | 0.72958 | 257 | 1.0532 | Lachnospiraceae Incertae Sedis |
| OTU192 | 0.00171 | 0.73229 | 258 | 1.05302 | Sphingomonas |
| OTU212 | 0.00169 | 0.73349 | 259 | 1.05067 | Coprobacillus |
| OTU312 | 0.00164 | 0.73726 | 260 | 1.05202 | Coriobacterineae |
| OTU55 | 0.00163 | 0.73794 | 261 | 1.04895 | Parabacteroides |
| OTU286 | 0.00163 | 0.73815 | 262 | 1.04524 | Hallella |
| OTU142 | 0.00158 | 0.74217 | 263 | 1.04693 | Lachnospiraceae Incertae Sedis |
| OTU106 | 0.00155 | 0.74467 | 264 | 1.04648 | Lachnospiraceae Incertae Sedis |
| OTU161 | 0.00144 | 0.75323 | 265 | 1.05452 | Prevotella |
| OTU165 | 0.00141 | 0.75569 | 266 | 1.05399 | Alistipes |
| OTU186 | 0.00139 | 0.75723 | 267 | 1.05218 | Faecalibacterium |
| OTU439 | 0.00136 | 0.76031 | 268 | 1.05251 | Algibacter |
| OTU291 | 0.00135 | 0.76100 | 269 | 1.04956 | Syntrophococcus |
| OTU108 | 0.00123 | 0.77123 | 270 | 1.05973 | Lachnospiraceae Incertae Sedis |
| OTU424 | 0.00123 | 0.77154 | 271 | 1.05624 | Streptococcus |
| OTU176 | 0.00120 | 0.77451 | 272 | 1.05641 | Erwinia |
| OTU119 | 0.00117 | 0.77710 | 273 | 1.05605 | Lachnobacterium |
| OTU338 | 0.00116 | 0.77791 | 274 | 1.0533 | Micrococcineae |
| OTU206 | 0.00106 | 0.78756 | 275 | 1.06249 | Paludibacter |
| OTU182 | 0.00105 | 0.78893 | 276 | 1.06048 | Lachnospiraceae Incertae Sedis |
| OTU118 | 0.00104 | 0.78945 | 277 | 1.05735 | Burkholderia |
| OTU57 | 0.00104 | 0.78976 | 278 | 1.05395 | Lachnospiraceae Incertae Sedis |
| OTU17 | 0.00098 | 0.79508 | 279 | 1.05725 | Escherichia |
| OTU60 | 0.00096 | 0.79778 | 280 | 1.05705 | Subdoligranulum |
| OTU89 | 0.00094 | 0.79996 | 281 | 1.05618 | Bacteroides |
| OTU111 | 0.00092 | 0.80186 | 282 | 1.05493 | Peptostreptococcaceae Incertae Sedis |
| OTU144 | 0.00088 | 0.80648 | 283 | 1.05726 | Dorea |
| OTU181 | 0.00087 | 0.80664 | 284 | 1.05375 | Bacteroides |
| OTU411 | 0.00081 | 0.81405 | 285 | 1.0597 | Faecalibacterium |
| OTU127 | 0.00080 | 0.81495 | 286 | 1.05715 | Lachnospiraceae Incertae Sedis |
| OTU91 | 0.00069 | 0.82817 | 287 | 1.07056 | Lactobacillus |
| OTU285 | 0.00068 | 0.82973 | 288 | 1.06886 | Butyrivibrio |
| OTU195 | 0.00067 | 0.83061 | 289 | 1.06628 | Pseudoalteromonas |
| OTU379 | 0.00067 | 0.83079 | 290 | 1.06284 | Roseburia |
| OTU266 | 0.00065 | 0.83282 | 291 | 1.06177 | Bacteroides |
| OTU145 | 0.00063 | 0.83611 | 292 | 1.06231 | Afipia |
| OTU56 | 0.00062 | 0.83641 | 293 | 1.05907 | Delftia |
| OTU76 | 0.00062 | 0.83735 | 294 | 1.05666 | Lachnobacterium |
| OTU292 | 0.00057 | 0.84278 | 295 | 1.05991 | Alistipes |
| OTU168 | 0.00056 | 0.84464 | 296 | 1.05865 | Roseburia |
| OTU179 | 0.00056 | 0.84494 | 297 | 1.05546 | Ruminococcaceae Incertae Sedis |
| OTU538 | 0.00046 | 0.85925 | 298 | 1.06974 | Lachnospiraceae Incertae Sedis |
| OTU319 | 0.00043 | 0.86444 | 299 | 1.07259 | Agrobacterium |
| OTU360 | 0.00042 | 0.86578 | 300 | 1.07068 | Faecalibacterium |
| OTU120 | 0.00041 | 0.86755 | 301 | 1.06931 | Micrococcineae |
| OTU188 | 0.00040 | 0.86888 | 302 | 1.0674 | Lachnospiraceae Incertae Sedis |
| OTU50 | 0.00040 | 0.86920 | 303 | 1.06427 | Sutterella |
| OTU387 | 0.00040 | 0.86939 | 304 | 1.061 | Coprococcus |
| OTU493 | 0.00038 | 0.87259 | 305 | 1.06141 | Lachnospiraceae Incertae Sedis |
| OTU167 | 0.00036 | 0.87483 | 306 | 1.06066 | Allobaculum |
| OTU375 | 0.00036 | 0.87558 | 307 | 1.05811 | Pseudomonas |
| OTU412 | 0.00035 | 0.87630 | 308 | 1.05554 | Sphingomonas |
| OTU250 | 0.00033 | 0.87983 | 309 | 1.05636 | Paludibacter |
| OTU409 | 0.00032 | 0.88166 | 310 | 1.05514 | Alkalilimnicola |
| OTU136 | 0.00032 | 0.88268 | 311 | 1.05298 | Micrococcineae |
| OTU51 | 0.00031 | 0.88342 | 312 | 1.05047 | Klebsiella |
| OTU373 | 0.00029 | 0.88727 | 313 | 1.05168 | Sporobacter |
| OTU164 | 0.00029 | 0.88754 | 314 | 1.04866 | Faecalibacterium |
| OTU115 | 0.00028 | 0.89031 | 315 | 1.04859 | Roseburia |
| OTU260 | 0.00028 | 0.89035 | 316 | 1.04532 | Erysipelotrichaceae Incertae Sedis |
| OTU491 | 0.00028 | 0.89058 | 317 | 1.04229 | Clostridiaceae 1 |
| OTU97 | 0.00027 | 0.89157 | 318 | 1.04016 | Pseudomonas |
| OTU408 | 0.00025 | 0.89598 | 319 | 1.04204 | Bryantella |
| OTU207 | 0.00023 | 0.90106 | 320 | 1.04466 | Succinispira |
| OTU107 | 0.00023 | 0.90113 | 321 | 1.04149 | Ruminococcus |
| OTU452 | 0.00020 | 0.90578 | 322 | 1.04362 | Butyrivibrio |
| OTU341 | 0.00020 | 0.90713 | 323 | 1.04193 | Prevotella |
| OTU287 | 0.00020 | 0.90727 | 324 | 1.03888 | Anaerovorax |
| OTU156 | 0.00019 | 0.90839 | 325 | 1.03696 | Lachnospiraceae Incertae Sedis |
| OTU216 | 0.00016 | 0.91636 | 326 | 1.04285 | Sphingomonas |
| OTU86 | 0.00016 | 0.91719 | 327 | 1.0406 | Fusobacterium |
| OTU92 | 0.00016 | 0.91754 | 328 | 1.03783 | Rubrobacterineae |
| OTU205 | 0.00013 | 0.92564 | 329 | 1.04381 | Erysipelotrichaceae Incertae Sedis |
| OTU180 | 0.00013 | 0.92568 | 330 | 1.04068 | Roseburia |
| OTU230 | 0.00012 | 0.92648 | 331 | 1.03844 | Butyrivibrio |
| OTU196 | 0.00012 | 0.92666 | 332 | 1.03552 | Bacteroides |
| OTU166 | 0.00012 | 0.92794 | 333 | 1.03383 | Lachnospiraceae Incertae Sedis |
| OTU139 | 0.00011 | 0.93013 | 334 | 1.03317 | Azonexus |
| OTU83 | 0.00011 | 0.93076 | 335 | 1.03078 | Dorea |
| OTU82 | 0.00010 | 0.93505 | 336 | 1.03245 | Roseburia |
| OTU254 | 0.00009 | 0.93617 | 337 | 1.03062 | Lachnospiraceae Incertae Sedis |
| OTU304 | 0.00009 | 0.93661 | 338 | 1.02806 | Faecalibacterium |
| OTU222 | 0.00009 | 0.93812 | 339 | 1.02667 | Prevotella |
| OTU5 | 0.00008 | 0.93979 | 340 | 1.02547 | Sphingomonas |
| OTU85 | 0.00008 | 0.94221 | 341 | 1.0251 | Bacteroides |
| OTU313 | 0.00006 | 0.94976 | 342 | 1.0303 | Enterobacter |
| OTU233 | 0.00006 | 0.94995 | 343 | 1.0275 | Syntrophococcus |
| OTU569 | 0.00005 | 0.95462 | 344 | 1.02955 | Erwinia |
| OTU463 | 0.00004 | 0.95591 | 345 | 1.02795 | Lachnospiraceae Incertae Sedis |
| OTU345 | 0.00004 | 0.95812 | 346 | 1.02735 | Butyrivibrio |
| OTU190 | 0.00004 | 0.96018 | 347 | 1.02659 | Ruminococcaceae Incertae Sedis |
| OTU68 | 0.00004 | 0.96091 | 348 | 1.02442 | Dorea |
| OTU519 | 0.00003 | 0.96198 | 349 | 1.02262 | Catonella |
| OTU44 | 0.00003 | 0.96309 | 350 | 1.02087 | Lachnospiraceae Incertae Sedis |
| OTU71 | 0.00003 | 0.96365 | 351 | 1.01856 | Lachnospiraceae Incertae Sedis |
| OTU64 | 0.00003 | 0.96397 | 352 | 1.016 | Erwinia |
| OTU464 | 0.00002 | 0.97445 | 353 | 1.02414 | Marinilabilia |
| OTU495 | 0.00001 | 0.97451 | 354 | 1.02131 | Streptococcus |
| OTU248 | 0.00001 | 0.97479 | 355 | 1.01873 | Lachnospiraceae Incertae Sedis |
| OTU70 | 0.00001 | 0.97564 | 356 | 1.01675 | Sphingobium |
| OTU160 | 0.00001 | 0.97732 | 357 | 1.01564 | Lachnospiraceae Incertae Sedis |
| OTU244 | 0.00001 | 0.97775 | 358 | 1.01325 | Prevotella |
| OTU272 | 0.00001 | 0.97876 | 359 | 1.01147 | Sporobacter |
| OTU267 | 0.00001 | 0.97889 | 360 | 1.0088 | Parabacteroides |
| OTU170 | 0.00001 | 0.98074 | 361 | 1.00791 | Bacteroides |
| OTU303 | 0.00001 | 0.98274 | 362 | 1.00717 | Faecalibacterium |
| OTU458 | 0.00000 | 0.98693 | 363 | 1.00868 | Roseburia |
| OTU270 | 0.00000 | 0.98704 | 364 | 1.00602 | Succinispira |
| OTU393 | 0.00000 | 0.98709 | 365 | 1.00331 | Micrococcineae |
| OTU400 | 0.00000 | 0.98754 | 366 | 1.00103 | Bryantella |
| OTU547 | 0.00000 | 0.98883 | 367 | 0.99961 | Subdoligranulum |
| OTU52 | 0.00000 | 0.99158 | 368 | 0.99966 | Lachnospiraceae Incertae Sedis |
| OTU69 | 0.00000 | 0.99172 | 369 | 0.9971 | Lachnospiraceae Incertae Sedis |
| OTU47 | 0.00000 | 0.99456 | 370 | 0.99725 | Succinispira |
| OTU437 | 0.00000 | 0.99660 | 371 | 0.9966 | Marinilabilia |

Taken together, these findings demonstrate that the development of adenomas is associated with changes in the relative abundance of various taxa, including pathogens, present in the gut mucosa and that these changes are distinct from those associated with obesity. Analogous to the mechanism suggested for inflammatory bowel diseases, a potential explanation for this observation could be that the presence of adenomas compromises gut mucosal immunity, leading to an increased relative abundance in known pathogens such as Pseudomonas, Helicobacter, Acinetobacter (Table 2 and 3) and other genera belonging to the phylum Proteobacteria (Figure. 2). For IBD, see Chichlowski, M. & Hale, L. P. Bacterial-mucosal interactions in inflammatory bowel disease: an alliance gone bad. Am J Physiol Gastrointest Liver Physiol 295, G1139-1149 (2008). This increased relative abundance of various taxa including pathogens is in turn responsible for an overall increase in microbial richness in cases compared to controls (FIG. 1).

Alternatively, the presence of these pathogens may directly increase the risk of adenoma development by changing the gut environment. For example, Helicobacter has a much higher relative abundance in cases vs. controls (Table 2 & 3) consistent with previous studies, which implicate the role of this bacterium in colorectal adenomas; a possible explanation for this association is that this microbe alters the pH of the gastrointestinal tract. See, Jones, M., Helliwell, P., Pritchard, C., Tharakan, J. & Mathew, J. Helicobacter pylori in colorectal neoplasms: is there an aetiological relationship? World J Surg Oncol 5, 51 (2007); Burnett-Hartman, A. N., Newcomb, P. A. & Potter, J. D. Infectious agents and colorectal cancer: a review of Helicobacter pylori, Streptococcus bovis, JC virus, and human papillomavirus. Cancer Epidemiol Biomarkers Prev 17, 2970-2979 (2008); Zumkeller, N., Brenner, H., Zwahlen, M. & Rothenbacher, D. Helicobacter pylori infection and colorectal cancer risk: a meta-analysis. Helicobacter 11, 75-80 (2006); Abbolito, M. R., et al. The association of Helicobacter pylori infection with low levels of urea and pH in the gastric juices. Ital J Gastroenterol 24, 389-392 (1992); and Chen, G., Fournier, R. L., Varanasi, S. & Mahama-Relue, P. A. Helicobacter pylori survival in gastric mucosa by generation of a pH gradient. Biophys J 73, 1081-1088 (1997).

Acidovorax spp, another member of the bacterial signature identified as significantly different between case and control in this study, is a flagellated, Gram-negative acid-degrading member of the phylum Proteobacteria. Although, not much is known about its clinical epidemiology and pathogenicity in humans, it has been associated with induction of local inflammation. Tanaka, N., et al. Flagellin from an incompatible strain of Acidovorax avenae mediates H₂O₂ generation accompanying hypersensitive cell death and expression of PAL, Cht-1, and PBZ1, but not of Lox in rice. Mol Plant Microbe Interact 16, 422-428 (2003); and Takakura, Y., et al. Expression of a bacterial flagellin gene triggers plant immune responses and confers disease resistance in transgenic rice plants. Mol Plant Pathol 9, 525-529 (2008).

Lactobacillus, another taxa found to be higher in cases than controls, is an acid producing bacteria known to lower gut pH and regulate the growth of other bacteria. Biasco, G., et al. Effect of lactobacillus acidophilus and bifidobacterium bifidum on rectal cell kinetics and fecal pH. Ital J Gastroenterol 23, 142 (1991). While Lactobacillus is generally considered a beneficial microbe its presence in this case may help to lower pH to create favorable conditions for bacterial dysbiosis. This is consistent with suggestions by Duncan and co-workers that bacteria that grow in acidic pH create an environment that can be exploited by more low pH-tolerant microbes. Gibson, G. R. & Roberfroid, M. B. Dietary modulation of the human colonic microbiota: introducing the concept of prebiotics. J Nutr 125, 1401-1412 (1995); Macfarlane, S., Macfarlane, G. T. & Cummings, J. H. Review article: prebiotics in the gastrointestinal tract. Aliment Pharmacol Ther 24, 701-714 (2006); and Duncan, S. H., Louis, P. & Flint, H. J. Lactate-utilizing bacteria, isolated from human feces, that produce butyrate as a major fermentation product. Appl Environ Microbiol 70, 5810-5817 (2004).

While further experiments will be required to determine if and how increased microbial richness causes the development of adenomas, the observation that the microbial signature associated with adenomas is largely distinct from that associated with obesity suggests that next-generation sequencing of microbial communities may have considerable value as a diagnostic that can separate risk-factors from the actual presence of adenomas.

Methods Summary:

Bacterial genomic DNA was extracted from mucosal biopsies using the Qiagen DNA isolation kit (cat #14123) per the manufacturer's recommended protocol (Qiagen Inc. Valencia, Calif.). The adherent mucosal microbiome was analyzed by Roche 454 titanium pyrosequencing of V1-V2 region (F8-R357) of the 16S rRNA gene from genomic DNAs. After initial data filtering, to remove low quality sequences and to trim primers, the RDP Classifier 2.0 was used to assign the reads to genus and phylum as well as the algorithm AbundantOTU (http://omics.informatics.indiana.edu/AbundantOTU/ and http://mendel.informatics.indiana.edu/˜yye/lab/mypaper/AbundantOTU-BIBM-Ye.pdf) to group the sequences into clusters in which every sequence within a cluster is on average 97% identical.

All analyses (with the exception of UNIFRAC and calculation of diversity indices which use unlogged counts) were performed on the log-normalized counts at the phylum, genus and OTU levels. Shannon-Wiener Diversity Index, H, was calculated using the equation, H=−Σ Pi (lnPi), where Pi is the proportion of each species (taxa) in the sample. Richness was calculated as the number of OTUs, genera or phyla observed in 1542 sequences (where 1542 is the number of sequences seen in the sample with the fewest sequences). For each sample, 1542 sequences were randomly chosen 1,000 times and the average number of OTUs, genera or phyla observed over these 1,000 permutations was reported as richness.

Evenness measures how evenly the individuals are distributed among the different species/taxa and is calculated by the following equation J=H′/Log (S) where H′ is Shannon diversity and S is the number of species or taxa in each sample. Wilcoxon-tests and Student's t-tests were performed to compare the mean similarities of the groups, case and control. The false discovery rate was set at 10% using the Benjamini and Hochberg procedure to avoid type 1 error due to multiple comparisons on a single data set. Benjamini et al., (2001).

Patient Characteristics:

Subjects were screening colonoscopy patients at UNC Hospitals who agreed to participate in the Diet and Health Study (DHS V) and the characteristics of these subjects are shown in Table 8. The enrollment procedure as well as colonoscopy and biopsy procedures and sample collection have been previously described. Keku, T. O., et al. Insulin resistance, apoptosis, and colorectal adenoma risk. Cancer Epidemiol Biomarkers Prev 14, 2076-2081 (2005); Shen, X. J., et al. Molecular characterization of mucosal adherent bacteria and associations with colorectal adenomas. Gut Microbes 1, 138-147 (2010). The study was approved by the Institutional Review Board (IRB) at the University of North Carolina, School of Medicine.

Table 8: Descriptive characteristics of the study participants, cases (33) and controls (38). p-Values are based on t-tests between case and control (age, WHR and caloric intake) or the Chi square test (% Male and %BMI). The *p.Value for BMI is from the chi-quare test comparing across the groups. Caloric intake is reported as kilocalories (kcal) and is based on responses from a food frequency questionnaire that was administered to subj ects during phone interviews. Keku T. O., Sandler R. S., Simmons J. G., Galanko J, Woosley J. T., Proffitt M, Omofoye O, McDoom M, Lund P. K. Local IGFBP-3 mRNA expression, apoptosis and risk of colorectal adenomas. BMC Cancer 8:143 (2008).

TABLE 8

| | Case | | Control | | |
|---|---|---|---|---|---|
| Characteristics | (n = 33) | | (n = 38) | | p-Value* |
| Age (mean, SEM) | 57.45 | (1.11) | 55.70 | (1.08) | 0.26 |
| Male (%) | 60.61 | | 50 | | 0.54 |
| WHR (mean, SEM) | 0.94 | (0.01) | 0.90 | (0.01) | 0.06 |
| BMI (%) | | | | | |
| Normal | 27.27 | | 48.65 | | 0.09 |
| Overweight | 48.48 | | 24.32 | | |
| Obese | 24.24 | | 27.03 | | |
| Caloric intake | 2053.78 | (149.9) | 2104.89 | (252.46) | 0.86 |
| (kcal) (mean, SEM) | | | | | |

DNA extraction and sequencing: Bacterial genomic DNA was extracted from mucosal biopsies. The biopsies ranged in weight between 10-20 mg. Two biopsies per subject were used for bacterial DNA extraction and these were placed in lysozyme (30 mg/ml; Sigma, St. Louis Mo.) for 30 minutes. The biopsy-lysozyme mixture was homogenized on a bead beater (Biospec Products Inc., Bartlesville, Okla.) at 4,800 rpm for 3 minutes at room temperature followed by DNA extraction using the Qiagen DNA isolation kit (cat #14123) per the manufacturer's recommended protocol. The mucosal adherent microbiome was analyzed by Roche 454 titanium pyrosequencing of 16S rRNA tags from genomic DNAs. Pyrosequencing was conducted at the University of Nebraska Lincoln Core for Applied Genomics and Ecology (CAGE). Margulies et al., Genome sequencing in microfabricated high-density picolitre reactors. Nature 437:376-80 (2005). Briefly, the V1-V2 region (F8-R357) of the 16S rRNA gene from mucosal biopsies was amplified, followed by titanium-based pyrosequence analyses. The 16S primers contained the Roche 454 Life Science's A or B Titanium sequencing adapter (italicized), followed immediately by a unique 8-base barcode sequence (BBBBBBBB) and finally the 5′ end of primer A-8FM, 5′-CCATCTCATCCCTGCGTGTCTCGACTCAGBBBBBBBBAGAGTTTGATCMTGGCTCAG-3′ (SEQ ID No. 1) and B-357R, 5′-CCTATCCCCTGTGTGCCTTGGCAGTCTCAGBBBBBBBBCTGCTGCCTYCCGTA-3′ (SEQ ID No. 2). Each DNA sample was amplified with uniquely bar-coded primers, which allowed mixing of PCR products from many samples in a single run.

Data Filtering:

Sample Filtering:

All the samples were screened for a batch effect that correlated with the date of submission to the sequencing center. Samples were shipped on 3 separate dates to the sequencing center. Samples shipped on one particular date were found to cluster separately from samples shipped on other dates. The DNA stocks of these 2 groups of samples were also stored in different freezers at the lab. In addition, the sum of Bacteroidetes and Firmicutes observed in samples shipped on this date was much lower than expected based on both previously published human gut microbial 454 datasets and internal 454 datasets. Sequences generated from samples sent to the sequencing center on this date were therefore removed from further analysis. Leek et al. recently showed the importance of screening high throughput datasets for batch effects and screening for batch effects indeed proved useful in removing the technical artifacts from the dataset. Leek et al., Tackling the widespread and critical impact of batch effects in high-throughput data. Nat Rev Genet 11:733-9 (2010). The descriptive characteristics and of the 71 samples, 33 cases and 38 controls selected after sample filtering, are shown in Table 8 above.

Sequence Filtering:

RDP Pipeline:

The first step in the data analysis process involved a preliminary QC (quality control) filter (downstream of the Roche-454 GS-FLX software filtering). Sequences were removed from the dataset if there were any Ns in the sequence or the 5′ primer did not exactly match the expected 5′ primer or if the average quality score was less than 20. Then the 5′ primer sequence was removed from the reads that have survived above filtering. Only trimmed filtered sequences with a length between 200-500 bp were kept in the data set for RDP analysis.

OTU Pipeline:

Sequences were removed from inclusion in the OTU dataset if there were any Ns in the trimmed sequence or if the 5′ primer did not exactly match the expected 5′ primer. As recommended by Kunin et al., sequences were end-trimmed with the Lucy algorithm at a threshold of 0.002 (quality score of 27). Leek et al. (2010); Kunin et al., Wrinkles in the rare biosphere: pyrosequencing errors can lead to artificial inflation of diversity estimates. Environ Microbiol 12:118-23 (2010). Only reads with trimmed lengths between 150 and 450 were retained for OTU analysis. Table 9 shows the characteristics and number of sequences removed by the RDP and OTU pipelines.

TABLE 9
454 dataset characteristics before and
after QC for RDP and OTU pipelines.

| | Original | After QC |
|---|---|---|
| RDP Pipeline | | |
| Total # of Sequences | 600354 | 598645 |
| Average/Sample | 8455.69 | 8431.62 |
| SD | 3840.73 | 3843.29 |
| Average Sequence Length | 343.131 | 343.575 |
| OTU Pipeline | | |
| Total # of Sequences | 600354 | 532506 |
| Average/Sample | 8455.69 | 7500.08 |
| SD | 3840.73 | 3578.55 |
| Average Sequence Length | 343.131 | 302.034 |

Bacterial Identification:

The sequences in the dataset were given taxonomic assignments based on two methods.

RDP Assignment Method:

Sequences that have been filtered using the RDP pipeline (Table 9) were submitted to the RDP Classifier 2.1 algorithm for taxonomic identification at various taxonomic levels. Sequences assigned in each sample to various taxa, from phylum level and genus level, were counted at the RDP confidence threshold of 80.

OTU Assignment Method:

OTU analysis is more sensitive to sequencing error and therefore additional QC steps were applied in the OTU analysis pipeline (Table 9). Kunin et al., (2010). Sequences filtered through the OTU pipeline were submitted to AbundantOTU (http://omics.informatics.indiana.edu/AbundantOTU/) for assignment of each sequence to operational taxonomic units (OTUs; 97% identity). Sequences assigned in each sample to various OTUs were counted and then normalized and log transformed (see Data Preprocessing), before proceeding to further downstream analyses. Consensus sequences generated by AbundantOTU during construction of OTUs were submitted to RDP classifier 2.1 to assign taxonomy to each of the OTU groups. Consensus sequences of the 613 OTUs generated by AbundantOTU (Consensus sequences 1-613, Seq. ID Nos. 11-623) were also submitted to ChimeraSlayer20 (http://microbiomeutil.sourceforge.net/) and the 9 consensus OTUs identified by chimera slayer as chimeras were removed from the dataset. Haas, B. J., et al. Chimeric 16S rRNA sequence formation and detection in Sanger and 454-pyrosequenced PCR amplicons. Genome Res (2011). In addition consensus sequences of 4 OTUs on BLAST (http://blast.ncbi.nlm.nih.gov/Blast.cgi) search against the Silva reference 16S database failed to match >97% sequence identity so these were also removed from further analysis. This left a total of 600 OTUs.

Richness and Evenness:

Shannon-Wiener Diversity Index, H, was calculated using the equation, H=−Σ Pi (lnPi), where Pi is the proportion of each species (taxa) in the sample. Richness was calculated as the number of OTUs, genera or phyla observed in 2,636 sequences (where 2,636 is the number of sequences seen in the sample with the fewest sequences). For each sample, 2,636 sequences were randomly chosen 1,000 times and the average number of OTUs, genera or phyla observed over these 1,000 permutations was reported as richness.

Evenness measures how evenly the individuals are distributed among the different species/taxa and is calculated by J=H′/Log (S) where H′ is Shannon diversity and S is the number of species or taxa in each sample. Wilcoxon-tests and Student's t-tests were performed to compare the mean similarities of the groups, case and control. The false discovery rate was set at 10% using the Benjamini and Hochberg procedure to avoid type 1 error due to multiple comparisons on a single data set. Benjamini & Hochberg, 1995.

Data Preprocessing:

Raw counts were normalized then log transformed using the normalization scheme mentioned below, before proceeding with the rest of the analyses.

LOG 10((Raw count/# of sequences in that sample)*Average # of sequences per sample+1).

Removal of Rare Taxa:

In order to minimize the number of null hypotheses needed to correct for multiple hypothesis testing, rarely occurring taxa were removed. Those that occurred in so few patients that they could not be significantly associated with case-control or obesity phenotypes. In all of the analyses (except richness calculations), only included taxa which occurred in at least 25% of all samples were included. For the RDP approach, 9 phyla and 100 genera met this criterion. For the OTU approach, 371 OTUs met this criterion.

Tree Generation:

For each of the 371 consensus sequences from OTUs that met the above criteria, BLASTN (http://blast.ncbi.nlm.nih.gov/Blast.cgi) was used to find the top 10 hits in the Silva reference tree release 104 (http://www.arb-silva.de/download/arb-files/). In this way, a set of 3,594 aligned sequences was identified to serve as the reference tree. The program align.seqs within MOTHUR (http://www.mothur.org/) was used to align the 371 AbundantOTU consensus sequences that passed all QC steps to these 3,594 aligned sequences as extracted from the Silva reference alignment. With custom Java code based on the Archaeopteryx code base (http://www.phylosoft.org/archaeopteryx/), all but the 3,594 sequences were removed from the Silva reference tree. The alignment of the 3,594 reference sequences plus the 371 AbundantOTU sequences was loaded onto the RaxXML EPA server (http://i12k-exelixis3.informatik.tu-muenchen.de/raxml) which uses maximum likelihood to place new sequences within a reference tree. Custom Java code (available upon request) was used to add RDP calls from each consensus sequence (FIG. 12-1-12-7) and significant differences (FIGS. 2 & 12-1-12-7) to the tree. Trees were visualized with Archaeopteryx. Leaf nodes in Supplementary FIG. 5 are labeled with the RDP call of the consensus sequence at 80%.

UniFrac Analysis:

The tree generated from the 371 OTU consensus sequences (using Rax XML EPA server described above) along with the environment file with the abundance information of each of the 371 OTUs within the case and control environments were submitted to UniFrac and Fast UniFrac to see if cases cluster separately from controls. Lozupone C, Knight R. UniFrac: a new phylogenetic method for comparing microbial communities. Appl Environ Microbiol 71:8228-35 (2005). 100 permutations were run on the abundance weighted tree using the UniFrac significance test.

Data Validation:

Real-Time Quantitative PCR Validation:

q-PCR primers were designed based on no less than 95% sequence similarity from bacterial 16s ribosomal DNA sequence alignments obtained from pyrosequencing. To measure the abundance of a specific taxon, three primer pairs where designed: one generic for all bacterial groups (Universal Primer): [EUB341-F 5′-CCTACGGGAGGCAGCAG-3′ (SEQ ID No. 3) EUB518-R 5′-ATTACCGCGGCTGCTGG-3′ (SEQ ID No. 4)] and three taxon-specific primer pairs: first for the Helicobacter genus (Heli_F 5′ AGTGGCGCACGGGTGAGTA 3′ (SEQ ID No. 5) Heli_R 5′ GTGTCCGTTCACCCTCTCA 3′ (SEQ ID No. 6)), the next one for the Acidovorax genus (Aci_F 5′-TGCTGACGAGTGGCGAAC-3′ (SEQ ID No. 7) Aci_R 5′-GTGGCTGGTCGTCCTCTC-3′ (SEQ ID No. 8)) and another for the Cloacibacterium genus (Clo_F 5′-TGCGGAACACGTGTGCAA-3′ (SEQ ID No. 9) Clo_R 5′-CCGTTACCTCACCAACTAGC-3′(SEQ ID No. 10)).

10 μL PCR reactions were prepared containing 100 ng of DNA extracted from colonic mucosal biopsies, 10 μM of each primer, and 5 μL of Fast-SYBR Green Master Mix (Applied Biosystems). Cycling conditions were: 1 cycle at 95° C. for 10 minutes followed by 45 cycles of 95° C. for 15 seconds, 60° C. for 1 minute, and 72° C. for 30 seconds. A single dissociation curve cycle was run as follows: 95° C. for 30 seconds, 60° C. for 30 minute, and 90° C. for 30 seconds. A pool of samples was prepared to serve as the standard for the qPCR by mixing equal volumes from each sample. Abundance of a specific taxon was calculated by the delta-delta threshold cycle (ΔΔCt) method in which: ΔΔCt=(CtTSE−CtUE)−(CtTSP−CtUP). Livak K J, Schmittgen T D. Analysis of relative gene expression data using real-time quantitative PCR and the 2 (-Delta Delta C(T)) Method. Methods 25:402-8 (2001).

Where: Ct_{TSE}: Ct of experimental samples for taxon-specific primers, Ct_{UE}: Ct of experimental samples for universal primer, Ct_{TSP}: Ct for DNA Pool for taxon-specific primers, Ct_{UP}: Ct for DNA pool for universal primers. Theoretically, the abundance of a taxon is 2^{-ddCt}.

Nucleotide sequence accession numbers: All gene sequences in this study are available in the Genbank® database under the accession # SRS 166138.1-172960.2. They are listed as Consensus Sequences 1-613 (SEQ ID Nos. 11-623) in the Sequence Listing below.

### 6.2. Fusobacterium Associated with Colorectal Adenomas and Cancer

Summary

The human gut microbiota is increasingly recognized as a player in colorectal cancer (CRC). While particular imbalances in the gut microbiota have been linked to colorectal adenomas and cancer, no specific bacterium has been identified as a risk factor. Recent studies have reported a high abundance of Fusobacterium in CRC tumor samples compared to normal subjects, but this observation has not been reported for adenomas, CRC precursors. The abundance of Fusobacterium nucleatum in the normal rectal mucosa of subjects with (n=48) and without adenomas (n=67) was assessed. DNA was extracted from rectal mucosal biopsies and measured bacterial levels by quantitative PCR of the 16S ribosomal RNA gene. Local cytokine gene expression was determined in mucosal biopsies by quantitative PCR. The mean log abundance of Fusobacterium or cytokine gene expression between cases and controls was compared by T-test. Logistic regression was used to compare tertiles of Fusobacterium. Adenoma subjects had a significantly higher abundance of F. nucleatum compared to controls (p=0.01). Compared to the lowest tertile, subjects with high abundance of Fusobacterium were significantly more likely to have adenomas (OR 3.66, 95% CI 1.37-9.74, ptrend 0.005). Cases but not controls had significant positive correlation between local cytokine gene expression and Fusobacterium abundance. Among cases, the correlation for local TNF-α and Fusobacterium was r=0.33, p=0.06 while it was 0.44, p=0.01 for Fusobacterium and IL-10. These results support a link between the abundance of Fusobacterium in colonic mucosa and adenomas. They also implicate mucosal inflammation in the Fusobacterium-adenoma association.

Introduction

The human intestinal microflora is a complex and diverse environment populated by hundreds of different bacterial species. The amount of bacterial cells in the gut outnumbers all other eukaryotic cells in the human body by a factor of 10. Chow, Host-Bacterial Symbiosis in Health and Disease, Adv Immunol. 2010; 107: 243-274; Savage, Microbial ecology of the gastrointestinal tract. Annual review of microbiology 1977; 31:107-33. These bacteria are regulated in the gut by the mucosal immune system, which is made up of a complex network of functions and immune responses aimed at maintaining a cooperative system between the intestinal microbiota and the host (Chow, 2010). In a healthy gut these bacteria maintain homeostasis with the host. However when an imbalance, or bacterial dysbiosis, occurs in the gut, the host experiences inflammation, and a loss of barrier function. Mutch, Impact of commensal microbiota on murine gastrointestinal tract gene ontologies, Physiol Genomics 2004 19(1):22-31; Arthur, The Struggle Within: Microbial Influences on Colorectal Cancer, Inflamm Bowel Dis. 2011 17(1):396-409.

Bacterial dysbiosis has been linked to several diseases including ulcerative colitis, IBD and colorectal cancer (CRC). Kaur, Intestinal dysbiosis in inflammatory bowel disease, 2011 Gut Microbes. 2011 July-August; 2(4):211-6; Marchesi J R, Dutilh B E, Hall N, Peters W H M, Roelofs R, et al. (2011) Towards the Human Colorectal Cancer Microbiome. PLoS ONE 6(5): e20447. doi:10.1371/journal.pone.0020447; Sasaki The role of bacteria in the pathogenesis of ulcerative colitis. J Signal Transduct. 2012:704953; Sobhani, Microbial dysbiosis in colorectal cancer (CRC) patients. PLoS One. 2011 January 27; 6(1):e16393; Wang, Gut bacterial translocation contributes to microinflammation in experimental uremia. Dig Dis Sci. 2012 May 22. [Epub ahead of print].

Current research is focused on identifying key players in this imbalance as well as their specific contribution to colorectal carcinogenesis. No single bacterial species has been identified as a risk factor for CRC, but recent studies report an increase in the abundance of Fusobacterium by direct examination of samples human colorectal tumors compared to controls (Marchesi 2011). Castellarin, Fusobacterium nucleatum infection is prevalent in human colorectal carcinoma Genome Res. 2012 22: 299-306; Kostic, Genomic analysis identifies association of Fusobacterium with colorectal carcinoma, Genome Res. 2012 22: 292-298. These studies report Fusobacterium in the actual tumor sample as opposed to studies of the mucosal lining biopsies taken distant from a tumor. While these studies suggest that Fusobacterium may be involved in the later stages of CRC, they did not examine their role in either the early stages of colorectal carcinogenesis (adenomas) or the intestinal lining distant from the actual CRC tumor. The data suggests data suggest a field effect—that the presence of Fusobacterium in the rectum reflects adenomas or CRC elsewhere in the colon. While the causes of colorectal cancer are not fully known, it is becoming increasingly clear that the gut microbiota provide an important contribution. Whether Fusobacterium nucleatum in normal rectal mucosal biopsies is associated with colorectal adenomas or whether this relationship is mediated by local inflammation was evaluated. Fusobacterium is more abundant in adenoma cases than controls and that local inflammation, specifically inflammatory cytokines IL-10 and TNFα, are associated with increased abundance of Fusobacterium in cases.

Results

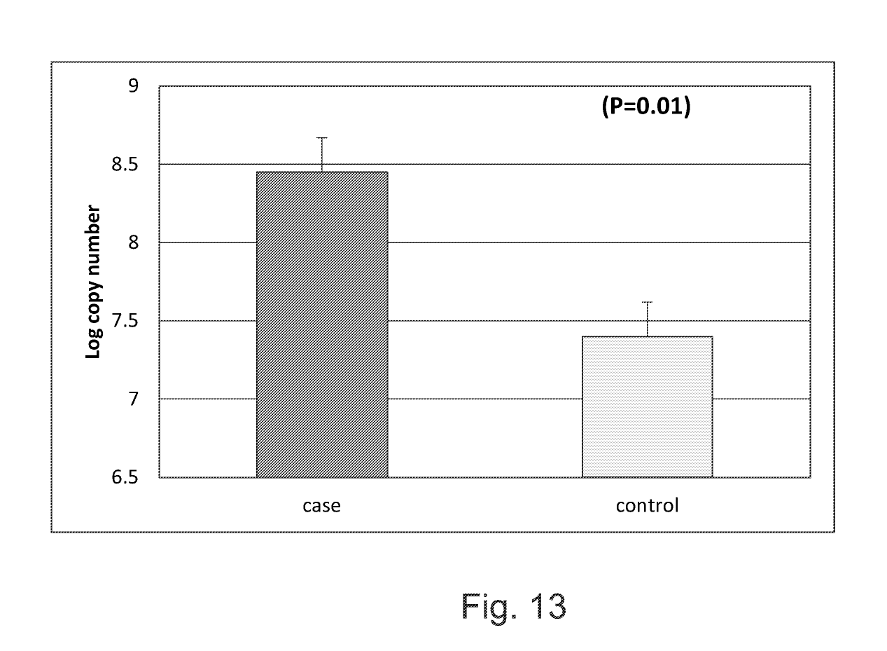

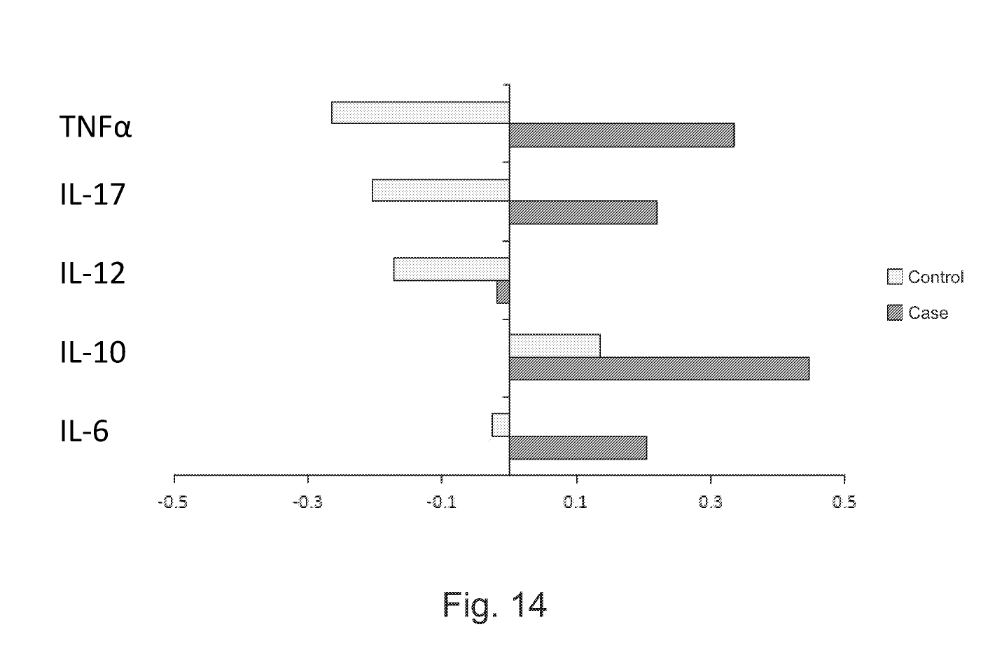

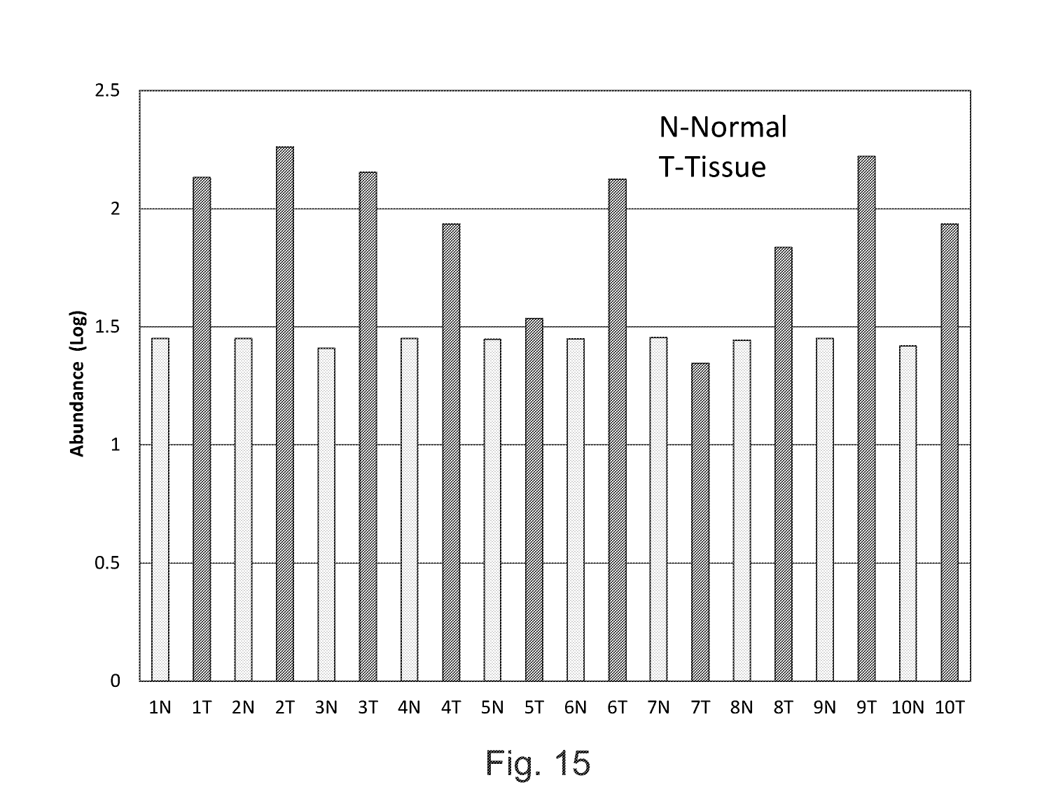

Fusobacterium abundance is higher in adenoma cases compared to controls. Adherent F. nucleatum in normal mucosal biopsies from 115 subjects, 48 cases and 67 controls were evaluated. Subject characteristics are shown in Table 10. All subjects were similar in age, with cases having a mean age of 56.38 and controls 55.90 years. There were no significant differences between adenoma cases and non-adenoma controls for several dietary factors evaluated including alcohol intake, caloric intake, waist-hip ratio, body mass index and total fat intake. The abundance of F. nucleatum was significantly higher in adenoma cases compared to controls (cases, mean log copy number and standard error, 8.44±0.38; controls 7.40±0.22 p=0.01) (FIG. 13). Compared to those with low copy number or abundance of Fusobacterium, those with high abundance of Fusobacterium are more likely to be adenoma cases (ptrend=0.005) (Table 11). The correlation between Fusobacterium abundance and the frequency and size (small, medium, large) of adenomas among cases was assessed also. There was no significant correlation between F. nucleatum and adenoma size or number of adenomas (data not shown).

Localization of Fusobacterium in Colonic Mucosal by FISH Analysis

Given that Fusobacterium was over-represented in cases compared to controls, we performed histological evaluation by FISH to localize Fusobacterium in colonic mucosal tissue sections. The results showed that Fusobacterium was localized in the mucus layer above the epithelium as well as within the colonic crypts.

There is a significant positive correlation between F. nucleatum abundance and local inflammation in cases. Correlation of local inflammatory cytokine gene expression and F. nucleatum abundance was analyzed separately for cases and controls. Analysis of cytokines IL-6, IL-10, IL-12, IL-17 and TNFα and F. nucleatum was observed to have a significant positive correlation with local inflammation in cases, but not controls (FIG. 3). A significant positive correlation was found between abundance of F. nucleatum and IL-10 (r=0.443 p=0.01). The correlation for TNFα (r=0.335 p=0.06) was borderline significant. Although the correlations for IL-6 and IL-17 were positive, they did not reach statistical significance.

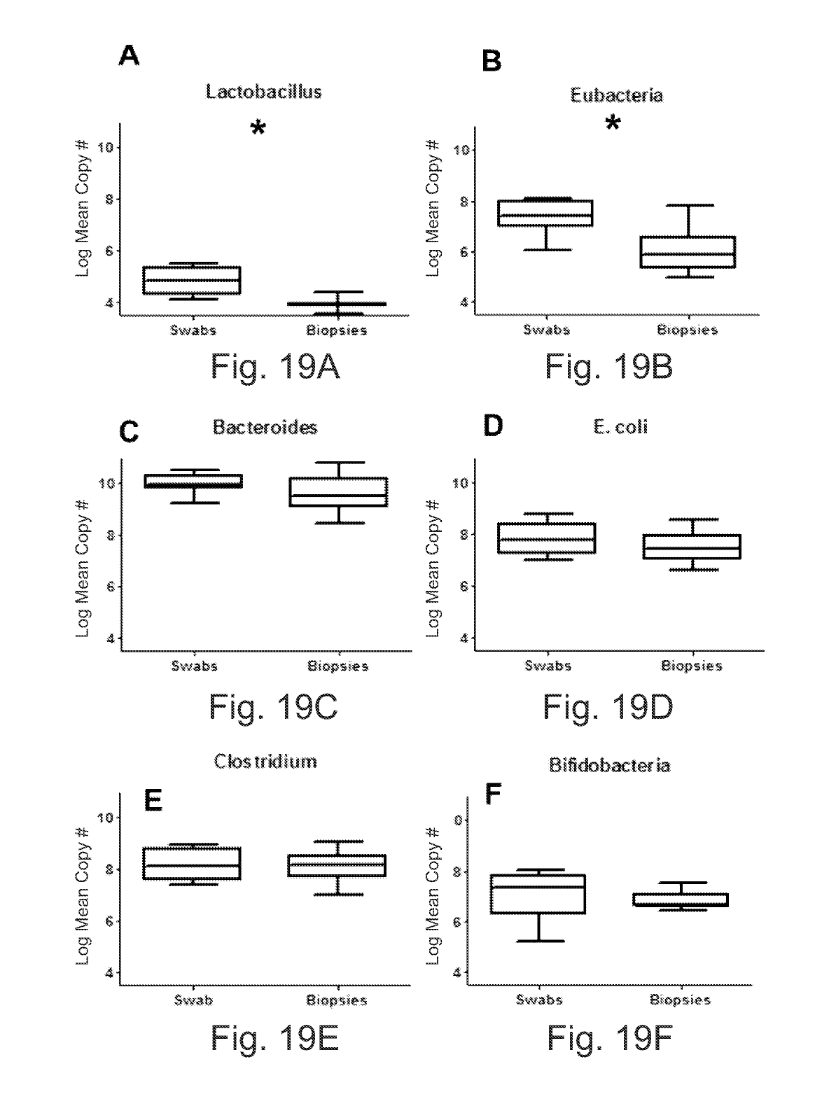

Analysis of colorectal tumors and matched normal tissue revealed higher F. nucleatum abundance in colon cancer tissue compared to normal tissue. Previous studies reported an association between F. nucleatum and colorectal cancer tumor biopsies. These results were reproduced by conducting high-throughput pyrosequence analysis on 19 matched samples, 10 tumor and 9 control non-malignant from adjacent mucosa. All subjects were Caucasian, predominantly female with ages ranging from 37-78 years. High-throughput sequencing revealed differences in abundance and richness in tumor compared to normal tissue. 13 phyla, 24 classes and 176 bacteria genera were identified. Overall, Shannon diversity and richness were higher in the tumor samples than matched normal tissue. Abundance of individual bacteria varied between groups. A reduced abundance of Bacteroidetes in tumor tissue compared to normal colon tissue was observed, however the distribution of the phylum Fusobacteria was higher in tumor tissue. The pyrosequencing results were validated by qPCR and a significantly positive correlation between the 2 methods (r=0.76, p=0.0001) was observed. The results showed a higher abundance of Fusobacterium in the CRC tissue compared to normal tissue. (FIG. 19)

qPCR Validation:

qPCR analysis of F. nucleatum in tumor versus normal tissue revealed a significant increase in abundance among colorectal cancer tissue compared to normal tissue, confirming previously reported results of higher Fusobacterium abundance in CRC patients. qPCR and pyrosequence data for Fusobacterium were compared and the relationship between tumor characteristics such as tumor location, treatment and F. nucleatum abundance was also evaluated for colorectal tumor samples. A significant association for tumor characteristics was not observed; however, higher abundance of F. nucleatum was found in the sigmoid than right side tumor location (Table 12).

Discussion

The human gut microbiota has been shown to have a dynamic and observable impact on the human host (Shen, 2010; Mutch, 2004). While many of these bacteria are commensal and facilitate the maintenance of a healthy and functioning gastrointestinal tract, current research has shown that interactions between the host and the bacteria colonizing the gut can contribute to various diseases including colorectal carcinogenesis (Shen, 2010). Hakansson and Molin, Gut microbiota and inflammation, 2011 Nutrients. 2011 3(6):637-82; Round J L, Mazmanian S K (2009) The gut microbiota shapes intestinal immune responses during health and disease. Nature Reviews Immunology 9: 313-323.

In particular, bacterial dysbiosis in the gut has been implicated in colorectal neoplasia, although no specific bacteria or bacterial signatures have been identified for colorectal adenomas have been reported previously (Sobhani, 2011; Marchesi, 2011). The abundance of Fusobacterium in relation to colorectal adenomas in a case-control study was evaluated and compared to controls, cases had significantly higher levels of Fusobacterium.

There has been a recent focus on Fusobacterium as it relates to human CRC. Fusobacterium nucleatum is a Gram-negative bacterium, which usually colonizes the oral cavity (Castellarin 2012). Swidsindki, Acute appendicitis is characterised by local invasion with Fusobacterium nucleatum/necrophorum, 2009, Gut. 2011 60(1):34-40. Recently, several groups identified Fusobacterium, particularly Fusobacterium nucleatum, in tumors of patients with colorectal carcinoma. Their findings reporting a link between colorectal tumor presence and high abundance of Fusobacteria, finding that the tumor microenvironment is characterized by a higher abundance of Fusobacteria than that of the normal colon (Castellarin 2012, Kostic 2011 Marchesi 2011). These results suggest F. nucleatum as potential biomarkers for colorectal carcinogenesis. However, it is not known whether F. nucleatum is associated with adenomas, early precursors of CRC. Several reports have shown early detection and/or removal of adenomas yields positive health benefits. Citarda, Efficacy in standard clinical practice of colonoscopic polypectomy in reducing colorectal cancer incidence, 2001 Gut. 2001 48(6):812-5; Fenoglio, The anatomical precursor of colorectal carcinoma, 1974 Cancer. 1974 34(3):suppl:819-23; Jaramillo, Small colorectal serrated adenomas: endoscopic findings, 1997, Endoscopy. 1997 29(1):1-3; Kapsoritakis, Diminutive polyps of large bowel should be an early target for endoscopic treatment, 2002 Dig Liver Dis. 2002 34(2): 137-40.

One purpose of this study was to identify the association between F. nucleatum and adenomas by quantifying its abundance in subjects with and without adenomatous polyps. Significant differences in bacterial richness between adenoma versus non-adenoma subjects were observed and there was a strong positive correlation between high abundance of F. nucleatum and the presence of colorectal adenomas (p=0.01). In particular those with high levels of Fusobacterium had about three and half fold increased risk of adenomas. It is interesting to observe increased F. nucleatum abundance in adenoma cases. As a CRC precursor, adenomas have become increasingly important in the study of colorectal carcinogenesis. Our results suggest that the changes in gut microflora are associated with the earliest stages of tumor development. Specifically that the normal mucosa rather than actual adenomas were studied. Our purpose was to demonstrate that the abundance of F. nucleatum in the gut is associated with adenoma status. While others observed a difference in Fusobacterium abundance between the colorectal tumor and adjacent non-neoplastic tissue (Kostic 2011, Castellarin 2012), it would also be beneficial in future studies to assess the actual adenomas, specifically, compared to normal rectal mucosa.

Our findings raise several important questions. Does Fusobacterium act alone or in concert with other bacteria to promote CRC? What are the mechanisms involved in this process? These questions will need to be addressed in future studies, particularly in animal models of CRC to uncover the mechanisms by which Fusobacterium and other bacteria promote colorectal adenomas and cancer.

Interestingly, intestinal inflammation has been repeatedly linked to the gut microbiota. Rogler et al. Microbiota in Chronic Mucosal Inflammation Int J Inflam. 2010; 2010: 395032; Tlaskalova-Hogenova, Commensal bacteria (normal microflora), mucosal immunity and chronic inflammatory and autoimmune diseases, 2004, Immunol Lett. 2004 93(2-3):97-108. Commensal gut bacteria interact with the host in a symbiotic way to facilitate the operation of the intestinal immune system. However, as reported by several studies, bacterial dysbiosis may lead to a breakdown in immune response and mucous production in the gut, ultimately disrupting the delicate homeostatic relationship between commensal bacteria and the human host (Arthur, 2011). Dharmani Chadee Biologic therapies against inflammatory bowel disease: a dysregulated immune system and the cross talk with gastrointestinal mucosa hold the key. Curr Mol Pharmacol. 2008 1(3):195-212. Uronis, Modulation of the intestinal microbiota alters colitis-associated colorectal cancer susceptibility, 2009, PLoS One. 2009 June 24; 4(6):e6026. Although F. nucleatum has been found to flourish primarily in the oral microbiome, it has also been observed to be a highly adherent bacterium (Weiss). Edwards, Fusobacterium nucleatum Transports Noninvasive Streptococcus cristatus into Human Epithelial Cells, 2006 Infect Immun. 2006 74(1):654-62; Han, Identification and Characterization of a Novel Adhesin Unique to Oral Fusobacteria, 2005 J Bacteriol. 2005 187(15):5330-40. The ability of F. nucleatum to attach to mucosal surfaces (Swidsinski, 2011) makes it an ideal candidate to study in relation to host immunity and adenomas.

By Fluorescent in Situ Hybridization (FISH) analysis, Fusobacterium was observed on the mucosal surface as well as within crypts. Specifically, FISH of colorectal biopsy sections targeting members of the Fusobacterium genus in mucus layer and crypts was performed. A pure E. coli culture preparation hybridized with general bacterial probe labeled with Cy3 and a pure Fusobacterium nucleatum culture preparation hybridized with Fusobacterium-specific probe labeled with Cy3 (red) served as positive controls. The Fusobacterium was localized within the mucus layer of colorectal section and simultaneously stained with DAPI. These FISH experiments showed that the Fusobacterium localized within the colorectal crypts of section (data not shown).

Uronis et al. successfully demonstrated a link between the microbiota, intestinal inflammation and increased risk of colitis-associated colorectal cancer (CAC) in a mouse model (Uronis, 2009). mRNA expression of local inflammatory cytokines IL-6, IL-10, IL-12, IL-17 and TNFα in normal rectal biopsies was assessed and their expression levels were correlated with abundance of F. nucleatum in our adenoma and non-adenoma subjects. There was a positive correlation between the gene expression of several local cytokines and F. nucleatum in adenoma cases, but not in controls. Specifically, similar to previously published findings (Dharmani et al 2011), a significant association between increased abundance of F. nucleatum and TNFα was observed. The increased abundance of F. nucleatum in adenoma cases coupled with positive correlation with local inflammation suggests that Fusobacteria may contribute to increased mucosal inflammation in adenoma subjects. This finding highlights the complex and multi-factorial relationship between the host and its enteric intestinal bacteria.

The relationship between F. nucleatum and adenoma size and frequency was also studied. However, there were no significant relationships observed between Fusobacterium and adenoma size (small, medium and large) or number of adenomas, suggesting that Fusobacterium richness in colonic mucosa may not have an impact on adenoma size or frequency.

Results for colorectal adenomas and increased Fusobacterium levels are similar to previously reported studies involving Fusobacterium and colorectal cancer (Kostic; Castellarin; Marchesi). The previously reported association between F. nucleatum and colorectal carcinoma was validated in a set of matched CRC tumor and normal human colon tissue samples. Using both pyrosequencing and qPCR analysis of the 16S bacterial rRNA gene these published results were successfully reproduced. Among CRC tumors and matched controls, F. nucleatum abundance was significantly higher in tumor tissue based on both qPCR as well as pyrosequence analysis, with a significant correlation between both methods (r=0.76, p=0.0001).

The fact that Fusobacterium is associated with colorectal adenomas implicates its involvement early in the carcinogenesis. Also, the results linking Fusobacterium and inflammation to adenomas suggest that this relationship may ultimately mediated by inflammation. Future studies in animal models of colorectal neoplasia could help to determine the mechanisms by which Fusobacterium and other bacteria promote cancer.

Materials and Methods

Study Population and Sampling:

Subjects were drawn from participants in the studies who underwent routine colonoscopy screening at UNC Hospitals, Chapel Hill, N.C. Eligible subjects 30 years of age or older gave written informed consent to provide colorectal biopsies as well as a phone interview involving questions about diet and lifestyle. At the time of the colonoscopy procedure, the research assistant obtained anthropometric measures to determine body mass index (BMI) and waist-hip ratio (WHR) (Shen, 2010; Section 6.1 above). Biopsy samples from a total of 115 randomly selected subjects (48 adenoma cases and 67 non-adenoma controls) were used in this study. Subjects with known or suspected colorectal cancer or with insufficient colon prep were excluded from the study. Before the endoscopy procedure was performed, biopsies were taken 8-12 cm from the anal verge of the normal rectal mucosa, and immediately flash frozen in liquid nitrogen. Biopsies were stored at −80° C. After completion of the endoscopy as well as the procedure report, participants with reported adenomas were classified as “cases” and those with no adenomas as “controls” (Section 6.1 above).

Additionally, matched tumor and normal tissue biopsies from 10 patients with colorectal cancer were obtained from UNC Tissue Procurement Facility to confirm previously reported studies. The study was approved by the Institutional Review Board at the University of North Carolina, School of Medicine.

Fusobacterium Culture:

Fusobacterium nucleatum subs. nucleatum ATCC® 25586™ was obtained and revived according to the manufacturer's instructions for use as a positive control. Reactivated bacteria were grown on reinforced clostridial media (Difco, Becton Dickinson, Franklin Lakes, N.J.) under anaerobic conditions at 37° C.

DNA Extraction:

DNA was extracted from normal rectal mucosal biopsies as well as matched tumor/normal tissue using the Qiagen DNeasy Blood and Tissue Kit (Cat#69504) which included a modified protocol with lysozyme and bead-beating (Shen, 2010; Section 6.1 above). F. nucleatum bacterial cells were centrifuged to form a pellet, re-suspended in kit-provided lysis buffer, and DNA extraction was performed using the same extraction method used for biopsies.

Quantitative Real-Time PCR (qPCR):

qPCR was performed to quantify the abundance of F. nucleatum. A standard curve was generated by amplifying the 16S rDNA region of F. nucleatum (ATCC® 25586™) using a 16S PCR with Fusobacterium-specific primers. Walter, Detection of Fusobacterium species in human feces using genus-specific PCR primers and denaturing gradient gel electrophoresis, Br J Biomed Sci. 2007; 64(2):74-7. The concentration of PCR product was checked by spectrophotometer and the number of fragment copies was calculated using the following formula:

$$\frac{x \frac{grams}{μ L} D N A}{( Length of fragment in base pairs )} \times ( 6.22 \times {10}^{23} ) = Copy \# ( \frac{Molecules}{μ L} )$$

Copy number was adjusted to a starting concentration of 1.00×10¹⁰ and serial dilutions were performed to create nine standards. 25 μl reactions were prepared containing template DNA, 10 μM primer mix, and Fast-SYBR Green Master Mix (Applied Biosystems). The qPCR was performed with an annealing temperature of 60° for 40 cycles. Finally, the copy number was calculated based on the standard curve, which was adjusted to a starting DNA concentration of 50 ng/μL using the following formula to the unadjusted values:

$$\frac{50 ng}{A / B} \times Unadjusted Copy \# ,$$

where A is the concentration of the template DNA and B is dilution; either 1:10.

qPCR was also performed for local mRNA expression of inflammatory cytokines IL-6, IL-10, IL-12, IL-17 and TNF-α using ready to use optimized primers (SA Biosciences). Expression of each inflammatory cytokine was assessed relative to the housekeeping gene hydroxymethylbilane synthase (HMBS). The qPCR was performed using SYBR Green Master Mix (Applied Biosystems) and each sample was run in duplicate. qPCR results were normalized using the expression of the HMBS gene. Jovov, Differential gene expression between African American and European American colorectal cancer patients, 2011, PLoS One. 2012; 7(1):e30168.

Fluorescence In Situ Hybridization (FISH):

FISH was performed on Carnoy's fixed mucosal biopsy sections using a universal bacteria probe and a Fusobacterium-specific probe. These assays used a previously described protocol (Shen, 2010).

TABLE 10
Characteristics of Study Participants.

| | Case | Control | |
|---|---|---|---|
| Characteristic | (n = 48) | (n = 67) | P-value |
| Age (mean, se) | 56.38 ± 0.92 | 55.90 ± 0.88 | 0.71 |
| Waist-Hip ratio (mean, se) | 0.94 ± 0.01 | 0.91 ± 0.01 | 0.14 |
| Body Mass Index (mean, se) | 27.40 ± 0.61 | 27.04 ± 0.66 | 0.70 |
| Alcohol Intake (mean, se) | 12.65 ± 1.94 | 21.17 ± 8.88 | 0.41 |
| Calories (mean, se) | 2108.70 ± 114.78 | 2140.38 ± 144.0 | 0.87 |
| Total Fat intake (mean, se) | 82.36 ± 5.31 | 79.36 ± 4.78 | 0.67 |
| Red meat intake (mean, se) | 1.59 ± 0.17 | 1.36 ± 0.14 | 0.30 |
| Dietary Fiber (mean, se) | 23.03 ± 1.28 | 25.58 ± 1.76 | 0.27 |

TABLE 11
Association between Fusobacterium abundance and colorectal
adenomas. Compared to subjects with a low copy number, subjects
with high abundance of Fusobacterium are more likely
to be adenoma cases than controls.

| | Case | Control | |
|---|---|---|---|
| Categories | (n = 48) | (n = 67) | OR (95% CI) |
| Tertile 1 | 8 | 23 | Reference |
| Tertile 2 | 12 | 22 | 1.57 (0.54-4.57) |
| Tertile 3 | 28 | 22 | 3.66 (1.37-9.74) |
| P trend | | | 0.005 |

TABLE 12
Relationship between Fusobacterium
and colorectal tumor characteristics

| | Fusobacterium | |
|---|---|---|
| | (copy #, | |
| Variable | mean, se) | P-value |
| Tumor Location | | |
| Right | 1.82 ± 0.13 | |
| Transverse | 1.94 ± 0.09 | NS |
| Sigmoid | 2.21 ± 0.31 | 0.04 Sigmoid vs. Right |
| Stage | | |
| T-2 | 1.83 ± 0.29 | |
| T-3 | 1.98 ± 0.11 | 0.56 |
| Adjuvant Therapy | | |
| N | 2.16 ± 0.03 | 0.20 |
| Y | 2.01 ± 0.10 | |

### 6.3. Signature of Rectal Mucosal Biopsies and Rectal Swabs

Summary

There is growing evidence the microbiota of the large bowel may influence the risk of developing colorectal cancer as well as other diseases including Type-1 Diabetes, Inflammatory Bowel Diseases and Irritable Bowel Syndrome. Current sampling methods to obtain microbial specimens, such as feces and mucosal biopsies, are inconvenient and unappealing to patients. Obtaining samples through rectal swabs could prove to be a quicker and relatively easier method, but it is unclear if swabs are an adequate substitute. We compared bacterial diversity and composition from rectal swabs and rectal mucosal biopsies in order to examine the viability of rectal swabs as an alternative to biopsies. Paired rectal swabs and mucosal biopsy samples were collected in un-prepped participants (n=11) and microbial diversity was characterized by Terminal Restriction Fragment Length polymorphism (T-RFLP) analysis and quantitative polymerase chain reaction (qPCR) of the 16S ribosomal RNA gene. Microbial community composition from swab samples was different from rectal mucosal biopsies (p=0.001). Overall the bacterial diversity was higher in swab samples than in biopsies as assessed by diversity indexes such as: richness (p=0.01), evenness (p=0.06) and Shannon's diversity (p=0.04). Analysis of specific bacterial groups by qPCR showed higher copy number of Lactobacillus (p=0.04) and Eubacteria (p=0.01) in swab samples compared to biopsies. Our findings suggest that rectal swabs and rectal mucosal samples provide different views of the microbiota in the large intestine.

Introduction

Increasing evidence suggests a role for the intestinal microbiota in colorectal cancer (CRC) (Sobhani et al. Microbial dysbiosis in colorectal cancer (CRC) patients. PloS one 2011; 6:e16393), colorectal adenomas (Shen 2010) and several other conditions such as Inflammatory Bowel Diseases (Ulcerative Colitis and Crohn's Disease)(Gersemann et al. Innate immune dysfunction in inflammatory bowel disease. Journal of internal medicine 2012), Irritable Bowel Syndrome (IBS)(Carroll et al. Luminal and mucosal-associated intestinal microbiota in patients with diarrhea-predominant irritable bowel syndrome. Gut pathogens 2010; 2:19), Obesity (Turnbaugh et al. An obesity-associated gut microbiome with increased capacity for energy harvest. Nature 2006; 444:1027-31) and Type-1 Diabetes (Brown et al. Gut microbiome metagenomics analysis suggests a functional model for the development of autoimmunity for type 1 diabetes. PloS one 2011; 6:e25792). The launch of the Human Microbiome Project and the advent of molecular techniques that reduce the bias imposed by culture-based methods has begun to improve our understanding of the role of the microbiota in common chronic diseases. Turnbaugh et al. The human microbiome project. Nature 2007; 449:804-10

Currently, gut bacterial diversity in the human colon is determined through analysis of the luminal content (stool) and mucosal biopsies. Colorectal biopsies capture the diversity of flora in the mucosal layer of the large intestine where adherent bacteria reside. Savage 1977; Sonnenburg et al. Getting a grip on things: how do communities of bacterial symbionts become established in our intestine? Nature immunology 2004; 5:569-73. The bacteria in this compartment are of interest because of their direct interaction with the host immune system, and by consequence, their possible direct link to disease development. Goto Y, Kiyono H. Epithelial barrier: an interface for the cross-communication between gut flora and immune system. Immunological reviews 2012; 245:147-63. Unfortunately, methods for obtaining colorectal biopsies such as sigmoidoscopy, anoscopy or colonoscopy are expensive and time consuming and may subject the patient to discomfort and inconveniences associated with the procedures. ACS. Colorectal Cancer Facts & Figures. In: Society AC, ed., 2011:1-30. Stool sampling, which does not pose a major risk to patients, is least liked because of the patient distaste for handling feces. A simpler, standardized, risk-free and inexpensive method to sample the gut bacteria would represent an important contribution.

In this Section, rectal swabs as a noninvasive low-risk sampling method and rectal mucosal biopsies obtained via unprepped, rigid sigmoidoscopy were assessed to study the bacterial community composition and diversity of the human gut using terminal restriction fragment length polymorphism (T-RFLP) and quantitative PCR (qPCR) of the bacterial 16S ribosomal RNA gene. It was hypothesized that rectal swabs have comparable bacterial diversity to rectal mucosal biopsies from the same participant.

Results

Study Population

The mean age of participants was 56.3 years±5.6. Forty-five percent of the participants were male, and the average body mass index (BMI) was 30.5±6.4 (Table 15 below). Rectal mucosal biopsies were obtained via rigid sigmodoscopy at approximately 10 cm from the anal verge while swabs were obtained 1-2 cm from the anal verge. Participants did not undergo colonic cleansing preparation prior to sample collection.

Analysis of T-RFLP Profiles Showed Overall Differences in Community Composition Between Swabs and Biopsy Samples.

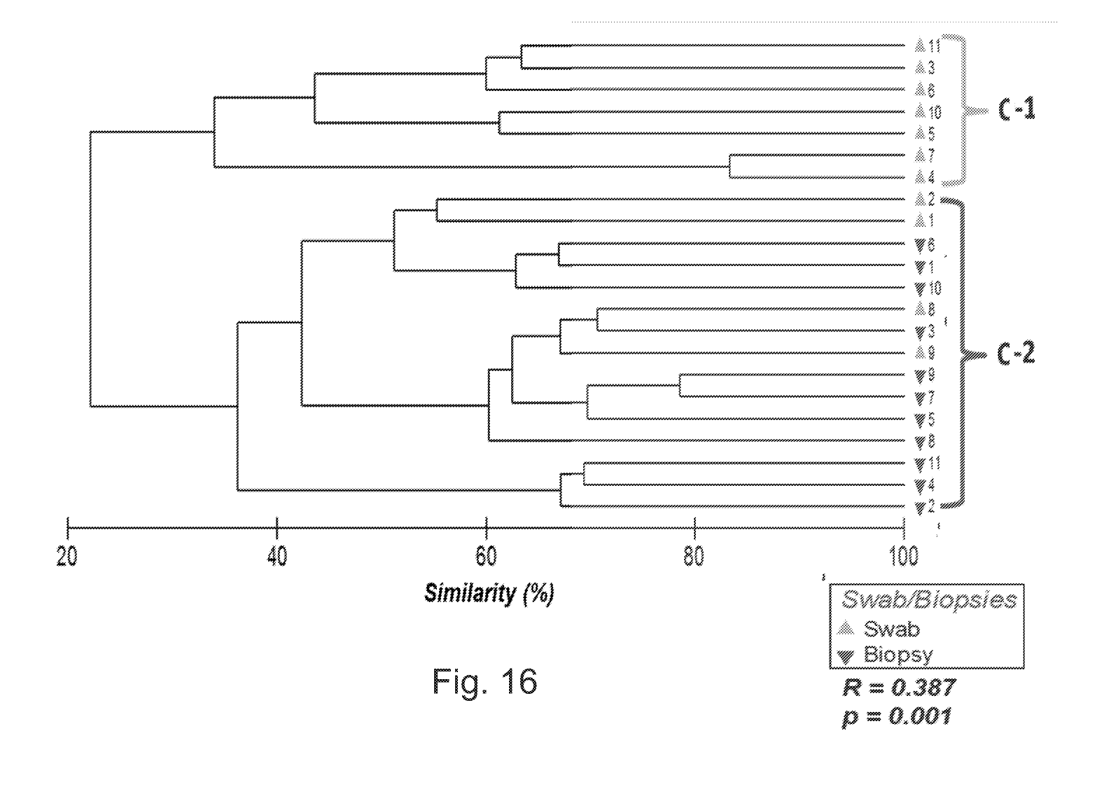

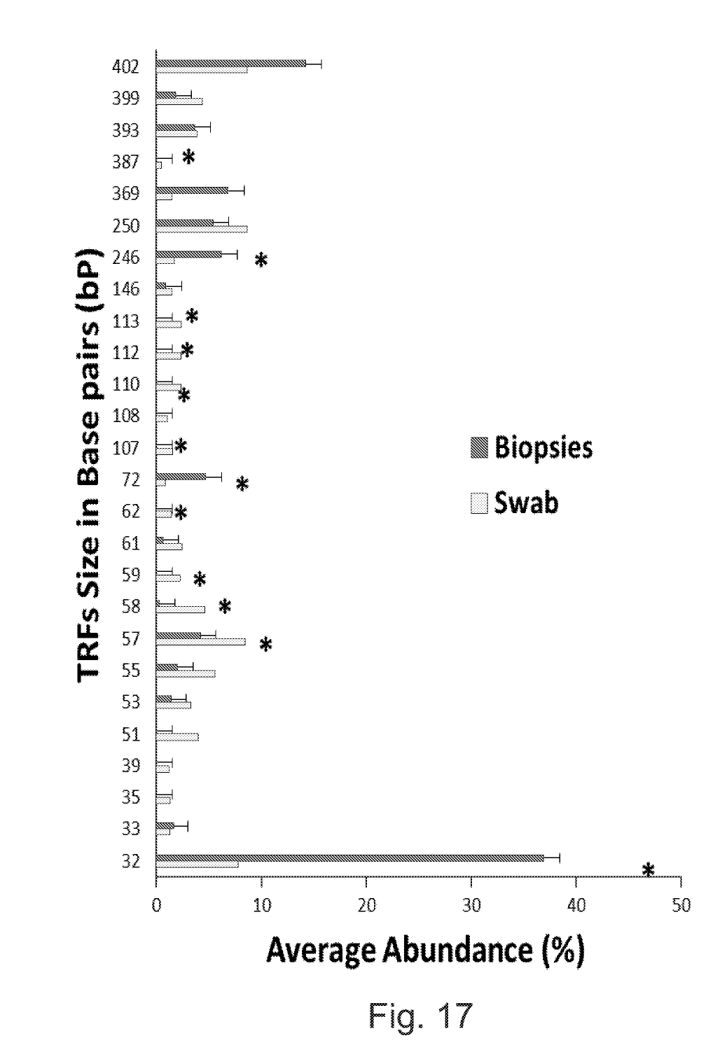

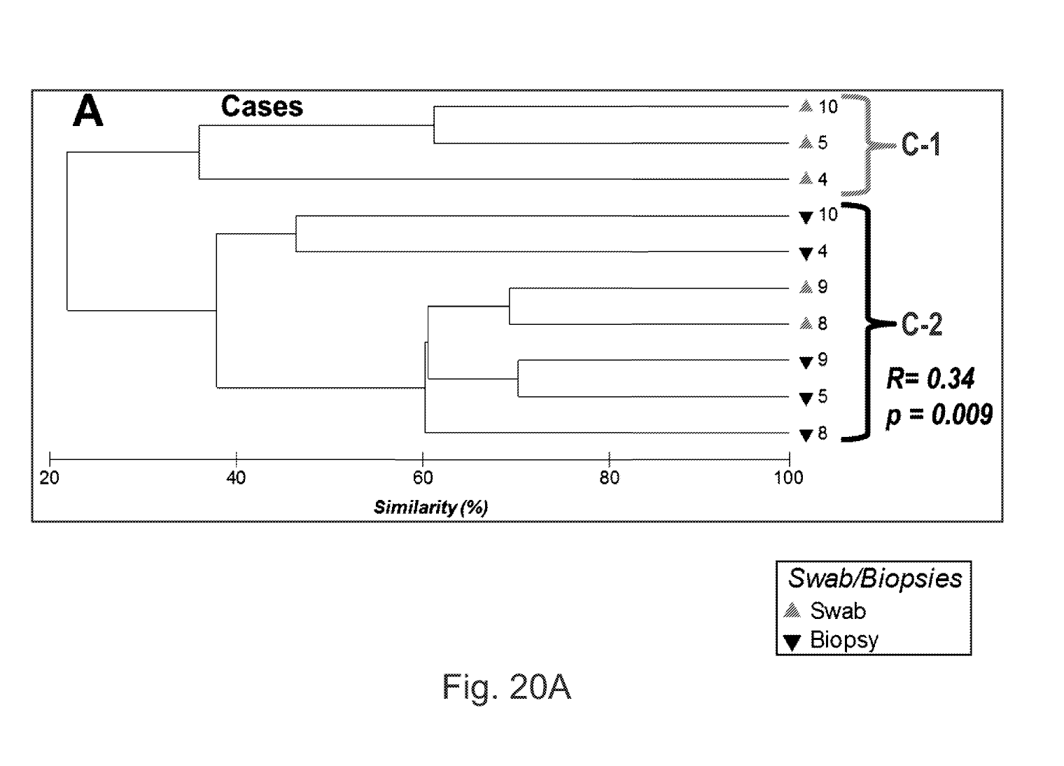

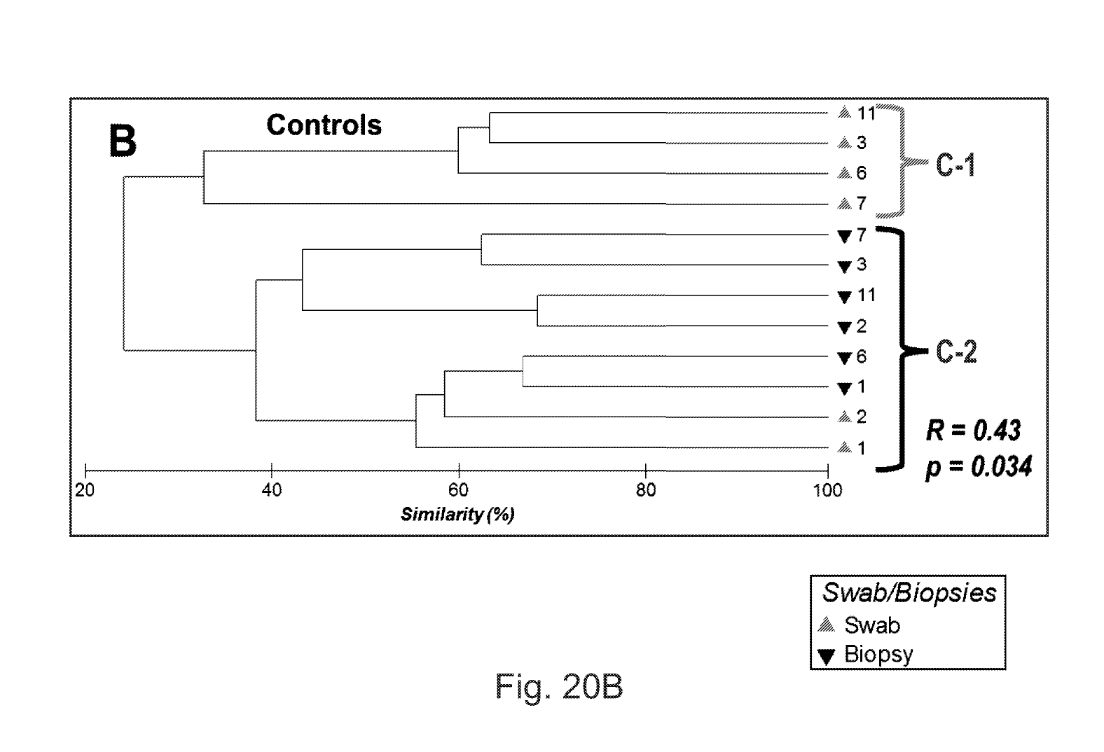

Hierarchical clustering of the 16S rRNA gene T-RFs based on Bray-Curtis similarities showed two main clusters suggesting differences in bacterial communities between samples collected from rectal swabs and biopsies (ANOSIM R=0.387, p=0.001) (FIG. 16). Cluster-1 was comprised entirely of rectal swab samples (100%) while cluster-2 was composed mainly of biopsy samples (73% biopsies and 27% swabs). The clusters were independent of adenoma status (FIG. 20).

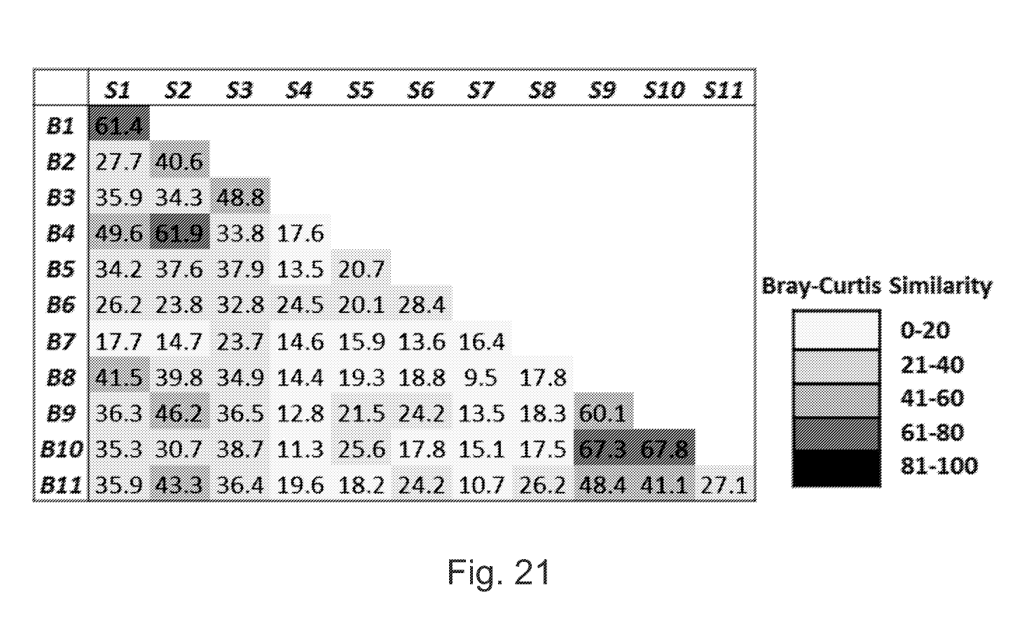

Using similarity percentage analysis (SIMPER), specific T-RFs contributed to the differences between swabs and biopsies were assessed. A total of 26 T-RFs accounted for the overall diversity for the two groups, with a higher number of unique T-RFs in rectal swab samples than rectal biopsies (FIG. 16). 16 T-RFs were unique to swab samples (107, 108, 110, 112, 113, 146, 35, 387, 39, 399, 51, 53, 58, 59, 61, 62), while 2 TRFs (369 and 72) were unique to biopsy samples. Distribution of T-RFs for each individual sample as well as Bray-Curtis similarities matrix showed marked differences between swabs and biopsies from the same participant (FIG. 21). Distribution of top contributing TRFs based on similarity percentage analysis (SIMPER). The swabs (S1-11) or the biopsy samples (B1-B11) collected from each of patient. Tables 13 and 14 lists the TRFs and the percentage contribution.

TABLE 13
Swabs and TRF contributions.

| | Spec.# | | | | | | | | | | |
|---|---|---|---|---|---|---|---|---|---|---|---|
| T-RF | S1 | S2 | S3 | S4 | S5 | S6 | S7 | S8 | S9 | S10 | S11 |
| 32.1B | 12.98 | 13.72 | 11.31 | 1.83 | 0.00 | 9.48 | 0.00 | 7.44 | 26.21 | 10.62 | 12.28 |
| 33.4B | 0.71 | 0.59 | 2.00 | 1.16 | 0.00 | 1.81 | 0.00 | 3.09 | 0.78 | 0.32 | 7.50 |
| 35.6B | 0.00 | 0.00 | 1.10 | 1.28 | 0.00 | 1.09 | 0.00 | 4.55 | 0.00 | 0.00 | 8.66 |
| 39.2B | 0.00 | 0.00 | 1.69 | 1.12 | 0.00 | 1.61 | 0.00 | 3.72 | 0.00 | 0.00 | 6.54 |
| 51.5G | 0.00 | 0.00 | 1.34 | 5.05 | 0.00 | 1.13 | 0.00 | 15.60 | 0.00 | 0.00 | 29.09 |
| 53.9B | 1.73 | 0.00 | 0.00 | 0.00 | 22.59 | 0.00 | 24.28 | 0.00 | 0.00 | 0.00 | 0.00 |
| 55.4B | 0.00 | 1.46 | 3.16 | 2.96 | 13.58 | 2.61 | 13.80 | 10.25 | 8.32 | 4.01 | 14.29 |
| 57.0B | 4.30 | 0.00 | 10.59 | 4.12 | 18.02 | 0.00 | 18.52 | 12.00 | 14.87 | 10.92 | 19.12 |
| 58.4B | 0.00 | 2.92 | 0.00 | 10.24 | 11.26 | 11.96 | 13.28 | 14.73 | 0.00 | 1.50 | 0.00 |
| 59.6G | 0.00 | 0.00 | 5.62 | 6.92 | 5.73 | 6.23 | 5.76 | 2.93 | 0.00 | 0.60 | 0.00 |
| 61.2B | 0.00 | 0.00 | 8.57 | 6.84 | 0.00 | 8.84 | 0.00 | 2.85 | 1.59 | 2.63 | 2.52 |
| 62.5B | 0.00 | 0.00 | 6.31 | 6.45 | 0.00 | 6.18 | 0.00 | 2.30 | 0.00 | 0.46 | 0.00 |
| 72.1G | 2.91 | 10.35 | 0.00 | 0.00 | 0.00 | 0.00 | 0.00 | 0.00 | 0.00 | 0.74 | 0.00 |
| 107.8B | 0.00 | 0.00 | 3.49 | 7.97 | 0.00 | 3.00 | 4.72 | 2.77 | 0.00 | 0.00 | 0.00 |
| 108.9G | 0.00 | 0.00 | 3.37 | 7.44 | 0.00 | 2.52 | 0.00 | 2.53 | 0.00 | 0.00 | 0.00 |
| 110.8G | 0.00 | 0.00 | 5.65 | 10.12 | 3.96 | 3.89 | 6.20 | 4.04 | 0.00 | 0.00 | 0.00 |
| 112.6G | 0.00 | 0.00 | 4.60 | 10.37 | 4.10 | 4.10 | 6.20 | 4.43 | 0.00 | 0.00 | 0.00 |
| 113.7B | 0.00 | 0.00 | 4.79 | 9.43 | 5.05 | 4.18 | 7.25 | 3.52 | 0.00 | 0.00 | 0.00 |
| 146.4G | 9.92 | 1.62 | 1.32 | 5.13 | 0.00 | 2.30 | 0.00 | 3.25 | 0.00 | 0.00 | 0.00 |
| 246.3B | 0.00 | 6.32 | 4.20 | 0.00 | 0.00 | 0.00 | 0.00 | 0.00 | 4.49 | 7.97 | 0.00 |
| 250.5B | 0.00 | 0.00 | 3.15 | 0.00 | 0.00 | 0.00 | 0.00 | 0.00 | 7.69 | 12.11 | 0.00 |
| 369.2B | 6.59 | 18.40 | 0.00 | 0.00 | 0.00 | 0.00 | 0.00 | 0.00 | 0.00 | 0.00 | 0.00 |
| 387.4G | 3.47 | 1.71 | 0.00 | 0.00 | 0.00 | 1.91 | 0.00 | 0.00 | 0.00 | 1.27 | 0.00 |
| 393.5G | 5.64 | 5.68 | 5.97 | 0.00 | 0.00 | 0.64 | 0.00 | 0.00 | 20.06 | 11.56 | 0.00 |
| 399.7G | 27.94 | 28.65 | 1.26 | 0.00 | 0.00 | 16.66 | 0.00 | 0.00 | 0.00 | 0.00 | 0.00 |
| 402.1G | 23.80 | 8.59 | 10.52 | 1.58 | 15.70 | 9.86 | 0.00 | 0.00 | 15.99 | 35.30 | 0.00 |

TABLE 14
Mucosal biopsies and TRF contributions.

| | Spec # | | | | | | | | | | |
|---|---|---|---|---|---|---|---|---|---|---|---|
| T-RF | B1 | B2 | B3 | B4 | B5 | B6 | B7 | B8 | B9 | B10 | B11 |
| 32.1B | 11.91 | 74.65 | 33.21 | 21.80 | 28.67 | 76.41 | 89.57 | 16.95 | 13.37 | 33.28 | 34.10 |
| 33.4B | 0.58 | 4.05 | 1.18 | 0.96 | 1.52 | 2.30 | 3.01 | 0.71 | 0.88 | 1.28 | 1.90 |
| 35.6B | 0.00 | 0.00 | 0.00 | 0.00 | 0.00 | 0.00 | 0.00 | 0.00 | 0.00 | 0.00 | 0.00 |
| 39.2B | 0.00 | 0.00 | 0.00 | 0.00 | 0.00 | 0.00 | 0.00 | 0.00 | 0.00 | 0.00 | 0.00 |
| 51.5G | 0.00 | 0.00 | 0.00 | 0.00 | 0.00 | 0.00 | 0.00 | 0.00 | 0.00 | 0.00 | 0.00 |
| 53.9B | 1.21 | 0.00 | 0.00 | 0.00 | 3.31 | 0.00 | 0.00 | 2.43 | 6.17 | 4.55 | 0.00 |
| 55.4B | 0.00 | 4.31 | 5.05 | 2.37 | 0.00 | 4.41 | 4.31 | 0.00 | 0.00 | 0.00 | 2.73 |
| 57.0B | 1.72 | 1.76 | 7.48 | 4.42 | 4.58 | 5.05 | 3.11 | 3.20 | 11.23 | 6.48 | 2.87 |
| 58.4B | 0.00 | 2.81 | 0.00 | 0.00 | 0.00 | 0.00 | 0.00 | 0.00 | 0.19 | 0.00 | 0.00 |
| 59.6G | 0.00 | 0.00 | 0.00 | 0.00 | 0.00 | 0.00 | 0.00 | 0.00 | 0.00 | 0.00 | 0.00 |
| 61.2B | 0.00 | 0.00 | 0.00 | 0.00 | 0.00 | 7.13 | 0.00 | 0.00 | 0.00 | 0.00 | 0.49 |
| 62.5B | 0.00 | 0.00 | 0.00 | 0.00 | 0.00 | 0.00 | 0.00 | 0.00 | 0.00 | 0.00 | 0.00 |
| 72.1G | 15.83 | 2.59 | 2.11 | 12.07 | 0.00 | 1.98 | 0.00 | 4.63 | 0.90 | 0.77 | 16.17 |
| 107.8B | 0.00 | 0.00 | 0.00 | 0.00 | 0.00 | 0.00 | 0.00 | 0.00 | 0.00 | 0.00 | 0.00 |
| 108.9G | 0.00 | 0.00 | 0.00 | 0.00 | 0.00 | 0.00 | 0.00 | 0.00 | 0.44 | 0.00 | 0.00 |
| 110.8G | 0.00 | 0.00 | 0.00 | 0.00 | 0.00 | 0.00 | 0.00 | 0.00 | 0.00 | 0.00 | 0.00 |
| 112.6G | 0.00 | 0.00 | 0.00 | 0.00 | 0.00 | 0.00 | 0.00 | 0.00 | 0.00 | 0.00 | 0.00 |
| 113.7B | 0.00 | 0.00 | 0.00 | 0.00 | 0.00 | 0.00 | 0.00 | 0.00 | 0.00 | 0.00 | 0.00 |
| 146.4G | 2.02 | 0.00 | 0.00 | 1.17 | 0.68 | 0.00 | 0.00 | 3.75 | 1.38 | 0.00 | 2.27 |
| 246.3B | 15.57 | 3.09 | 8.41 | 5.15 | 14.49 | 0.00 | 0.00 | 14.23 | 7.95 | 6.15 | 1.94 |
| 250.5B | 0.00 | 0.00 | 8.26 | 0.41 | 9.30 | 0.00 | 0.00 | 19.29 | 16.66 | 13.22 | 0.74 |
| 369.2B | 26.46 | 4.77 | 0.00 | 20.99 | 0.00 | 0.00 | 0.00 | 0.00 | 0.00 | 0.00 | 30.35 |
| 387.4G | 0.00 | 0.00 | 0.00 | 0.00 | 0.00 | 0.00 | 0.00 | 0.00 | 0.00 | 0.00 | 0.00 |
| 393.5G | 2.07 | 0.00 | 11.80 | 0.00 | 5.57 | 0.00 | 0.00 | 3.36 | 14.98 | 8.63 | 0.39 |
| 399.7G | 3.73 | 0.00 | 2.52 | 15.12 | 0.00 | 0.00 | 0.00 | 0.00 | 2.97 | 0.00 | 0.00 |
| 402.1G | 18.91 | 1.97 | 19.98 | 15.54 | 31.89 | 2.72 | 0.00 | 31.45 | 22.88 | 25.64 | 6.05 |

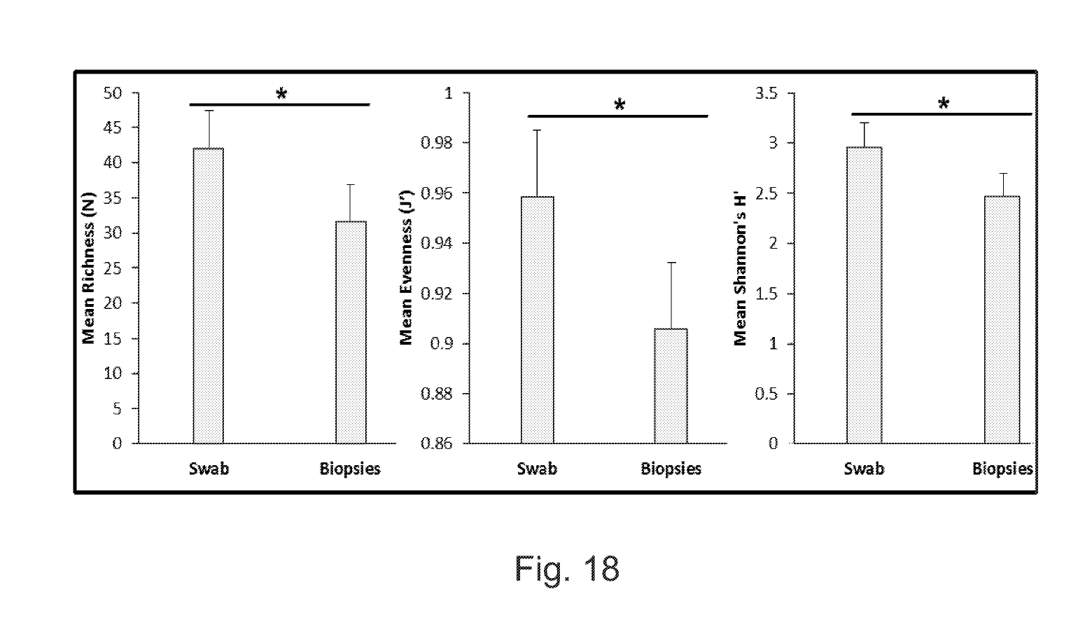

Measures of microbial diversity were also assessed namely richness (N), evenness (J′) and Shannon's H (diversity) and observed that overall diversity measures were higher in rectal swabs compared to rectal biopsies (FIG. 18). Altogether, the T-RFLP results demonstrate that the bacterial community composition from rectal swabs and rectal biopsies are different.

Quantitative PCR Showed Differences in Abundance of Specific Bacterial Groups Between Swabs and Biopsy Samples.

Bacterial genera common in the human gut were quantified by qPCR of the bacterial 16S rRNA gene. All quantified bacterial groups (Clostridium spp., Bifidobacterium spp., Bacteroides spp., Lactobacillus spp. and E. coli,) and Eubacteria bacterial groups (as assessed by Universal 16S rRNA primers) showed higher abundance in swab specimens compared to biopsy samples. However, statistically significant differences were only observed for Lactobacillus spp. and Eubacteria (FIG. 19).

Discussion and Conclusions

The association between colorectal adenomas and dysbiosis of gut microbes has been previously reported and could serve as the basis to identify microbial signatures that could lead to the development of tests to identify individuals at risk of developing colorectal cancer. Shen 2010; Section 6.1 above. Biopsies collected during colonoscopy, as well as stool samples, are the current methods to characterize the microbiota of the large intestine. A simple, standardized, risk-free and inexpensive method to assess bacterial community composition of the gut could lower the risks and inconvenience associated with collection of these samples. In the present study, the bacterial composition of rectal swabs and rectal mucosal biopsies collected during an un-prepped sigmoidoscopy from 11 participants was systematically compared. The bacterial community composition from these two sampling sites was compared to determine whether rectal swabs could be a viable alternative to currently used methods.

16S rRNA gene T-RFLP fingerprinting analysis was used to reveal significant differences in the bacteria community profiles of samples collected via rectal swabs versus mucosal biopsies. Similarly, bacterial diversity indexes showed significant differences between the two sampling sites. Swab samples had higher bacterial abundance and diversity compared to rectal mucosal biopsies. Durban et al. compared bacterial community composition of stool samples and rectal mucosal biopsies obtained from an un-prepped population of healthy participants. Durban et al. Assessing gut microbial diversity from feces and rectal mucosa. Microbial ecology 2011; 61:123-33. They reported that fecal and mucosal bacterial diversity from the same subject are different. In a study that compared healthy subjects to IBS subjects, Carroll et al. observed reduced bacterial abundance and diversity in mucosal samples compared to stool samples from the same subjects. Carroll 2010. These findings are compatible with the reports of Carroll et al. and Durban et al. although we extended those findings to rectal swabs compared to biopsies. Similar to these and previous studies, our results suggest that different niches within the large intestine possess distinct bacterial populations. Hong et al. Pyrosequencing-based analysis of the mucosal microbiota in healthy individuals reveals ubiquitous bacterial groups and micro-heterogeneity. PloS one 2011; 6:e25042.

It is believed that this is the first study to compare gut microbial composition of samples collected via rectal swabs versus rectal biopsies. Additionally, investigating noninvasive alternatives for stratification of risk for colorectal cancer has the potential to increase screening rate and screening compliance among the population at risk since some participants may prefer to utilize easier and more convenient screening methods. DeBourcy et al. Community-based preferences for stool cards versus colonoscopy in colorectal cancer screening. Journal of general internal medicine 2008; 23:169-74; Wolf et al. Patient preferences and adherence to colorectal cancer screening in an urban population. American journal of public health 2006; 96:809-11.

T-RFLP analysis showed statistically significant differences in the bacterial profiles from rectal swabs and mucosal biopsies. These results suggest that a quick and inexpensive fingerprinting technique could be efficiently used to compare bacterial community profiles before investing additional costs and time with more advanced sequencing technologies.

The samples were obtained from un-prepped participants, which may be a problem because it could increase the chances of contamination of rectal swabs with luminal content. Since previous studies have observed that the luminal cavity and the colonic mucosa contain distinct bacterial communities, use of un-prepped participants for sampling may have mixed those two bacterial communities. Durban 2011; Eckburg et al. Diversity of the human intestinal microbial flora. Science 2005; 308:1635-8; Lepage et al. Biodiversity of the mucosa-associated microbiota is stable along the distal digestive tract in healthy individuals and patients with IBD. Inflammatory bowel diseases 2005; 11:473-80; Zoetendal et al. Mucosa-associated bacteria in the human gastrointestinal tract are uniformly distributed along the colon and differ from the community recovered from feces. Applied and environmental microbiology 2002; 68:3401-7. Another source of swab contamination may have been from local skin flora due to inadvertent swab contact with adjacent skin prior to insertion through the anus. Finally, future studies may include a larger study population that samples several sites such as luminal, rectal swabs and biopsies in order to get a better picture of the microbial populations in the large intestine. Moreover, the use of a sleeve to introduce the swab may reduce the contamination by local flora. Alternatively, computational or analytical methods may be used to remove the bacterial species/signatures from either luminal or local skin associated species.

In summary, the data suggests that the bacterial diversity in samples collected via rectal swabs and mucosal biopsies are different. While differences in bacterial community composition can be attributed to a whole array of factors, including host genetics and the environment, our sampling scheme enabled us to observe the diversity associated with two different sampling locations. Our results suggest potential differences in the niches within the human large intestine in relation to bacterial communities. Moreover, the differences in bacterial community composition observed may suggest that both, swab sampling and biopsy collection, are needed in order to get the full spectrum of the microbial community composition of the gut. Characterizing these unique bacterial communities of the large intestine is a first step toward understanding the complex association between bacterial diversity in the gut and intestine and disease development.

Methods

Study Population and Sampling:

Study population included 11 participants enrolled as part of an ongoing studies at UNC Hospitals. Eligibility criteria included: good general health, age 40-80 years, willingness to follow the study protocol and provision of informed consent. As part of the study protocol, two swab samples were collected for each participant prior to sigmoidoscopy. Swab specimens were collected by inserting a sterile cotton-tipped swab 1-2 cm beyond the anus and rotating for several seconds. Swabs were then placed into sterile phosphate buffered saline (PBS), vortexed for at least 2 minutes to ensure release of bacteria and stored at −80° C. until further processing. Rectal mucosal biopsies were obtained through a rigid disposable sigmoidoscope (Welch Allyn KleenSpec Disposable Sigmoidoscope with Obturator) coated with gel and inserted to approximately 10 cm with the participant in the left lateral position. Disposable flexible biopsy forceps (Olympus EndoJaw Alligator Jaw-Step, Shinjuku, Tokyo, Japan) were used to obtain single mucosal pinches from two separate sites. Biopsy samples were rinsed in sterile PBS as previously described above, snap-frozen, and then stored at −80° C. until further processing. All samples for this study were collected prior to initiating treatment for all participants. Swab samples for two participants were excluded from qPCR analysis because of insufficient DNA. The study was approved by the Institutional Review Board (IRB) at the University of North Carolina School of Medicine.

DNA Extractions and Terminal Restriction Fragments Length Polymorphisms (T-RFLPs):

T-RFLP is a fingerprinting method to assess bacterial composition in gut samples. Samples were treated with lysozyme followed by bead beating on a bullet blender homogenizer (Next Advance, Inc. Averill Park, N.Y.), using a modified protocol. Savage 1977. DNA extraction was performed using Qiagen's DNeasy Blood & Tissue kit (Cat #69504, Maryland, USA). T-RFLP profiles were collected on both biopsy and swab samples following a previously described protocol described by Shen et al. 2010. Swab samples for two participants were excluded from qPCR analysis because of insufficient DNA.

Quantitative PCR (qPCR) to Assess Specific Bacteria Known to be Present in the Human Gut:

Bacterial genera common in the human gut as described by previous studies were quantified using primers for PCR amplification of the 16S ribosomal RNA (rRNA) gene for specific bacteria groups. Carroll 2010. Quantified bacterial groups included: Clostridium spp., Bifidobacteria spp., Bacteroides spp., and Lactobacillus spp. and E. coli. Additionally, universal 16S rRNA primers were used to capture all bacterial diversity for each sample henceforth referred as Eubacteria. Modifications to the original protocol by Carroll et al.⁴ included: the use of Fast SYBR Green Master Mix (Applied Biosystems, P/N: 4385614, California, USA) and dilution of template DNA to a 1:10 (Clostridium, Bifidobacteria, Lactobacillus and Eubacteria) and 1:100 (Bifidobacteria and E. coli). Finally, the copy number for group-specific bacterial 16S ribosomal RNA gene was calculated based on a standard curve, which was adjusted to a starting DNA concentration of 50 ng/μL using the following formula to the unadjusted values:

$$[ \frac{50 ng}{A / B} ] \times Unadjusted Copy \#$$

A is the concentration of the template DNA and B is the dilution factor; either 1:10 or 1:100. Swab samples for two participants were excluded from qPCR analysis because of insufficient DNA leaving 9 swab samples for analysis.

Data Analysis:

T-RFLP profiles from swabs and biopsies were compared to determine bacterial community composition and diversity. The T-RF (phylotype) peaks size and area were determined by GeneMapper (Applied Biosystems Inc.). Peak area and fluorescence data were normalized and processed as described by Abdo et al. Abdo Z, Schuette U M, Bent S J, Williams C J, Forney U, Joyce P. Statistical methods for characterizing diversity of microbial communities by analysis of terminal restriction fragment length polymorphisms of 16S rRNA genes. Environmental microbiology 2006; 8:929-38. The contribution of individual T-RFs was calculated as a proportion of the total T-RF peak area for each sample. For this analysis, these proportions were used rather than absolute numbers. The data matrix was used to generate Bray-Curtis similarities and hierarchical clustering to observe grouping of samples based on TRF abundance. The similarities between groups (rectal swab/biopsy) were compared by analysis of similarities (ANOSIM), a non-parametric test, where the significance is computed by permutation of group membership with 999 replicates. The test statistic R, which measures the strength of the correlations ranges from −1 to 1. An R value of 1 signifies differences between groups while an R value of 0 signifies that the groups are identical.

To determine the specific phylotypes that contributed to the differences in bacterial composition between swabs and biopsies similarity percentage (SIMPER) was used to compute the proportions of phylotypes for each group. Differences in bacterial richness (measure of the number of phylotypes) evenness (measure of how evenly the individuals are distributed among different phylotypes) and Shannon diversity index (measure of diversity) as well as mean bacterial 16S gene copy number between rectal swabs and biopsies were evaluated by t-test. The data analysis protocol has been previously described Shen et al. 2010 and was performed with the Primer 6 statistical package (PRIMER E, Plymouth, United Kingdom).

TABLE 15
Characteristic of Study Population (N = 11) Rectal
mucosal biopsies and rectal swabs were collected for all
participants. Swab samples for two participants were excluded
from qPCR analysis because of insufficient DNA.

| Characteristic | Mean (se)* or percent |
|---|---|
| Age, yrs. | 56.3 (5.1) |
| Adenomas (%) | 54.5 |
| Sex - Male (%) | 45.5 |
| Body mass index (BMI) | 30.5 (6.4) |
| Waist-hip-ratio (WHR) | 0.97 (0.04) |
| Race - White (%) | 81.8 |

*se-standard error

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

What is claimed is:

1. A method for detecting colorectal adenoma in a patient which comprises:
(a) obtaining a suitable patient sample;
(b) measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(c) comparing the patient sample levels with levels associated with a control sample, wherein elevated levels are indicative of whether or not colorectal adenoma is present or absent in the patient.

2. The method of claim 1, wherein the bacteria are selected from the group consisting of Acidovorax, Acinetobacter, Aquabacterium, Azonexus, Cloacibacterium, Dechloromonas, Delftia, Fusobacterium, Helicobacter, Lactobacillus, Lactococcus, Leuconostoc, Sphingobium, Stenotrophomonas, Succinivibrio, Turicibacter, and Weissella.

3. The method of claim 1, further comprising measuring levels of Bacteroides, Bifidobacteriaceae, Dorea, or Streptococcus, wherein decreased levels of Bacteroides, Bifidobacteriaceae, Dorea, or Streptococcus, are indicative of whether or not adenoma is present or absent in the patient.

4. The method of claim 1, wherein the bacteria levels are measured using bacterial nucleic acids.

5. The method of claim 4, wherein the bacterial nucleic acids are 16S rRNA genes.

6. The method of claim 4, wherein the bacterial nucleic acids are measured using terminal restriction fragment length polymorphism (T-RFLP).

7. The method of claim 4, wherein the bacterial nucleic acids are measured by fluorescence in-situ hybridization (FISH).

8. The method of claim 4, wherein the bacterial nucleic acids are measured by polymerase chain reaction (PCR).

9. The method of claim 4, wherein the bacterial nucleic acids are measured by pyrosequencing.

10. The method of claim 4, wherein the bacterial nucleic acids are measured by a microarray.

11. The method of claim 1, wherein the bacteria in the patient sample are cultured prior to measuring the levels.

12. The method of claim 1, wherein the bacteria levels are measured using antibodies.

13. The method of claim 1, wherein the patient sample is a fecal sample.

14. The method of claim 1, wherein the patient sample is a biopsy sample.

15. The method of claim 14, wherein the biopsy sample is a mucosal biopsy sample.

16. The method of claim 1, wherein the patient sample is a sample obtained by a rectal swab.

17. The method of claim 1, wherein the colorectal adenoma is an adenocarcinoma.

18. A method for determining whether or not a patient should have a colonoscopy which comprises:
(a) obtaining a suitable patient sample;
(b) measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(c) comparing the patient sample levels with levels associated with a control sample, wherein elevated levels are indicative of whether or not the patient should have a colonoscopy.

19. A method for monitoring a patient for colorectal adenoma recurrence which comprises:
(a) obtaining a suitable patient sample;
(b) measuring a level of five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Camobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(c) comparing the patient sample levels with levels associated with appropriate controls, wherein elevated levels are indicative of adenoma recurrence in the patient.

20. A method for monitoring the progress of a treatment protocol for a patient which comprises:
(a) obtaining a suitable patient sample;
(b) measuring a level of five or more bacteria selected from group consisting of Acidovorax, Acinetobacter, Agrobacterium, Akkermansia, Alistipes, Allobaculum, Aquabacterium, Azonexus, Bacillaceae_{—}1, Bryantella, Camobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(c) comparing the patient sample levels with levels associated with appropriate controls, wherein modulated levels are indicative of the progress of the treatment for the patient.

21. A kit for detecting colorectal adenoma in a patient sample which comprises:
(a) a means for measuring a level of five more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(b) instructions for comparing the patient sample levels with levels associated with healthy patient controls, wherein elevated levels are indicative of whether or not colorectal adenoma is present or absent in the patient.

22. A kit comprising:
(a) a reagent selected from a group consisting of:
(i) nucleic acid probes capable of specifically hybridizing with nucleic acids from five or more bacteria selected from a group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella;
(ii) a pair of nucleic acid primers capable of PCR amplification of five or more said bacteria; and
(iii) four or more antibodies specific for said bacteria; and
(b) instructions for use in measuring levels in a tissue sample from a patient suspected of having colorectal adenoma.

23. A method of identifying a compound that prevents or treats colorectal adenomas, the method comprising the steps of:
(a) contacting a tissue or an animal model with a compound;
(b) measuring a level of four or more bacteria selected from group consisting of Acidovorax, Acinetobacter, Agrobacterium, Akkermansia, Alistipes, Allobaculum, Aquabacterium, Azonexus, Bacillaceae_{—}1, Bryantella, Carnobacteriaceae_{—}1, Chryseobacterium, Chryseomonas, Cloacibacterium, Comamonas, Dechloromonas, Delftia, Enterobacter, Erwinia, Exiguobacterium, Flavimonas, Fusobacterium, Gp1, Gp2, Helicobacter, Lactobacillus, Lactococcus, Leuconostoc, Methylobacterium, Micrococcineae, Novosphingobium, Pantoea, Pseudomonas, Pseudoxanthomonas, Roseburia, Rubrobacterineae, Serratia, Shinella, Sphingobium, Staphylococcus, Stenotrophomonas, Succinivibrio, Sutterella, Syntrophococcus, Turicibacter, Variovorax, and Weissella; and
(c) determining a functional effect of the compound on the bacteria levels, thereby identifying a compound that prevents or treats colorectal adenomas.
